SEQUENCE LISTING (211 sequences)

SEQ ID NO: 1 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 2 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 3 - protein, 4 aa, synthetic construct
  REGION 1..4: note = Description of Artificial Sequence: Synthetic peptide
TGGG

SEQ ID NO: 4 - protein, 11 aa, synthetic construct
  REGION 1..11: note = Description of Artificial Sequence: Synthetic peptide
TGGGGSGGGGS

SEQ ID NO: 5 - protein, 16 aa, synthetic construct
  REGION 1..16: note = Description of Artificial Sequence: Synthetic peptide
TGGGGSGGGGSGGGGS

SEQ ID NO: 6 - protein, 21 aa, synthetic construct
  REGION 1..21: note = Description of Artificial Sequence: Synthetic peptide
TGGGGSGGGGSGGGGSGGGGS

SEQ ID NO: 7 - protein, 10 aa, synthetic construct
  REGION 1..10: note = Description of Artificial Sequence: Synthetic peptide
TGGGPKSCDK

SEQ ID NO: 8 - DNA, 1248 bp, synthetic construct
  misc_feature 1..1248: note = Description of Artificial Sequence: Synthetic polynucleotide
  source 1..1248: mol_type = other DNA
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 9 - protein, 415 aa, synthetic construct
  REGION 1..415: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 1284 bp, synthetic construct
  misc_feature 1..1284: note = Description of Artificial Sequence: Synthetic polynucleotide
  source 1..1284: mol_type = other DNA
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 11 - protein, 427 aa, synthetic construct
  REGION 1..427: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 1299 bp, synthetic construct
  misc_feature 1..1299: note = Description of Artificial Sequence: Synthetic polynucleotide
  source 1..1299: mol_type = other DNA
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggaggttctggaggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 13 - protein, 432 aa, synthetic construct
  REGION 1..432: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 1269 bp, synthetic construct
  misc_feature 1..1269: note = Description of Artificial Sequence: Synthetic polynucleotide
  source 1..1269: mol_type = other DNA
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 15 - protein, 422 aa, synthetic construct
  REGION 1..422: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 1266 bp, synthetic construct
  misc_feature 1..1266: note = Description of Artificial Sequence: Synthetic polynucleotide
  source 1..1266: mol_type = other DNA
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggacccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 17 - protein, 421 aa, synthetic construct
  REGION 1..421: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 162 aa, Homo sapiens
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 19 - protein, 5 aa, synthetic construct
  REGION 1..5: note = Description of Artificial Sequence: Synthetic peptide
GGGGS

SEQ ID NO: 20 - protein, 162 aa, Homo sapiens; identical to SEQ ID NO: 18

SEQ ID NO: 21 - protein, 10 aa, synthetic construct
  REGION 1..10: note = Description of Artificial Sequence: Synthetic peptide
  REGION 1..10: note = See specification as filed for detailed description of substitutions and preferred embodiments
GGGGSGGGGS

SEQ ID NO: 22 - protein, 22 aa, unidentified
  REGION 1..22: note = Description of Unknown: Leader sequence
MGRGLLRGLWPLHIVLWTRIAS

SEQ ID NO: 23 - protein, 22 aa, unidentified
  REGION 1..22: note = Description of Unknown: Tissue plasminogen activator peptide
MDAMKRGLCCVLLLCGAVFVSP

SEQ ID NO: 24 - protein, 21 aa, Apis sp.
MKFLVNVALVFMVVYISYIYA

SEQ ID NO: 25 - protein, 26 aa, synthetic construct
  REGION 1..26: note = Description of Artificial Sequence: Synthetic peptide
TGGGGSGGGGSGGGGSGGGGSGGGGS

SEQ ID NO: 26 - protein, 31 aa, synthetic construct
  REGION 1..31: note = Description of Artificial Sequence: Synthetic polypeptide
TGGGGSGGGGSGGGGSGGGGSGGGGSGGGGS

SEQ ID NO: 27 - protein, 137 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 28 - protein, 131 aa, Homo sapiens
QKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 29 - protein, 125 aa, Homo sapiens
DMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 30 - protein, 131 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 31 - protein, 125 aa, Homo sapiens
QKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 32 - protein, 119 aa, Homo sapiens
DMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 33 - protein, 156 aa, Homo sapiens
QKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 34 - protein, 156 aa, Homo sapiens
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 35 - protein, 150 aa, Homo sapiens
QKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 36 - protein, 137 aa, synthetic construct
  REGION 1..137: note = Description of Artificial Sequence: Synthetic polypeptide
TIPPHVQKSVNNDMIVTDNNGAVKFPQLCKFCDVRFSTCDNQKSCMSNCSITSICEKPQEVCVAVWRKNDENITLETVCHDPKLPYHKFILEDAASPKCIMKEKKKPGETFFMCSCSSDECNDNIIFSEEYNTSNPD

SEQ ID NO: 37 - protein, 162 aa, synthetic construct
  REGION 1..162: note = Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHKFILEDAASPKCIMKEKKKPGETFFMCSCSSDECNDNIIFSEEYNTSNPD

SEQ ID NO: 38 - protein, 131 aa, synthetic construct
  REGION 1..131: note = Description of Artificial Sequence: Synthetic polypeptide
TIPPHVQKSVNNDMIVTDNNGAVKFPQLCKFCDVRFSTCDNQKSCMSDCSITSICEKPQEVCVAVWRKNDENITLETVCHDPKLPYHDFILEDAASPKCIMKEKKKPGETFFMCSCSSDECNDNIIFSEEY

SEQ ID NO: 39 - protein, 156 aa, synthetic construct
  REGION 1..156: note = Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSDCSITSICEKPQEVCVAVWRKNDENITLETVCHDPKLPYHDFILEDAASPKCIMKEKKKPGETFFMCSCSSDECNDNIIFSEEY

SEQ ID NO: 40 - protein, 36 aa, synthetic construct
  REGION 1..36: note = Description of Artificial Sequence: Synthetic polypeptide
TGGGGSGGGGSGGGGSGGGGSGGGGSGGGGSGGGGS

SEQ ID NO: 41 - protein, 36 aa, Homo sapiens
GRCKIRHIGSNNRLQRSTCQNTGWESAHVMKTPGFR

SEQ ID NO: 42 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 43 - protein, 233 aa, Homo sapiens
GGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 437 aa, synthetic construct
  REGION 1..437: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 442 aa, synthetic construct
  REGION 1..442: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - DNA, 1314 bp, synthetic construct
  misc_feature 1..1314: note = Description of Artificial Sequence: Synthetic polynucleotide
  source 1..1314: mol_type = other DNA
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggaggaggttctggtggtggaggttctggaggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 47 - DNA, 1329 bp, synthetic construct
  misc_feature 1..1329: note = Description of Artificial Sequence: Synthetic polynucleotide
  source 1..1329: mol_type = other DNA
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggaggtggaagtggtggaggaggttctggtggtggaggttctggaggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 48 - skipped

SEQ ID NO: 49 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 512 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 51 - protein, 115 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 52 - protein, 100 aa, synthetic construct
  REGION 1..100: note = Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEA

SEQ ID NO: 53 - protein, 512 aa, synthetic construct
  REGION 1..512: note = Description of Artificial Sequence: Synthetic polypeptide
MTAPWVALALLWGSLCAGSGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 54 - protein, 115 aa, synthetic construct
  REGION 1..115: note = Description of Artificial Sequence: Synthetic polypeptide
GRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPT

SEQ ID NO: 55 - protein, 100 aa, synthetic construct
  REGION 1..100: note = Description of Artificial Sequence: Synthetic polypeptide
GRGEAETRECIYYNANWELERTNQSGLERCEGEQDKRLHCYASWANSSGTIELVKKGCWLDDFNCYDRQECVATEENPQVYFCCCEGNFCNERFTHLPEA

SEQ ID NO: 56 - DNA, 1536 bp, Homo sapiens
  source 1..1536: mol_type = other DNA
atgacggcgccctgggtggccctcgccctcctctggggatcgctgtgcgccggctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccaccctgctcacggtgctggcctactcactgctgcccatcgggggcctttccctcatcgtcctgctggccttttggatgtaccggcatcgcaagcccccctacggtcatgtggacatccatgaggaccctgggcctccaccaccatcccctctggtgggcctgaagccactgcagctgctggagatcaaggctcgggggcgctttggctgtgtctggaaggcccagctcatgaatgactttgtagctgtcaagatcttcccactccaggacaagcagtcgtggcagagtgaacgggagatcttcagcacacctggcatgaagcacgagaacctgctacagttcattgctgccgagaagcgaggctccaacctcgaagtagagctgtggctcatcacggccttccatgacaagggctccctcacggattacctcaaggggaacatcatcacatggaacgaactgtgtcatgtagcagagacgatgtcacgaggcctctcatacctgcatgaggatgtgccctggtgccgtggcgagggccacaagccgtctattgcccacagggactttaaaagtaagaatgtattgctgaagagcgacctcacagccgtgctggctgactttggcttggctgttcgatttgagccagggaaacctccaggggacacccacggacaggtaggcacgagacggtacatggctcctgaggtgctcgagggagccatcaacttccagagagatgccttcctgcgcattgacatgtatgccatggggttggtgctgtgggagcttgtgtctcgctgcaaggctgcagacggacccgtggatgagtacatgctgccctttgaggaagagattggccagcacccttcgttggaggagctgcaggaggtggtggtgcacaagaagatgaggcccaccattaaagatcactggttgaaacacccgggcctggcccagctttgtgtgaccatcgaggagtgctgggaccatgatgcagaggctcgcttgtccgcgggctgtgtggaggagcgggtgtccctgattcggaggtcggtcaacggcactacctcggactgtctcgtttccctggtgacctctgtcaccaatgtggacctgccccctaaagagtcaagcatc

SEQ ID NO: 57 - DNA, 345 bp, Homo sapiens
  source 1..345: mol_type = unassigned DNA
gggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccacc

SEQ ID NO: 58 - protein, 225 aa, Homo sapiens; identical to SEQ ID NO: 49

SEQ ID NO: 59 - protein, 223 aa, Homo sapiens; identical to SEQ ID NO: 42

SEQ ID NO: 60 - protein, 232 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 61 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 62 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - skipped

SEQ ID NO: 64 - protein, 4 aa, synthetic construct
  REGION 1..4: note = Description of Artificial Sequence: Synthetic peptide
GGGG

SEQ ID NO: 65 - protein, 5 aa, synthetic construct
  REGION 1..5: note = Description of Artificial Sequence: Synthetic peptide
TGGGG

SEQ ID NO: 66 - protein, 5 aa, synthetic construct
  REGION 1..5: note = Description of Artificial Sequence: Synthetic peptide
SGGGG

SEQ ID NO: 67 - protein, 4 aa, synthetic construct
  REGION 1..4: note = Description of Artificial Sequence: Synthetic peptide
SGGG

SEQ ID NO: 68 - protein, 225 aa, synthetic construct
  REGION 1..225: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 69 - protein, 225 aa, synthetic construct
  REGION 1..225: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 70 - protein, 225 aa, synthetic construct
  REGION 1..225: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 225 aa, synthetic construct
  REGION 1..225: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 72 - protein, 225 aa, synthetic construct
  REGION 1..225: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 225 aa, synthetic construct
  REGION 1..225: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 74 - protein, 228 aa, synthetic construct
  REGION 1..228: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 75 - protein, 228 aa, synthetic construct
  REGION 1..228: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPPSEELALNELVTLTCLVKGFYPSDIAVEWESNGQELPREKYLTWAPVLDSDGSFFLYSILRVAAEDWKKGDTFSCSVMHEALHNHYTQKSLDRSPGK

SEQ ID NO: 76 - protein, 261 aa, synthetic construct
  REGION 1..261: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSAQLEKELQALEKENAQLEWELQALEKELAQGAT

SEQ ID NO: 77 - protein, 261 aa, synthetic construct
  REGION 1..261: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSAQLKKKLQALKKKNAQLKWKLQALKKKLAQGAT

SEQ ID NO: 78 - protein, 225 aa, synthetic construct
  REGION 1..225: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTENQVSLWCLVKGFYPSDIAVEWESNGQPENNYKTTPPVLDSDGSFFLYSKLTVDKSRWQQGNVFSCSVMHEALHNHYTQDSLSLSPGK

SEQ ID NO: 79 - protein, 225 aa, synthetic construct
  REGION 1..225: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESRGQPENNYKTTPPVLDSRGSFFLVSKLTVDKSRWQQGNVFSCSVMHEALHNHYTQKSLSLSPGK

SEQ ID NO: 80 - protein, 225 aa, synthetic construct
  REGION 1..225: note = Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRYTQKSLSLSPGK

SEQ ID NO: 81 - skipped

SEQ ID NO: 82 - protein, 385 aa, synthetic construct
  REGION 1..385: note = Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 83 - DNA, 1158 bp, synthetic construct
  misc_feature 1..1158: note = Description of Artificial Sequence: Synthetic polynucleotide
  source 1..1158: mol_type = other DNA
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaggttctggaggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggaaggagatgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 84 - protein, 360 aa, synthetic construct
  REGION 1..360: note = Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 85 - protein, 432 aa, synthetic construct
  REGION 1..432: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 86 - DNA, 1332 bp, synthetic construct
  misc_feature 1..1332: note = Description of Artificial Sequence: Synthetic polynucleotide
  source 1..1332: mol_type = other DNA
atggatgcgatgaaacgcggcctgtgctgcgtgctgctgctgtgcggcgcggtgtttgtgagcccgggcgccaccattccgccgcatgtgcagaaaagcgatgtggaaatggaagcgcagaaagatgaaattatttgcccgagctgcaaccgcaccgcgcatccgctgcgccatattaacaacgatatgattgtgaccgataacaacggcgcggtgaaatttccgcagctgtgcaaattttgcgatgtgcgctttagcacctgcgataaccagaaaagctgcatgagcaactgcagcattaccagcatttgcgaaaaaccgcaggaagtgtgcgtggcggtgtggcgcaaaaacgatgaaaacattaccctggaaaccgtgtgccatgatccgaaactgccgtatcatgattttattctggaagatgcggcgagcccgaaatgcattatgaaagaaaaaaaaaaaccgggcgaaaccttttttatgtgcagctgcagcagcgatgaatgcaacgataacattatttttagcgaagaatataacaccagcaacccggataccggtggcggcggcagcggcggcggcggcagcggcggcggcggcagcggcggcggcggcagcacccatacctgcccgccgtgcccggcgccggaactgctgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgaagaaatgaccaaaaaccaggtgagcctgacctgcctggtgaaaggcttttatccgagcgatattgcggtggaatgggaaagcaacggccagccggaaaacaactatgataccaccccgccggtgctggatagcgatggcagcttttttctgtatagcgatctgaccgtggataaaagccgctggcagcagggcaacgtgtttagctgcagcgtgatgcatgaagcgctgcataaccattatacccagaaaagcctgagcctgagcccgggcgatgatgatgataaagcgcatcatcatcatcatcattaa

SEQ ID NO: 87 - protein, 408 aa, synthetic construct
  REGION 1..408: note = Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 88 - protein, 385 aa, synthetic construct
  REGION 1..385: note = Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - DNA, 1158 bp, synthetic construct
  misc_feature 1..1158: note = Description of Artificial Sequence: Synthetic polynucleotide
  source 1..1158: mol_type = other DNA
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaggttctggaggtggaggaagtggtggaggtggttctggaggtggtggaagtactcacacatgcccaccgtgcccagcacctgaactcctgggggggccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 90 - protein, 360 aa, synthetic construct
  REGION 1..360: note = Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 432 aa, synthetic construct
  REGION 1..432: note = Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 92 - DNA, 1299 bp, synthetic construct
  misc_feature 1..1299: note = Description of Artificial Sequence: Synthetic polynucleotide
  source 1..1299: mol_type = other DNA
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggaggttctggaggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 93 - protein, 408 aa, synthetic construct
  REGION 1..408: note = Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 94 - protein, 410 aa, synthetic construct
  REGION 1..410: note = Description of Artificial Sequence: Synthetic polypeptide
GATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 409 aa, synthetic construct
  REGION 1..409: note = Description of Artificial Sequence: Synthetic polypeptide
ATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 408 aa, synthetic construct
  REGION 1..408: note = Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 407 aa, synthetic construct
  REGION 1..407: note = Description of Artificial Sequence: Synthetic polypeptide
IPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 406 aa, synthetic construct
  REGION 1..406: note = Description of Artificial Sequence: Synthetic polypeptide
PPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 405 aa, synthetic construct
  REGION 1..405: note = Description of Artificial Sequence: Synthetic polypeptide
PHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 404 aa, synthetic construct
  REGION 1..404: note = Description of Artificial Sequence: Synthetic polypeptide
HVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 150 aa, Rattus sp.
MTAPWAALALLWGSLCAGSGRGEAETRECIYYNANWELERTNQSGLERCEGEQDKRLHCYASWPNSSGTIELVKKGCWLDDFNCYDRQECVATEENPQVYFCCCEGNFCNERFTHLPEPGGPEVTYEPPPTAPTLLTVLAYSLLPIGGLS

SEQ ID NO: 102 - protein, 150 aa, Sus scrofa
MTAPWAALALLWGSLCVGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLS

SEQ ID NO: 103 - protein, 150 aa, Mus musculus
MTAPWAALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEPGGPEVTYEPPPTAPTLLTVLAYSLLPIGGLS

SEQ ID NO: 104 - protein, 150 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLS

SEQ ID NO: 105 - protein, 150 aa, Xenopus sp.
MGASVALTFLLLLATFRAGSGHDEVETRECIYYNANWELEKTNQSGVERLVEGKKDKRLHCYASWRNNSGFIELVKKGCWLDDFNCYDRQECIAKEENPQVFFCCCEGNYCNKKFTHLPEVETFDPKPQPSASVLNILIYSLLPIVGLSM

SEQ ID NO: 106 - protein, 150 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLI

SEQ ID NO: 107 - protein, 154 aa, synthetic construct
  REGION 1..154: note = Description of Artificial Sequence: Synthetic polypeptide
  MOD_RES 8: note = Thr, Ala or absent
  MOD_RES 121: note = Pro, Ala, Val or Met
MTAPWAAXLALLWGSLCAGSGRGEAETRECIYYNANWELERTNQSGLERLCEGEQDKRLHCYASWRNSSGTIELVKKGCWLDDFNCYDRQECVATEENPQVYFCCCEGNFCNERFTHLPEXGGPEVTYEPKPPTAPTLLTVLAYSLLPIGGLSM

SEQ ID NO: 108 - protein, 150 aa, Bos taurus
MTAPWAALALLWGSLCAGSGRGEAETRECIYYNANWELERTNQSGLERCEGERDKRLHCYASWRNSSGTIELVKKGCWLDDFNCYDRQECVATEENPQVYFCCCEGNFCNERFTHLPEAGGPEVTYEPPPTAPTLLTVLAYSLLPVGGLS

SEQ ID NO: 109 - protein, 107 aa, synthetic construct
  REGION 1..107: note = Description of Artificial Sequence: Synthetic polypeptide
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPT

SEQ ID NO: 110 - protein, 317 aa, Homo sapiens
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 111 - protein, 288 aa, Homo sapiens
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 112 - protein, 344 aa, Homo sapiens
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 113 - protein, 315 aa, Homo sapiens
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 114 - protein, 12 aa, Homo sapiens
KKCRMNKKNKPR

SEQ ID NO: 115 - skipped

SEQ ID NO: 116 - protein, 65 aa, Homo sapiens
GNCWLRQAKNGRCQVLYKTELSKEECCSTGRLSTSWTEEDVNDNTLFKWMIFNGGAPNCIPCKET

SEQ ID NO: 117 - protein, 63 aa, synthetic construct
  REGION 1..63: note = Description of Artificial Sequence: Synthetic polypeptide
CWLRQAKNGRCQVLYKTELSKEECCSTGRLSTSWTEEDVNDNTLFKWMIFNGGAPNCIPCKET

SEQ ID NO: 118 - protein, 70 aa, Homo sapiens
CENVDCGPGKKCRMNKKNKPRCVCAPDCSNITWKGPVCGLDGKTYRNECALLKARCKEQPELEVQYQGRC

SEQ ID NO: 119 - protein, 133 aa, synthetic construct
  REGION 1..133: note = Description of Artificial Sequence: Synthetic polypeptide
CWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRC

SEQ ID NO: 120 - protein, 206 aa, synthetic construct
  REGION 1..206: note = Description of Artificial Sequence: Synthetic polypeptide
CWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCENVDCGPGKKCRMNKKNKPRCVCAPDCSNITWKGPVCGLDGKTYRNECALLKARCKEQPELEVQYQGRC

SEQ ID NO: 121 - protein, 145 aa, Homo sapiens
CENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKC

SEQ ID NO: 122 - protein, 208 aa, synthetic construct
  REGION 1..208: note = Description of Artificial Sequence: Synthetic polypeptide
CWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKC

SEQ ID NO: 123 - protein, 320 aa, Homo sapiens
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSIS

SEQ ID NO: 124 - protein, 291 aa, Homo sapiens
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSIS

SEQ ID NO: 125 - protein, 289 aa, synthetic construct
  REGION 1..289: note = Description of Artificial Sequence: Synthetic polypeptide
CWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSIS

SEQ ID NO: 126 - protein, 225 aa, Homo sapiens; identical to SEQ ID NO: 49

SEQ ID NO: 127 - protein, 223 aa, Homo sapiens; identical to SEQ ID NO: 42

SEQ ID NO: 128 - protein, 4 aa, synthetic construct
  REGION 1..4: note = Description of Artificial Sequence: Synthetic peptide
TGGG

SEQ ID NO: 129 - protein, 21 aa, synthetic construct
  REGION 1..21: note = Description of Artificial Sequence: Synthetic peptide
GAPGGGGGAAAAAGGGGGGAP

SEQ ID NO: 130 - protein, 39 aa, synthetic construct
  REGION 1..39: note = Description of Artificial Sequence: Synthetic polypeptide
GAPGGGGGAAAAAGGGGGGAPGGGGGAAAAAGGGGGGAP

SEQ ID NO: 131 - protein, 57 aa, synthetic construct
  REGION 1..57: note = Description of Artificial Sequence: Synthetic polypeptide
GAPGGGGGAAAAAGGGGGGAPGGGGGAAAAAGGGGGGAPGGGGGAAAAAGGGGGGAP

SEQ ID NO: 132 - protein, 9 aa, synthetic construct
  REGION 1..9: note = Description of Artificial Sequence: Synthetic peptide
ALEVLFQGP

SEQ ID NO: 133-134 - skipped (2 entries)

SEQ ID NO: 135 - protein, 544 aa, synthetic construct
  REGION 1..544: note = Description of Artificial Sequence: Synthetic polypeptide
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNTGGGVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 136 - DNA, 1641 bp, synthetic construct
  misc_feature 1..1641: note = Description of Artificial Sequence: Synthetic polynucleotide
  source 1..1641: mol_type = other DNA
atggtccgcgcgaggcaccagccgggtgggctttgcctcctgctgctgctgctctgccagttcatggaggaccgcagtgcccaggctgggaactgctggctccgtcaagcgaagaacggccgctgccaggtcctgtacaagaccgaactgagcaaggaggagtgctgcagcaccggccggctgagcacctcgtggaccgaggaggacgtgaatgacaacacactcttcaagtggatgattttcaacgggggcgcccccaactgcatcccctgtaaagaaacgtgtgagaacgtggactgtggacctgggaaaaaatgccgaatgaacaagaagaacaaaccccgctgcgtctgcgccccggattgttccaacatcacctggaagggtccagtctgcgggctggatgggaaaacctaccgcaatgaatgtgcactcctaaaggcaagatgtaaagagcagccagaactggaagtccagtaccaaggcagatgtaaaaagacttgtcgggatgttttctgtccaggcagctccacatgtgtggtggaccagaccaataatgcctactgtgtgacctgtaatcggatttgcccagagcctgcttcctctgagcaatatctctgtgggaatgatggagtcacctactccagtgcctgccacctgagaaaggctacctgcctgctgggcagatctattggattagcctatgagggaaagtgtatcaaagcaaagtcctgtgaagatatccagtgcactggtgggaaaaaatgtttatgggatttcaaggttgggagaggccggtgttccctctgtgatgagctgtgccctgacagtaagtcggatgagcctgtctgtgccagtgacaatgccacttatgccagcgagtgtgccatgaaggaagctgcctgctcctcaggtgtgctactggaagtaaagcactccggatcttgcaacaccggtggtggagtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagaattc

SEQ ID NO: 137 - protein, 515 aa, synthetic construct
  REGION 1..515: note = Description of Artificial Sequence: Synthetic polypeptide
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNTGGGVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 138 - protein, 513 aa, synthetic construct
  REGION 1..513: note = Description of Artificial Sequence: Synthetic polypeptide
CWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNTGGGVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 139 - protein, 571 aa, synthetic construct
  REGION 1..571: note = Description of Artificial Sequence: Synthetic polypeptide
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWTGGGVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 140 - DNA, 1722 bp, synthetic construct
  misc_feature 1..1722: note = Description of Artificial Sequence: Synthetic polynucleotide
  source 1..1722: mol_type = other DNA
atggtccgcgcgaggcaccagccgggtgggctttgcctcctgctgctgctgctctgccagttcatggaggaccgcagtgcccaggctgggaactgctggctccgtcaagcgaagaacggccgctgccaggtcctgtacaagaccgaactgagcaaggaggagtgctgcagcaccggccggctgagcacctcgtggaccgaggaggacgtgaatgacaacacactcttcaagtggatgattttcaacgggggtgcccccaactgcatcccctgtaaagaaacgtgtgagaacgtggactgtggacctgggaaaaaatgccgaatgaacaagaagaacaaaccccgctgcgtctgcgccccggattgttccaacatcacctggaagggtccagtctgcgggctggatgggaaaacctaccgcaatgaatgtgcactcctaaaggcaagatgtaaagagcagccagaactggaagtccagtaccaaggcagatgtaaaaagacttgtcgggatgttttctgtccaggcagctccacatgtgtggtggaccagaccaataatgcctactgtgtgacctgtaatcggatttgcccagagcctgcttcctctgagcaatatctctgtgggaatgatggagtcacctactccagtgcctgccacctgagaaaggctacctgcctgctgggcagatctattggattagcctatgagggaaagtgtatcaaagcaaagtcctgtgaagatatccagtgcactggtgggaaaaaatgtttatgggatttcaaggttgggagaggccggtgttccctctgtgatgagctgtgccctgacagtaagtcggatgagcctgtctgtgccagtgacaatgccacttatgccagcgagtgtgccatgaaggaagctgcctgctcctcaggtgtgctactggaagtaaagcactccggatcttgcaactccatttcggaagacaccgaggaagaggaggaagatgaagaccaggactacagctttcctatatcttctattctagagtggaccggtggtggagtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagaattc

SEQ ID NO: 141 - protein, 542 aa, synthetic construct
  REGION 1..542: note = Description of Artificial Sequence: Synthetic polypeptide
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWTGGGVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 142 - protein, 540 aa, synthetic construct
  REGION 1..540: note = Description of Artificial Sequence: Synthetic polypeptide
CWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWTGGGVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 143 - protein, 549 aa, synthetic construct
  REGION 1..549: note = Description of Artificial Sequence: Synthetic polypeptide
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 520 aa, synthetic construct
  REGION 1..520: note = Description of Artificial Sequence: Synthetic polypeptide
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 518 aa, synthetic construct
  REGION 1..518: note = Description of Artificial Sequence: Synthetic polypeptide
CWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 547 aa, synthetic construct
  REGION 1..547: note = Description of Artificial Sequence: Synthetic polypeptide
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISTGGGVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 147 - protein, 518 aa, synthetic construct
  REGION 1..518: note = Description of Artificial Sequence: Synthetic polypeptide
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISTGGGVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 148 - protein, 516 aa, synthetic construct
  REGION 1..516: note = Description of Artificial Sequence: Synthetic polypeptide
CWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISTGGGVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 149 - protein, 375 aa, Homo sapiens
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 150 - protein, 407 aa, Homo sapiens
MVLAAPLLLGFLLLALELRPRGEAAEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 151 - protein, 5 aa, synthetic construct
  REGION 1..5: note = Description of Artificial Sequence: Synthetic peptide
SYWMQ

SEQ ID NO: 152 - protein, 17 aa, synthetic construct
  REGION 1..17: note = Description of Artificial Sequence: Synthetic peptide
AIYPGDGDTRYTQKFKG

SEQ ID NO: 153 - protein, 13 aa, synthetic construct
  REGION 1..13: note = Description of Artificial Sequence: Synthetic peptide
ARMGGYDRYYFDY

SEQ ID NO: 154 - protein, 17 aa, synthetic construct
  REGION 1..17: note = Description of Artificial Sequence: Synthetic peptide
KSSQSLLNSANQKNYLA

SEQ ID NO: 155 - protein, 7 aa, synthetic construct
  REGION 1..7: note = Description of Artificial Sequence: Synthetic peptide
FASTRES

SEQ ID NO: 156 - protein, 9 aa, synthetic construct
  REGION 1..9: note = Description of Artificial Sequence: Synthetic peptide
QQHYNTPLT

SEQ ID NO: 157 - protein, 10 aa, synthetic construct
  REGION 1..10: note = Description of Artificial Sequence: Synthetic peptide
GYTFTSYWMQ

SEQ ID NO: 158 - protein, 9 aa, synthetic construct
  REGION 1..9: note = Description of Artificial Sequence: Synthetic peptide
AIYPGDGDT

SEQ ID NO: 159 - protein, 13 aa, synthetic construct
  REGION 1..13: note = Description of Artificial Sequence: Synthetic peptide
ARMGGYDRYYFDY

SEQ ID NO: 160 - protein, 17 aa, synthetic construct
  REGION 1..17: note = Description of Artificial Sequence: Synthetic peptide
KSSQSLLNSANQKNYLA

SEQ ID NO: 161 - protein, 7 aa, synthetic construct
  REGION 1..7: note = Description of Artificial Sequence: Synthetic peptide
FASTRES

SEQ ID NO: 162 - protein, 9 aa, synthetic construct
  REGION 1..9: note = Description of Artificial Sequence: Synthetic peptide
QQHYNTPLT

SEQ ID NO: 163 - protein, 224 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 164 - protein, 546 aa, synthetic construct
  REGION 1..546: note = Description of Artificial Sequence: Synthetic polypeptide
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 22 aa, synthetic construct
  REGION 1..22: note = Description of Artificial Sequence: Synthetic peptide
AGGGGSGGGGSGGGGSGGGGSG

SEQ ID NO: 166 - DNA, 1641 bp, synthetic construct
  misc_feature 1..1641: note = Description of Artificial Sequence: Synthetic polynucleotide
  source 1..1641: mol_type = other DNA
atggtccgcgcgaggcaccagccgggtgggctttgcctcctgctgctgctgctctgccagttcatggaggaccgcagtgcccaggctgggaactgctggctccgtcaagcgaagaacggccgctgccaggtcctgtacaagaccgaactgagcaaggaggagtgctgcagcaccggccggctgagcacctcgtggaccgaggaggacgtgaatgacaacacactcttcaagtggatgattttcaacgggggcgcccccaactgcatcccctgtaaagaaacgtgtgagaacgtggactgtggacctgggaaaaaatgccgaatgaacaagaagaacaaaccccgctgcgtctgcgccccggattgttccaacatcacctggaagggtccagtctgcgggctggatgggaaaacctaccgcaatgaatgtgcactcctaaaggcaagatgtaaagagcagccagaactggaagtccagtaccaaggcagatgtaaaaagacttgtcgggatgttttctgtccaggcagctccacatgtgtggtggaccagaccaataatgcctactgtgtgacctgtaatcggatttgcccagagcctgcttcctctgagcaatatctctgtgggaatgatggagtcacctactccagtgcctgccacctgagaaaggctacctgcctgctgggcagatctattggattagcctatgagggaaagtgtatcaaagcaaagtcctgtgaagatatccagtgcactggtgggaaaaaatgtttatgggatttcaaggttgggagaggccggtgttccctctgtgatgagctgtgccctgacagtaagtcggatgagcctgtctgtgccagtgacaatgccacttatgccagcgagtgtgccatgaaggaagctgcctgctcctcaggtgtgctactggaagtaaagcactccggatcttgcaacaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 167 - protein, 120 aa, synthetic construct
  REGION 1..120: note = Description of Artificial Sequence: Synthetic polypeptide
QVQLQQSGAELARPGASVKLSCKASGYTFTSYWMQWVKQRPGQGLEWIGAIYPGDGDTRYTQKFKGKATLTADKSSSTAYMQLSSLASEDSAVYYCARMGGYDRYYFDYWGQGTTLTVSS

SEQ ID NO: 168 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 169 - protein, 20 aa, synthetic construct
  REGION 1..20: note = Description of Artificial Sequence: Synthetic peptide
GGGSGGGGSGGGGSGGGGSG

SEQ ID NO: 170 - protein, 162 aa, Homo sapiens; identical to SEQ ID NO: 18

SEQ ID NO: 171 - protein, 512 aa, synthetic construct
  REGION 1..512: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGTGGGGSGGGGSGGGGSGGGGSTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 172 - protein, 631 aa, synthetic construct
  REGION 1..631: note = Description of Artificial Sequence: Synthetic polypeptide
QVQLQQSGAELARPGASVKLSCKASGYTFTSYWMQWVKQRPGQGLEWIGAIYPGDGDTRYTQKFKGKATLTADKSSSTAYMQLSSLASEDSAVYYCARMGGYDRYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGTGGGGSGGGGSGGGGSGGGGSTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 173 - protein, 651 aa, synthetic construct
  REGION 1..651: note = Description of Artificial Sequence: Synthetic polypeptide
MGWSCIMLFLAATATGAHSQVQLQQSGAELARPGASVKLSCKASGYTFTSYWMQWVKQRPGQGLEWIGAIYPGDGDTRYTQKFKGKATLTADKSSSTAYMQLSSLASEDSAVYYCARMGGYDRYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGTGGGGSGGGGSGGGGSGGGGSTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 174 - protein, 113 aa, synthetic construct
  REGION 1..113: note = Description of Artificial Sequence: Synthetic polypeptide
DIVMTQSPSSLAMSVGQKVTMSCKSSQSLLNSANQKNYLAWYQQKPGQSPKLLVYFASTRESGVPDRFIGSGSGTDFTLTISSVQAEDLADYFCQQHYNTPLTFGAGTKLELK

SEQ ID NO: 175 - protein, 107 aa, synthetic construct [sequence omitted]

SEQ ID NO: 176 - protein, 19 aa, unidentified
  REGION 1..19: note = Description of Unknown: Leader sequence
MGWSCIMLFLAATATGAHS

SEQ ID NO: 177 - protein, 8 aa, synthetic construct
  REGION 1..8: note = Description of Artificial Sequence: Synthetic peptide
DYKDDDDK

SEQ ID NO: 178 - protein, 6 aa, synthetic construct
  REGION 1..6: note = Description of Artificial Sequence: Synthetic 6xHis tag
HHHHHH

SEQ ID NO: 179 - protein, 9 aa, synthetic construct
  REGION 1..9: note = Description of Artificial Sequence: Synthetic peptide
YPYDVPDYA

SEQ ID NO: 180 - protein, 701 aa, synthetic construct
  REGION 1..701: note = Description of Artificial Sequence: Synthetic polypeptide
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAGGGGSGGGGSGGGGSGGGGSGTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 181 - protein, 700 aa, synthetic construct
  REGION 1..700: note = Description of Artificial Sequence: Synthetic polypeptide
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 182 - protein, 220 aa, synthetic construct
  REGION 1..220: note = Description of Artificial Sequence: Synthetic polypeptide
DIVMTQSPSSLAMSVGQKVTMSCKSSQSLLNSANQKNYLAWYQQKPGQSPKLLVYFASTRESGVPDRFIGSGSGTDFTLTISSVQAEDLADYFCQQHYNTPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - protein, 551 aa, synthetic construct
  REGION 1..551: note = Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 184 - protein, 551 aa, synthetic construct
  REGION 1..551: note = Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 185 - protein, 445 aa, synthetic construct
  REGION 1..445: note = Description of Artificial Sequence: Synthetic polypeptide
MDAMKRGLCCVLLLCGAVFVSPGASNTKVDKRVTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGGGGSGGGGSGTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 186 - DNA, 1656 bp, synthetic construct
  misc_feature 1..1656: note = Description of Artificial Sequence: Synthetic polynucleotide
  source 1..1656: mol_type = other DNA
atggatgcaatgaagagagggctctgctgtgtgctgctgctgtgtggagcagtcttcgtttcgcccggcgccagcggtaggggcgaagcagaaacccgcgaatgtatatattataatgctaattgggaacttgaaaggacaaatcaatccggacttgaacgttgtgaaggggaacaagataaacgactccattgttatgcatcatggagaaattcttccggaactattgaattggtaaagaaaggatgttggttggacgattttaattgttacgaccgccaagaatgcgttgcaacagaagaaaatcctcaagtttatttctgttgttgcgaggggaacttttgtaatgaaaggtttacccatctccctgaagcaggcggacctgaggtgacatatgaaccaccacctactgctccaaccggtggcggtacccatacctgtccgccatgtcccgctcccgagctacttggcggcccctctgtattcttgtttccgcctaagccgaaagatactttgatgatttcacgaactccagaagttacctgtgtagtagtcgatgttagtcatgaagatcccgaagtaaaatttaattggtatgttgatggggtagaagttcacaacgctaaaaccaaacctcgagaagaacaatataattccacctatcgcgttgtttctgtgctgacagtgttgcatcaagattggcttaacgggaaagaatataaatgtaaagtgtctaataaggctcttcctgctccgattgaaaagactattagtaaggcaaagggtcaaccacgtgagccccaagtatatacattgccgcccagtcgagaagaaatgacgaagaatcaagtttctttgacttgtctcgtgaagggattttacccatcagatattgctgtcgaatgggaatctaacggtcaaccagaaaataattataaaacgactccacctgtcctcgatagcgatggatctttctttctgtactccaaactgactgttgataaatcccggtggcaacaaggtaatgttttcagttgtagcgttatgcacgaagcactacataatcattatacacaaaagtcactgtctctcagtcccggagcaggcggcggtggctcaggcggtggtggttcaggcggcggcgggtcaggcggtggtgggagcgggactattcccccacatgtccaaaagtcagacgttgagatggaagctcaaaaggacgagataatatgtccttcctgcaacagaaccgcacaccctctcaggcacataaacaatgatatgatcgtgacagataataatggcgctgtgaaattcccccagctctgcaagttctgcgacgttcgcttcagcacttgcgataatcaaaagtcttgtatgtctaattgttccattactagcatttgcgagaaaccccaagaggtgtgcgtcgccgtctggcggaagaacgatgaaaatattaccctcgaaacggtgtgtcacgatccgaaactgccatatcacgatttcatcttggaagacgcagcctcaccgaaatgtatcatgaaagagaagaagaaaccaggggaaaccttctttatgtgctcttgctccagcgacgaatgtaacgataatattattttcagtgaggagtacaatacttctaacccagattag

SEQ ID NO: 187 - DNA, 1656 bp, synthetic construct
  misc_feature 1..1656: note = Description of Artificial Sequence: Synthetic polynucleotide
  source 1..1656: mol_type = other DNA
atggatgcaatgaagagagggctctgctgtgtgctgctgctgtgtggagcagtcttcgtttcgcccggcgccagtggccggggtgaagccgaaactcgcgaatgtatttattataatgctaattgggaactcgaacgtacaaatcaatctgggctcgaacggtgtgagggtgaacaagataaaagactccattgttatgcttcttggagaaattctagcggaacaattgaactggttaagaagggttgttggctggacgattttaattgttatgaccgccaagaatgcgtcgcaacagaagaaaatcctcaagtttacttttgttgttgcgagggtaacttttgtaatgaaaggtttacgcacctgcctgaagcaggcgggcctgaggtgacatatgaaccgccaccaaccgctccaaccggtggcggtacccatacctgtccaccatgtcctgccccagagctgctaggtgggccaagcgtgtttctgtttccacctaagccaaaagatactctgatgatatctaggactccagaagtgacctgtgtcgttgttgatgtttctcacgaagatccagaagtgaaatttaattggtatgtcgatggagttgaagtccacaacgctaaaactaaaccaagagaggaacaatataattctacatatagggttgtgagtgtgctgacagtgttgcaccaggattggttgaacggtaaagaatataaatgtaaagtgtctaataaggctttgcccgctcctattgaaaagacgataagcaaggctaagggccaaccacgcgagcctcaagtctatacacttccaccctgtagggaagaaatgaccaagaatcaagtgtccttgtggtgtcttgttaaggggttttacccatctgatattgcagtcgaatgggaatcaaacggccaacccgaaaataattataaaactactccgccagtcttggattctgatggaagcttcttcctatactcaaaactaactgttgataaatcacgttggcaacaaggaaatgtgttttcctgttcagtcatgcacgaagccctgcataatcattatactcagaaatcattgagtttgtcaccaggagctggaggaggtggaagtggtggtggtggctctggcggcggcggctccggcggcggtgggtcaggaactataccccctcatgtgcaaaagtccgatgtcgagatggaagctcaaaaggacgagattatttgtccttcctgcaaccgcacggcacaccctctccgccacatcaacaatgatatgatcgtgaccgataataatggggccgtgaaattcccgcagctttgcaagttctgcgacgttcgtttctctacttgcgataatcaaaagtcttgtatgtcaaattgttctattacaagcatttgcgaaaagcctcaagaggtgtgcgtcgcagtgtggcgcaagaacgatgaaaatatcacgcttgaaactgtgtgtcacgatccgaaacttccatatcacgatttcatcctagaggacgcagcaagccccaaatgtatcatgaaagagaagaagaaacccggagaaaccttcttcatgtgctcatgctcttccgacgaatgtaacgataatattatatttagcgaggagtacaatacttcaaaccccgattag

SEQ ID NO: 188 - DNA, 1338 bp, synthetic construct
  misc_feature 1..1338: note = Description of Artificial Sequence: Synthetic polynucleotide
  source 1..1338: mol_type = other DNA
atggatgcaatgaagagagggctctgctgtgtgctgctgctgtgtggagcagtcttcgtttcgcccggcgccagcaacaccaaggtggacaagagagttaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtgctggtggtggaggttctggaggtggaggaagtggtggaggtggttctggaggtggtggttccggaacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgactga

SEQ ID NO: 189 - protein, 9 aa, synthetic construct
  REGION 1..9: note = Description of Artificial Sequence: Synthetic peptide
SNTKVDKRV

SEQ ID NO: 190 - protein, 343 aa, synthetic construct
  REGION 1..343: note = Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 343 aa, synthetic construct
  REGION 1..343: note = Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 192 - protein, 238 aa, synthetic construct
  REGION 1..238: note = Description of Artificial Sequence: Synthetic polypeptide
SNTKVDKRVTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 193 - protein, 445 aa, synthetic construct; identical to SEQ ID NO: 185
  REGION 1..445: note = Description of Artificial Sequence: Synthetic polypeptide

SEQ ID NO: 194 - DNA, 1338 bp, synthetic construct; identical to SEQ ID NO: 188
  misc_feature 1..1338: note = Description of Artificial Sequence: Synthetic polynucleotide
  source 1..1338: mol_type = other DNA

SEQ ID NO: 195 - protein, 526 aa, synthetic construct
  REGION 1..526: note = Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 196 - protein, 526 aa, synthetic construct
  REGION 1..526: note = Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 197 - protein, 420 aa, synthetic construct
  REGION 1..420: note = Description of Artificial Sequence: Synthetic polypeptide
NTKVDKRVTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGGGGSGGGGSGTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 198 - protein, 420 aa, synthetic construct; identical to SEQ ID NO: 197
  REGION 1..420: note = Description of Artificial Sequence: Synthetic polypeptide

SEQ ID NO: 199 - protein, 20 aa, synthetic construct
  REGION 1..20: note = Description of Artificial Sequence: Synthetic peptide
  REGION 1..20: note = MISC_FEATURE - This sequence may encompass 1-4 "Gly Gly Gly Gly Ser" repeating units
  REGION 1..20: note = See specification as filed for detailed description of substitutions and preferred embodiments
GGGGSGGGGSGGGGSGGGGS

SEQ ID NO: 200 - protein, 15 aa, synthetic construct
  REGION 1..15: note = Description of Artificial Sequence: Synthetic peptide
  REGION 1..15: note = See specification as filed for detailed description of substitutions and preferred embodiments
GGGGSGGGGSGGGGS

SEQ ID NO: 201 - protein, 21 aa, synthetic construct
  REGION 1..21: note = Description of Artificial Sequence: Synthetic peptide
GGGGSGGGGSGGGGSGGGGSG

SEQ ID NO: 202 - protein, 17 aa, synthetic construct
  REGION 1..17: note = Description of Artificial Sequence: Synthetic peptide
NTSNPDTGGGTHTCPPC

SEQ ID NO: 203 - protein, 24 aa, synthetic construct
  REGION 1..24: note = Description of Artificial Sequence: Synthetic peptide
NTSNPDTGGGGSGGGGSTHTCPPC

SEQ ID NO: 204 - protein, 29 aa, synthetic construct
  REGION 1..29: note = Description of Artificial Sequence: Synthetic peptide
NTSNPDTGGGGSGGGGSGGGGSTHTCPPC

SEQ ID NO: 205 - protein, 34 aa, synthetic construct
  REGION 1..34: note = Description of Artificial Sequence: Synthetic polypeptide
NTSNPDTGGGGSGGGGSGGGGSGGGGSTHTCPPC

SEQ ID NO: 206 - protein, 23 aa, synthetic construct
  REGION 1..23: note = Description of Artificial Sequence: Synthetic peptide
NTSNPDTGGGPKSCDKTHTCPPC

SEQ ID NO: 207 - protein, 115 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 208 - protein, 20 aa, synthetic construct
  REGION 1..20: note = Description of Artificial Sequence: Synthetic peptide
GGGGSGGGGSGGGGSGGGGS

SEQ ID NO: 209 - protein, 20 aa, synthetic construct
  REGION 1..20: note = Description of Artificial Sequence: Synthetic peptide
  REGION 1..20: note = See specification as filed for detailed description of substitutions and preferred embodiments
GGGGSGGGGSGGGGSGGGGS

SEQ ID NO: 210 - protein, 20 aa, synthetic construct
  REGION 1..20: note = Description of Artificial Sequence: Synthetic peptide
  REGION 1..20: note = MISC_FEATURE - This sequence may encompass 1-4 "Gly Gly Gly Gly Ser" repeating units
  REGION 1..20: note = See specification as filed for detailed description of substitutions and preferred embodiments
GGGGSGGGGSGGGGSGGGGS

SEQ ID NO: 211 - protein, 5 aa, synthetic construct
  REGION 1..5: note = Description of Artificial Sequence: Synthetic peptide
  REGION 1..5: note = See specification as filed for detailed description of substitutions and preferred embodiments
GGGGS

(19) United States
(12) Patent Application Publication
(10) Pub. No.: US 2025/0333477 A1
(43) Pub. Date: Oct. 30, 2025

(54) NOVEL BINDERS OF TGFB-SUPERFAMILY LIGANDS AND USES THEREOF

(71) Applicant: Acceleron Pharma Inc., Rahway, NJ (US)

(72) Inventors: Ravindra Kumar, Concord, MA (US); Dianne S. Sako, Federal Way, WA (US); Roselyne Castonguay, Watertown, MA (US); Tzu-Hsing Kuo, Chestnut Hill, MA (US)

(73) Assignee: Acceleron Pharma Inc., Rahway, NJ (US)

(21) Appl. No.: 18/924,487

(22) Filed: Oct. 23, 2024

Related U.S. Application Data
(63) Continuation of application No. 17/052,783, filed on Nov. 3, 2020, now Pat. No. 12,168,683.
(60) Provisional application No. 62/666,548, filed on May 3, 2018.
(60) Provisional application No. 62/779,992, filed on Dec. 14, 2018.

(51) Int. Cl. C07K 14/71; A61K 38/00; C07K 14/47; C07K 16/22

(52) U.S. Cl. CPC C07K 14/71; C07K 14/4703; C07K 16/22; A61K 38/00; C07K 2317/52; C07K 2319/02; C07K 2319/30

(57) ABSTRACT

In certain aspects, the disclosure provides multispecific binders (e.g., ActRIIB:TβRII heteromultimers comprising an ActRIIB polypeptide and a TβRII polypeptide). In some embodiments, the disclosure provides for novel binders comprising a TβRII polypeptide and a heterologous portion. The disclosure further provides that such multispecific binders (e.g., ActRIIB:TβRII heteromultimer) may be used to treat various disorders or conditions.

### CROSS-REFERENCE TO RELATED APPLICATIONS

This application is a continuation of U.S. patent application Ser. No. 17/052,783, filed Nov. 3, 2020, which is a national stage application of International Application No. PCT/US2019/030475, filed May 2, 2019, which claims the benefit of priority from U.S. Provisional Application No. 62/666,548, filed on May 3, 2018, and from U.S. Provisional Application No. 62/779,992, filed on Dec. 14, 2018. The foregoing applications are incorporated herein by reference in their entirety.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Oct. 18, 2024, is named 25499_US_CNT_SL.XML and is 314,433 bytes in size.

### BACKGROUND OF THE INVENTION

The transforming growth factor-beta (TGFβ) superfamily contains a variety of growth factors that share common sequence elements and structural motifs. These proteins are known to exert biological effects on a large variety of cell types in both vertebrates and invertebrates. Members of the superfamily perform important functions during embryonic development in pattern formation and tissue specification and can influence a variety of differentiation processes, including adipogenesis, myogenesis, chondrogenesis, cardiogenesis, hematopoiesis, neurogenesis, and epithelial cell differentiation. The family is divided into two general phylogenetic clades: the more recently evolved members of the superfamily, which includes TGFβs, activins, and nodal and the clade of more distantly related proteins of the superfamily, which includes a number of BMPs and GDFs [Hinck (2012) FEBS Letters 586:1860-1870]. TGFβ family members have diverse, often complementary biological effects. By manipulating the activity of a member of the TGFβ family, it is often possible to cause significant physiological changes in an organism. For example, the Piedmontese and Belgian Blue cattle breeds carry a loss-of-function mutation in the GDF8 (also called myostatin) gene that causes a marked increase in muscle mass [Grobet et al. (1997) Nat Genet 17(1):71-4]. Furthermore, in humans, inactive alleles of GDF8 are associated with increased muscle mass and, reportedly, exceptional strength [Schuelke et al. (2004) N Engl J Med 350:2682-8].

Changes in various tissues may be achieved by enhancing or inhibiting intracellular signaling (e.g., SMAD 1, 2, 3, 5, and/or 8) that is mediated by ligands of the TGFβ family. Thus, there is a need for agents that regulate the activity of various ligands of the TGFβ superfamily.

### SUMMARY OF THE INVENTION

The TGFβ superfamily is comprised of over 30 secreted factors including TGFβs, activins, nodals, bone morphogenetic proteins (BMPs), growth and differentiation factors (GDFs), and anti-Mullerian hormone (AMH) [Weiss et al. (2013) Developmental Biology, 2(1): 47-63]. The TGFβ family can be divided into two phylogenetic branches based on the type I receptors they bind and the Smad proteins they activate. One is the more recently evolved branch, which includes, e.g., the TGFβs, activins, GDF8, GDF11, GDF9, BMP3 and nodal, which signal through type I receptors that activate Smads 2 and 3 [Hinck (2012) FEBS Letters 586:1860-1870]. The other branch comprises the more distantly related proteins of the superfamily and includes, e.g., BMP2, BMP4, BMP5, BMP6, BMP7, BMP8a, BMP8b, BMP9, BMP10, GDF1, GDF5, GDF6, and GDF7, which signal through Smads 1, 5, and 8. In part, the present disclosure provides ActRIIB:TβRII heteromultimers that can antagonize a broad range of Smad 2/3 activating ligands. For example, the disclosure demonstrates that an ActRIIB:TβRII heterodimer inhibits TGFβ1, TGFβ3, activin A, activin B, GDF8, GDF11, and BMP10-signaling pathways in a cell-based assay. In contrast, ActRIIB and TβRII homodimers alone inhibit a smaller subset of Smad 2/3 activating ligands. Moreover, the data demonstrate that the ActRIIB:TβRII heterodimer is a surprisingly more selective Smad 2/3 ligand antagonists than merely combining the antagonistic profiles of ActRIIB and TβRII homodimer ligand traps. For example, the ActRIIB:TβRII heterodimer inhibited activin A, activin B, GDF8, GDF11, and BMP10-signaling pathways similarly to an ActRIIB homodimer. However, ActRIIB:TβRII heterodimer inhibition of BMP9 signaling pathways is significantly reduced compared to the ActRIIB homodimer. ActRIIB:TβRII heteromultimers therefore are more selective antagonists of Smad 2/3 activating ligands compared to ActRIIB homodimers. Accordingly, an ActRIIB:TβRII heteromultimer will be more useful than an ActRIIB or TβRII homodimer, or combination thereof, in certain applications where such broad, yet selective, Smad 2/3 antagonism is advantageous. Examples include therapeutic applications where it is desirable to antagonize one or more of TGFβ1, TGFβ3, activin (e.g., activin A, activin B, and activin AB), GDF8, and GDF11 with decreased antagonism of BMP9.

In some embodiments, the disclosure provides for a multispecific binder of TGFβ-superfamily ligands. In some embodiments, the multispecific binder protein is capable of binding to a) at least one of TGFβ1 and TGFβ3, and b) at least one of activin A, activin B, activin AB, GDF11, and GDF8. In some embodiments, the multispecific binder comprises: a) a first portion that is capable of binding to TGFβ1 and/or TGFβ3; and b) a second portion that is capable of binding to at least one of activin A, activin B, activin AB, GDF11, and GDF8. In some embodiments, the multispecific binder is a heteromultimer comprising an ActRIIB polypeptide and a TβRII polypeptide. In some embodiments, the multispecific binder comprises a TβRII polypeptide and a follistatin or a follistatin-like protein domain. In some embodiments, the multispecific binder comprises a TβRII polypeptide and an antibody or antigen-binding fragment, wherein the antibody or antigen-binding fragment is capable of binding to one or more of activin A, activin B, activin AB, GDF11, and/or GDF8. In particular embodiments, the multispecific binder comprises a TβRII polypeptide and an antibody or antigen-binding fragment, wherein the antibody or antigen-binding fragment is capable of binding to GDF8.

In some embodiments, the disclosure provides for a heteromultimer comprising an ActRIIB polypeptide and a TβRII polypeptide. In some embodiments, the ActRIIB polypeptide comprises an amino acid sequence that is at least 75% identical to: a) a sequence beginning at any one of positions 20 to 29 of SEQ ID NO: 50, and ending at any one of positions 109 to 134 of SEQ ID NO: 50; b) a sequence beginning at position 20 of SEQ ID NO: 50, and ending at position 134 of SEQ ID NO: 50; c) a sequence beginning at position 29 of SEQ ID NO: 50 and ending at position 109 of SEQ ID NO: 50; d) a sequence beginning at position 25 of SEQ ID NO: 50 and ending at position 131 of SEQ ID NO: 50; e) the sequence of SEQ ID NO: 51; f) the sequence of SEQ ID NO: 52; g) the sequence of SEQ ID NO: 54; h) the sequence of SEQ ID NO: 55; or i) the sequence of SEQ ID NO: 109. In some embodiments, the ActRIIB polypeptide comprises an amino acid sequence that is at least 90% identical to: a) a sequence beginning at any one of positions 20 to 29 of SEQ ID NO: 50, and ending at any one of positions 109 to 134 of SEQ ID NO: 50; b) a sequence beginning at position 20 of SEQ ID NO: 50, and ending at position 134 of SEQ ID NO: 50; c) a sequence beginning at position 29 of SEQ ID NO: 50 and ending at position 109 of SEQ ID NO: 50; d) a sequence beginning at position 25 of SEQ ID NO: 50 and ending at position 131 of SEQ ID NO: 50; e) the sequence of SEQ ID NO: 51; f) the sequence of SEQ ID NO: 52; g) the sequence of SEQ ID NO: 54; h) the sequence of SEQ ID NO: 55; or i) the sequence of SEQ ID NO: 109. In some embodiments, the ActRIIB polypeptide comprises an amino acid sequence that is at least 95% identical to: a) a sequence beginning at any one of positions 20 to 29 of SEQ ID NO: 50, and ending at any one of positions 109 to 134 of SEQ ID NO: 50; b) a sequence beginning at position 20 of SEQ ID NO: 50, and ending at position 134 of SEQ ID NO: 50; c) a sequence beginning at position 29 of SEQ ID NO: 50 and ending at position 109 of SEQ ID NO: 50; d) a sequence beginning at position 25 of SEQ ID NO: 50 and ending at position 131 of SEQ ID NO: 50; e) the sequence of SEQ ID NO: 51; f) the sequence of SEQ ID NO: 52; g) the sequence of SEQ ID NO: 54; h) the sequence of SEQ ID NO: 55; or i) the sequence of SEQ ID NO: 109. In some embodiments, the ActRIIB polypeptide comprises a amino acid sequence is selected from: a) a sequence beginning at any one of positions 20 to 29 of SEQ ID NO: 50, and ending at any one of positions 109 to 134 of SEQ ID NO: 50; b) a sequence beginning at position 20 of SEQ ID NO: 50, and ending at position 134 of SEQ ID NO: 50; c) a sequence beginning at position 29 of SEQ ID NO: 50 and ending at position 109 of SEQ ID NO: 50; d) a sequence beginning at position 25 of SEQ ID NO: 50 and ending at position 131 of SEQ ID NO: 50; e) the sequence of SEQ ID NO: 51; f) the sequence of SEQ ID NO: 52; g) the sequence of SEQ ID NO: 54; h) the sequence of SEQ ID NO: 55; and i) the sequence of SEQ ID NO: 109. In some embodiments, the ActRIIB polypeptide is a fusion protein comprising: a) a ActRIIB portion comprising an extracellular domain of ActRIIB; and b) a heterologous portion. In some embodiments, the ActRIIB portion comprises an amino acid sequence that is at least 75% identical to: a) a sequence beginning at any one of positions 20 to 29 of SEQ ID NO: 50, and ending at any one of positions 109 to 134 of SEQ ID NO: 50; b) a sequence beginning at position 20 of SEQ ID NO: 50, and ending at position 134 of SEQ ID NO: 50; c) a sequence beginning at position 29 of SEQ ID NO: 50 and ending at position 109 of SEQ ID NO: 50; d) a sequence beginning at position 25 of SEQ ID NO: 50 and ending at position 131 of SEQ ID NO: 50; e) the sequence of SEQ ID NO: 51; f) the sequence of SEQ ID NO: 52; g) the sequence of SEQ ID NO: 54; h) the sequence of SEQ ID NO: 55; or i) the sequence of SEQ ID NO: 109. In some embodiments, the ActRIIB portion comprises an amino acid sequence that is at least 90% identical to: a) a sequence beginning at any one of positions 20 to 29 of SEQ ID NO: 50, and ending at any one of positions 109 to 134 of SEQ ID NO: 50; b) a sequence beginning at position 20 of SEQ ID NO: 50, and ending at position 134 of SEQ ID NO: 50; c) a sequence beginning at position 29 of SEQ ID NO: 50 and ending at position 109 of SEQ ID NO: 50; d) a sequence beginning at position 25 of SEQ ID NO: 50 and ending at position 131 of SEQ ID NO: 50; e) the sequence of SEQ ID NO: 51; f) the sequence of SEQ ID NO: 52; g) the sequence of SEQ ID NO: 54; h) the sequence of SEQ ID NO: 55; or i) the sequence of SEQ ID NO: 109. In some embodiments, the ActRIIB portion comprises an amino acid sequence that is at least 95% identical to: a) a sequence beginning at any one of positions 20 to 29 of SEQ ID NO: 50, and ending at any one of positions 109 to 134 of SEQ ID NO: 50; b) a sequence beginning at position 20 of SEQ ID NO: 50, and ending at position 134 of SEQ ID NO: 50; c) a sequence beginning at position 29 of SEQ ID NO: 50 and ending at position 109 of SEQ ID NO: 50; d) a sequence beginning at position 25 of SEQ ID NO: 50 and ending at position 131 of SEQ ID NO: 50; e) the sequence of SEQ ID NO: 51; f) the sequence of SEQ ID NO: 52; g) the sequence of SEQ ID NO: 54; h) the sequence of SEQ ID NO: 55; or i) the sequence of SEQ ID NO: 109. In some embodiments, the ActRIIB portion comprises an amino acid sequence selected from: a) a sequence beginning at any one of positions 20 to 29 of SEQ ID NO: 50, and ending at any one of positions 109 to 134 of SEQ ID NO: 50; b) a sequence beginning at position 20 of SEQ ID NO: 50, and ending at position 134 of SEQ ID NO: 50; c) a sequence beginning at position 29 of SEQ ID NO: 50 and ending at position 109 of SEQ ID NO: 50; d) a sequence beginning at position 25 of SEQ ID NO: 50 and ending at position 131 of SEQ ID NO: 50; e) the sequence of SEQ ID NO: 51; f) the sequence of SEQ ID NO: 52; g) the sequence of SEQ ID NO: 54; h) the sequence of SEQ ID NO: 55; and i) the sequence of SEQ ID NO: 109. In some embodiments, the heterologous portion comprises a first or second member of an interaction pair. In some embodiments, the heterologous portion comprises one or more amino acid modifications that promotes heterodimer formation. In some embodiments, the heterologous portion is an immunoglobulin Fc domain. In some embodiments, the immunoglobulin Fc domain is a human immunoglobulin Fc domain. In some embodiments, the immunoglobulin Fc domain is an immunoglobulin G1Fc domain. In some embodiments, the immunoglobulin Fc domain comprises an amino acid sequence that is at least 75% identical to: a) the amino acid sequence of SEQ ID NO: 68, wherein the sequence comprises a lysine (K) at position 356 and a K at position 399 based on the amino acid positioning of EU numbering scheme of Kabat; b) the amino acid sequence of SEQ ID NO: 69, wherein the sequence comprises a aspartic acid (D) at position 392 and a D at position 409 based on the amino acid positioning of EU numbering scheme of Kabat; c) the amino acid sequence of SEQ ID NO: 72, wherein the sequence comprises a cysteine (C) at position 354 and a tryptophan (W) at position 366 based on the amino acid positioning of EU numbering scheme of Kabat; or d) the amino acid sequence of SEQ ID NO: 73, wherein the sequence comprises a C at position 349, a serine (S) at position 366, an alanine (A) at position 368, and a valine at position 407 based on the amino acid positioning of EU numbering scheme of Kabat. In some embodiments, the immunoglobulin Fc domain comprises an amino acid sequence that is at least 95% identical to: a) the amino acid sequence of SEQ ID NO: 68, wherein the sequence comprises a lysine (K) at position 356 and a K at position 399 based on the amino acid positioning of EU numbering scheme of Kabat; b) the amino acid sequence of SEQ ID NO: 69, wherein the sequence comprises a aspartic acid (D) at position 392 and a D at position 409 based on the amino acid positioning of EU numbering scheme of Kabat; c) the amino acid sequence of SEQ ID NO: 72, wherein the sequence comprises a cysteine (C) at position 354 and a tryptophan (W) at position 366 based on the amino acid positioning of EU numbering scheme of Kabat; or d) the amino acid sequence of SEQ ID NO: 73, wherein the sequence comprises a C at position 349, a serine (S) at position 366, an alanine (A) at position 368, and a valine at position 407 based on the amino acid positioning of EU numbering scheme of Kabat. In some embodiments, the immunoglobulin Fc domain comprises an amino acid sequence selected from: a) the amino acid sequence of SEQ ID NO: 68; b) the amino acid sequence of SEQ ID NO: 69; c) the amino acid sequence of SEQ ID NO: 72; and d) the amino acid sequence of SEQ ID NO: 73. In some embodiments, the fusion protein further comprises a linker domain portion positioned between the ActRIIB portion and the heterologous portion. In some embodiments, the linker is between 10 and 25 amino acids in length. In some embodiments, the linker comprises an amino acid sequence selected from: a) (GGGGS)ₙ, wherein n=≥2; b) (GGGGS)ₙ, wherein n=≥3; c) (GGGGS)ₙ, wherein n=≥4; and d) the amino acid sequence of any one of SEQ ID Nos: 4-7, 19, 21, 25, 26, 40, and 63-67. In some embodiments, the linker comprises (GGGGS)ₙ, wherein n≠≥5. In some embodiments, the ActRIIB fusion protein comprises an amino acid sequence that is at least 75%, 80%, 90%, 95%, or 99% identical to the amino acid sequence of SEQ ID NO: 84. In some embodiments, the ActRIIB fusion protein comprises the amino acid sequence of SEQ ID NO: 84. In some embodiments, the ActRIIB fusion protein comprises an amino acid sequence that is at least 75%, 80%, 90%, 95%, or 99% identical to the amino acid sequence of SEQ ID NO: 90. In some embodiments, the ActRIIB fusion protein comprises the amino acid sequence of SEQ ID NO: 90. In some embodiments, the ActRIIB polypeptide consists of or consists essentially of: a) an ActRIIB polypeptide portion comprising an amino acid sequence that is at least 85%, 90%, 95%, 97%, or 99% identical to the amino acid sequence of SEQ ID NO: 51 and no more than 10, 9, 8, 7, 6, 5, 4, 3, 2 or 1 additional amino acids; b) a linker portion comprising an amino acid sequence that is at least 85%, 90%, 95%, 97%, or 99% identical to the amino acid sequence of SEQ ID NO: 6 and no more than 5, 4, 3, 2 or 1 additional amino acids; c) a heterologous portion comprising an amino acid sequence that is at least 85%, 90%, 95%, 97%, or 99% identical to an amino acid sequence selected from SEQ ID NOs: 68, 69, 72, or 73 and no more than 25, 20, 15, 10, 5, 4, 3, 2, or 1 additional amino acids; and d) optionally a leader sequence (e.g., SEQ ID NO: 23). In some embodiments, the ActRIIB polypeptide consists of or consists essentially of: a) an ActRIIB polypeptide portion comprising the amino acid sequence of SEQ ID NO: 51 and no more than 10, 9, 8, 7, 6, 5, 4, 3, 2 or 1 additional amino acids; b) a linker portion comprising the amino acid sequence of SEQ ID NO: 6 and no more than 5, 4, 3, 2 or 1 additional amino acids; c) a heterologous portion comprising an amino acid sequence selected from SEQ ID NOs: 68, 69, 72, or 73 and no more than 25, 20, 15, 10, 5, 4, 3, 2, or 1 additional amino acids; and d) optionally a leader sequence (e.g., SEQ ID NO: 23). In some embodiments, the ActRIIB polypeptide comprises: a) an ActRIIB polypeptide portion comprising an amino acid sequence that is at least 85%, 90%, 95%, 97%, or 99% identical to the sequence of SEQ ID NO: 51; b) a heterologous portion, wherein the heterologous portion comprises an amino acid sequence that is at least 85%, 90%, 95%, 97%, or 99% identical to an amino acid sequence selected from SEQ ID NOs: 68, 69, 72, or 73; and c) a linker portion connecting the ActRIIB polypeptide portion and the heterologous portion; wherein the linker comprises an amino acid sequence that is at least 85%, 90%, 95%, 97%, or 99% identical to the amino acid sequence of SEQ ID NO: 6. In some embodiments, the ActRIIB polypeptide comprises: a) an ActRIIB polypeptide portion comprising the amino acid sequence of SEQ ID NO: 51; b) a heterologous portion comprising an amino acid sequence selected from SEQ ID NOs: 68, 69, 72, or 73; and c) a linker portion connecting the ActRIIB polypeptide portion and the heterologous portion; wherein the linker comprises the amino acid sequence of SEQ ID NO: 6. In some embodiments, the ActRIIB polypeptide or ActRIIB fusion protein does not comprise an acidic amino acid at the residue corresponding to position 79 of SEQ ID NO: 50. In some embodiments, the ActRIIB polypeptide or ActRIIB fusion protein does not comprise a D at the residue corresponding to position 79 of SEQ ID NO: 50. In some embodiments, the TβRII polypeptide comprises an amino acid sequence that is at least 75% identical to: a) a sequence beginning at any one of positions 23 to 35 of SEQ ID NO: 1, and ending at any one of positions 153 to 159 of SEQ ID NO: 1; b) a sequence beginning at any one of positions 23 to 60 of SEQ ID NO: 2, and ending at any one of positions 178 to 184 of SEQ ID NO: 2; c) the sequence of SEQ ID NO: 18; d) the sequence of SEQ ID NO: 27; or e) the sequence of any one of SEQ ID NOs: 28, 29, 30, 31, 32, 33, 34, 35, 36, 37, 38; and 39. In some embodiments, the TβRII polypeptide comprises an amino acid sequence that is at least 90% identical to: a) a sequence beginning at any one of positions 23 to 35 of SEQ ID NO: 1, and ending at any one of positions 153 to 159 of SEQ ID NO: 1; b) a sequence beginning at any one of positions 23 to 60 of SEQ ID NO: 2, and ending at any one of positions 178 to 184 of SEQ ID NO: 2; c) the sequence of SEQ ID NO: 18; d) the sequence of SEQ ID NO: 27; or e) the sequence of any one of SEQ ID NOs: 28, 29, 30, 31, 32, 33, 34, 35, 36, 37, 38; and 39. In some embodiments, the TβRII polypeptide comprises an amino acid sequence that is at least 95% identical to: a) a sequence beginning at any one of positions 23 to 35 of SEQ ID NO: 1, and ending at any one of positions 153 to 159 of SEQ ID NO: 1; b) a sequence beginning at any one of positions 23 to 60 of SEQ ID NO: 2, and ending at any one of positions 178 to 184 of SEQ ID NO: 2; c) the sequence of SEQ ID NO: 18; d) the sequence of SEQ ID NO: 27; or e) the sequence of any one of SEQ ID NOs: 28, 29, 30, 31, 32, 33, 34, 35, 36, 37, 38; and 39. In some embodiments, the TβRII polypeptide comprises a amino acid sequence is selected from: a) a sequence beginning at any one of positions 23 to 35 of SEQ ID NO: 1, and ending at any one of positions 153 to 159 of SEQ ID NO: 1; b) a sequence beginning at any one of positions 23 to 60 of SEQ ID NO: 2, and ending at any one of positions 178 to 184 of SEQ ID NO: 2; c) the sequence of SEQ ID NO: 18; d) the sequence of SEQ ID NO: 27; and e) the sequence of any one of SEQ ID NOs: 28, 29, 30, 31, 32, 33, 34, 35, 36, 37, 38; and 39. In some embodiments, the TβRII polypeptide comprises an amino acid sequence that is at least 85%, 90%, 95%, 97%, or 99% identical to the sequence of SEQ ID NO: 18. In some embodiments, the TβRII polypeptide comprises the amino acid sequence of SEQ ID NO: 18. In some embodiments, the TβRII polypeptide is a fusion protein comprising: a) a TβRII portion comprising an extracellular domain of TβRII; and b) a heterologous portion. In some embodiments, the TβRII portion comprises an amino acid sequence that is at least 75% identical to: a) a sequence beginning at any one of positions 23 to 35 of SEQ ID NO: 1, and ending at any one of positions 153 to 159 of SEQ ID NO: 1; b) a sequence beginning at any one of positions 23 to 60 of SEQ ID NO: 2, and ending at any one of positions 178 to 184 of SEQ ID NO: 2; c) the sequence of SEQ ID NO: 18; d) the sequence of SEQ ID NO: 27; or e) the sequence of any one of SEQ ID NOs: 28, 29, 30, 31, 32, 33, 34, 35, 36, 37, 38; and 39. In some embodiments, the TβRII portion comprises an amino acid sequence that is at least 90% identical to: a) a sequence beginning at any one of positions 23 to 35 of SEQ ID NO: 1, and ending at any one of positions 153 to 159 of SEQ ID NO: 1; b) a sequence beginning at any one of positions 23 to 60 of SEQ ID NO: 2, and ending at any one of positions 178 to 184 of SEQ ID NO: 2; c) the sequence of SEQ ID NO: 18; d) the sequence of SEQ ID NO: 27; or e) the sequence of any one of SEQ ID NOs: 28, 29, 30, 31, 32, 33, 34, 35, 36, 37, 38; and 39. In some embodiments, the TβRII portion comprises an amino acid sequence that is at least 95% identical to: a) a sequence beginning at any one of positions 23 to 35 of SEQ ID NO: 1, and ending at any one of positions 153 to 159 of SEQ ID NO: 1; b) a sequence beginning at any one of positions 23 to 60 of SEQ ID NO: 2, and ending at any one of positions 178 to 184 of SEQ ID NO: 2; c) the sequence of SEQ ID NO: 18; d) the sequence of SEQ ID NO: 27; or e) the sequence of any one of SEQ ID NOs: 28, 29, 30, 31, 32, 33, 34, 35, 36, 37, 38; and 39. In some embodiments, the TβRII portion comprises an amino acid sequence selected from: a) a sequence beginning at any one of positions 23 to 35 of SEQ ID NO: 1, and ending at any one of positions 153 to 159 of SEQ ID NO: 1; b) a sequence beginning at any one of positions 23 to 60 of SEQ ID NO: 2, and ending at any one of positions 178 to 184 of SEQ ID NO: 2; c) the sequence of SEQ ID NO: 18; d) the sequence of SEQ ID NO: 27; or e) the sequence of any one of SEQ ID NOs: 28, 29, 30, 31, 32, 33, 34, 35, 36, 37, 38; and 39. In some embodiments, the heterologous portion comprises a first or second member of an interaction pair. In some embodiments, the heterologous portion comprises one or more amino acid modifications that promotes heterodimer formation. In some embodiments, the heterologous portion is an immunoglobulin Fc domain. In some embodiments, the immunoglobulin Fc domain is a human immunoglobulin Fc domain. In some embodiments, the immunoglobulin Fc domain is an immunoglobulin G1Fc domain. In some embodiments, the immunoglobulin Fc domain comprises an amino acid sequence that is at least 75% identical to: a) the amino acid sequence of SEQ ID NO: 68, wherein the sequence comprises a lysine (K) at position 356 and a K at position 399 based on the amino acid positioning of the EU numbering scheme of Kabat; b) the amino acid sequence of SEQ ID NO: 69, wherein the sequence comprises a aspartic acid (D) at position 392 and a D at position 409 based on the amino acid positioning of the EU numbering scheme of Kabat; c) the amino acid sequence of SEQ ID NO: 72, wherein the sequence comprises a cysteine (C) at position 354 and a tryptophan (W) at position 366 based on the amino acid positioning of the EU numbering scheme of Kabat; or d) the amino acid sequence of SEQ ID NO: 73, wherein the sequence comprises a C at position 349, a serine (S) at position 366, an alanine (A) at position 368, and a valine at position 407 based on the amino acid positioning of the EU numbering scheme of Kabat. In some embodiments, the immunoglobulin Fc domain comprises an amino acid sequence that is at least 95% identical to: a) the amino acid sequence of SEQ ID NO: 68, wherein the sequence comprises a lysine (K) at position 356 and a K at position 399 based on the amino acid positioning of the EU numbering scheme of Kabat; b) the amino acid sequence of SEQ ID NO: 69, wherein the sequence comprises a aspartic acid (D) at position 392 and a D at position 409 based on the amino acid positioning of the EU numbering scheme of Kabat; c) the amino acid sequence of SEQ ID NO: 72, wherein the sequence comprises a cysteine (C) at position 354 and a tryptophan (W) at position 366 based on the amino acid positioning of the EU numbering scheme of Kabat; or d) the amino acid sequence of SEQ ID NO: 73, wherein the sequence comprises a C at position 349, a serine (S) at position 366, an alanine (A) at position 368, and a valine at position 407 based on the amino acid positioning of the EU numbering scheme of Kabat. In some embodiments, the immunoglobulin Fc domain comprises an amino acid sequence selected from: a) the amino acid sequence of SEQ ID NO: 68; b) the amino acid sequence of SEQ ID NO: 69; c) the amino acid sequence of SEQ ID NO: 72; and d) the amino acid sequence of SEQ ID NO: 73. In some embodiments, the fusion protein further comprises a linker domain portion positioned between the TβRII portion and the heterologous portion. In some embodiments, the linker is between 10 and 25 amino acids in length. In some embodiments, the linker comprises an amino acid sequence selected from: a) (GGGGS)ₙ, wherein n=≥2; b) (GGGGS)ₙ, wherein n=≥3; c) (GGGGS)ₙ, wherein n=≥4; and d) the amino acid sequence of any one of SEQ ID Nos: 4-7, 19, 21, 25, 26, 40, and 63-67. In some embodiments, the linker comprises (GGGGS)ₙ, wherein n≠≥5. In some embodiments, the TβRII fusion protein comprises an amino acid sequence that is at least 75%, 80%, 90%, 95%, or 99% identical to the amino acid sequence of SEQ ID NO: 87. In some embodiments, the TβRII fusion protein comprises the amino acid sequence of SEQ ID NO: 87. In some embodiments, the TβRII fusion protein comprises an amino acid sequence that is at least 75%, 80%, 90%, 95%, or 99% identical to the amino acid sequence of SEQ ID NO: 93. In some embodiments, the TβRII fusion protein comprises the amino acid sequence of SEQ ID NO: 93. In some embodiments, the TβRII polypeptide consists of or consists essentially of: a) an TβRII polypeptide portion comprising an amino acid sequence that is at least 85%, 90%, 95%, 97%, or 99% identical to the amino acid sequence of SEQ ID NO: 18 and no more than 10, 9, 8, 7, 6, 5, 4, 3, 2 or 1 additional amino acids; b) a linker portion comprising an amino acid sequence that is at least 85%, 90%, 95%, 97%, or 99% identical to the amino acid sequence of SEQ ID NO: 6 and no more than 5, 4, 3, 2 or 1 additional amino acids; c) a heterologous portion comprising an amino acid sequence that is at least 85%, 90%, 95%, 97%, or 99% identical to an amino acid sequence selected from SEQ ID NOs: 68, 69, 72, or 73 and no more than 25, 20, 15, 10, 5, 4, 3, 2, or 1 additional amino acids; and d) optionally a leader sequence (e.g., SEQ ID NO: 23). In some embodiments, the TβRII polypeptide consists of or consists essentially of: a) an TβRII polypeptide portion comprising the amino acid sequence of SEQ ID NO: 18 and no more than 10, 9, 8, 7, 6, 5, 4, 3, 2 or 1 additional amino acids; b) a linker portion comprising the amino acid sequence of SEQ ID NO: 6 and no more than 5, 4, 3, 2 or 1 additional amino acids; c) a heterologous portion comprising an amino acid sequence selected from SEQ ID NOs: 68, 69, 72, or 73 and no more than 25, 20, 15, 10, 5, 4, 3, 2, or 1 additional amino acids; and d) optionally a leader sequence (e.g., SEQ ID NO: 23). In some embodiments, the TβRII polypeptide comprises: a) an TβRII polypeptide portion comprising an amino acid sequence that is at least 85%, 90%, 95%, 97%, or 99% identical to the sequence of SEQ ID NO: 18; b) a heterologous portion, wherein the heterologous portion comprises an amino acid sequence that is at least 85%, 90%, 95%, 97%, or 99% identical to an amino acid sequence selected from SEQ ID NOs: 68, 69, 72, or 73; and c) a linker portion connecting the TβRII polypeptide portion and the heterologous portion; wherein the linker comprises an amino acid sequence that is at least 85%, 90%, 95%, 97%, or 99% identical to the amino acid sequence of SEQ ID NO: 6. In some embodiments, the TβRII polypeptide comprises: a) an TβRII polypeptide portion comprising the amino acid sequence of SEQ ID NO: 18; b) a heterologous portion comprising an amino acid sequence selected from SEQ ID NOs: 68, 69, 72, or 73; and c) a linker portion connecting the TβRII polypeptide portion and the heterologous portion; wherein the linker comprises the amino acid sequence of SEQ ID NO: 6. In some embodiments, the heteromultimer comprises one or more modified amino acid residues selected from: a glycosylated amino acid, a PEGylated amino acid, a farnesylated amino acid, an acetylated amino acid, a biotinylated amino acid, and an amino acid conjugated to a lipid moiety. In some embodiments, the heteromultimer is glycosylated. In some embodiments, the heteromultimer has a glycosylation pattern characteristic of expression of the polypeptide in CHO cells. In some embodiments, the heteromultimer has a glycosylation pattern characteristic of expression of the polypeptide in CHO cells. In some embodiments, the heteromultimer binds to one or more of: GDF11, GDF8, activin A, activin B, BMP10, TGFβ1, and TGFβ3. In some embodiments, the heteromultimer inhibits on or more of GDF11, GDF8, activin A, activin B, BMP10, TGFβ1, and TGFβ3 signaling as determined using a reporter gene assay. In some embodiments, the heteromultimer is a heterodimer. In some embodiments, the heteromultimer is isolated. In some embodiments, the heteromultimer is isolated.

In some embodiments, the disclosure provides for an isolated polynucleotide comprising a coding sequence for any of the ActRIIB polypeptides or fusion proteins disclosed herein. In some embodiments, the disclosure provides for an isolated polynucleotide comprising a coding sequence for any of the TβRII polypeptides or fusion proteins disclosed herein. In some embodiments, the disclosure provides for an isolated polynucleotide comprising a coding sequence for any of the ActRIIB polypeptides or fusion proteins disclosed herein and any of the TβRII polypeptides or fusion proteins disclosed herein. In some embodiments, the disclosure provides for a recombinant polynucleotide comprising a promotor sequence operably linked to any of the polynucleotides disclosed herein. In some embodiments, the disclosure provides for a cell comprising the any of the polynucleotides disclosed herein. In some embodiments, the cell is a CHO cell.

In some embodiments, the disclosure provides for a pharmaceutical preparation comprising any of the polypeptides/heteromultimers disclosed herein and a pharmaceutically acceptable excipient.

In some embodiments, the disclosure provides for a method of making a heteromultimer comprising an ActRIIB polypeptide and a TβRII polypeptide comprising culturing a cell under conditions suitable for expression of an ActRIIB polypeptide and a TβRII polypeptide, wherein the cell comprises any one or more of the polynucleotides disclosed herein.

In some embodiments, the disclosure provides for a method of making a heteromultimer comprising an ActRIIB polypeptide and a TβRII polypeptide comprising culturing a cell under conditions suitable for expression of an ActRIIB polypeptide and a TβRII polypeptide, wherein the cell comprises any of the polynucleotides disclosed herein.

In some embodiments, the disclosure provides for a method of making a heteromultimer comprising an TβRII polypeptide and an ActRIIB polypeptide comprising: a) culturing a first cell under conditions suitable for expression of an TβRII polypeptide, wherein the first cell comprises any of the recombinant polynucleotides disclosed herein; b) recovering the TβRII polypeptide so expressed; c) culturing a second cell under conditions suitable for expression of an ActRIIB polypeptide, wherein the second cell comprises any of the recombinant polynucleotides disclosed herein; d) recovering the ActRIIB polypeptide so expressed; e) combining the recovered TβRII polypeptide and the recovered ActRIIB polypeptide under conditions suitable for ActRIIB:TβRII heteromultimer formation.

In some embodiments, the disclosure provides for a method of modulating the response of a cell to a TGFβ superfamily member, the method comprising exposing the cell to any of the heteromultimers disclosed herein. In some embodiments, the disclosure provides for a method of treating a disease or condition associated with a TGFβ superfamily member in a patient in need thereof, the method comprising administering to the patient an effective amount of any of the heteromultimers disclosed herein or any of the pharmaceutical preparations disclosed herein. In some embodiments, the disclosure provides for a method of treating a muscle-related disease or condition in a patient in need thereof, the method comprising administering to the patient an effective amount of any of the heteromultimers disclosed herein or any of the pharmaceutical preparations disclosed herein. In some embodiments, the muscle-related disease or condition is selected from: muscular dystrophy, Duchene muscular dystrophy, Becker muscular dystrophy, Charcot-Marie-Tooth, facioscapulohumeral muscular dystrophy, amyotrophic lateral sclerosis, and sarcopenia. In some embodiments, the disclosure provides for a method of treating a pulmonary-related disease or condition in a patient in need thereof, the method comprising administering to the patient an effective amount of any of the heteromultimers disclosed herein or any of the pharmaceutical preparations disclosed herein. In some embodiments, the pulmonary-related disease or condition is selected from pulmonary hypertension, pulmonary arterial hypertension, and idiopathic pulmonary fibrosis. In some embodiments, the disclosure provides for a method of treating a cancer in a patient in need thereof, the method comprising administering to the patient an effective amount of any of the heteromultimers disclosed herein or any of the pharmaceutical preparations disclosed herein. In some embodiments, the disclosure provides for a method of treating a kidney-related disease or condition in a patient in need thereof, the method comprising administering to the patient an effective amount of any of the heteromultimers disclosed herein or any of the pharmaceutical preparations disclosed herein. In some embodiments, the kidney-related disease or condition is selected from: Alport syndrome, chronic kidney disease, polycystic kidney disease and renal fibrosis. In some embodiments, the disclosure provides for a method of treating a anemia or an anemia-related disease or condition in a patient in need thereof, the method comprising administering to the patient an effective amount of any of the heteromultimers disclosed herein or any of the pharmaceutical preparations disclosed herein. In some embodiments, the anemia-related disease or condition is selected from: thalassemia, myelodysplastic syndrome, myelofibrosis, and sickle cell disease.

In some embodiments, the disclosure provides for a multispecific binder protein comprising a TβRII polypeptide and a follistatin polypeptide. In some embodiments, the TβRII polypeptide comprises an amino acid sequence that is at least 80%, 85%, 90%, 91%, 92%, 93%, 94%, 95%, 96%, 97%, 98%, 99%, or 100% identical to the amino acid sequence of SEQ ID NO: 170, or a biologically active fragment thereof. In some embodiments, the follistatin polypeptide comprises an amino acid sequence that is at least 80%, 85%, 90%, 91%, 92%, 93%, 94%, 95%, 96%, 97%, 98%, 99%, or 100% identical to the amino acid sequence of SEQ ID NO: 111, or a biologically active fragment thereof. In some embodiments, the binder protein further comprises a heterologous portion. In some embodiments, the heterologous portion is an Fc domain. In some embodiments, the Fc domain comprises an amino acid sequence that is at least 80%, 85%, 90%, 91%, 92%, 93%, 94%, 95%, 96%, 97%, 98%, 99%, or 100% identical to the amino acid sequence of SEQ ID NO: 163. In some embodiments, the heterologous portion is between the follistatin polypeptide and the TβRII polypeptide. In some embodiments, the heterologous portion is conjugated to the follistatin polypeptide directly. In some embodiments, the heterologous portion is conjugated to the follistatin polypeptide by means of a linker. In some embodiments, the linker comprises the amino acid sequence of SEQ ID NO: 3. In some embodiments, the heterologous portion is conjugated to the TβRII polypeptide directly. In some embodiments, the heterologous portion is conjugated to the TβRII polypeptide by means of a linker. In some embodiments, the linker conjugating the heterologous portion to the TβRII polypeptide comprises an amino acid sequence that is at least 80%, 85%, 90%, 91%, 92%, 93%, 94%, 95%, 96%, 97%, 98%, 99%, or 100% identical to the amino acid sequence of SEQ ID NO: 165. In some embodiments, the protein comprises, from N-terminus to C-terminus: the follistatin polypeptide, the heterologous domain, and the TβRII polypeptide. In some embodiments, the protein comprises a leader sequence. In some embodiments, the leader sequence comprises an amino acid sequence that is at least 80%, 85%, 90%, 91%, 92%, 93%, 94%, 95%, 96%, 97%, 98%, 99%, or 100% identical to the amino acid sequence of SEQ ID NO: 23. In some embodiments, the binder protein comprises an amino acid sequence that is at least 80%, 85%, 90%, 91%, 92%, 93%, 94%, 95%, 96%, 97%, 98%, 99%, or 100% identical to the amino acid sequence of SEQ ID NO: 164. In some embodiments, the binder protein comprises an amino acid sequence that is at least 80%, 85%, 90%, 91%, 92%, 93%, 94%, 95%, 96%, 97%, 98%, 99%, or 100% identical to the amino acid sequence of SEQ ID NO: 180 or 181.

In some embodiments, the disclosure provides for a multispecific binder protein comprising a TβRII polypeptide and an antibody or antigen-binding fragment capable of binding to GDF8. In some embodiments, the TβRII polypeptide comprises an amino acid sequence that is at least 80%, 85%, 90%, 91%, 92%, 93%, 94%, 95%, 96%, 97%, 98%, 99%, or 100% identical to the amino acid sequence of SEQ ID NO: 170, or a biologically active fragment thereof. In some embodiments, the antibody or antigen-binding fragment comprises a variable heavy chain and a variable light chain. In some embodiments, the variable heavy chain comprises CDRs having the amino acid sequence of SEQ ID NOs: 151-153. In some embodiments, the variable light chain comprises CDRs having the amino acid sequence of SEQ ID NOs: 154-156. In some embodiments, the variable heavy chain comprises an amino acid sequence that is at least 80%, 85%, 90%, 91%, 92%, 93%, 94%, 95%, 96%, 97%, 98%, 99%, or 100% identical to the amino acid sequence of SEQ ID NO: 167. In some embodiments, the variable light chain comprises an amino acid sequence that is at least 80%, 85%, 90%, 91%, 92%, 93%, 94%, 95%, 96%, 97%, 98%, 99%, or 100% identical to the amino acid sequence of SEQ ID NO: 174. In some embodiments, the antibody or antigen-binding fragment comprises an amino acid sequence that is at least 80%, 85%, 90%, 91%, 92%, 93%, 94%, 95%, 96%, 97%, 98%, 99%, or 100% identical to the amino acid sequence of SEQ ID NO: 168, or a biologically active fragment thereof. In some embodiments, the antibody or antigen-binding fragment comprises an amino acid sequence that is at least 80%, 85%, 90%, 91%, 92%, 93%, 94%, 95%, 96%, 97%, 98%, 99%, or 100% identical to the amino acid sequence of SEQ ID NO: 167, or a biologically active fragment thereof. In some embodiments, the antibody or antigen-binding fragment comprises an amino acid sequence that is at least 80%, 85%, 90%, 91%, 92%, 93%, 94%, 95%, 96%, 97%, 98%, 99%, or 100% identical to the amino acid sequence of SEQ ID NO: 171, or a biologically active fragment thereof. In some embodiments, the protein comprises an amino acid sequence that is at least 80%, 85%, 90%, 91%, 92%, 93%, 94%, 95%, 96%, 97%, 98%, 99%, or 100% identical to the amino acid sequence of SEQ ID NO: 172. In some embodiments, the protein comprises an amino acid sequence that is at least 80%, 85%, 90%, 91%, 92%, 93%, 94%, 95%, 96%, 97%, 98%, 99%, or 100% identical to the amino acid sequence of SEQ ID NO: 175. In some embodiments, the protein comprises an amino acid sequence that is at least 80%, 85%, 90%, 91%, 92%, 93%, 94%, 95%, 96%, 97%, 98%, 99%, or 100% identical to the amino acid sequence of SEQ ID NO: 182. In some embodiments, the protein comprises an amino acid sequence that is at least 80%, 85%, 90%, 91%, 92%, 93%, 94%, 95%, 96%, 97%, 98%, 99%, or 100% identical to the amino acid sequence of SEQ ID NO: 172, and wherein the protein further comprises an amino acid sequence that is at least 80%, 85%, 90%, 91%, 92%, 93%, 94%, 95%, 96%, 97%, 98%, 99%, or 100% identical to the amino acid sequence of SEQ ID NO: 182. In some embodiments, the protein comprises a leader sequence. In some embodiments, the leader sequence comprises an amino acid sequence that is at least 80%, 85%, 90%, 91%, 92%, 93%, 94%, 95%, 96%, 97%, 98%, 99%, or 100% identical to the amino acid sequence of SEQ ID NO: 176. In some embodiments, the antibody or antigen-binding fragment is also capable of binding to GDF11 and/or activin.

In some embodiments, the disclosure provides for a polynucleotide or collection of polynucleotides capable of expressing any of the multispecific binder proteins disclosed herein. In some embodiments, the disclosure provides for a vector or collection of vectors comprising any of the polynucleotides disclosed herein. In some embodiments, the disclosure provides for a host cell comprising and capable of expressing any of the polynucleotides or vectors disclosed herein. In some embodiments, the disclosure provides for a pharmaceutical composition comprising any of the multispecific binders disclosed herein and a pharmaceutically acceptable carrier.

In some embodiments, the disclosure provides for a method of treating a subject having a muscle disorder with any of the multispecific binders disclosed herein. In some embodiments, the subject has muscular dystrophy. In some embodiments, the subject has Duchenne Muscular Dystrophy. In some embodiments, the subject has Becker Muscular Dystrophy. In some embodiments, the disorder is associated with muscle fibrosis. In some embodiments, the disorder is associated with muscle loss or muscle wasting.

In some embodiments, the disclosure provides for a fusion protein comprising an ActRIIB polypeptide and a TβRII polypeptide. In some embodiments, the ActRIIB polypeptide comprises an amino acid sequence that is at least 70%, 75%, 80%, 85%, 90%, 91%, 92%, 93%, 94%, 95%, 97%, 98%, 99% or 100% identical to the amino acid sequence of SEQ ID NO: 51 or 52. In some embodiments, the TβRII polypeptide comprises an amino acid sequence that is at least 70%, 75%, 80%, 85%, 90%, 91%, 92%, 93%, 94%, 95%, 97%, 98%, 99% or 100% identical to the amino acid sequence of SEQ ID NO: 170. In some embodiments, the ActRIIB polypeptide portion is N-terminal to the TβRII polypeptide portion. In some embodiments, the ActRIIB polypeptide portion is C-terminal to the TβRII polypeptide portion. In some embodiments, a heterologous portion and/or one or more linker portions separate the ActRIIB and TβRII polypeptide portions in the fusion protein. In some embodiments, the heterologous portion is an Fc polypeptide portion comprising an amino acid sequence that is at least 70%, 75%, 80%, 85%, 90%, 91%, 92%, 93%, 94%, 95%, 97%, 98%, 99% or 100% identical to the amino acid sequence of SEQ ID NO: 163. In some embodiments, the heterologous portion is an Fc polypeptide portion comprising an amino acid sequence that is at least 70%, 75%, 80%, 85%, 90%, 91%, 92%, 93%, 94%, 95%, 97%, 98%, 99% or 100% identical to the amino acid sequence of SEQ ID NO: 72 or 73 (which may optionally lack the C-terminal lysine residue). In some embodiments, the TβRII polypeptide portion is fused to the Fc portion by means of a linker. In some embodiments, the TβRII polypeptide portion is fused to the Fc portion by means of a glycine-serine-rich linker. In some embodiments, the linker comprises an amino acid sequence that is at least 70%, 75%, 80%, 85%, 90%, 91%, 92%, 93%, 94%, 95%, 97%, 98%, 99% or 100% identical to the amino acid sequence of SEQ ID NO: 165. In some embodiments, the ActRIIB polypeptide portion is fused to the Fc portion by means of a linker. In some embodiments, the ActRIIB polypeptide portion is fused to the Fc portion by means of a linker comprising a GGG linker. In some embodiments, the fusion protein comprises a signal sequence. In some embodiments, the signal sequence comprises an amino acid sequence that is at least 70%, 75%, 80%, 85%, 90%, 91%, 92%, 93%, 94%, 95%, 97%, 98%, 99% or 100% identical to the amino acid sequence of SEQ ID NO: 23. In some embodiments, the fusion protein comprises an amino acid sequence that is at least 70%, 75%, 80%, 85%, 90%, 91%, 92%, 93%, 94%, 95%, 97%, 98%, 99% or 100% identical to the amino acid sequence of SEQ ID NO: 183 or 195. In some embodiments, the fusion protein is a unit of a multimer. In some embodiments, the multimer is a homodimer. In some embodiments, the multimer is a heteromultimer, wherein the fusion protein is one unit of the heteromultimer, and wherein the heteromultimer comprises a second protein unit. In some embodiments, the second protein unit comprises an ActRIIB polypeptide portion but lacks a TβRII polypeptide portion. In some embodiments, the second protein unit comprises a TβRII polypeptide portion but lacks an ActRIIB polypeptide portion. In some embodiments, each unit of the heteromultimer comprises a member of an interaction pair. In some embodiments, the members of the interaction pair comprise an Fc domain. In some embodiments, the Fc domains comprise amino acid modifications that promote heteromultimer formation and/or to inhibit homomultimer formation. In some embodiments, the Fe domains have been modified to include one or more “knob-in-hole” mutations. In some embodiments, the fusion protein comprises an amino acid sequence that is at least 70%, 75%, 80%, 85%, 90%, 91%, 92%, 93%, 94%, 95%, 97%, 98%, 99% or 100% identical to the amino acid sequence of SEQ ID NO: 184 or 196. In some embodiments, the second unit of the heteromultimer comprises a TβRII polypeptide portion but lacks an ActRIIB polypeptide portion, wherein the second protein unit comprises an amino acid sequence that is at least 70%, 75%, 80%, 85%, 90%, 91%, 92%, 93%, 94%, 95%, 97%, 98%, 99% or 100% identical to the amino acid sequence of SEQ ID NO: 185 or 197. In some embodiments, the fusion protein comprises the amino acid sequence of SEQ ID NO: 184 or 196 and wherein the second protein unit comprises the amino acid sequence of SEQ ID NO: 185 or 197.

In some embodiments, the disclosure provides for a fusion protein comprising a TβRII polypeptide portion and a heterologous portion, wherein the TβRII polypeptide is C-terminal to a heterologous portion. In some embodiments, a linker connects the TβRII portion to the heterologous portion. In some embodiments, the linker comprises an amino acid sequence that is at least 70%, 75%, 80%, 85%, 90%, 91%, 92%, 93%, 94%, 95%, 97%, 98%, 99% or 100% identical to the amino acid sequence of SEQ ID NO: 165. In some embodiments, the heterologous portion is an Fc portion. In some embodiments, the Fc portion comprises an amino acid sequence that is at least 70%, 75%, 80%, 85%, 90%, 91%, 92%, 93%, 94%, 95%, 97%, 98%, 99% or 100% identical to SEQ ID NO: 73 (which may optionally lack the C-terminal lysine residue), or functional fragments thereof. In some embodiments, the TβRII portion comprises an amino acid sequence that is at least 70%, 75%, 80%, 85%, 90%, 91%, 92%, 93%, 94%, 95%, 97%, 98%, 99% or 100% identical to SEQ ID NO: 170, or functional fragments thereof. In some embodiments, the fusion protein comprises an amino acid sequence that is at least 70%, 75%, 80%, 85%, 90%, 91%, 92%, 93%, 94%, 95%, 97%, 98%, 99% or 100% identical to SEQ ID NO: 193 or 198. In some embodiments, the fusion protein is part of a homodimer. In some embodiments, the homodimer comprises two fusion proteins each comprising the amino acid sequence of SEQ ID NO: 193 or 198. In some embodiments, the fusion protein is a monomer. In some embodiments, the fusion protein comprises the amino acid sequence of SEQ ID NO: 193 or 198. In some embodiments, the fusion protein does not comprise any additional ligand-binding domains. In some embodiments, the fusion protein does not comprise an ActRIIB portion, an antibody portion, an antigen-binding portion, or a follistatin portion.

In some embodiments, the disclosure provides for an isolated polynucleotide encoding any of the fusion proteins disclosed herein.

In some embodiments, the disclosure provides for a recombinant polynucleotide comprising a promotor sequence operably linked to any of the polynucleotides disclosed herein.

In some embodiments, the disclosure provides for a cell comprising any of the polynucleotides disclosed herein. In some embodiments, the cell is a CHO cell.

In some embodiments, the disclosure provides for a pharmaceutical preparation comprising any of the fusion proteins disclosed herein and a pharmaceutically acceptable excipient.

In some embodiments, the disclosure provides for a method of modulating the response of a cell to a TGFβ superfamily member, the method comprising exposing the cell to any of the fusion proteins disclosed herein.

In some embodiments, the disclosure provides for a method of treating a disease or condition associated with a TGFβ superfamily member in a patient in need thereof, the method comprising administering to the patient an effective amount of any of the fusion proteins disclosed herein.

In some embodiments, the disclosure provides for a method of treating a muscle-related disease or condition in a patient in need thereof, the method comprising administering to the patient an effective amount of any of the fusion proteins disclosed herein. In some embodiments, the muscle-related disease or condition is selected from: muscular dystrophy, Duchene muscular dystrophy, Becker muscular dystrophy, Charcot-Marie-Tooth, facioscapulohumeral muscular dystrophy, amyotrophic lateral sclerosis, and sarcopenia.

In some embodiments, the disclosure provides for a method of treating a pulmonary-related disease or condition in a patient in need thereof, the method comprising administering to the patient an effective amount of any of the fusion proteins disclosed herein. In some embodiments, the pulmonary-related disease or condition is selected from interstitial lung disease, pulmonary hypertension, pulmonary arterial hypertension, and idiopathic pulmonary fibrosis.

In some embodiments, the disclosure provides for a method of treating a cancer in a patient in need thereof, the method comprising administering to the patient an effective amount of the fusion protein of any of the fusion proteins disclosed herein.

In some embodiments, the disclosure provides for a method of treating a kidney-related disease or condition in a patient in need thereof, the method comprising administering to the patient an effective amount of any of the fusion proteins disclosed herein. In some embodiments, the kidney-related disease or condition is selected from: Alport syndrome, chronic kidney disease, polycystic kidney disease and renal fibrosis.

In some embodiments, the disclosure provides for a method of treating an anemia or an anemia-related disease or condition in a patient in need thereof, the method comprising administering to the patient an effective amount of any of the fusion proteins disclosed herein. In some embodiments, the anemia-related disease or condition is selected from: thalassemia, myelodysplastic syndrome, myelofibrosis, and sickle cell disease.

In some embodiments, the disclosure provides for a method of treating a fibrotic or sclerotic disease or condition in a patient in need thereof, the method comprising administering to the patient an effective amount of any of the fusion proteins disclosed herein. In some embodiments, the fibrotic or sclerotic disease or condition is any one or more of systemic sclerosis, diffuse systemic sclerosis, systemic sclerosis-interstitial lung disease, myelofibrosis, progressive systemic sclerosis (PSS), or idiopathic pulmonary fibrosis.

### BRIEF DESCRIPTION OF THE DRAWINGS

FIG. 1 shows the amino acid sequence of native precursor for the B (short) isoform of human TGFβ receptor type II (hTβRII) (NP_003233.4) (SEQ ID NO: 1). Solid underline indicates the processed extracellular domain (ECD) (residues 23-159), and double underline indicates valine that is replaced in the A (long) isoform. Dotted underline denotes leader (residues 1-22).

FIG. 2 shows the amino acid sequence of native precursor for the A (long) isoform of human TβRII (NP_001020018.1) (SEQ ID NO: 2). Solid underline indicates the processed ECD (residues 23-184), and double underline indicates the splice-generated isoleucine substitution. Dotted underline denotes leader (residues 1-22).

FIG. 3 shows a comparison of the linker sequences of five different TβRII constructs.

FIGS. 4A and 4B show in tabular form the binding affinity between TGFβ1 and TGFβ3 and one of several different TβRII-Fc fusion protein constructs.

FIGS. 5A and 5C graph the results from reporter gene assays testing the affinity of TGFβ1 for one of several different TβRII-Fc fusion protein constructs. FIGS. 5B and 5D graph the results from reporter gene assays testing the affinity of the TGFβ3 for one of several different TβRII-Fc fusion protein constructs. FIGS. 5E and 5F provide IC₅₀ data from these same experiments in tabular form.

FIG. 6 shows multiple sequence alignment of Fc domains from human IgG isotypes using Clustal 2.1. Hinge regions are indicated by dotted underline. Double underline indicates examples of positions engineered in IgG1 Fc to promote asymmetric chain pairing and the corresponding positions with respect to other isotypes IgG2, IgG3 and IgG4.

FIG. 7 shows an alignment of extracellular domains of human ActRIIA and human ActRIIB with the residues that are deduced herein, based on composite analysis of multiple ActRIIB and ActRIIA crystal structures, to directly contact ligand indicated with boxes.

FIG. 8 shows a multiple sequence alignment of various vertebrate ActRIIB precursor proteins (rat (SEQ ID No: 101); pig (SEQ ID NO: 102); mouse (SEQ ID NO: 103); human (SEQ ID NO: 104); cow (SEQ ID NO: 108); and xenopus (SEQ ID NO:105)) without their intracellular domains human ActRIIA precursor protein (SEQ ID NO: 106) without its intracellular domain, and a consensus ActRII precursor protein (SEQ ID NO: 107).

FIGS. 9A-9D show schematic examples of heteromeric protein complexes comprising an TβRII polypeptide (e.g. a polypeptide that is at least 70%, 75%, 80%, 85%, 90%, 91%, 92%, 93%, 94%, 95%, 97%, 98%, 99% or 100% identical to an extracellular domain of an TβRII protein from humans or other species such as those described herein, e.g., SEQ ID Nos: 18, 27, and 28-39) and an ActRIIB polypeptide (e.g. a polypeptide that is at least 70%, 75%, 80%, 85%, 90%, 91%, 92%, 93%, 94%, 95%, 97%, 98%, 99% or 100% identical to an extracellular domain of an ActRIIB protein from humans or other species such as those described herein, e.g., SEQ ID Nos: 51, 52, 54, 55, and 109).

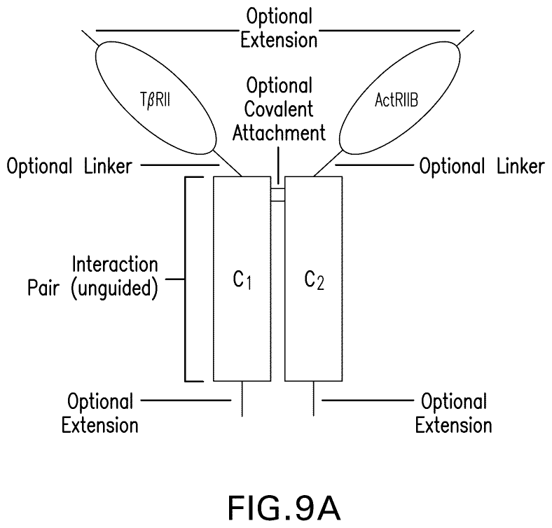

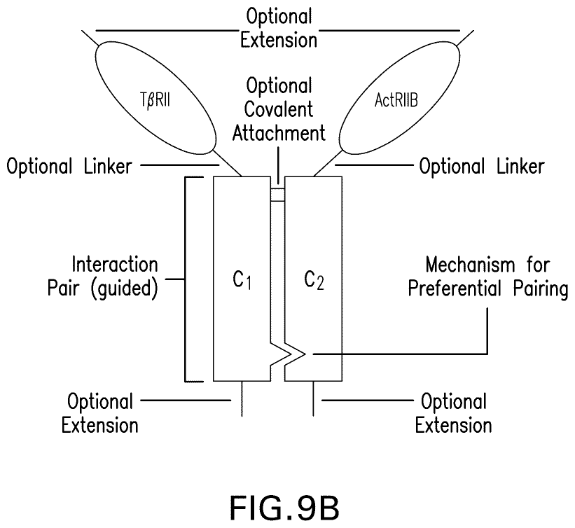

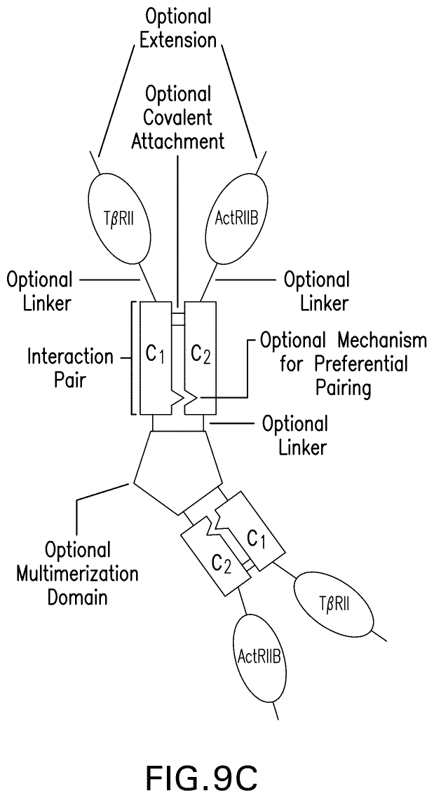

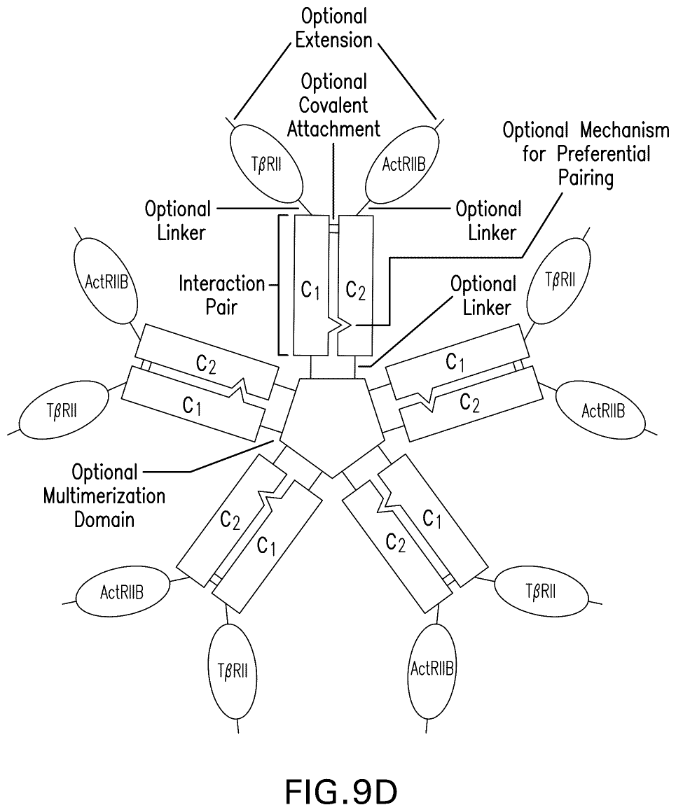

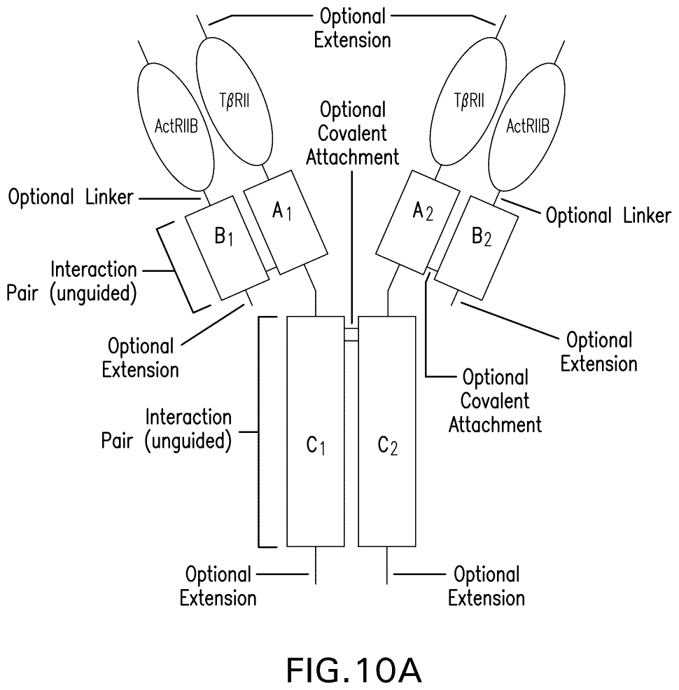

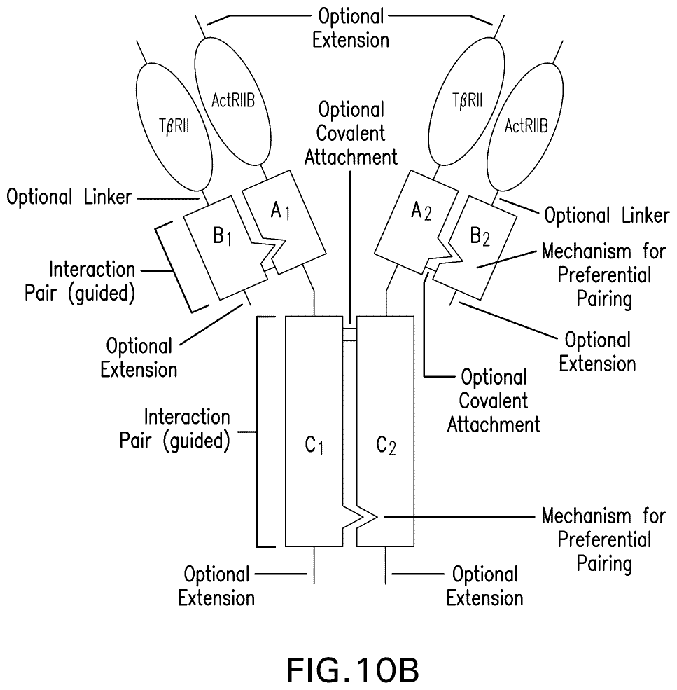

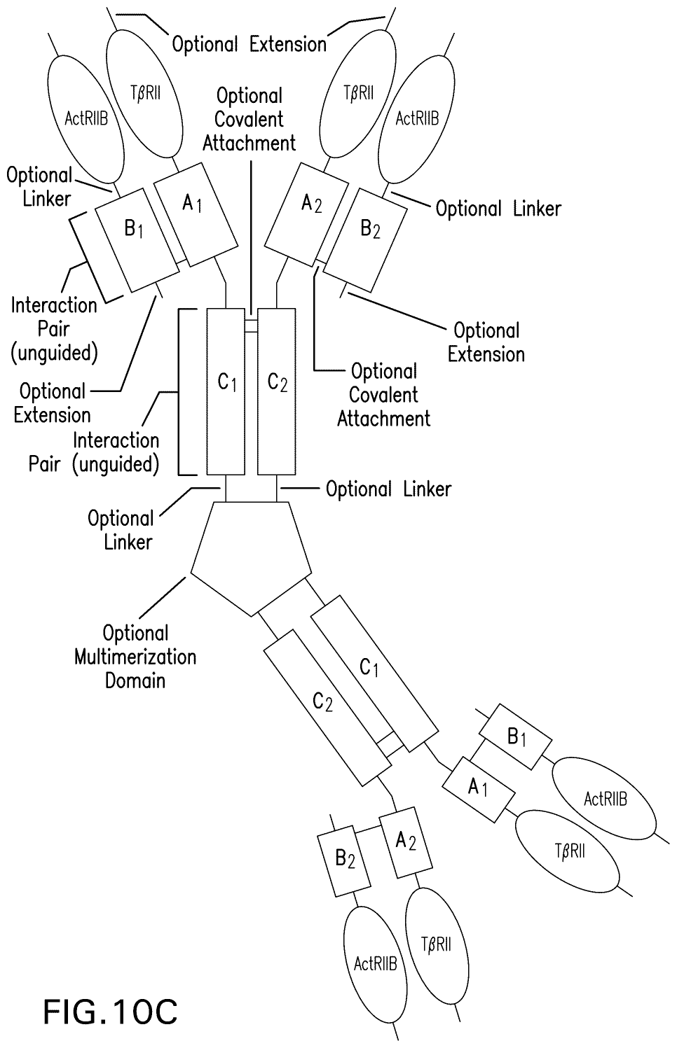

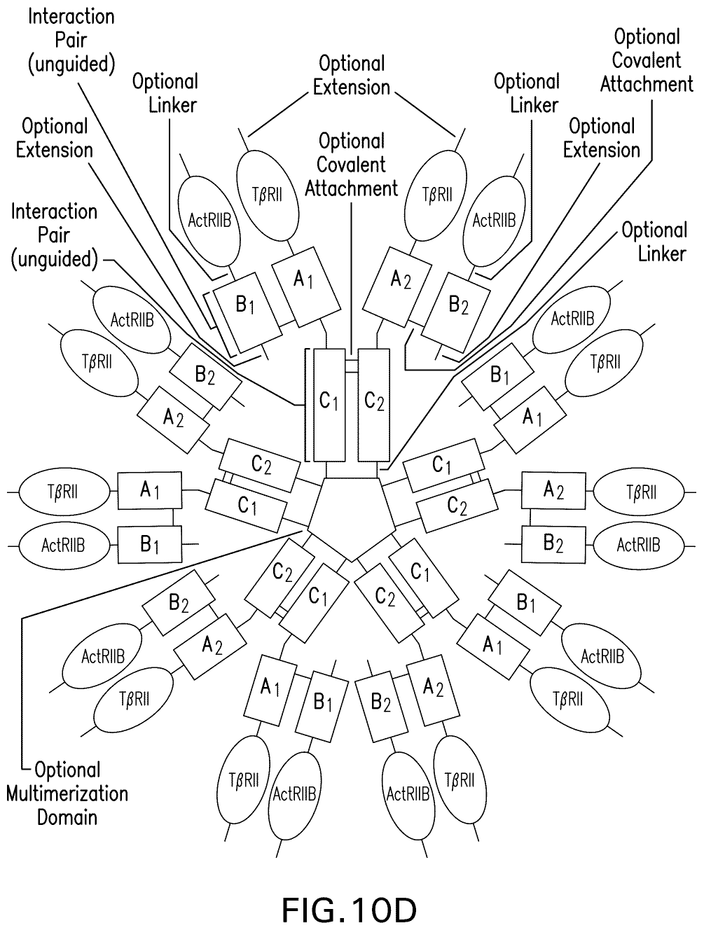

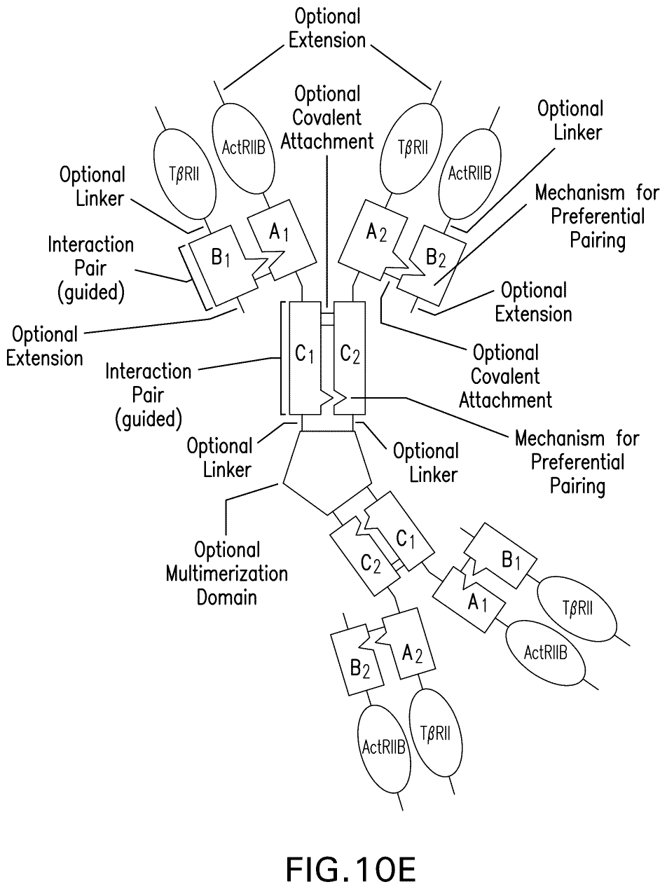

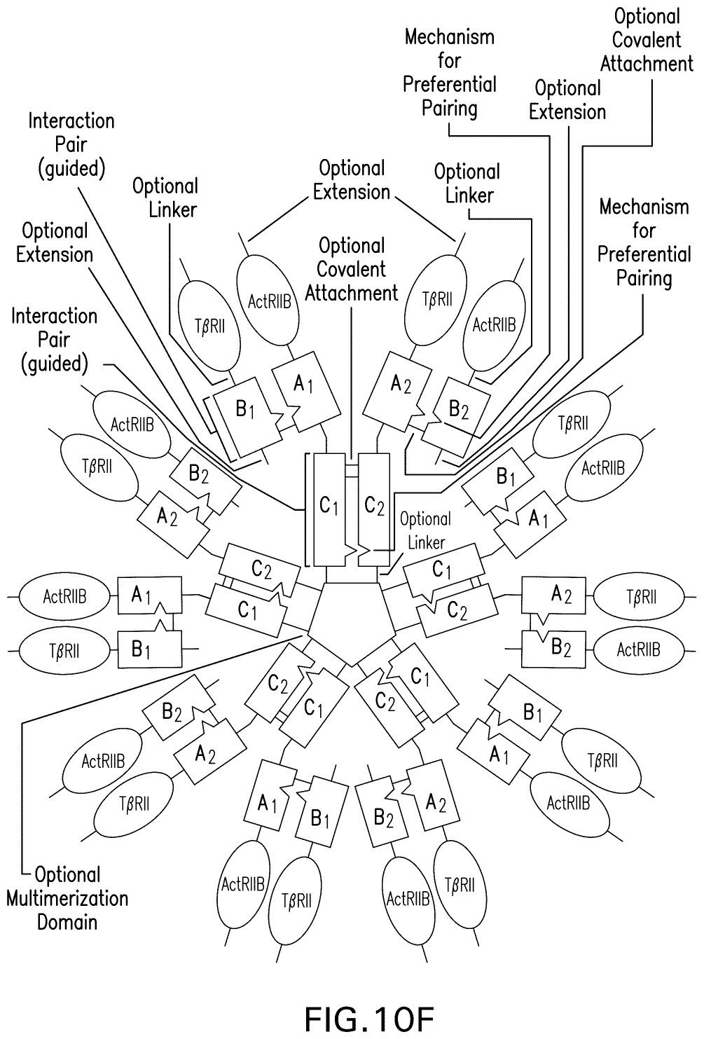

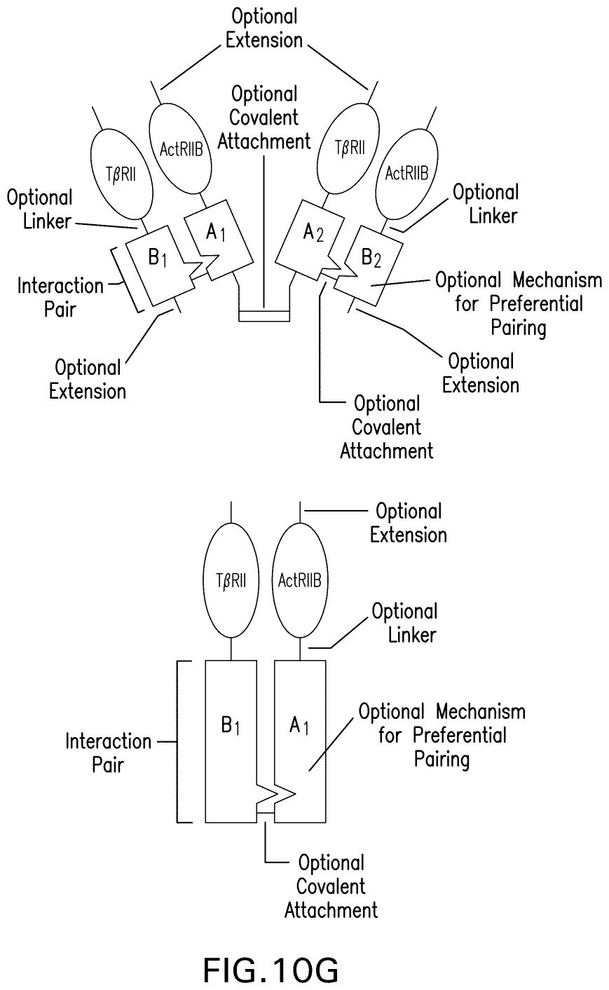

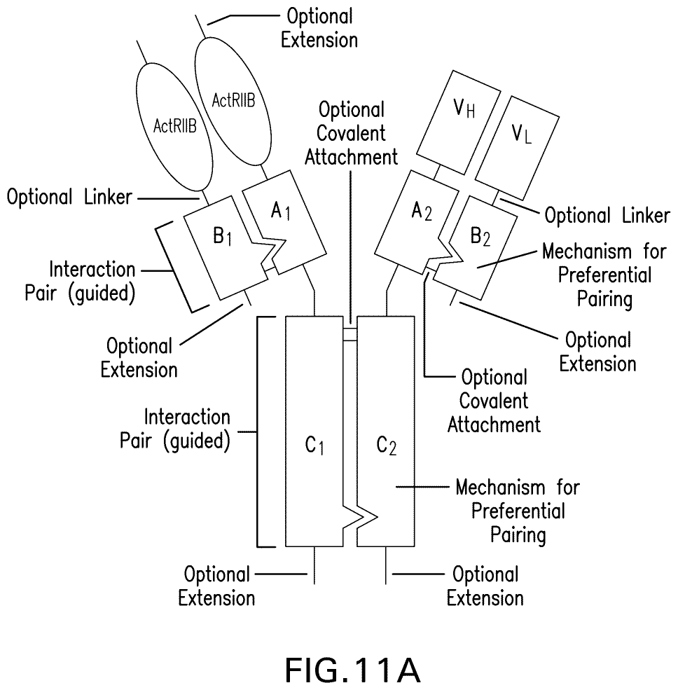

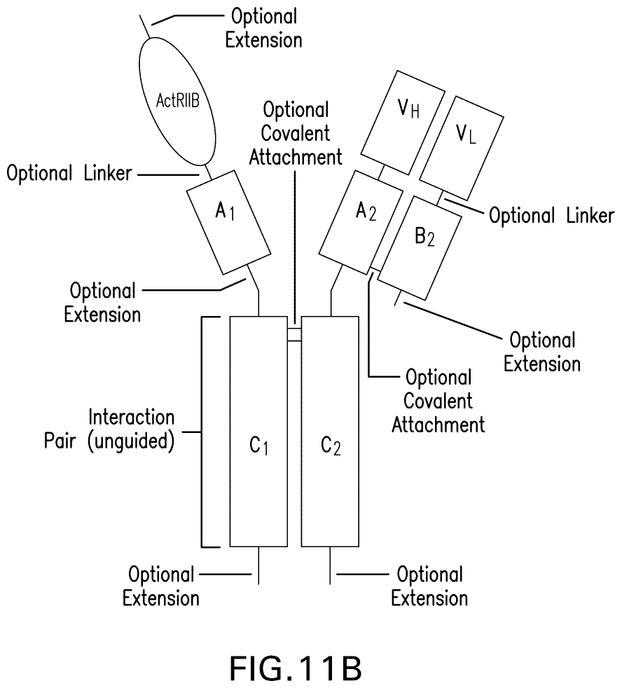

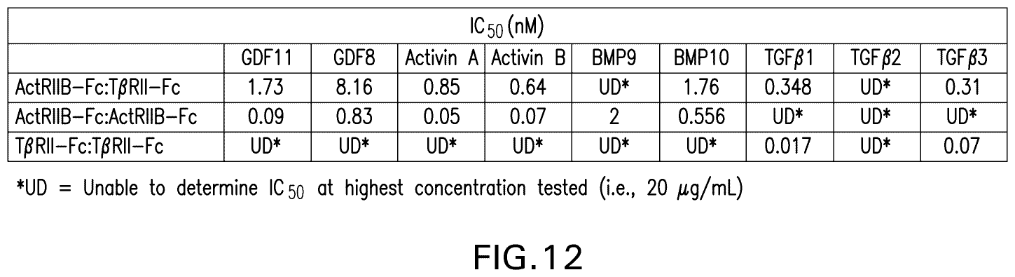

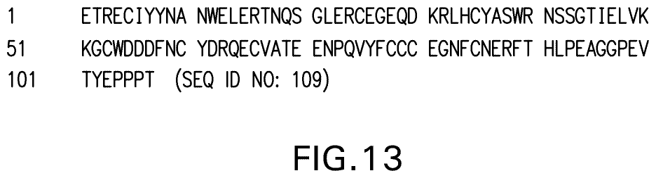

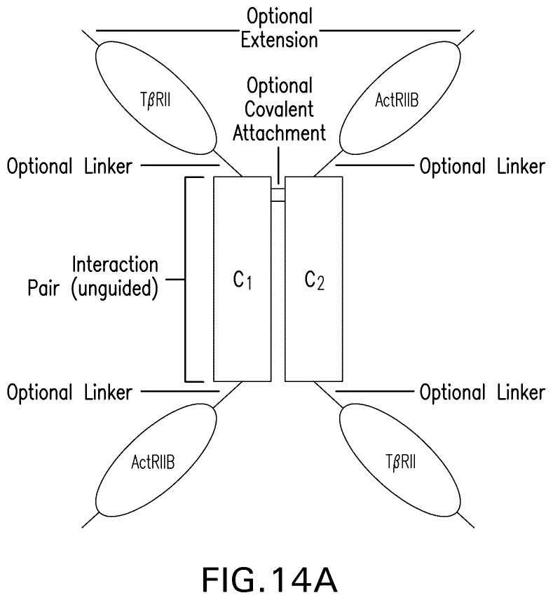

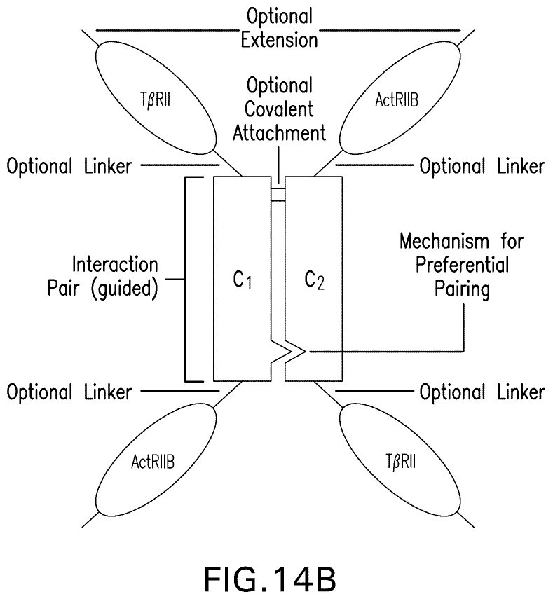

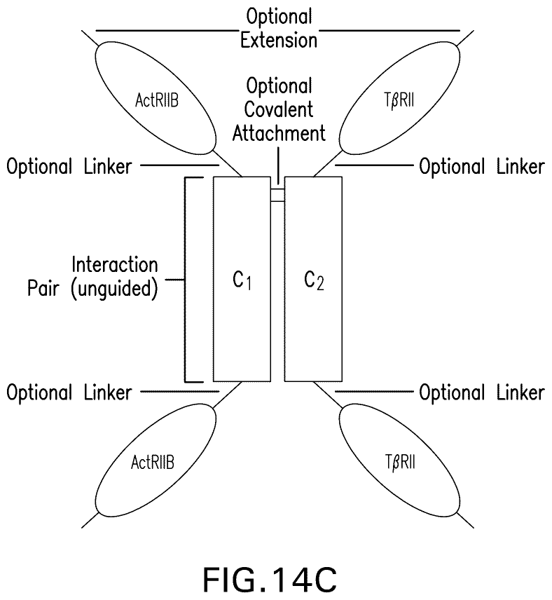

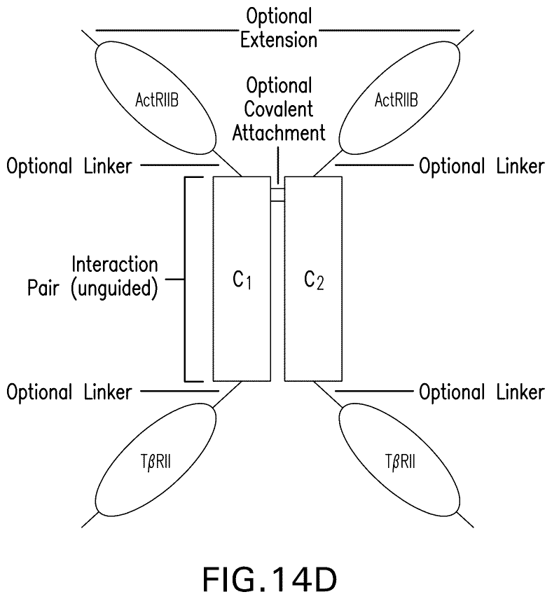

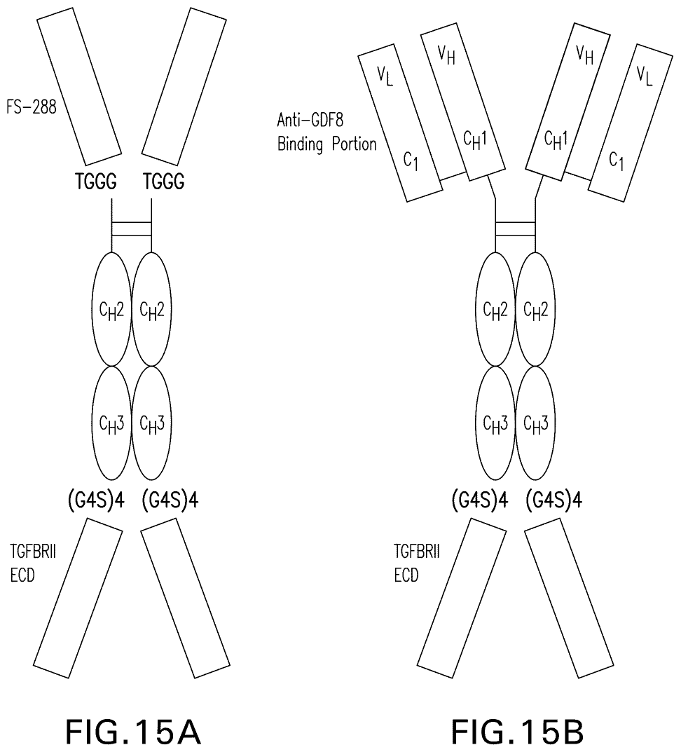

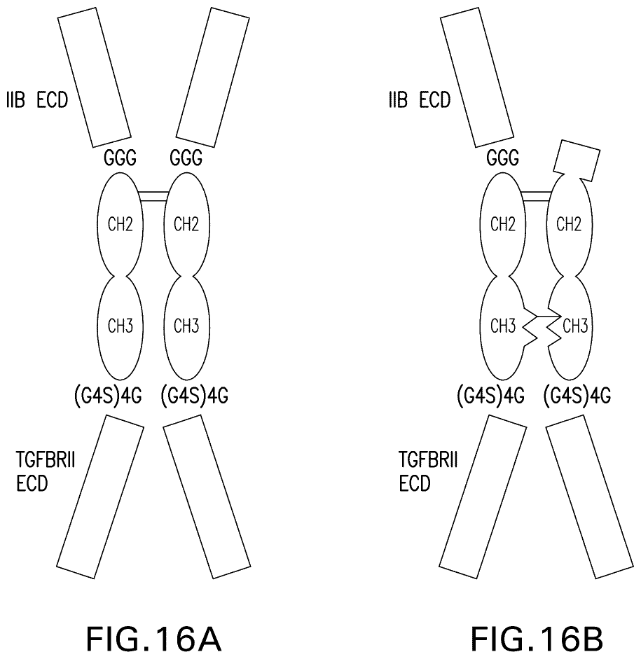

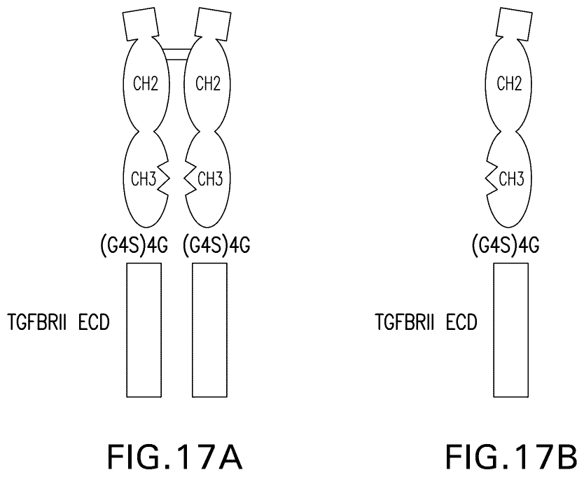

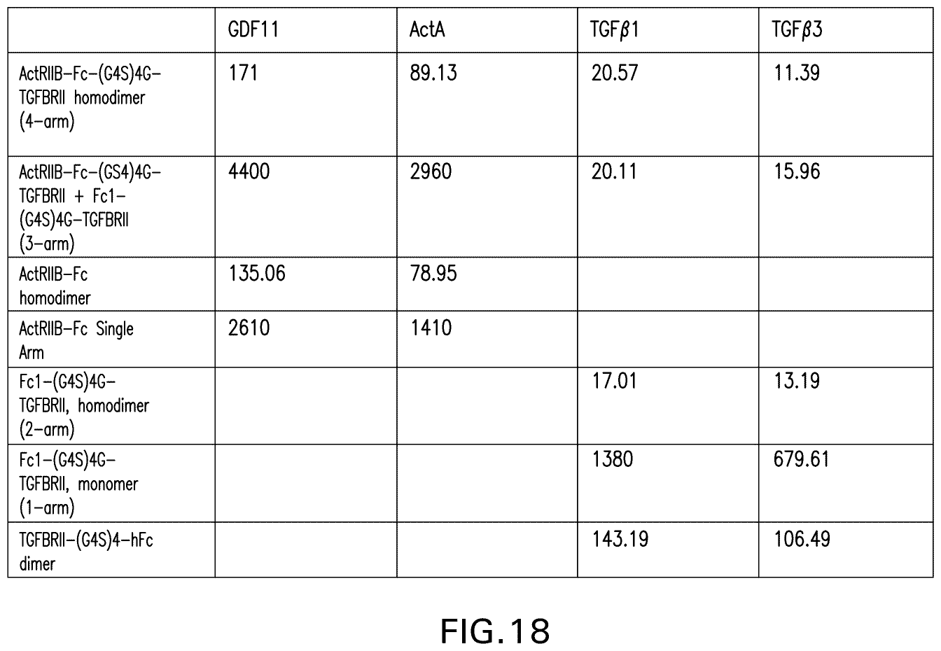

In the illustrated embodiments, the TβRII polypeptide (from left to right) is part of a fusion polypeptide that comprises a first member of an interaction pair (“C₁”), and the ActRIIB polypeptide is part of a fusion polypeptide that comprises a second member of an interaction pair (“C₂”). Suitable interaction pairs included, for example, heavy chain and/or light chain immunoglobulin interaction pairs, truncations, and variants thereof such as those described herein [e.g., Spiess et al (2015) Molecular Immunology 67(2A): 95-106]. In each fusion polypeptide, a linker may be positioned between the TβRII or ActRIIB polypeptide and the corresponding member of the interaction pair. The first and second members of the interaction pair may be unguided, meaning that the members of the pair may associate with each other or self-associate without substantial preference, and they may have the same or different amino acid sequences. See FIG. 9A. Alternatively, the interaction pair may be a guided (asymmetric) pair, meaning that the members of the pair associate preferentially with each other rather than self-associate. See FIG. 9B. Complexes of higher order can be envisioned. See FIGS. 9C and 9D.

FIGS. 10A-10G show schematic examples of heteromeric protein complexes comprising two TβRII polypeptides (e.g. polypeptide that are independently at least 70%, 75%, 80%, 85%, 90%, 91%, 92%, 93%, 94%, 95%, 97%, 98%, 99% or 100% identical to an extracellular domain of an TβRII protein from humans or other species such as those described herein, e.g., SEQ ID Nos: 18, 27, and 28-39) and two ActRIIB polypeptides (e.g. two polypeptides that are independently at least 70%, 75%, 80%, 85%, 90%, 91%, 92%, 93%, 94%, 95%, 97%, 98%, 99% or 100% identical to an extracellular domain of an ActRIIB protein from humans or other species such as those described herein, e.g., SEQ ID Nos: 51, 52, 54, 55, and 109).

In the illustrated embodiment 10A, the first TβRII polypeptide (from left to right) is part of a fusion polypeptide that comprises a first member of an interaction pair (“C₁”) and further comprises an additional first member of an interaction pair (“A₁”); and the second TβRII polypeptide is part of a fusion polypeptide that comprises a second member of an interaction pair (“C₂”) and further comprises an first member of an interaction pair (“A₂”). The first ActRIIB polypeptide (from left to right) is part of a fusion polypeptide that comprises a second member of an interaction pair (“B₁”); and the second ActRIIB polypeptide is part of a fusion polypeptide that comprises a second member of an interaction pair (“B₂”). A₁ and A₂ may be the same or different; B₁ and B₂ may be the same or different, and C₁ and C₂ may be the same or different. In each fusion polypeptide, a linker may be positioned between the TβRII or ActRIIB polypeptide and the corresponding member of the interaction pair as well as between interaction pairs. FIG. 10A is an example of an association of unguided interaction pairs, meaning that the members of the pair may associate with each other or self-associate without substantial preference and may have the same or different amino acid sequences.

In the illustrated embodiment 10B, the first ActRIIB polypeptide (from left to right) is part of a fusion polypeptide that comprises a first member of an interaction pair (“C₁”) and further comprises an additional first member of an interaction pair (“A₁”); and the second ActRIIB polypeptide is part of a fusion polypeptide that comprises a second member of an interaction pair (“B₂”). The first TβRII polypeptide (from left to right) is part of a fusion polypeptide that comprises a second member of an interaction pair (“B₁”); and the second TβRII polypeptide is part of a fusion polypeptide that comprises a second member of an interaction pair (“C₂”) and further comprises a first member of an interaction pair (“A₂”). In each fusion polypeptide, a linker may be positioned between the TβRII or ActRIIB polypeptide and the corresponding member of the interaction pair as well as between interaction pairs. FIG. 10B is an example of an association of guided (asymmetric) interaction pairs, meaning that the members of the pair associate preferentially with each other rather than self-associate.

Suitable interaction pairs included, for example, heavy chain and/or light chain immunoglobulin interaction pairs, truncations, and variants thereof as described herein [e.g., Spiess et al (2015) Molecular Immunology 67(2A): 95-106]. Complexes of higher order can be envisioned. See FIG. 9C-9F. Using similar methods (particularly those that employ light and/or heavy chain immunoglobulins, truncations, or variants thereof), interaction pairs may be used to produce ActRIIB:TβRII heterodimers that resemble antibody Fab and F(ab′)₂ complexes [e.g., Spiess et al (2015) Molecular Immunology 67(2A): 95-106]. See FIG. 10G.

FIGS. 11A and 11B show schematic examples of a heteromeric protein complex comprising an antigen-binding domain of antibody that binds to one or more of TGFβ1, TGFβ2, TGFβ3 and at least one ActRIIB polypeptide domain (e.g. a polypeptide that is at least 70%, 75%, 80%, 85%, 90%, 91%, 92%, 93%, 94%, 95%, 97%, 98%, 99% or 100% identical to an extracellular domain of an ActRIIB protein from humans or other species as such as those described herein, e.g., SEQ ID Nos: 51, 52, 54, 55, and 109). In the illustrated embodiments, the first ActRIIB polypeptide is part of a fusion polypeptide that comprises a first member of an interaction pair (“C₁”), and further comprises an additional first member of an interaction pair (“A₁”). The second ActRIIB polypeptide is part of a fusion polypeptide that comprises a second member of an interaction pair (“B₁”). The variable heavy chain (V_{H}) polypeptide is part of a fusion polypeptide that comprises a second member of an interaction pair (“C₂”), and further comprises a first member of an interaction pair (“A₂”). The variable light chain (V_{L}) polypeptide is part of a fusion polypeptide that comprises a second member of an interaction pair (“B₂”). In each fusion polypeptide, a linker may be positioned between the first or second ActRIIB polypeptide and the corresponding member of the interaction pair, between interaction pairs, and between the V_{H} and V_{L} polypeptides and a member of the interaction pair. A₁ and A₂ may be the same or different; B₁ and B₂ may be the same or different, and C₁ and C₂ may be the same or different. Suitable interaction pairs included, for example, constant heavy chain and/or light chain immunoglobulin interaction pairs, truncations, and variants thereof as described herein [e.g., Spiess et al (2015) Molecular Immunology 67(2A): 95-106]. FIG. 11A is an example of a heterodimer comprising a first and second ActRIIB extracellular domain. FIG. 11B is an example of a heteromultimer comprising a single ActRIIB extracellular domain.

FIG. 12 shows comparative ActRIIB-Fc:TβRII-Fc heterodimer compared to an ActRIIB-Fc:ActRIIB-Fc homodimer and TβRII-Fc:TβRII-Fc homodimer. IC₅₀ data was determined by an A-204 Reporter Gene Assay as described herein. ActRIIB-Fc:TβRII-Fc heterodimer inhibits activin A, activin B, GDF8, GDF11, and BMP10-signaling pathways similarly to the ActRIIB-Fc:ActRIIB-Fc homodimer. However, ActRIIB-Fc:TβRII-Fc heterodimer inhibition of BMP9 signaling pathways is significantly reduced compared to the ActRIIB-Fc:ActRIIB-Fc homodimer. These data demonstrate that ActRIIB-Fc:TβRII-Fc heterodimers are more selective antagonists of activin A, activin B, GDF8, GDF11 and BMP10 compared to corresponding ActRIIB-Fc:ActRIIB-Fc homodimers. In addition the ActRIIB-Fc:TβRII-Fc heterodimer inhibits TGFβ1 and TGFβ3 signaling pathways similarly to the TβRII-Fc:TβRII-Fc homodimer.

FIG. 13 shows the amino acid sequence for a truncated, variant ActRIIB (25-131, L79D) domain (SEQ ID NO: 109).

FIGS. 14A-14D show schematic examples of heteromeric protein complexes comprising an TβRII polypeptide (e.g., a polypeptide that is at least 70%, 75%, 80%, 85%, 90%, 91%, 92%, 93%, 94%, 95%, 97%, 98%, 99% or 100% identical to an extracellular domain of an TβRII protein from humans or other species such as those described herein, e.g., SEQ ID Nos: 18, 27, and 28-39) and an ActRIIB polypeptide (e.g. a polypeptide that is at least 70%, 75%, 80%, 85%, 90%, 91%, 92%, 93%, 94%, 95%, 97%, 98%, 99% or 100% identical to an extracellular domain of an ActRIIB protein from humans or other species such as those described herein, e.g., SEQ ID NOs: 51, 52, 53, 54, and 109).

In the illustrated embodiments, the TβRII:ActRIIB single-chain polypeptide (is part of a fusion polypeptide that comprises a first member of an interaction pair (“C₁”), and the ActRIIB:TβRII polypeptide is part of a fusion polypeptide that comprises a second member of an interaction pair (“C₂”). Suitable interaction pairs included, for example, heavy chain and/or light chain immunoglobulin interaction pairs, truncations, and variants thereof such as those described herein [e.g., Spiess et al (2015) Molecular Immunology 67(2A): 95-106]. In each fusion polypeptide, a linker may be positioned between the TβRII and/or ActRIIB polypeptide and the corresponding member of the interaction pair. The first and second members of the interaction pair may be unguided, meaning that the members of the pair may associate with each other or self-associate without substantial preference, and they may have the same or different amino acid sequences. See FIG. 14A. Alternatively, the interaction pair may be a guided (asymmetric) pair, meaning that the members of the pair associate preferentially with each other rather than self-associate. See FIG. 14B. Additional protein complexes can be envisioned. See FIGS. 14C and 14D.

FIG. 15A shows a schematic example of a representative multispecific binder comprising a TβRII (referred to here as a TGFBRII) polypeptide (e.g., a polypeptide that is at least 70%, 75%, 80%, 85%, 90%, 91%, 92%, 93%, 94%, 95%, 97%, 98%, 99% or 100% identical to SEQ ID NO: 170) and a follistatin polypeptide (e.g. a polypeptide that is at least 70%, 75%, 80%, 85%, 90%, 91%, 92%, 93%, 94%, 95%, 97%, 98%, 99% or 100% identical to SEQ ID NO: 111). FIG. 15B shows a schematic example of multispecific binder comprising a TβRII polypeptide (e.g., a polypeptide that is at least 70%, 75%, 80%, 85%, 90%, 91%, 92%, 93%, 94%, 95%, 97%, 98%, 99% or 100% identical to SEQ ID NO: 170) and a GDF8 antigen binding fragment (e.g. a polypeptide comprising the heavy chain and light chain CDRs of SEQ ID NOs: 151-156).

FIG. 16A shows a simplified schematic of a representative “four arm” homodimer comprising two fusion proteins, with each fusion protein comprising an ActRIIB extracellular domain (IIB ECD), a GGG linker, an Fc portion comprising CH2-CH3 Fc domains, a (G4S)4G linker, and a TGFβRII extracellular domain (TGFβRII ECD). FIG. 16B shows a simplified schematic of a representative “three-arm” heteromultimer comprising two fusion proteins, where the first fusion protein comprises an ActRIIB extracellular domain (IIB ECD), a GGG linker, an Fc portion comprising CH2-CH3 Fc domains with “knob substitutions”, a (G4S)4G linker, and a TGFβRII extracellular domain (TGFβRII ECD); and where the second fusion protein comprises an Fc portion comprising CH2-CH3 Fe domains with “hole substitutions”, a (G4S)4G linker, and a TGFβRII extracellular domain (TGFβRII ECD).

FIG. 17A shows a simplified schematic of a representative “two-arm” homodimer comprising two fusion proteins, with each fusion protein comprising (from N-terminus to C-terminus) an Fc portion comprising CH2-CH3 Fe domains with “hole substitutions,” and a TGFβRII extracellular domain (TGFβRII ECD). FIG. 17B shows a simplified schematic of a representative “single-arm” comprising only a single fusion protein, with the fusion protein comprising (from N-terminus to C-terminus) an Fc portion comprising CH2-CH3 Fc domains with “hole substitutions,” and a TGFβRII extracellular domain (TGFβRII ECD).

FIG. 18 is a table providing IC₅₀ data (in pM) for different constructs against GDF11, Activin A, TGFβ1 or TGFβ3.

### DETAILED DESCRIPTION OF THE INVENTION

### 1. Overview

In some embodiments, the disclosure provides for novel binders of TGFβ-superfamily ligands. In some embodiments, the disclosure provides for a multispecific binder of TGFβ-superfamily ligands. In some embodiments, the multispecific binder protein is capable of binding to a) at least one of TGFβ1 and TGFβ3, and b) at least one of activin A, activin B, activin AB, GDF11, and GDF8. In some embodiments, the multispecific binder comprises: a) a first portion that is capable of binding to TGFβ1 and/or TGFβ3; and b) a second portion that is capable of binding to at least one of activin A, activin B, activin AB, GDF11, and GDF8. In some embodiments, the multispecific binder is a heteromultimer comprising an ActRIIB polypeptide and a TβRII polypeptide. In some embodiments, the multispecific binder comprises a TβRII polypeptide and a follistatin or a follistatin-like protein domain. In some embodiments, the multispecific binder comprises a TβRII polypeptide and an antibody or antigen-binding fragment, wherein the antibody or antigen-binding fragment is capable of binding to one or more of activin A, activin B, activin AB, GDF11, and/or GDF8. In particular embodiments, the multispecific binder comprises a TβRII polypeptide and an antibody or antigen-binding fragment, wherein the antibody or antigen-binding fragment is capable of binding to GDF8.

In some embodiments, the disclosure provides heteromultimers that comprise an ActRIIB polypeptide and a TβRII polypeptide. Preferably, such ActRIIB polypeptides comprise a ligand-binding domain of an ActRIIB receptor and such TβRII polypeptides comprise a ligand-binding domain of a TβRII receptor. In certain preferred embodiments, ActRIIB:TβRII heteromultimers of the disclosure are soluble. In certain preferred embodiments, ActRIIB:TβRII heteromultimers of the disclosure have an altered TGFβ superfamily ligand specificity compared to a corresponding sample of a homomultimer (e.g., an ActRIIB:TβRII heterodimer compared to an ActRIIB:ActRIIB homodimer or an TβRII:TβRII homodimer).

The TGFβ superfamily is comprised of over 30 secreted factors including TGFβs, activins, nodals, bone morphogenetic proteins (BMPs), growth and differentiation factors (GDFs), and anti-Mullerian hormone (AMH) [Weiss et al. (2013) Developmental Biology, 2(1): 47-63]. Members of the superfamily, which are found in both vertebrates and invertebrates, are ubiquitously expressed in diverse tissues and function during the earliest stages of development throughout the lifetime of an animal. Indeed, TGFβ superfamily proteins are key mediators of stem cell self-renewal, gastrulation, differentiation, organ morphogenesis, and adult tissue homeostasis. Consistent with this ubiquitous activity, aberrant TGFβ superfamily signaling is associated with a wide range of human pathologies including, for example, autoimmune disease, cardiovascular disease, fibrotic disease, and cancer.

Ligands of the TGFβ superfamily share the same dimeric structure in which the central 3-½ turn helix of one monomer packs against the concave surface formed by the beta-strands of the other monomer. The majority of TGFβ family members are further stabilized by an intermolecular disulfide bond. This disulfide bonds traverses through a ring formed by two other disulfide bonds generating what has been termed a ‘cysteine knot’ motif [Lin et al. (2006) Reproduction 132: 179-190; and Hinck et al. (2012) FEBS Letters 586: 1860-1870].

TGFβ superfamily signaling is mediated by heteromeric complexes of type I and type II serine/threonine kinase receptors, which phosphorylate and activate downstream SMAD proteins (e.g., SMAD proteins 1, 2, 3, 5, and 8) upon ligand stimulation [Massagué (2000) Nat. Rev. Mol. Cell Biol. 1:169-178]. These type I and type II receptors are transmembrane proteins, composed of a ligand-binding extracellular domain with cysteine-rich region, a transmembrane domain, and a cytoplasmic domain with predicted serine/threonine kinase specificity. In general, type I receptors mediate intracellular signaling while the type II receptors are required for binding TGFβ superfamily ligands. Type I and II receptors form a stable complex after ligand binding, resulting in phosphorylation of type I receptors by type II receptors.

The TGFβ family can be divided into two phylogenetic branches based on the type I receptors they bind and the Smad proteins they activate. One is the more recently evolved branch, which includes, e.g., the TGFβs, activins, GDF8, GDF9, GDF11, BMP3 and nodal, which signal through type I receptors that activate Smads 2 and 3 [Hinck (2012) FEBS Letters 586:1860-1870]. The other branch comprises the more distantly related proteins of the superfamily and includes, e.g., BMP2, BMP4, BMP5, BMP6, BMP7, BMP8a, BMP8b, BMP9, BMP10, GDF1, GDF5, GDF6, and GDF7, which signal through Smads 1, 5, and 8.

TGFβ isoforms are the founding members of the TGFβ superfamily, of which there are 3 known isoforms in mammals designated as TGFβ1, TGFβ2 and TGFβ3. Mature bioactive TGFβ ligands function as homodimers and predominantly signal through the type I receptor ALK5, but have also been found to additionally signal through ALK1 in endothelial cells [Goumans et al. (2003) Mol Cell 12(4): 817-828]. TGFβ1 is the most abundant and ubiquitously expressed isoform. TGFβ1 is known to have an important role in wound healing, and mice expressing a constitutively active TGFβ1 transgene develop fibrosis [Clouthier et al. (1997) J Clin. Invest. 100(11): 2697-2713]. TGFβ1 is also involved in T cell activation and maintenance of T regulatory cells [Li et al. (2006) Immunity 25(3): 455-471]. TGFβ2 expression was first described in human glioblastoma cells, and is occurs in neurons and astroglial cells of the embryonic nervous system. TGFβ2 is known to suppress interleukin-2-dependent growth of T lymphocytes. TGFβ3 was initially isolated from a human rhabdomyosarcoma cell line and since has been found in lung adenocarcinoma and kidney carcinoma cell lines. TGFβ3 is known to be important for palate and lung morphogenesis [Kubiczkova et al. (2012) Journal of Translational Medicine 10:183].

Activins are members of the TGFβ superfamily and were initially discovered as regulators of secretion of follicle-stimulating hormone, but subsequently various reproductive and non-reproductive roles have been characterized. There are three principal activin forms (A, B, and AB) that are homo/heterodimers of two closely related β subunits (β_{A}β_{A}, β_{B}β_{B}, and β_{A}β_{B}, respectively). The human genome also encodes an activin C and an activin E, which are primarily expressed in the liver, and heterodimeric forms containing β_{C} or β_{E} are also known. In the TGFβ superfamily, activins are unique and multifunctional factors that can stimulate hormone production in ovarian and placental cells, support neuronal cell survival, influence cell-cycle progress positively or negatively depending on cell type, and induce mesodermal differentiation at least in amphibian embryos [DePaolo et al. (1991) Proc Soc Ep Biol Med. 198:500-512; Dyson et al. (1997) Curr Biol. 7:81-84; and Woodruff (1998) Biochem Pharmacol. 55:953-963]. In several tissues, activin signaling is antagonized by its related heterodimer, inhibin. For example, in the regulation of follicle-stimulating hormone (FSH) secretion from the pituitary, activin promotes FSH synthesis and secretion, while inhibin reduces FSH synthesis and secretion. Other proteins that may regulate activin bioactivity and/or bind to activin include follistatin (FS), follistatin-related protein (FSRP, also known as FLRG or FSTL3), and α₂-macroglobulin.

As described herein, agents that bind to “activin A” are agents that specifically bind to the β_{A} subunit, whether in the context of an isolated β_{A} subunit or as a dimeric complex (e.g., a β_{A}β_{A} homodimer or a β_{A}β_{B} heterodimer). In the case of a heterodimer complex (e.g., a β_{A}β_{B} heterodimer), agents that bind to “activin A” are specific for epitopes present within the β_{A} subunit, but do not bind to epitopes present within the non-β_{A} subunit of the complex (e.g., the β_{B} subunit of the complex). Similarly, agents disclosed herein that antagonize (inhibit) “activin A” are agents that inhibit one or more activities as mediated by a β_{A} subunit, whether in the context of an isolated β_{A} subunit or as a dimeric complex (e.g., a β_{A}β_{A} homodimer or a β_{A}β_{B} heterodimer). In the case of β_{A}β_{B} heterodimers, agents that inhibit “activin A” are agents that specifically inhibit one or more activities of the β_{A} subunit, but do not inhibit the activity of the non-β_{A} subunit of the complex (e.g., the β_{B} subunit of the complex). This principle applies also to agents that bind to and/or inhibit “activin B”, “activin C”, and “activin E”. Agents disclosed herein that antagonize “activin AB” are agents that inhibit one or more activities as mediated by the β_{A} subunit and one or more activities as mediated by the β_{B} subunit. The same principle also applies to agent that bind to and/or inhibit “activin AC”, “activin BC”, “activin AE”, and “activin BE”.

The BMPs and GDFs together form a family of cysteine-knot cytokines sharing the characteristic fold of the TGFβ superfamily [Rider et al. (2010) Biochem J., 429(1):1-12]. This family includes, for example, BMP2, BMP4, BMP6, BMP7, BMP2a, BMP3, BMP3b (also known as GDF10), BMP4, BMP5, BMP6, BMP7, BMP8, BMP8a, BMP8b, BMP9 (also known as GDF2), BMP10, BMP11 (also known as GDF11), BMP12 (also known as GDF7), BMP13 (also known as GDF6), BMP14 (also known as GDF5), BMP15, GDF1, GDF3 (also known as VGR2), GDF8 (also known as myostatin), GDF9, GDF15, and decapentaplegic. Besides the ability to induce bone formation, which gave the BMPs their name, the BMP/GDFs display morphogenetic activities in the development of a wide range of tissues. BMP/GDF homo- and hetero-dimers interact with combinations of type I and type II receptor dimers to produce multiple possible signaling complexes, leading to the activation of one of two competing sets of SMAD transcription factors. BMP/GDFs have highly specific and localized functions. These are regulated in a number of ways, including the developmental restriction of BMP/GDF expression and through the secretion of several specific BMP antagonist proteins that bind with high affinity to the cytokines. Curiously, a number of these antagonists resemble TGFβ superfamily ligands.

Growth and differentiation factor-8 (GDF8) is also known as myostatin. GDF8 is a negative regulator of skeletal muscle mass and is highly expressed in developing and adult skeletal muscle. The GDF8 null mutation in transgenic mice is characterized by a marked hypertrophy and hyperplasia of skeletal muscle [McPherron et al. Nature (1997) 387:83-90]. Similar increases in skeletal muscle mass are evident in naturally occurring mutations of GDF8 in cattle and, strikingly, in humans [Ashmore et al. (1974) Growth, 38:501-507; Swatland and Kieffer, J. Anim. Sci. (1994) 38:752-757; McPherron and Lee, Proc. Natl. Acad. Sci. USA (1997) 94:12457-12461; Kambadur et al. Genome Res. (1997) 7:910-915; and Schuelke et al. (2004) N Engl J Med, 350:2682-8]. Studies have also shown that muscle wasting associated with HIV-infection in humans is accompanied by increases in GDF8 protein expression [Gonzalez-Cadavid et al., PNAS (1998) 95:14938-43]. In addition, GDF8 can modulate the production of muscle-specific enzymes (e.g., creatine kinase) and modulate myoblast cell proliferation [International Patent Application Publication No. WO 00/43781]. The GDF8 propeptide can noncovalently bind to the mature GDF8 domain dimer, inactivating its biological activity [Miyazono et al. (1988) J. Biol. Chem., 263: 6407-6415; Wakefield et al. (1988) J. Biol. Chem., 263; 7646-7654; and Brown et al. (1990) Growth Factors, 3: 35-43]. Other proteins which bind to GDF8 or structurally related proteins and inhibit their biological activity include follistatin, and potentially, follistatin-related proteins [Gamer et al. (1999) Dev. Biol., 208: 222-232].

GDF11, also known as BMP11, is a secreted protein that is expressed in the tail bud, limb bud, maxillary and mandibular arches, and dorsal root ganglia during mouse development [McPherron et al. (1999) Nat. Genet., 22: 260-264; and Nakashima et al. (1999) Mech. Dev., 80: 185-189]. GDF11 plays a unique role in patterning both mesodermal and neural tissues [Gamer et al. (1999) Dev Biol., 208:222-32]. GDF11 was shown to be a negative regulator of chondrogenesis and myogenesis in developing chick limb [Gamer et al. (2001) Dev Biol., 229:407-20]. The expression of GDF11 in muscle also suggests its role in regulating muscle growth in a similar way to GDF8. In addition, the expression of GDF11 in brain suggests that GDF11 may also possess activities that relate to the function of the nervous system. Interestingly, GDF11 was found to inhibit neurogenesis in the olfactory epithelium [Wu et al. (2003) Neuron., 37:197-207]. Hence, inhibitors GDF11 may have in vitro and in vivo applications in the treatment of diseases such as muscle diseases and neurodegenerative diseases (e.g., amyotrophic lateral sclerosis).

BMP7, also called osteogenic protein-1 (OP-1), is well known to induce cartilage and bone formation. In addition, BMP7 regulates a wide array of physiological processes. For example, BMP7 may be the osteoinductive factor responsible for the phenomenon of epithelial osteogenesis. It is also found that BMP7 plays a role in calcium regulation and bone homeostasis. Like activin, BMP7 binds to type II receptors, ActRIIA and ActRIIB. However, BMP7 and activin recruit distinct type I receptors into heteromeric receptor complexes. The major BMP7 type I receptor observed was ALK2, while activin bound exclusively to ALK4 (ActRIIB). BMP7 and activin elicited distinct biological responses and activated different SMAD pathways [Macias-Silva et al. (1998) J Biol Chem. 273:25628-36].

As described herein, comparative inhibition data demonstrated that an ActRIIB:TβRII heterodimer can antagonize a broad range of Smad 2/3 activating ligands. For example, the disclosure demonstrates that an ActRIIB:TβRII heterodimer inhibits TGFβ1, TGFβ3, activin A, activin B, GDF8, GDF11, and BMP10-signaling pathways in a cell-based assay. In contrast, ActRIIB and TβRII homodimers alone inhibit a smaller subset of Smad 2/3 activating ligands. Moreover, the data demonstrate that the ActRIIB:TβRII heterodimer is a surprisingly more selective Smad 2/3 ligand antagonists that merely combining the antagonistic profiles of ActRIIB and TβRII homodimer ligand traps. For example, the ActRIIB:TβRII heterodimer inhibited activin A, activin B, GDF8, GDF11, and BMP10-signaling pathways similarly to an ActRIIB homodimer. However, ActRIIB:TβRII heterodimer inhibition of BMP9 signaling pathways is significantly reduced compared to the ActRIIB homodimer. ActRIIB:TβRII heteromultimers therefore are more selective antagonists of Smad 2/3 activating ligands compared to ActRIIB homodimers. Accordingly, an ActRIIB:TβRII heterodimer will be more useful than an ActRIIB or TβRII homodimer, or combination thereof, in certain applications where such broad, yet selective, Smad 2/3 antagonism is advantageous.

The terms used in this specification generally have their ordinary meanings in the art, within the context of this invention and in the specific context where each term is used. Certain terms are discussed below or elsewhere in the specification, to provide additional guidance to the practitioner in describing the compositions and methods of the invention and how to make and use them. The scope or meaning of any use of a term will be apparent from the specific context in which the term is used.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions.

The term “sequence similarity,” in all its grammatical forms, refers to the degree of identity or correspondence between nucleic acid or amino acid sequences that may or may not share a common evolutionary origin. However, in common usage and in the instant application, the term “homologous,” when modified with an adverb such as “highly,” may refer to sequence similarity and may or may not relate to a common evolutionary origin.

“Percent (%) sequence identity” or “percent (%) identical” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

“Agonize”, in all its grammatical forms, refers to the process of activating a protein and/or gene (e.g., by activating or amplifying that protein's gene expression or by inducing an inactive protein to enter an active state) or increasing a protein's and/or gene's activity.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity.

The terms “about” and “approximately” as used in connection with a numerical value throughout the specification and the claims denotes an interval of accuracy, familiar and acceptable to a person skilled in the art.

Numeric ranges disclosed herein are inclusive of the numbers defining the ranges.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification, the word “comprise” or variations such as “comprises” or “comprising” will be understood to imply the inclusion of a stated integer or groups of integers but not the exclusion of any other integer or group of integers. As used herein, the term “comprises” also encompasses the use of the narrower terms “consisting” and “consisting essentially of.”

The term “consisting essentially of” is limited to the specified materials or steps and those that do not materially affect the basic and novel characteristics of the invention(s) disclosed herein.

The term “appreciable affinity” as used herein means binding with a dissociation constant (K_{D}) of less than 50 nM.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

The terms “heteromer” or “heteromultimer” is a complex comprising at least a first polypeptide chain and a second polypeptide chain, wherein the second polypeptide chain differs in amino acid sequence from the first polypeptide chain by at least one amino acid residue. The heteromer can comprise a “heterodimer” formed by the first and second polypeptide chains or can form higher order structures where one or more polypeptide chains in addition to the first and second polypeptide chains are present. Exemplary structures for the heteromultimer include heterodimers, heterotrimers, heterotetramers and further oligomeric structures. Heterodimers are designated herein as X:Y or equivalently as X-Y, where X represents a first polypeptide chain and Y represents a second polypeptide chain. Higher-order heteromers and oligomeric structures are designated herein in a corresponding manner. In certain embodiments a heteromultimer is recombinant (e.g., one or more polypeptide components may be a recombinant protein), isolated and/or purified.

As used herein, the term “capable of” (e.g., capable of binding to) means that something has the ability to perform a particular action, but does not necessarily need to be performing that action at any particular point in time. For example, if a protein is “capable of binding to a ligand”, this would mean that the protein has the capability to bind to the ligand under physiological conditions, but is not required to be binding to the ligand at any particular point in time. Unless explicitly indicated otherwise herein, the term “binds to” means that something is “capable of binding to.”

### 2. Novel Binder of TGFβ-Superfamily Ligands

In some embodiments, the disclosure provides for novel binders of TGFβ-superfamily ligands. In some embodiments, the binder is capable of binding to at least one of TGFβ1 and TGFβ3. In some embodiments, the binder comprises a TβRII polypeptide and a heterologous domain (e.g., an Fc domain).

In some embodiments, the disclosure provides for a multispecific binder of TGFβ-superfamily ligands. In some embodiments, the multispecific binder is capable of binding to a) at least one of TGFβ1 and TGFβ3, and b) at least one of activin A, activin B, activin AB, GDF11, and GDF8. In some embodiments, the multispecific binder comprises: a) a first portion that is capable of binding to TGFβ1 and/or TGFβ3; and b) a second portion that is capable of binding to at least one of activin A, activin B, activin AB, GDF11, and GDF8. In some embodiments, the multispecific binder is a heteromultimer comprising an ActRIIB polypeptide and a TβRII polypeptide. In some embodiments, the multispecific binder comprises a TβRII polypeptide and a follistatin or a follistatin-like protein domain. In some embodiments, the multispecific binder comprises a TβRII polypeptide and an antibody or antigen-binding fragment, wherein the antibody or antigen-binding fragment is capable of binding to one or more of activin A, activin B, activin AB, GDF11, and/or GDF8. In particular embodiments, the multispecific binder comprises a TβRII polypeptide and an antibody or antigen-binding fragment, wherein the antibody or antigen-binding fragment is capable of binding to GDF8.

### A. ActRIIB and TβRII Polypeptides and Heteromultimers Thereof

In certain aspects, the present disclosure relates to heteromultimers comprising one or more ActRIIB receptor polypeptides (e.g., SEQ ID NOs: 51, 52, 54, 55, 82, 84, 88, 90, and 109) and one or more TβRII receptor polypeptides (e.g., SEQ ID NOs: 9, 11, 13, 15, 17, 18, 27, 28, 29, 30, 31, 32, 33, 34, 35, 36, 37, 38, 39, 44, 45, 85, 87, 91, 93, 94, 95, 96, 97, 98, 99, and 100) which are generally referred to herein as “ActRIIB:TβRII heteromultimer complexes” or “ActRIIB:TβRII heteromultimers”. Preferably, ActRIIB:TβRII heteromultimers of the disclosure are soluble, for example, a heteromultimer may comprises a soluble portion (domain) of a TβRII receptor and a soluble portion (domain) of an ActRIIB receptor. In general, the extracellular domains of TβRII and ActRIIB correspond to a soluble portion of these receptors. Therefore, in some embodiments, heteromultimers of the disclosure comprise an extracellular domain of a TβRII receptor and an extracellular domain of an ActRIIB receptor. Example extracellular domains TβRII and ActRIIB receptors are disclosed herein and such sequences, as well as fragments, functional variants, and modified forms thereof, may be used in accordance with the inventions of the disclosure (e.g., ActRIIB:TβRII heteromultimer compositions and uses thereof). ActRIIB:TβRII heteromultimers of the disclosure include, e.g., heterodimers, heterotrimers, heterotetramers and higher order oligomeric structures. See, e.g., FIGS. 9 and 10. In certain preferred embodiments, heteromultimers of the disclosure are ActRIIB:TβRII heterodimers. Preferably, ActRIIB:TβRII heteromultimers of the disclosure bind to one or more TGFβ superfamily ligands. In some embodiments, ActRIIB:TβRII heteromultimers may bind to one or more of activin (e.g., activin A, activin B, activin C, activin E, activin AC, activin AB, activin BC, activin AE, and activin BE), GDF8, GDF11, BMP10, TGFβ1, and TGFβ3. In some embodiments, ActRIIB:TβRII heteromultimers do not bind to, or no not substantially bind to BMP9 (e.g., have indeterminate Kₐ or K_{d} due to the transient nature of the interaction between BMP9 and an ActRIIB:TβRII heteromultimer). In some embodiments, ActRIIB:TβRII heteromultimers may be used to inhibit (antagonize) signaling (e.g., Smad 2/3) mediated by one or more TGFβ superfamily ligands. In particular, ActRIIB:TβRII heteromultimers of the disclosure may be used to inhibit intracellular signaling by one or more TGFβ superfamily ligands in, for example, a cell-based assay such as those described herein. For example, ActRIIB:TβRII heteromultimers may inhibit signaling mediated by one or more of activin (e.g., activin A, activin B, activin C, activin E, activin AC, activin AB, activin BC, activin AE, and activin BE), GDF8, GDF11, BMP10, TGFβ1, and TGFβ3 in a cell-based assay.

As used herein, the term “TβRII” refers to a family of transforming growth factor beta receptor II (TβRII) proteins from any species and variants derived from such TβRII proteins by mutagenesis or other modification. Reference to TβRII herein is understood to be a reference to any one of the currently identified forms. Members of the TβRII family are generally transmembrane proteins, composed of a ligand-binding extracellular domain comprising a cysteine-rich region, a transmembrane domain, and a cytoplasmic domain with predicted serine/threonine kinase activity. The term “TβRII polypeptide” includes polypeptides comprising any naturally occurring polypeptide of an TβRII family member as well as any variants thereof (including mutants, fragments, fusions, and peptidomimetic forms) that retain a useful activity.

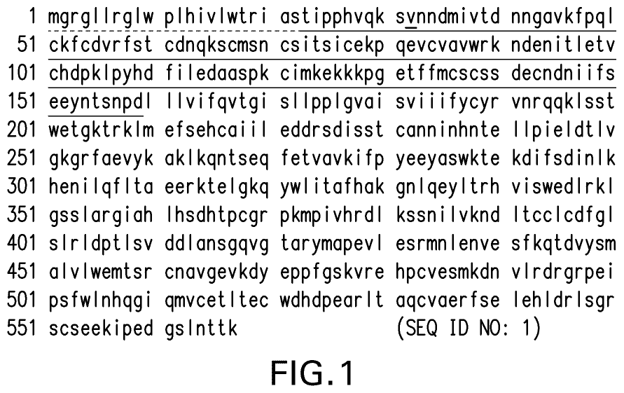

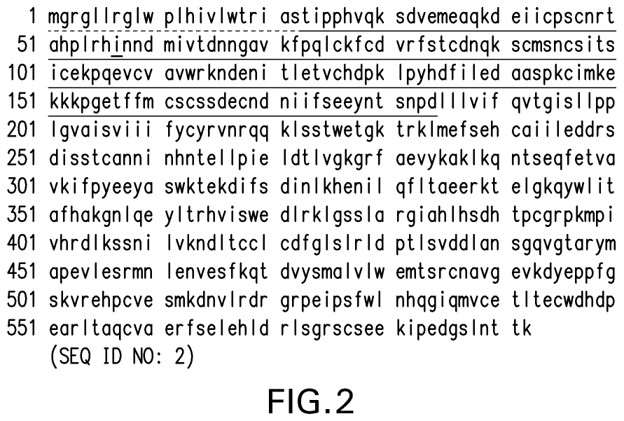

As described above, human TβRII occurs naturally in at least two isoforms—A (long) and B (short)—generated by alternative splicing in the extracellular domain (ECD) (FIGS. 1 and 2 and SEQ ID NOS: 1 and 2). SEQ ID NO: 27, which corresponds to residues 23-159 of SEQ ID NO: 1, depicts the native full-length extracellular domain of the short isoform of TβRII. SEQ ID NO: 18, which corresponds to residues 23-184 of SEQ ID NO: 2, depicts the native full-length extracellular domain of the long isoform of TβRII. Unless noted otherwise, amino acid position numbering with regard to variants based on the TβRII short and long isoforms refers to the corresponding position in the native precursors, SEQ ID NO: 1 and SEQ ID NO: 2, respectively.

In certain embodiments, the disclosure provides variant TβRII polypeptides. A TβRII polypeptide of the disclosure may bind to and inhibit the function of a TGFβ superfamily member, such as but not limited to, TGFβ1 or TGFβ3. TβRII polypeptides may include a polypeptide consisting of, or comprising, an amino acid sequence at least 70% identical, and optionally at least 70%, 75%, 80%, 85%, 90%, 91%, 92%, 93%, 94%, 95%, 96%, 97%, 98%, 99%, or 100% identical to a truncated ECD domain of a naturally occurring TβRII polypeptide, whose C-terminus occurs at any of amino acids 153-159 of SEQ ID NO: 1. TβRII polypeptides may include a polypeptide consisting of, or comprising, an amino acid sequence at least 70% identical, and optionally at least 70%, 75%, 80%, 85%, 90%, 91%, 92%, 93%, 94%, 95%, 96%, 97%, 98%, 99%, or 100% identical to a truncated ECD domain of a naturally occurring TβRII polypeptide, whose C-terminus occurs at any of amino acids 178-184 of SEQ ID NO: 2. In particular embodiments, the TβRII polypeptides comprise an amino acid sequence at least 70% identical, and optionally at least 70%, 75%, 80%, 85%, 90%, 91%, 92%, 93%, 94%, 95%, 96%, 97%, 98%, 99%, or 100% identical to the amino acid sequence of SEQ ID NO: 18. Optionally, a TβRII polypeptide does not include more than 5 consecutive amino acids, or more than 10, 20, 30, 40, 50, 52, 60, 70, 80, 90, 100, 150 or 200 or more consecutive amino acids from a sequence consisting of amino acids 160-567 of SEQ ID NO: 1 or from a sequence consisting of amino acids 185-592 of SEQ ID NO: 2. In some embodiments, the TβRII polypeptide does not include amino acids 160-567 of SEQ ID NO: 1. In some embodiments, the TβRII polypeptide does not include amino acids 1-22 of SEQ ID NO: 1. In some embodiments, the TβRII polypeptide does not include amino acids 1-22 and 160-567 of SEQ ID NO: 1. In some embodiments, the TβRII polypeptide does not include amino acids 185-592 of SEQ ID NO: 2. In some embodiments, the TβRII polypeptide does not include amino acids 1-22 of SEQ ID NO: 2. In some embodiments, the TβRII polypeptide does not include amino acids 1-22 and 185-592 of SEQ ID NO: 2. The unprocessed TβRII polypeptide may either include or exclude any signal sequence, as well as any sequence N-terminal to the signal sequence. As elaborated herein, the N-terminus of the processed TβRII polypeptide may occur at any of amino acids 23-35 of SEQ ID NO: 1 or 23-60 of SEQ ID NO: 2. Examples of processed TβRII polypeptides include, but are not limited to, amino acids 23-159 of SEQ ID NO: 1 (set forth in SEQ ID NO: 27), amino acids 29-159 of SEQ ID NO: 1 (set forth in SEQ ID NO: 28), amino acids 35-159 of SEQ ID NO: 1 (set forth in SEQ ID NO: 29), amino acids 23-153 of SEQ ID NO: 1 (set forth in SEQ ID NO: 30), amino acids 29-153 of SEQ ID NO: 1 (set forth in SEQ ID NO: 31), amino acids 35-153 of SEQ ID NO: 1 (set forth in SEQ ID NO: 32), amino acids 23-184 of SEQ ID NO: 2 (set forth in SEQ ID NO: 18), amino acids 29-184 of SEQ ID NO: 2 (set forth in SEQ ID NO: 33), amino acids 60-184 of SEQ ID NO: 2 (set forth in SEQ ID NO: 29), amino acids 23-178 of SEQ ID NO: 2 (set forth in SEQ ID NO: 34), amino acids 29-178 of SEQ ID NO: 2 (set forth in SEQ ID NO: 35), and amino acids 60-178 of SEQ ID NO: 2 (set forth in SEQ ID NO: 32). It will be understood by one of skill in the art that corresponding variants based on the long isoform of TβRII will include nucleotide sequences encoding the 25-amino acid insertion along with a conservative Val-Ile substitution at the flanking position C-terminal to the insertion. The TβRII polypeptides accordingly may include isolated extracellular portions of TβRII polypeptides, including both the short and the long isoforms, variants thereof (including variants that comprise, for example, no more than 2, 3, 4, 5, 10, 15, 20, 25, 30, or 35 amino acid substitutions in the sequence corresponding to amino acids 23-159 of SEQ ID NO: 1 or amino acids 23-184 of SEQ ID NO: 2), fragments thereof, and fusion proteins comprising any of the foregoing, but in each case preferably any of the foregoing TβRII polypeptides will retain substantial affinity for at least one of, or both of, TGFβ1 or TGFβ3. Generally, a TβRII polypeptide will be designed to be soluble in aqueous solutions at biologically relevant temperatures, pH levels, and osmolarity.

In some embodiments, the variant TβRII polypeptides of the disclosure comprise one or more mutations in the extracellular domain that confer an altered ligand binding profile. A TβRII polypeptide may include one, two, five or more alterations in the amino acid sequence relative to the corresponding portion of a naturally occurring TβRII polypeptide. In some embodiments, the mutation results in a substitution, insertion, or deletion at the position corresponding to position 70 of SEQ ID NO: 1. In some embodiments, the mutation results in a substitution, insertion, or deletion at the position corresponding to position 110 of SEQ ID NO: 1. Examples include, but are not limited to, an N to D substitution or a D to K substitution in the positions corresponding to positions 70 and 110, respectively, of SEQ ID NO: 1. Examples of such variant TβRII polypeptides include, but are not limited to, the sequences set forth in SEQ ID NOs: 36-39. A TβRII polypeptide may comprise a polypeptide or portion thereof that is encoded by any one of SEQ ID NOs: 8, 10, 12, 14, 16, 46 or 47, or silent variants thereof or nucleic acids that hybridize to the complement thereof under stringent hybridization conditions. In particular embodiments, a TβRII polypeptide may comprise a polypeptide or portion thereof that is encoded by any one of SEQ ID NO: 12, or silent variants thereof or nucleic acids that hybridize to the complement thereof under stringent hybridization conditions.

In some embodiments, the variant TβRII polypeptides of the disclosure further comprise an insertion of 36 amino acids (SEQ ID NO: 41) between the pair of glutamate residues (positions 151 and 152 of SEQ ID NO: 1, or positions 176 and 177 of SEQ ID NO: 2) located near the C-terminus of the human TβRII ECD, as occurs naturally in the human TβRII isoform C (Konrad et al., BMC Genomics 8:318, 2007).

It has been demonstrated that TβRII polypeptides can be modified to selectively antagonize TβRII ligands. The N70 residue represents a potential glycosylation site. In some embodiments, the TβRII polypeptides are aglycosylated. In some embodiments, the TβRII polypeptides are aglycosylated or have reduced glycosylation at position Asn157. In some embodiments, the TβRII polypeptides are aglycosylated or have reduced glycosylation at position Asn73.

In certain embodiments, a TβRII polypeptide binds to TGFβ1, and the TβRII polypeptide does not show substantial binding to TGFβ3. In certain embodiments, a TβRII polypeptide binds to TGFβ3, and the TβRII polypeptide does not show substantial binding to TGFβ 1. Binding may be assessed using purified proteins in solution or in a surface plasmon resonance system, such as a Biacore™ system.

In certain embodiments, a TβRII polypeptide inhibits TGFβ1 cellular signaling, and the TβRII polypeptide has an intermediate or limited inhibitory effect on TGFβ3 signaling. In certain embodiments, a TβRII polypeptide inhibits TGFβ3 cellular signaling, and the TβRII polypeptide has an intermediate or limited inhibitory effect on TGFβ1 signaling. Inhibitory effect on cell signaling can be assayed by methods known in the art.

Taken together, an active portion of a TβRII polypeptide may comprise amino acid sequences 23-153, 23-154, 23-155, 23-156, 23-157, or 23-158 of SEQ ID NO: 1, as well as variants of these sequences starting at any of amino acids 24-35 of SEQ ID NO: 1. Similarly, an active portion of a TβRII polypeptide may comprise amino acid sequences 23-178, 23-179, 23-180, 23-181, 23-182, or 23-183 of SEQ ID NO: 2, as well as variants of these sequences starting at any of amino acids 24-60 of SEQ ID NO: 2. Exemplary TβRII polypeptides comprise amino acid sequences 29-159, 35-159, 23-153, 29-153 and 35-153 of SEQ ID NO: 1 or amino acid sequences 29-184, 60-184, 23-178, 29-178 and 60-178 of SEQ ID NO: 2. Variants within these ranges are also contemplated, particularly those having at least 70%, 75%, 80%, 85%, 90%, 91%, 92%, 93%, 94%, 95%, 96%, 97%, 98%, or 99% identity to the corresponding portion of SEQ ID NO: 1 or SEQ ID NO: 2. A TβRII polypeptide may be selected that does not include the sequence consisting of amino acids 160-567 of SEQ ID NO: 1 or amino acids 185-592 of SEQ ID NO: 2. In particular embodiments, the TβRII polypeptides comprise an amino acid sequence at least 70% identical, and optionally at least 70%, 75%, 80%, 85%, 90%, 91%, 92%, 93%, 94%, 95%, 96%, 97%, 98%, 99%, or 100% identical to the amino acid sequence of SEQ ID NO: 18.

As used herein, the term “ActRIIB” refers to a family of activin receptor type IIB (ActRIIB) proteins from any species and variants derived from such ActRIIB proteins by mutagenesis or other modification. Reference to ActRIIB herein is understood to be a reference to any one of the currently identified forms. Members of the ActRIIB family are generally transmembrane proteins, composed of a ligand-binding extracellular domain comprising a cysteine-rich region, a transmembrane domain, and a cytoplasmic domain with predicted serine/threonine kinase activity.

The term “ActRIIB polypeptide” includes polypeptides comprising any naturally occurring polypeptide of an ActRIIB family member as well as any variants thereof (including mutants, fragments, fusions, and peptidomimetic forms) that retain a useful activity. Examples of such variant ActRIIB polypeptides are provided throughout the present disclosure as well as in International Patent Application Publication Nos. WO 2006/012627, WO 2008/097541, and WO 2010/151426, which are incorporated herein by reference in their entirety. Numbering of amino acids for all ActRIIB-related polypeptides described herein is based on the numbering of the human ActRIIB precursor protein sequence provided below (SEQ ID NO: 50), unless specifically designated otherwise.

The human ActRIIB precursor protein sequence is as follows:

| (SEQ ID NO: 50) | |
|---|---|
| 1 | MTAPWVALAL LWGSLCAGSG RGEAETRECI YYNANWELER TNQSGLERCE |
| 51 | GEQDKRLHCY ASWRNSSGTI ELVKKGCWLD DFNCYDRQEC VATEENPQVY |
| 101 | FCCCEGNFCN ERFTHLPEAG GPEVTYEPPP TAPTLLTVLA YSLLPIGGLS |
| 151 | LIVLLAFWMY RHRKPPYGHV DIHEDPGPPP PSPLVGLKPL QLLEIKARGR |
| 201 | FGCVWKAQLM NDFVAVKIFP LQDKQSWQSE REIFSTPGMK HENLLQFIAA |
| 251 | EKRGSNLEVE LWLITAFHDK GSLTDYLKGN IITWNELCHV AETMSRGLSY |
| 301 | LHEDVPWCRG EGHKPSIAHR DFKSKNVLLK SDLTAVLADF GLAVRFEPGK |
| 351 | PPGDTHGQVG TRRYMAPEVL EGAINFQRDA FLRIDMYAMG LVLWELVSRC |
| 401 | KAADGPVDEY MLPFEEEIGQ HPSLEELQEV VVHKKMRPTI KDHWLKHPGL |
| 451 | AQLCVTIEEC WDHDAEARLS AGCVEERVSL IRRSVNGTTS DCLVSLVTSV |
| 501 | TNVDLPPKES SI |

The signal peptide is indicated with a single underline; the extracellular domain is indicated in bold font; and the potential, endogenous N-linked glycosylation sites are indicated with a double underline.

The processed extracellular ActRIIB polypeptide sequence is as follows:

(SEQ ID NO: 51)
GRGEAETRECIYYNANWELERTNQSGLERCEGEQDKRLHCYASWRNSSGT
IELVKKGCWLDDFNCYDRQECVATEENPQVYFCCCEGNFCNERFTHLPEA
GGPEVTYEPPPTAPT

In some embodiments, the protein may be produced with an “SGR . . . ” sequence at the N-terminus. The C-terminal “tail” of the extracellular domain is indicated by a single underline. The sequence with the “tail” deleted (a Δ15 sequence) is as follows:

(SEQ ID NO: 52)
GRGEAETRECIYYNANWELERTNQSGLERCEGEQDKRLHCYASWRNSSGT
IELVKKGCWLDDFNCYDRQECVATEENPQVYFCCCEGNFCNERFTHLPEA

A form of ActRIIB with an alanine at position 64 of SEQ ID NO: 1 (A64) is also reported in the literature. See, e.g., Hilden et al. (1994) Blood, 83(8): 2163-2170. Applicants have ascertained that an ActRIIB-Fc fusion protein comprising an extracellular domain of ActRIIB with the A64 substitution has a relatively low affinity for activin and GDF11. By contrast, the same ActRIIB-Fc fusion protein with an arginine at position 64 (R64) has an affinity for activin and GDF11 in the low nanomolar to high picomolar range. Therefore, sequences with an R64 are used as the “wild-type” reference sequence for human ActRIIB in this disclosure.

The form of ActRIIB with an alanine at position 64 is as follows:

| (SEQ ID NO: 53) | |
|---|---|
| 1 | MTAPWVALAL LWGSLCAGSG RGEAETRECI YYNANWELER TNQSGLERCE |
| 51 | GEQDKRLHCY ASWANSSGTI ELVKKGCWLD DFNCYDRQEC VATEENPQVY |
| 101 | FCCCEGNFCN ERFTHLPEAG GPEVTYEPPP TAPTLLTVLA YSLLPIGGLS |
| 151 | LIVLLAFWMY RHRKPPYGHV DIHEDPGPPP PSPLVGLKPL QLLEIKARGR |
| 201 | FGCVWKAQLM NDFVAVKIFP LQDKQSWQSE REIFSTPGMK HENLLQFIAA |
| 251 | EKRGSNLEVE LWLITAFHDK GSLTDYLKGN IITWNELCHV AETMSRGLSY |
| 301 | LHEDVPWCRG EGHKPSIAHR DFKSKNVLLK SDLTAVLADF GLAVRFEPGK |
| 351 | PPGDTHGQVG TRRYMAPEVL EGAINFQRDA FLRIDMYAMG LVLWELVSRC |
| 401 | KAADGPVDEY MLPFEEEIGQ HPSLEELQEV VVHKKMRPTI KDHWLKHPGL |
| 451 | AQLCVTIEEC WDHDAEARLS AGCVEERVSL IRRSVNGTTS DCLVSLVTSV |
| 501 | TNVDLPPKES SI |

The signal peptide is indicated by single underline and the extracellular domain is indicated by bold font.

The processed extracellular ActRIIB polypeptide sequence of the alternative A64 form is as follows:

(SEQ ID NO: 54)
GRGEAETRECIYYNANWELERTNQSGLERCEGEQDKRLHCYASWANSSGT
IELVKKGCWLDDFNCYDRQECVATEENPQVYFCCCEGNFCNERFTHLPEA
GGPEVTYEPPPTAPT

In some embodiments, the protein may be produced with an “SGR . . . ” sequence at the N-terminus. The C-terminal “tail” of the extracellular domain is indicated by single underline. The sequence with the “tail” deleted (a Δ15 sequence) is as follows:

(SEQ ID NO: 55)
GRGEAETRECIYYNANWELERTNQSGLERCEGEQDKRLHCYASWANSSGT
IELVKKGCWLDDFNCYDRQECVATEENPQVYFCCCEGNFCNERFTHLPEA

A nucleic acid sequence encoding the human ActRIIB precursor protein is shown below (SEQ ID NO: 56), representing nucleotides 25-1560 of Genbank Reference Sequence NM_001106.3, which encode amino acids 1-513 of the ActRIIB precursor. The sequence as shown provides an arginine at position 64 and may be modified to provide an alanine instead. The signal sequence is underlined.

| (SEQ ID NO: 56) | |
|---|---|
| 1 | ATGACGGCGC CCTGGGTGGC CCTCGCCCTC CTCTGGGGAT CGCTGTGCGC |
| 51 | CGGCTCTGGG CGTGGGGAGG CTGAGACACG GGAGTGCATC TACTACAACG |
| 101 | CCAACTGGGA GCTGGAGCGC ACCAACCAGA GCGGCCTGGA GCGCTGCGAA |
| 151 | GGCGAGCAGG ACAAGCGGCT GCACTGCTAC GCCTCCTGGC GCAACAGCTC |
| 201 | TGGCACCATC GAGCTCGTGA AGAAGGGCTG CTGGCTAGAT GACTTCAACT |
| 251 | GCTACGATAG GCAGGAGTGT GTGGCCACTG AGGAGAACCC CCAGGTGTAC |
| 301 | TTCTGCTGCT GTGAAGGCAA CTTCTGCAAC GAACGCTTCA CTCATTTGCC |
| 351 | AGAGGCTGGG GGCCCGGAAG TCACGTACGA GCCACCCCCG ACAGCCCCCA |
| 401 | CCCTGCTCAC GGTGCTGGCC TACTCACTGC TGCCCATCGG GGGCCTTTCC |
| 451 | CTCATCGTCC TGCTGGCCTT TTGGATGTAC CGGCATCGCA AGCCCCCCTA |
| 501 | CGGTCATGTG GACATCCATG AGGACCCTGG GCCTCCACCA CCATCCCCTC |
| 551 | TGGTGGGCCT GAAGCCACTG CAGCTGCTGG AGATCAAGGC TCGGGGGCGC |
| 601 | TTTGGCTGTG TCTGGAAGGC CCAGCTCATG AATGACTTTG TAGCTGTCAA |
| 651 | GATCTTCCCA CTCCAGGACA AGCAGTCGTG GCAGAGTGAA CGGGAGATCT |
| 701 | TCAGCACACC TGGCATGAAG CACGAGAACC TGCTACAGTT CATTGCTGCC |
| 751 | GAGAAGCGAG GCTCCAACCT CGAAGTAGAG CTGTGGCTCA TCACGGCCTT |
| 801 | CCATGACAAG GGCTCCCTCA CGGATTACCT CAAGGGGAAC ATCATCACAT |
| 851 | GGAACGAACT GTGTCATGTA GCAGAGACGA TGTCACGAGG CCTCTCATAC |
| 901 | CTGCATGAGG ATGTGCCCTG GTGCCGTGGC GAGGGCCACA AGCCGTCTAT |
| 951 | TGCCCACAGG GACTTTAAAA GTAAGAATGT ATTGCTGAAG AGCGACCTCA |
| 1001 | CAGCCGTGCT GGCTGACTTT GGCTTGGCTG TTCGATTTGA GCCAGGGAAA |
| 1051 | CCTCCAGGGG ACACCCACGG ACAGGTAGGC ACGAGACGGT ACATGGCTCC |
| 1101 | TGAGGTGCTC GAGGGAGCCA TCAACTTCCA GAGAGATGCC TTCCTGCGCA |
| 1151 | TTGACATGTA TGCCATGGGG TTGGTGCTGT GGGAGCTTGT GTCTCGCTGC |
| 1201 | AAGGCTGCAG ACGGACCCGT GGATGAGTAC ATGCTGCCCT TTGAGGAAGA |
| 1251 | GATTGGCCAG CACCCTTCGT TGGAGGAGCT GCAGGAGGTG GTGGTGCACA |
| 1301 | AGAAGATGAG GCCCACCATT AAAGATCACT GGTTGAAACA CCCGGGCCTG |
| 1351 | GCCCAGCTTT GTGTGACCAT CGAGGAGTGC TGGGACCATG ATGCAGAGGC |
| 1401 | TCGCTTGTCC GCGGGCTGTG TGGAGGAGCG GGTGTCCCTG ATTCGGAGGT |
| 1451 | CGGTCAACGG CACTACCTCG GACTGTCTCG TTTCCCTGGT GACCTCTGTC |
| 1501 | ACCAATGTGG ACCTGCCCCC TAAAGAGTCA AGCATC |

A nucleic acid sequence encoding processed extracellular human ActRIIB polypeptide is as follows (SEQ ID NO: 57). The sequence as shown provides an arginine at position 64, and may be modified to provide an alanine instead.

| (SEQ ID NO: 57) | |
|---|---|
| 1 | GGGCGTGGGG AGGCTGAGAC ACGGGAGTGC ATCTACTACA ACGCCAACTG |
| 51 | GGAGCTGGAG CGCACCAACC AGAGCGGCCT GGAGCGCTGC GAAGGCGAGC |
| 101 | AGGACAAGCG GCTGCACTGC TACGCCTCCT GGCGCAACAG CTCTGGCACC |
| 151 | ATCGAGCTCG TGAAGAAGGG CTGCTGGCTA GATGACTTCA ACTGCTACGA |
| 201 | TAGGCAGGAG TGTGTGGCCA CTGAGGAGAA CCCCCAGGTG TACTTCTGCT |
| 251 | GCTGTGAAGG CAACTTCTGC AACGAACGCT TCACTCATTT GCCAGAGGCT |
| 301 | GGGGGCCCGG AAGTCACGTA CGAGCCACCC CCGACAGCCC CCACC |

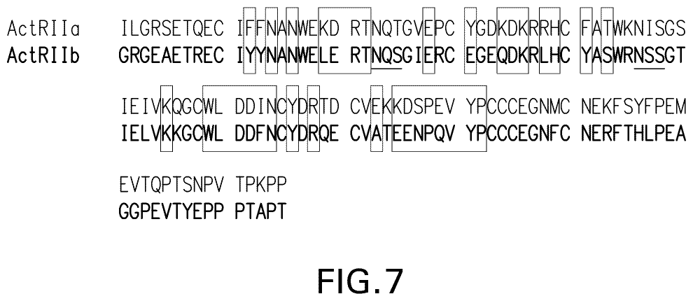

An alignment of the amino acid sequences of human ActRIIB extracellular domain and human ActRIIA extracellular domain are illustrated in FIG. 7. This alignment indicates amino acid residues within both receptors that are believed to directly contact ActRII ligands. For example, the composite ActRII structures indicated that the ActRIIB-ligand binding pocket is defined, in part, by residues Y31, N33, N35, L38 through T41, E47, E50, Q53 through K55, L57, H58, Y60, S62, K74, W78 through N83, Y85, R87, A92, and E94 through F10. At these positions, it is expected that conservative mutations will be tolerated.

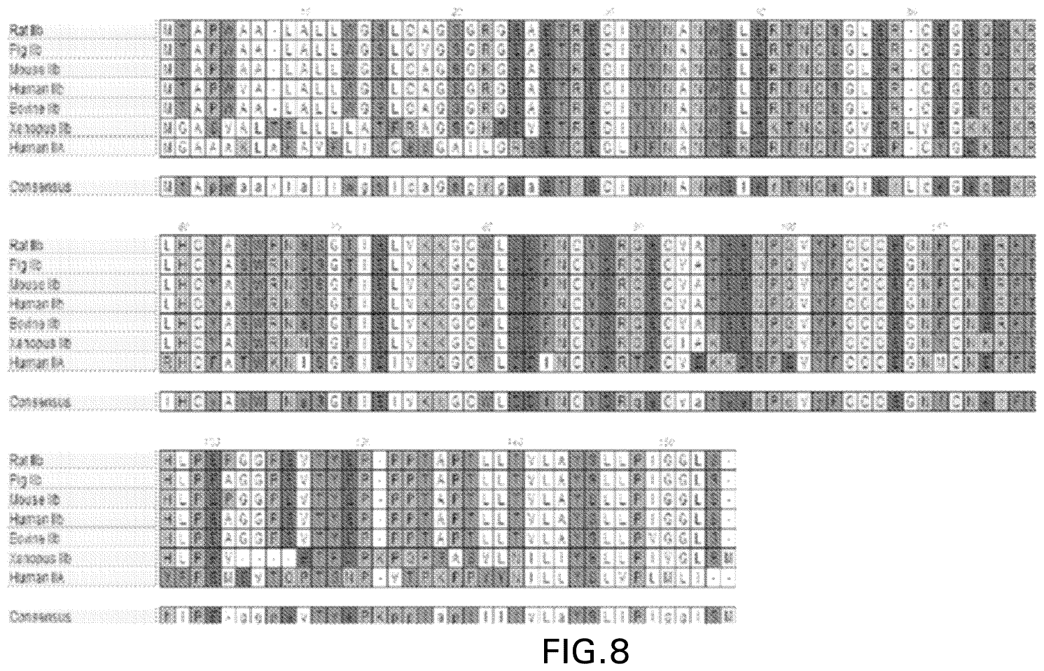

In addition, ActRIIB is well-conserved among vertebrates, with large stretches of the extracellular domain completely conserved. For example, FIG. 8 depicts a multi-sequence alignment of a human ActRIIB extracellular domain compared to various ActRIIB orthologs. Many of the ligands that bind to ActRIIB are also highly conserved. Accordingly, from these alignments, it is possible to predict key amino acid positions within the ligand-binding domain that are important for normal ActRIIB-ligand binding activities as well as to predict amino acid positions that are likely to be tolerant of substitution without significantly altering normal ActRIIB-ligand binding activities. Therefore, an active, human ActRIIB variant polypeptide useful in accordance with the presently disclosed methods may include one or more amino acids at corresponding positions from the sequence of another vertebrate ActRIIB, or may include a residue that is similar to that in the human or other vertebrate sequences. Without meaning to be limiting, the following examples illustrate this approach to defining an active ActRIIB variant. L46 in the human extracellular domain (SEQ ID NO: 104) is a valine in Xenopus ActRIIB (SEQ ID NO: 105), and so this position may be altered, and optionally may be altered to another hydrophobic residue, such as V, I or F, or a non-polar residue such as A. E52 in the human extracellular domain is a K in Xenopus, indicating that this site may be tolerant of a wide variety of changes, including polar residues, such as E, D, K, R, H, S, T, P, G, Y and probably A. T93 in the human extracellular domain is a K in Xenopus, indicating that a wide structural variation is tolerated at this position, with polar residues favored, such as S, K, R, E, D, H, G, P, G and Y. F108 in the human extracellular domain is a Y in Xenopus, and therefore Y or other hydrophobic group, such as I, V or L should be tolerated. E111 in the human extracellular domain is K in Xenopus, indicating that charged residues will be tolerated at this position, including D, R, K and H, as well as Q and N. R112 in the human extracellular domain is K in Xenopus, indicating that basic residues are tolerated at this position, including R and H. A at position 119 in the human extracellular domain is relatively poorly conserved, and appears as P in rodents and V in Xenopus, thus essentially any amino acid should be tolerated at this position.

Moreover, ActRII proteins have been characterized in the art in terms of structural and functional characteristics, particularly with respect to ligand binding [Attisano et al. (1992) Cell 68(1):97-108; Greenwald et al. (1999) Nature Structural Biology 6(1): 18-22; Allendorph et al. (2006) PNAS 103(20: 7643-7648; Thompson et al. (2003) The EMBO Journal 22(7): 1555-1566; as well as U.S. Pat. Nos. 7,709,605, 7,612,041, and 7,842,663]. In addition to the teachings herein, these references provide ample guidance for how to generate ActRIIB variants that retain one or more normal activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ActRIIB, as demarcated by the outermost of these conserved cysteines, corresponds to positions 29-109 of SEQ ID NO: 50 (ActRIIB precursor). Thus, the structurally less-ordered amino acids flanking these cysteine-demarcated core sequences can be truncated by about 1, 2, 3, 4, 5, 6, 7, 8, 9, 10, 11, 12, 13, 14, 15, 16, 17, 18, 19, 20, 21, 22, 23, 24, 25, 26, 27, or 28 residues at the N-terminus and/or by about 1, 2, 3, 4, 5, 6, 7, 8, 9, 10, 11, 12, 13, 14, 15, 16, 17, 18, 19, 20, 21, 22, 23, 24, or 25 residues at the C-terminus without necessarily altering ligand binding. Exemplary ActRIIB extracellular domains for N-terminal and/or C-terminal truncation include SEQ ID NOs: 51, 52, 54, 55, and 109.

Attisano et al. showed that a deletion of the proline knot at the C-terminus of the extracellular domain of ActRIIB reduced the affinity of the receptor for activin. An ActRIIB-Fc fusion protein containing amino acids 20-119 of present SEQ ID NO: 50, “ActRIIB(20-119)-Fc”, has reduced binding to GDF11 and activin relative to an ActRIIB(20-134)-Fc, which includes the proline knot region and the complete juxtamembrane domain (see, e.g., U.S. Pat. No. 7,842,663). However, an ActRIIB(20-129)-Fc protein retains similar, but somewhat reduced activity, relative to the wild-type, even though the proline knot region is disrupted.

Thus, ActRIIB extracellular domains that stop at amino acid 134, 133, 132, 131, 130 and 129 (with respect to SEQ ID NO: 50) are all expected to be active, but constructs stopping at 134 or 133 may be most active. Similarly, mutations at any of residues 129-134 (with respect to SEQ ID NO: 50) are not expected to alter ligand-binding affinity by large margins. In support of this, it is known in the art that mutations of P129 and P130 (with respect to SEQ ID NO: 50) do not substantially decrease ligand binding. Therefore, an ActRIIB polypeptide of the present disclosure may end as early as amino acid 109 (the final cysteine), however, forms ending at or between 109 and 119 (e.g., 109, 110, 111, 112, 113, 114, 115, 116, 117, 118, or 119) are expected to have reduced ligand binding. Amino acid 119 (with respect to present SEQ ID NO: 50) is poorly conserved and so is readily altered or truncated. ActRIIB polypeptides ending at 128 (with respect to SEQ ID NO: 50) or later should retain ligand-binding activity. ActRIIB polypeptides ending at or between 119 and 127 (e.g., 119, 120, 121, 122, 123, 124, 125, 126, or 127), with respect to SEQ ID NO: 50, will have an intermediate binding ability. Any of these forms may be desirable to use, depending on the clinical or experimental setting.

At the N-terminus of ActRIIB, it is expected that a protein beginning at amino acid 29 or before (with respect to SEQ ID NO: 50) will retain ligand-binding activity. Amino acid 29 represents the initial cysteine. An alanine-to-asparagine mutation at position 24 (with respect to SEQ ID NO: 50) introduces an N-linked glycosylation sequence without substantially affecting ligand binding [U.S. Pat. No. 7,842,663]. This confirms that mutations in the region between the signal cleavage peptide and the cysteine cross-linked region, corresponding to amino acids 20-29, are well tolerated. In particular, ActRIIB polypeptides beginning at position 20, 21, 22, 23, and 24 (with respect to SEQ ID NO: 50) should retain general ligand-biding activity, and ActRIIB polypeptides beginning at positions 25, 26, 27, 28, and 29 (with respect to SEQ ID NO: 50) are also expected to retain ligand-biding activity. It has been demonstrated, e.g., U.S. Pat. No. 7,842,663, that, surprisingly, an ActRIIB construct beginning at 22, 23, 24, or 25 will have the most activity.

Taken together, a general formula for an active portion (e.g., ligand-binding portion) of ActRIIB comprises amino acids 29-109 of SEQ ID NO: 50. Therefore ActRIIB polypeptides may, for example, comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIB beginning at a residue corresponding to any one of amino acids 20-29 (e.g., beginning at any one of amino acids 20, 21, 22, 23, 24, 25, 26, 27, 28, or 29) of SEQ ID NO: 50 and ending at a position corresponding to any one amino acids 109-134 (e.g., ending at any one of amino acids 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 50. Other examples include polypeptides that begin at a position from 20-29 (e.g., any one of positions 20, 21, 22, 23, 24, 25, 26, 27, 28, or 29) or 21-29 (e.g., any one of positions 21, 22, 23, 24, 25, 26, 27, 28, or 29) of SEQ ID NO: 50 and end at a position from 119-134 (e.g., any one of positions 119, 120, 121, 122, 123, 124, 125, 126, 127, 128, 129, 130, 131, 132, 133, or 134), 119-133 (e.g., any one of positions 119, 120, 121, 122, 123, 124, 125, 126, 127, 128, 129, 130, 131, 132, or 133), 129-134 (e.g., any one of positions 129, 130, 131, 132, 133, or 134), or 129-133 (e.g., any one of positions 129, 130, 131, 132, or 133) of SEQ ID NO: 50. Other examples include constructs that begin at a position from 20-24 (e.g., any one of positions 20, 21, 22, 23, or 24), 21-24 (e.g., any one of positions 21, 22, 23, or 24), or 22-25 (e.g., any one of positions 22, 22, 23, or 25) of SEQ ID NO: 50 and end at a position from 109-134 (e.g., any one of positions 109, 110, 111, 112, 113, 114, 115, 116, 117, 118, 119, 120, 121, 122, 123, 124, 125, 126, 127, 128, 129, 130, 131, 132, 133, or 134), 119-134 (e.g., any one of positions 119, 120, 121, 122, 123, 124, 125, 126, 127, 128, 129, 130, 131, 132, 133, or 134) or 129-134 (e.g., any one of positions 129, 130, 131, 132, 133, or 134) of SEQ ID NO: 50. Variants within these ranges are also contemplated, particularly those having at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 50.

The variations described herein may be combined in various ways. In some embodiments, ActRIIB variants comprise no more than 1, 2, 5, 6, 7, 8, 9, 10 or 15 conservative amino acid changes in the ligand-binding pocket, and zero, one, or more non-conservative alterations at positions 40, 53, 55, 74, 79 and/or 82 in the ligand-binding pocket. Sites outside the binding pocket, at which variability may be particularly well tolerated, include the amino and carboxy termini of the extracellular domain (as noted above), and positions 42-46 and 65-73 (with respect to SEQ ID NO: 50). An asparagine-to-alanine alteration at position 65 (N65A) actually improves ligand binding in the A64 background, and is thus expected to have no detrimental effect on ligand binding in the R64 background [U.S. Pat. No. 7,842,663]. This change probably eliminates glycosylation at N65 in the A64 background, thus demonstrating that a significant change in this region is likely to be tolerated. While an R64A change is poorly tolerated, R64K is well-tolerated, and thus another basic residue, such as H may be tolerated at position 64 [U.S. Pat. No. 7,842,663]. Additionally, the results of the mutagenesis program described in the art indicate that there are amino acid positions in ActRIIB that are often beneficial to conserve. With respect to SEQ ID NO: 50, these include position 80 (acidic or hydrophobic amino acid), position 78 (hydrophobic, and particularly tryptophan), position 37 (acidic, and particularly aspartic or glutamic acid), position 56 (basic amino acid), position 60 (hydrophobic amino acid, particularly phenylalanine or tyrosine). Thus, the disclosure provides a framework of amino acids that may be conserved in ActRIIB polypeptides. Other positions that may be desirable to conserve are as follows: position 52 (acidic amino acid), position 55 (basic amino acid), position 81 (acidic), 98 (polar or charged, particularly E, D, R or K), all with respect to SEQ ID NO: 50.

In some embodiments, ActRIIB polypeptides of the disclosure comprise the naturally occurring leucine at the position 79 with respect to SEQ ID NO: 50. In some embodiments, ActRIIB polypeptides of the disclosure comprise an acidic amino acid (e.g., a naturally occurring D or E amino acid residue or an artificial acidic amino acid) at the position 79 with respect to SEQ ID NO: 50. In alternative embodiments, ActRIIB polypeptides of the disclosure do not comprise an acidic amino acid (e.g., a naturally occurring D or E amino acid residue or an artificial acidic amino acid) at the position 79 with respect to SEQ ID NO: 50.

As described above, the disclosure provides TβRII or ActRIIB polypeptides sharing a specified degree of sequence identity or similarity to a naturally occurring TβRII or ActRIIB polypeptide. To determine the percent identity of two amino acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues at corresponding amino acid positions are then compared. When a position in the first sequence is occupied by the same amino acid residue as the corresponding position in the second sequence, then the molecules are identical at that position (as used herein amino acid “identity” is equivalent to amino acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity and similarity between two sequences can be accomplished using a mathematical algorithm (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

In one embodiment, the percent identity between two amino acid sequences is determined using the Needleman and Wunsch (J Mol. Biol. (48):444-453 (1970)) algorithm which has been incorporated into the GAP program in the GCG software package (available at http://www.gcg.com). In a specific embodiment, the following parameters are used in the GAP program: either a Blosum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (Devereux, J., et al., Nucleic Acids Res. 12(1):387 (1984)) (available at http://www.gcg.com). Exemplary parameters include using a NWSgapdna.CMP matrix and a gap weight of 40, 50, 60, 70, or 80 and a length weight of 1, 2, 3, 4, 5, or 6. Unless otherwise specified, percent identity between two amino acid sequences is to be determined using the GAP program using a Blosum 62 matrix, a GAP weight of 10 and a length weight of 3, and if such algorithm cannot compute the desired percent identity, a suitable alternative disclosed herein should be selected.

In another embodiment, the percent identity between two amino acid sequences is determined using the algorithm of E. Myers and W. Miller (CABIOS, 4:11-17 (1989)) which has been incorporated into the ALIGN program (version 2.0), using a PAM120 weight residue table, a gap length penalty of 12 and a gap penalty of 4.

Another embodiment for determining the best overall alignment between two amino acid sequences can be determined using the FASTDB computer program based on the algorithm of Brutlag et al. (Comp. App. Biosci., 6:237-245 (1990)). In a sequence alignment the query and subject sequences are both amino acid sequences. The result of said global sequence alignment is presented in terms of percent identity. In one embodiment, amino acid sequence identity is performed using the FASTDB computer program based on the algorithm of Brutlag et al. (Comp. App. Biosci., 6:237-245 (1990)). In a specific embodiment, parameters employed to calculate percent identity and similarity of an amino acid alignment comprise: Matrix=PAM 150, k-tuple=2, Mismatch Penalty=1, Joining Penalty=20, Randomization Group Length=0, Cutoff Score=1, Gap Penalty=5 and Gap Size Penalty=0.05.

Polypeptides of the disclosure (e.g., TβRII or ActRIIB polypeptides) may additionally include any of various leader sequences at the N-terminus. Such a sequence would allow the peptides to be expressed and targeted to the secretion pathway in a eukaryotic system. See, e.g., Ernst et al., U.S. Pat. No. 5,082,783 (1992). Alternatively, a native signal sequence (e.g., native TβRII or ActRIIB signal sequence) may be used to effect extrusion from the cell. Possible leader sequences include native leaders, tissue plasminogen activator (TPA) and honeybee mellitin (SEQ ID NOs. 22-24, respectively). Examples of TβRII-Fc and ActRIIB-Fc fusion proteins incorporating a TPA leader sequence include SEQ ID NOs: 11, 13, 15, 17, 82, 85, 88, and 91. Processing of signal peptides may vary depending on the leader sequence chosen, the cell type used and culture conditions, among other variables, and therefore actual N-terminal start sites for processed polypeptides may shift by 1, 2, 3, 4 or 5 amino acids in either the N-terminal or C-terminal direction. It will be understood by one of skill in the art that corresponding variants based on the long isoform of TβRII will include the 25-amino acid insertion along with a conservative Val-Ile substitution at the flanking position C-terminal to the insertion.

In certain embodiments, the present disclosure contemplates specific mutations of the polypeptides (e.g., TβRII or ActRIIB polypeptides) so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine (or asparagine-X-serine) (where “X” is any amino acid) which is specifically recognized by appropriate cellular glycosylation enzymes. The alteration may also be made by the addition of, or substitution by, one or more serine or threonine residues to the sequence of the wild-type polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. These methods are described in WO 87/05330 published Sep. 11, 1987, and in Aplin and Wriston (1981) CRC Crit. Rev. Biochem., pp. 259-306, incorporated by reference herein. Removal of one or more carbohydrate moieties present on a polypeptide may be accomplished chemically and/or enzymatically. Chemical deglycosylation may involve, for example, exposure of the polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Chemical deglycosylation is further described by Hakimuddin et al. (1987) Arch. Biochem. Biophys. 259:52 and by Edge et al. (1981) Anal. Biochem. 118:131. Enzymatic cleavage of carbohydrate moieties on polypeptides can be achieved by the use of a variety of endo- and exo-glycosidases as described by Thotakura et al. (1987) Meth. Enzymol. 138:350. The sequence of a polypeptide may be adjusted, as appropriate, depending on the type of expression system used, as mammalian, yeast, insect and plant cells may all introduce differing glycosylation patterns that can be affected by the amino acid sequence of the peptide. In general, polypeptides (e.g., TβRII or ActRIIB polypeptides) for use in humans will be expressed in a mammalian cell line that provides proper glycosylation, such as HEK293 or CHO cell lines, although other mammalian expression cell lines, yeast cell lines with engineered glycosylation enzymes, and insect cells are expected to be useful as well.

This disclosure further contemplates a method of generating mutants, particularly sets of combinatorial mutants of a polypeptide (e.g., TβRII or ActRIIB polypeptides as well as heteromultimers thereof), as well as truncation mutants; pools of combinatorial mutants are especially useful for identifying functional variant sequences. The purpose of screening such combinatorial libraries may be to generate, for example, polypeptide variants which can act as either agonists or antagonist, or alternatively, which possess novel activities all together. A variety of screening assays are provided below, and such assays may be used to evaluate variants. For example, a ActRIIB:TβRII heteromultimer comprising an ActRIIB and/or TβRII polypeptide variant may be screened for ability to bind to an AcRIIB or TβRII ligand, to prevent binding of an ActRIIB or TβRII ligand to an ActRIIB or TβRII polypeptide or to interfere with signaling caused by an ActRIIB or TβRII ligand.

Combinatorially-derived variants can be generated which have a selective or generally increased potency relative to a polypeptide (e.g., TβRII or ActRIIB polypeptides) comprising an extracellular domain of a naturally occurring polypeptide. Likewise, mutagenesis can give rise to variants which have serum half-lives dramatically different than the corresponding wild-type polypeptide. For example, the altered protein can be rendered either more stable or less stable to proteolytic degradation or other processes which result in destruction of, or otherwise elimination or inactivation of, a native TβRII polypeptide. Such variants, and the genes which encode them, can be utilized to alter TβRII polypeptide levels by modulating the half-life of the TβRII polypeptides. For instance, a short half-life can give rise to more transient biological effects and can allow tighter control of recombinant polypeptide levels within the patient. In an Fc fusion protein, mutations may be made in the linker (if any) and/or the Fc portion to alter the half-life of the protein.

A combinatorial library may be produced by way of a degenerate library of genes encoding a library of polypeptides which each include at least a portion of potential polypeptide (e.g., TβRII or ActRIIB polypeptides) sequences. For instance, a mixture of synthetic oligonucleotides can be enzymatically ligated into gene sequences such that the degenerate set of potential polypeptide nucleotide sequences are expressible as individual polypeptides, or alternatively, as a set of larger fusion proteins (e.g., for phage display).

There are many ways by which the library of potential polypeptide (e.g., TβRII or ActRIIB polypeptide) variants can be generated from a degenerate oligonucleotide sequence. Chemical synthesis of a degenerate gene sequence can be carried out in an automatic DNA synthesizer, and the synthetic genes then be ligated into an appropriate vector for expression. The synthesis of degenerate oligonucleotides is well known in the art (see for example, Narang, SA (1983) Tetrahedron 39:3; Itakura et al., (1981) Recombinant DNA, Proc. 3rd Cleveland Sympos. Macromolecules, ed. AG Walton, Amsterdam: Elsevier pp273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, polypeptide (e.g., TβRII or ActRIIB polypeptide) variants can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, NY; and Greener et al., (1994) Strategies in Mol Biol 7:32-34). Linker scanning mutagenesis, particularly in a combinatorial setting, is an attractive method for identifying truncated (bioactive) forms of polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of polypeptides (e.g., TβRII or ActRIIB polypeptides). The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Preferred assays include ligand binding assays and ligand-mediated cell signaling assays.

In certain embodiments, the polypeptides (e.g., TβRII or ActRIIB polypeptides) of the disclosure may further comprise post-translational modifications in addition to any that are naturally present in the native polypeptides. Such modifications include, but are not limited to, acetylation, carboxylation, glycosylation, phosphorylation, lipidation, pegylation (polyethylene glycol) and acylation. As a result, the modified polypeptides may contain non-amino acid elements, such as polyethylene glycols, lipids, mono- or poly-saccharides, and phosphates. Effects of such non-amino acid elements on the functionality of a polypeptide may be tested as described herein for other polypeptide variants. When a polypeptide is produced in cells by cleaving a nascent form of the polypeptide, post-translational processing may also be important for correct folding and/or function of the protein. Different cells (such as CHO, HeLa, MDCK, 293, WI38, NIH-3T3 or HEK-293) have specific cellular machinery and characteristic mechanisms for such post-translational activities and may be chosen to ensure the correct modification and processing of the polypeptides.

In certain aspects, the disclosure provides for fusion proteins (e.g., TβRII or ActRIIB fusion proteins), and in some embodiments, a first portion (e.g., a TβRII or ActRIIB polypeptide portion) is connected to a heterologous portion (e.g., Fc portion) by means of a linker. In some embodiments, the linkers are glycine and serine rich linkers. Other near neutral amino acids, such as, but not limited to, Thr, Asn, Pro and Ala, may also be used in the linker sequence. In some embodiments, the linker comprises various permutations of amino acid sequences containing Gly and Ser. In some embodiments, the linker is greater than 10 amino acids in length. In further embodiments, the linkers have a length of at least 12, 15, 20, 21, 25, 30, 35, 40, 45 or 50 amino acids. In some embodiments, the linker is less than 40, 35, 30, 25, 22 or 20 amino acids. In some embodiments, the linker is 10-50, 10-40, 10-30, 10-25, 10-21, 10-15, 10, 15-25, 17-22, 20, or 21 amino acids in length. In some preferred embodiments, the linker comprises the amino acid sequence GlyGlyGlyGlySer (GGGGS) (SEQ ID NO: 19), or repetitions thereof (GGGGS)n, where n≥2. In particular embodiments n≥3, or n=3-10. The application teaches the surprising finding that proteins comprising a TβRII portion and a heterologous portion fused together by means of a (GGGGS)₄ linker were associated with a stronger affinity for TGFβ1 and TGFβ3 as compared to a TβRII fusion protein where n<4. As such, in preferred embodiments, n≥4, or n=4-10. The application also teaches that proteins comprising (GGGGS)n linkers in which n>4 had similar inhibitory properties as proteins having the (GGGGS)₄ linker. As such, in some embodiments, n is not greater than 4 in a (GGGGS)n linker. In some embodiments, n=4-10, 4-9, 4-8, 4-7, 4-6, 4-5, 5-8, 5-7, or 5-6. In some embodiments, n=3, 4, 5, 6, or 7. In particular embodiments, n=4. In some embodiments, a linker comprising a (GGGGS)ₙ sequence also comprises an N-terminal threonine. In some embodiments, the linker is any one of the following:

(SEQ ID NO: 21)
GGGGSGGGGS
(SEQ ID NO: 4)
TGGGGSGGGGS
(SEQ ID NO: 5)
TGGGGSGGGGSGGGGS
(SEQ ID NO: 6)
TGGGGSGGGGSGGGGSGGGGS
(SEQ ID NO: 25)
TGGGGSGGGGSGGGGSGGGGSGGGGS
(SEQ ID NO: 26)
TGGGGSGGGGSGGGGSGGGGSGGGGSGGGGS
or
(SEQ ID NO: 40)
TGGGGSGGGGSGGGGSGGGGSGGGGSGGGGSGGGGS.

In some embodiments, the linker comprises the amino acid sequence of TGGGPKSCDK (SEQ ID NO: 7). In some embodiments, the linker is any one of SEQ ID NOs: 21, 4-7, 25-26 or 40 lacking the N-terminal threonine. In some embodiments, a linker may be rich in glycine (e.g., 2-10, 2-5, 2-4, 2-3 glycine residues) and may, for example, contain a single sequence of threonine/serine and glycines or repeating sequences of threonine/serine and/or glycines, e.g., GGG (SEQ ID NO: 63), GGGG (SEQ ID NO: 64), TGGGG (SEQ ID NO: 65), SGGGG (SEQ ID NO: 66), or SGGG (SEQ ID NO: 67) singlets, or repeats. In some embodiments, the linker does not comprise the amino acid sequence of SEQ ID NO: 26 or 40.

In some embodiments, the TβRII polypeptides comprise an amino acid sequence that is at least 80%, 85%, 90%, 92%, 94%, 95%, 97%, 99% or 100% identical to the amino acid sequence of any one of SEQ ID NOs: 94-100, or biologically active fragments thereof. In some embodiments, the TβRII polypeptides comprise an amino acid sequence that is at least 80%, 85%, 90%, 92%, 94%, 95%, 97%, 99% or 100% identical to the amino acid sequence of SEQ ID NO: 94, or biologically active fragments thereof. In some embodiments, the TβRII polypeptides comprise an amino acid sequence that is at least 80%, 85%, 90%, 92%, 94%, 95%, 97%, 99% or 100% identical to the amino acid sequence of SEQ ID NO: 98, or biologically active fragments thereof.

In some embodiments, the disclosure provides for fusion proteins comprising any of the TβRII polypeptides disclosed herein (e.g., a TβRII comprising the amino acid sequence of SEQ ID NO: 170) and any of the heterologous portions disclosed herein (e.g., any of the Fc portions disclosed herein). In some embodiments, the TβRII portion is N-terminal to the heterologous portion (e.g., Fc portion). In some embodiments, the TβRII portion is C-terminal to the heterologous portion (e.g., Fc portion). In some embodiments, the TβRII portion is C-terminal to the heterologous portion (e.g., Fc portion), and a linker is used to fuse the TβRII portion to the heterologous portion (e.g., Fc portion). In some embodiments, the linker is any of the linkers disclosed herein. In some embodiments, the linker comprises an amino acid sequence that is at least 70%, 75%, 80%, 85%, 90%, 91%, 92%, 93%, 94%, 95%, 97%, 98%, 99% or 100% identical to the amino acid sequence of SEQ ID NO: 165. In some embodiments, the heterologous portion is an Fc portion. In some embodiments, the Fc portion comprises an amino acid sequence that is at least 70%, 75%, 80%, 85%, 90%, 91%, 92%, 93%, 94%, 95%, 97%, 98%, 99% or 100% identical to SEQ ID NO: 73 (which may optionally lack the C-terminal lysine residue), or functional fragments thereof. In some embodiments, the TβRII portion comprises an amino acid sequence that is at least 70%, 75%, 80%, 85%, 90%, 91%, 92%, 93%, 94%, 95%, 97%, 98%, 99% or 100% identical to SEQ ID NO: 170, or functional fragments thereof. In some embodiments, the fusion protein comprises nine amino acids from CH1 (e.g., SNTKVDKRV-SEQ ID NO: 189), followed by a linker (e.g., TGGG), followed by an Fc portion (e.g., SEQ ID NO: 73), followed by a linker (e.g., SEQ ID NO: 165), followed by a TGFBRII polypeptide portion (e.g., SEQ ID NO: 170). In some embodiments, the fusion protein comprises an amino acid sequence that is at least 70%, 75%, 80%, 85%, 90%, 91%, 92%, 93%, 94%, 95%, 97%, 98%, 99% or 100% identical to SEQ ID NO: 193. In some embodiments, the fusion protein is part of a homodimer, wherein each subunit of the homodimer comprises an amino acid sequence that is at least 70%, 75%, 80%, 85%, 90%, 91%, 92%, 93%, 94%, 95%, 97%, 98%, 99% or 100% identical to SEQ ID NO: 193. In some embodiments, the fusion protein is a monomer comprising an amino acid sequence that is at least 70%, 75%, 80%, 85%, 90%, 91%, 92%, 93%, 94%, 95%, 97%, 98%, 99% or 100% identical to SEQ ID NO: 193. In some embodiments, the fusion protein comprises an amino acid sequence that is at least 70%, 75%, 80%, 85%, 90%, 91%, 92%, 93%, 94%, 95%, 97%, 98%, 99% or 100% identical to SEQ ID NO: 198. In some embodiments, the fusion protein is part of a homodimer, wherein each subunit of the homodimer comprises an amino acid sequence that is at least 70%, 75%, 80%, 85%, 90%, 91%, 92%, 93%, 94%, 95%, 97%, 98%, 99% or 100% identical to SEQ ID NO: 198. In some embodiments, the fusion protein is a monomer comprising an amino acid sequence that is at least 70%, 75%, 80%, 85%, 90%, 91%, 92%, 93%, 94%, 95%, 97%, 98%, 99% or 100% identical to SEQ ID NO: 198. In some embodiments, the Fc-TβRII fusion protein does not comprise any additional binding domains (e.g., does not comprise an ActRIIB portion, an antibody portion, an antigen-binding portion, or a follistatin portion). In some embodiments, the disclosure provides for a nucleic acid encoding any of the Fc-TβRII fusion proteins disclosed herein. In some embodiments, the nucleic acid comprises a nucleotide sequence that is at least 70%, 75%, 80%, 85%, 90%, 91%, 92%, 93%, 94%, 95%, 97%, 98%, 99% or 100% identical to SEQ ID NO: 194, or fragments thereof.

```
(SEQ ID NO: 198)
NTKVD KRVTGGGTHT CPPCPAPELL GGPSVFLFPP KPKDTLMISR
TPEVTCVVVD VSHEDPEVKF NWYVDGVEVH NAKTKPREEQ YNSTYRVVSV
LTVLHQDWLN GKEYKCKVSN KALPAPIEKT ISKAKGQPRE PQVCTLPPSR
EEMTKNQVSL SCAVKGFYPS DIAVEWESNG QPENNYKTTP PVLDSDGSFF
LVSKLTVDKS RWQQGNVFSC SVMHEALHNH YTQKSLSLSP GAGGGGSGGG
GSGGGGSGGG GSGTIPPHVQ KSDVEMEAQK DEITCPSCNR TAHPLRHINN
DMIVTDNNGA VKFPQLCKFC DVRFSTCDNQ KSCMSNCSIT SICEKPQEVC
VAVWRKNDEN ITLETVCHDP KLPYHDFILE DAASPKCIMK EKKKPGETFF
MCSCSSDECN DNIIFSEEYN TSNPD
(SEQ ID NO: 194)
      1 ATGGATGCAA TGAAGAGAGG GCTCTGCTGT GTGCTGCTGC TGTGTGGAGC
    51 AGTCTTCGTT TCGCCCGGCG CCAGCAACAC CAAGGTGGAC AAGAGAGTTA
  101 CCGGTGGTGG AACTCACACA TGCCCACCGT GCCCAGCACC TGAACTCCTG
  151 GGGGGACCGT CAGTCTTCCT CTTCCCCCCA AAACCCAAGG ACACCCTCAT
  201 GATCTCCCGG ACCCCTGAGG TCACATGCGT GGTGGTGGAC GTGAGCCACG
  251 AAGACCCTGA GGTCAAGTTC AACTGGTACG TGGACGGCGT GGAGGTGCAT
  301 AATGCCAAGA CAAAGCCGCG GGAGGAGCAG TACAACAGCA CGTACCGTGT
  351 GGTCAGCGTC CTCACCGTCC TGCACCAGGA CTGGCTGAAT GGCAAGGAGT
  401 ACAAGTGCAA GGTCTCCAAC AAAGCCCTCC CAGCCCCCAT CGAGAAAACC
  451 ATCTCCAAAG CCAAAGGGCA GCCCCGAGAA CCACAGGTGT GCACCCTGCC
  501 CCCATCCCGG GAGGAGATGA CCAAGAACCA GGTCAGCCTG TCCTGCGCCG
  551 TCAAAGGCTT CTATCCCAGC GACATCGCCG TGGAGTGGGA GAGCAATGGG
  601 CAGCCGGAGA ACAACTACAA GACCACGCCT CCCGTGCTGG ACTCCGACGG
  651 CTCCTTCTTC CTCGTGAGCA AGCTCACCGT GGACAAGAGC AGGTGGCAGC
  701 AGGGGAACGT CTTCTCATGC TCCGTGATGC ATGAGGCTCT GCACAACCAC
  751 TACACGCAGA AGAGCCTCTC CCTGTCTCCG GGTGCTGGTG GTGGAGGTTC
  801 TGGAGGTGGA GGAAGTGGTG GAGGTGGTTC TGGAGGTGGT GGTTCCGGAA
  851 CGATCCCACC GCACGTTCAG AAGTCGGATG TGGAAATGGA GGCCCAGAAA
  901 GATGAAATCA TCTGCCCCAG CTGTAATAGG ACTGCCCATC CACTGAGACA
  951 TATTAATAAC GACATGATAG TCACTGACAA CAACGGTGCA GTCAAGTTTC
1001 CACAACTGTG TAAATTTTGT GATGTGAGAT TTTCCACCTG TGACAACCAG
1051 AAATCCTGCA TGAGCAACTG CAGCATCACC TCCATCTGTG AGAAGCCACA
1101 GGAAGTCTGT GTGGCTGTAT GGAGAAAGAA TGACGAGAAC ATAACACTAG
1151 AGACAGTTTG CCATGACCCC AAGCTCCCCT ACCATGACTT TATTCTGGAA
1201 GATGCTGCTT CTCCAAAGTG CATTATGAAG GAAAAAAAAA AGCCTGGTGA
1251 GACTTTCTTC ATGTGTTCCT GTAGCTCTGA TGAGTGCAAT GACAACATCA
1301 TCTTCTCAGA AGAATATAAC ACCAGCAATC CTGACTGA
```

In certain aspects, functional variants or modified forms of the TβRII or ActRIIB polypeptides include fusion proteins having at least a portion of the TβRII or ActRIIB polypeptides and one or more heterologous portions. Well-known examples of such heterologous portions include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin. A heterologous portion may be selected so as to confer a desired property. For example, some heterologous portions are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners. As another example, a heterologous portion may be selected so as to facilitate detection of the TβRII or ActRIIB polypeptides. Examples of such detection domains include the various fluorescent proteins (e.g., GFP) as well as “epitope tags,” which are usually short peptide sequences for which a specific antibody is available. Well known epitope tags for which specific monoclonal antibodies are readily available include FLAG, influenza virus haemagglutinin (HA), and c-myc tags. In some cases, the heterologous portions have a protease cleavage site, such as for Factor Xa or Thrombin, which allows the relevant protease to partially digest the fusion proteins and thereby liberate the recombinant proteins therefrom. The liberated proteins can then be isolated from the heterologous portion by subsequent chromatographic separation. In certain preferred embodiments, a TβRII or ActRIIB polypeptide is fused with a domain that stabilizes the TβRII or ActRIIB polypeptide in vivo (a “stabilizer” domain). By “stabilizing” is meant anything that increases serum half life, regardless of whether this is because of decreased destruction, decreased clearance by the kidney, or other pharmacokinetic effect. Fusions with the Fc portion of an immunoglobulin are known to confer desirable pharmacokinetic properties on a wide range of proteins. Likewise, fusions to human serum albumin can confer desirable properties. Other types of heterologous portions that may be selected include multimerizing (e.g., dimerizing, tetramerizing) domains and functional domains.

It is understood that different elements of the fusion proteins may be arranged in any manner that is consistent with the desired functionality. For example, a TβRII or ActRIIB polypeptide may be placed C-terminal to a heterologous domain, or, alternatively, a heterologous domain may be placed C-terminal to a TβRII or ActRIIB polypeptide. The TβRII or ActRIIB polypeptide domain and the heterologous domain need not be adjacent in a fusion protein, and additional domains or amino acid sequences may be included C- or N-terminal to either domain or between the domains.

As used herein, the term “immunoglobulin Fc domain” or simply “Fc” is understood to mean the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In a preferred embodiment the immunoglobulin Fc region comprises at least an immunoglobulin hinge region a CH2 domain and a CH3 domain, and preferably lacks the CH1 domain. In some embodiments, the immunoglobulin Fc region is a human immunoglobulin Fc region.

In one embodiment, the class of immunoglobulin from which the heavy chain constant region is derived is IgG (Igγ) (γ subclasses 1, 2, 3, or 4).

An example of a native amino acid sequence that may be used for the Fc portion of human IgG1 (G1Fc) is shown below (SEQ ID NO: 58). Dotted underline indicates the hinge region, and solid underline indicates positions with naturally occurring variants. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 58. Naturally occurring variants in G1Fc would include E134D and M136L according to the numbering system used in SEQ ID NO: 58 (see Uniprot P01857).

| (SEQ ID NO: 58) | |
|---|---|
| 1 | 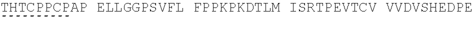 |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |

Optionally, the IgG1 Fc domain has one or more mutations at residues such as Asp-265, lysine 322, and Asn-434. In certain cases, the mutant IgG1 Fc domain having one or more of these mutations (e.g., Asp-265 mutation) has reduced ability of binding to the Fcγ receptor relative to a wild-type Fc domain. In other cases, the mutant Fc domain having one or more of these mutations (e.g., Asn-434 mutation) has increased ability of binding to the MHC class I-related Fc-receptor (FcRN) relative to a wild-type IgG1 Fc domain.

An example of a native amino acid sequence that may be used for the Fc portion of human IgG2 (G2Fc) is shown below (SEQ ID NO: 59). Dotted underline indicates the hinge region and double underline indicates positions where there are data base conflicts in the sequence (according to UniProt P01859). In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 59.

| (SEQ ID NO: 59) | |
|---|---|
| 1 | 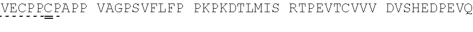 |
| 51 | FNWYVDGVEV HNAKTKPREE QFNSTFRVVS VLTVVHQDWL NGKEYKCKVS |
| 101 | NKGLPAPIEK TISKTKGQPR EPQVYTLPPS REEMTKNQVS LTCLVKGFYP |
| 151 | SDIAVEWESN GQPENNYKTT PPMLDSDGSF FLYSKLTVDK SRWQQGNVFS |
| 201 | CSVMHEALHN HYTQKSLSLS PGK |

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 60) contains a short hinge region consisting of a single 15-residue segment, whereas the second G3Fc sequence (SEQ ID NO: 61) contains a full-length hinge region. In each case, dotted underline indicates the hinge region, and solid underline indicates positions with naturally occurring variants according to UniProt P01859. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NOs: 60 or 61.

| (SEQ ID NO: 60) | |
|---|---|
| 1 | 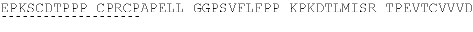 |
| 51 | VSHEDPEVQF KWYVDGVEVH NAKTKPREEQ YNSTFRVVSV LTVLHQDWLN |
| 101 | GKEYKCKVSN KALPAPIEKT ISKTKGQPRE PQVYTLPPSR EEMTKNQVSL |
| 151 | TCLVKGFYPS DIAVEWESSG QPENNYNTTP PMLDSDGSFF LYSKLTVDKS |
| 201 | RWQQGNIFSC SVMHEALHNR FTQKSLSLSP GK |
| (SEQ ID NO: 61) | |
| 1 | 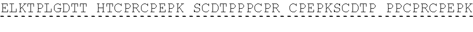 |
| 51 | 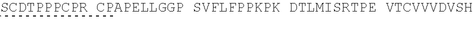 |
| 101 | EDPEVQFKWY VDGVEVHNAK TKPREEQYNS TFRVVSVLTV LHQDWLNGKE |
| 151 | YKCKVSNKAL PAPIEKTISK TKGQPREPQV YTLPPSREEM TKNQVSLTCL |
| 201 | VKGFYPSDIA VEWESSGQPE NNYNTTPPML DSDGSFFLYS KLTVDKSRWQ |
| 251 | QGNIFSCSVM HEALHNRFTQ KSLSLSPGK |

Naturally occurring variants in G3Fc (for example, see Uniprot P01860) include E68Q, P76L, E79Q, Y81F, D97N, N100D, T124A, S169N, S169del, F221Y when converted to the numbering system used in SEQ ID NO: 60, and the present disclosure provides fusion proteins comprising G3Fc domains containing one or more of these variations. In addition, the human immunoglobulin IgG3 gene (IGHG3) shows a structural polymorphism characterized by different hinge lengths [see Uniprot P01859]. Specifically, variant WIS is lacking most of the V region and all of the CH1 region. It has an extra interchain disulfide bond at position 7 in addition to the 11 normally present in the hinge region. Variant ZUC lacks most of the V region, all of the CH1 region, and part of the hinge. Variant OMM may represent an allelic form or another gamma chain subclass. The present disclosure provides additional fusion proteins comprising G3Fc domains containing one or more of these variants.

An example of a native amino acid sequence that may be used for the Fc portion of human IgG4 (G4Fc) is shown below (SEQ ID NO: 62). Dotted underline indicates the hinge region. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 62.

| (SEQ ID NO: 62) | |
|---|---|
| 1 | 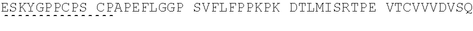 |
| 51 | EDPEVQFNWY VDGVEVHNAK TKPREEQFNS TYRVVSVLTV LHQDWLNGKE |
| 101 | YKCKVSNKGL PSSIEKTISK AKGQPREPQV YTLPPSQEEM TKNQVSLTCL |
| 151 | VKGFYPSDIA VEWESNGQPE NNYKTTPPVL DSDGSFFLYS RLTVDKSRWQ |
| 201 | EGNVFSCSVM HEALHNHYTQ KSLSLSLGK |

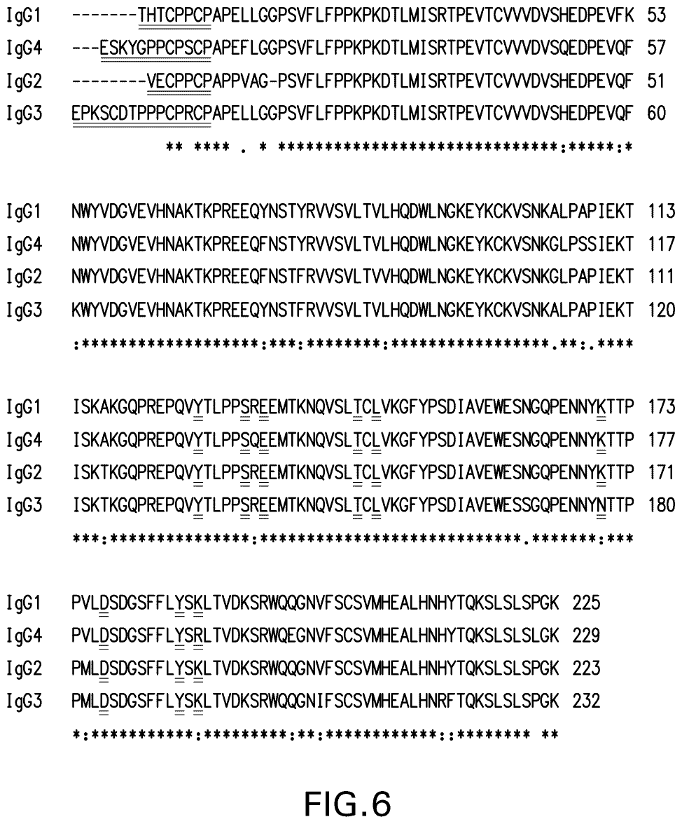

A variety of engineered mutations in the Fc domain are presented herein with respect to the G1Fc sequence (SEQ ID NO: 58), and analogous mutations in G2Fc, G3Fc, and G4Fc can be derived from their alignment with G1Fc in FIG. 6. Due to unequal hinge lengths, analogous Fc positions based on isotype alignment (FIG. 6) possess different amino acid numbers in SEQ ID NOs: 58, 59, 60, 61, and 62. It can also be appreciated that a given amino acid position in an immunoglobulin sequence consisting of hinge, C_{H}2, and C_{H}3 regions (e.g., SEQ ID NOs: 58, 59, 60, 61, and 62) will be identified by a different number than the same position when numbering encompasses the entire IgG1 heavy-chain constant domain (consisting of the C_{H}1, hinge, C_{H}2, and C_{H}3 regions) as in the Uniprot database.

Other classes of immunoglobulin, IgA (Ig□), IgD (Igδ), IgE (Igε) and IgM (Igμ), may be used. The choice of appropriate immunoglobulin heavy chain constant region is discussed in detail in U.S. Pat. Nos. 5,541,087 and 5,726,044. The choice of particular immunoglobulin heavy chain constant region sequences from certain immunoglobulin classes and subclasses to achieve a particular result is considered to be within the level of skill in the art. The portion of the DNA construct encoding the immunoglobulin Fc region preferably comprises at least a portion of a hinge domain, and preferably at least a portion of a CH₃ domain of Fc gamma or the homologous domains in any of IgA, IgD, IgE, or IgM.

Furthermore, it is contemplated that substitution or deletion of amino acids within the immunoglobulin heavy chain constant regions may be useful in the practice of the methods and compositions disclosed herein. One example would be to introduce amino acid substitutions in the upper CH2 region to create an Fc variant with reduced affinity for Fc receptors (Cole et al. (1997) J. Immunol. 159:3613).

For example, the application further provides Fc fusion proteins with engineered or variant Fc regions. Such antibodies and Fc fusion proteins may be useful, for example, in modulating effector functions, such as, antigen-dependent cytotoxicity (ADCC) and complement-dependent cytotoxicity (CDC). Additionally, the modifications may improve the stability of the antibodies and Fc fusion proteins. Amino acid sequence variants of the antibodies and Fc fusion proteins are prepared by introducing appropriate nucleotide changes into the DNA, or by peptide synthesis. Such variants include, for example, deletions from, and/or insertions into and/or substitutions of, residues within the amino acid sequences of the antibodies and Fc fusion proteins disclosed herein. Any combination of deletion, insertion, and substitution is made to arrive at the final construct, provided that the final construct possesses the desired characteristics. The amino acid changes also may alter post-translational processes of the antibodies and Fc fusion proteins, such as changing the number or position of glycosylation sites.

Antibodies and Fc fusion proteins with reduced effector function may be produced by introducing changes in the amino acid sequence, including, but are not limited to, the Ala-Ala mutation described by Bluestone et al. (see WO 94/28027 and WO 98/47531; also see Xu et al. 2000 Cell Immunol 200; 16-26). Thus, in certain embodiments, Fc fusion proteins of the disclosure with mutations within the constant region including the Ala-Ala mutation may be used to reduce or abolish effector function. According to these embodiments, antibodies and Fc fusion proteins may comprise a mutation to an alanine at position 234 or a mutation to an alanine at position 235, or a combination thereof. In one embodiment, the antibody or Fc fusion protein comprises an IgG4 framework, wherein the Ala-Ala mutation would describe a mutation(s) from phenylalanine to alanine at position 234 and/or a mutation from leucine to alanine at position 235. In another embodiment, the antibody or Fc fusion protein comprises an IgG1 framework, wherein the Ala-Ala mutation would describe a mutation(s) from leucine to alanine at position 234 and/or a mutation from leucine to alanine at position 235. The antibody or Fc fusion protein may alternatively or additionally carry other mutations, including the point mutation K322A in the CH2 domain (Hezareh et al. 2001 J Virol. 75: 12161-8).

In particular embodiments, the antibody or Fc fusion protein may be modified to either enhance or inhibit complement dependent cytotoxicity (CDC). Modulated CDC activity may be achieved by introducing one or more amino acid substitutions, insertions, or deletions in an Fc region (see, e.g., U.S. Pat. No. 6,194,551). Alternatively or additionally, cysteine residue(s) may be introduced in the Fc region, thereby allowing interchain disulfide bond formation in this region. The homodimeric antibody thus generated may have improved or reduced internalization capability and/or increased or decreased complement-mediated cell killing. See Caron et al., J. Exp Med. 176:1191-1195 (1992) and Shopes, B. J. Immunol. 148:2918-2922 (1992), WO99/51642, Duncan & Winter Nature 322: 738-40 (1988); U.S. Pat. Nos. 5,648,260; 5,624,821; and WO94/29351.

In certain preferred embodiments, heteromultimers described herein comprise at least one TβRII polypeptide associated, covalently or non-covalently, with at least one ActRIIB polypeptide. Preferably, polypeptides disclosed herein form heterodimeric complexes, although higher order heteromultimeric complexes are also included such as, but not limited to, heterotrimers, heterotetramers, and further oligomeric structures (see, e.g., FIGS. 9 and 10). In some embodiments, TβRII and/or ActRIIB polypeptides comprise at least one multimerization domain. As disclosed herein, the term “multimerization domain” refers to an amino acid or sequence of amino acids that promote covalent or non-covalent interaction between at least a first polypeptide and at least a second polypeptide. Polypeptides disclosed herein may be joined covalently or non-covalently to a multimerization domain. Preferably, a multimerization domain promotes interaction between a first polypeptide (e.g., a TβRII polypeptide) and a second polypeptide (e.g., an ActRIIB polypeptide) to promote heteromultimer formation (e.g., heterodimer formation), and optionally hinders or otherwise disfavors homomultimer formation (e.g., homodimer formation), thereby increasing the yield of desired heteromultimer (see, e.g., FIGS. 9 and 10).

Many methods known in the art can be used to generate ActRIIB:TβRII heteromultimers. For example, non-naturally occurring disulfide bonds may be constructed by replacing on a first polypeptide (e.g., a TβRII polypeptide) a naturally occurring amino acid with a free thiol-containing residue, such as cysteine, such that the free thiol interacts with another free thiol-containing residue on a second polypeptide (e.g., an ActRIIB polypeptide) such that a disulfide bond is formed between the first and second polypeptides. Additional examples of interactions to promote heteromultimer formation include, but are not limited to, ionic interactions such as described in Kjaergaard et al., WO2007147901; electrostatic steering effects such as described in Kannan et al., U.S. Pat. No. 8,592,562; coiled-coil interactions such as described in Christensen et al., U.S.20120302737; leucine zippers such as described in Pack & Plueckthun,(1992) Biochemistry 31: 1579-1584; and helix-turn-helix motifs such as described in Pack et al., (1993) Bio/Technology 11: 1271-1277. Linkage of the various segments may be obtained via, e.g., covalent binding such as by chemical cross-linking, peptide linkers, disulfide bridges, etc., or affinity interactions such as by avidin-biotin or leucine zipper technology.

In certain aspects, a multimerization domain may comprise one component of an interaction pair. In some embodiments, the polypeptides disclosed herein may form protein complexes comprising a first polypeptide covalently or non-covalently associated with a second polypeptide, wherein the first polypeptide comprises the amino acid sequence of a TβRII polypeptide and the amino acid sequence of a first member of an interaction pair; and the second polypeptide comprises the amino acid sequence of an ActRIIB polypeptide and the amino acid sequence of a second member of an interaction pair. The interaction pair may be any two polypeptide sequences that interact to form a complex, particularly a heterodimeric complex although operative embodiments may also employ an interaction pair that can form a homodimeric complex. One member of the interaction pair may be fused to a TβRII or ActRIIB polypeptide as described herein, including for example, a polypeptide sequence comprising, consisting essentially of, or consisting of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the sequence of any one of SEQ ID NOs: 18, 27, 28, 29, 30, 31, 32, 33, 34, 35, 36, 37, 38, 39, 51, 52, 54, 55, and 109. An interaction pair may be selected to confer an improved property/activity such as increased serum half-life, or to act as an adaptor on to which another moiety is attached to provide an improved property/activity. For example, a polyethylene glycol moiety may be attached to one or both components of an interaction pair to provide an improved property/activity such as improved serum half-life.

The first and second members of the interaction pair may be an asymmetric pair, meaning that the members of the pair preferentially associate with each other rather than self-associate. Accordingly, first and second members of an asymmetric interaction pair may associate to form a heterodimeric complex (see, e.g., FIGS. 9 and 10). Alternatively, the interaction pair may be unguided, meaning that the members of the pair may associate with each other or self-associate without substantial preference and thus may have the same or different amino acid sequences. Accordingly, first and second members of an unguided interaction pair may associate to form a homodimer complex or a heterodimeric complex. Optionally, the first member of the interaction pair (e.g., an asymmetric pair or an unguided interaction pair) associates covalently with the second member of the interaction pair. Optionally, the first member of the interaction pair (e.g., an asymmetric pair or an unguided interaction pair) associates non-covalently with the second member of the interaction pair.

A problem that arises in large-scale production of asymmetric immunoglobulin-based proteins from a single cell line is known as the “chain association issue”. As confronted prominently in the production of bispecific antibodies, the chain association issue concerns the challenge of efficiently producing a desired multichain protein from among the multiple combinations that inherently result when different heavy chains and/or light chains are produced in a single cell line [Klein et al (2012) mAbs 4:653-663]. This problem is most acute when two different heavy chains and two different light chains are produced in the same cell, in which case there are a total of 16 possible chain combinations (although some of these are identical) when only one is typically desired. Nevertheless, the same principle accounts for diminished yield of a desired multichain fusion protein that incorporates only two different (asymmetric) heavy chains.

Various methods are known in the art that increase desired pairing of Fc-containing fusion polypeptide chains in a single cell line to produce a preferred asymmetric fusion protein at acceptable yields [Klein et al (2012) mAbs 4:653-663; and Spiess et al (2015) Molecular Immunology 67(2A): 95-106]. Methods to obtain desired pairing of Fc-containing chains include, but are not limited to, charge-based pairing (electrostatic steering), “knobs-into-holes” steric pairing, SEEDbody pairing, and leucine zipper-based pairing [Ridgway et al (1996) Protein Eng 9:617-621; Merchant et al (1998) Nat Biotech 16:677-681; Davis et al (2010) Protein Eng Des Sel 23:195-202; Gunasekaran et al (2010); 285:19637-19646; Wranik et al (2012) J Biol Chem 287:43331-43339; U.S. Pat. No. 5,932,448; WO 1993/011162; WO 2009/089004, and WO 2011/034605]. As described herein, these methods may be used to generate ActRIIB-Fc:TβRII-Fc heteromultimer. See FIGS. 9 and 10.

For example, one means by which interaction between specific polypeptides may be promoted is by engineering protuberance-into-cavity (knob-into-holes) complementary regions such as described in Arathoon et al., U.S. Pat. No. 7,183,076 and Carter et al., U.S. Pat. No. 5,731,168. “Protuberances” are constructed by replacing small amino acid side chains from the interface of the first polypeptide (e.g., a first interaction pair) with larger side chains (e.g., tyrosine or tryptophan). Complementary “cavities” of identical or similar size to the protuberances are optionally created on the interface of the second polypeptide (e.g., a second interaction pair) by replacing large amino acid side chains with smaller ones (e.g., alanine or threonine). Where a suitably positioned and dimensioned protuberance or cavity exists at the interface of either the first or second polypeptide, it is only necessary to engineer a corresponding cavity or protuberance, respectively, at the adjacent interface.

At neutral pH (7.0), aspartic acid and glutamic acid are negatively charged and lysine, arginine, and histidine are positively charged. These charged residues can be used to promote heterodimer formation and at the same time hinder homodimer formation. Attractive interactions take place between opposite charges and repulsive interactions occur between like charges. In part, protein complexes disclosed herein make use of the attractive interactions for promoting heteromultimer formation (e.g., heterodimer formation), and optionally repulsive interactions for hindering homodimer formation (e.g., homodimer formation) by carrying out site directed mutagenesis of charged interface residues.

For example, the IgG1 CH3 domain interface comprises four unique charge residue pairs involved in domain-domain interactions: Asp356-Lys439′, Glu357-Lys370′, Lys392-Asp399′, and Asp399-Lys409′ [residue numbering in the second chain is indicated by (')]. It should be noted that the numbering scheme used here to designate residues in the IgG1 CH3 domain conforms to the EU numbering scheme of Kabat. Due to the 2-fold symmetry present in the CH3-CH3 domain interactions, each unique interaction will represented twice in the structure (e.g., Asp-399-Lys409′ and Lys409-Asp399′). In the wild-type sequence, K409-D399′ favors both heterodimer and homodimer formation. A single mutation switching the charge polarity (e.g., K409E; positive to negative charge) in the first chain leads to unfavorable interactions for the formation of the first chain homodimer. The unfavorable interactions arise due to the repulsive interactions occurring between the same charges (negative-negative; K409E-D399′ and D399-K409E′). A similar mutation switching the charge polarity (D399K′; negative to positive) in the second chain leads to unfavorable interactions (K409′-D399K′ and D399K-K409′) for the second chain homodimer formation. But, at the same time, these two mutations (K409E and D399K′) lead to favorable interactions (K409E-D399K′ and D399-K409′) for the heterodimer formation.

The electrostatic steering effect on heterodimer formation and homodimer discouragement can be further enhanced by mutation of additional charge residues which may or may not be paired with an oppositely charged residue in the second chain including, for example, Arg355 and Lys360. The table below lists possible charge change mutations that can be used, alone or in combination, to enhance ActRIIB:TβRII heteromultimer formation.

Examples of Pair-Wise Charged Residue Mutations
to Enhance Heterodimer Formation

| | | Interacting | Corresponding |
|---|---|---|---|
| Position in | Mutation in | position in | mutation in |
| first chain | first chain | second chain | second chain |
| Lys409 | Asp or Glu | Asp399′ | Lys, Arg, or His |
| Lys392 | Asp or Glu | Asp399′ | Lys, Arg, or His |
| Lys439 | Asp or Glu | Asp356′ | Lys, Arg, or His |
| Lys370 | Asp or Glu | Glu357′ | Lys, Arg, or His |
| Asp399 | Lys, Arg, or His | Lys409′ | Asp or Glu |
| Asp399 | Lys, Arg, or His | Lys392′ | Asp or Glu |
| Asp356 | Lys, Arg, or His | Lys439′ | Asp or Glu |
| Glu357 | Lys, Arg, or His | Lys370′ | Asp or Glu |

In some embodiments, one or more residues that make up the CH3-CH3 interface in a fusion protein of the instant application are replaced with a charged amino acid such that the interaction becomes electrostatically unfavorable. For example, a positive-charged amino acid in the interface (e.g., a lysine, arginine, or histidine) is replaced with a negatively charged amino acid (e.g., aspartic acid or glutamic acid). Alternatively, or in combination with the forgoing substitution, a negative-charged amino acid in the interface is replaced with a positive-charged amino acid. In certain embodiments, the amino acid is replaced with a non-naturally occurring amino acid having the desired charge characteristic. It should be noted that mutating negatively charged residues (Asp or Glu) to His will lead to increase in side chain volume, which may cause steric issues. Furthermore, His proton donor- and acceptor-form depends on the localized environment. These issues should be taken into consideration with the design strategy. Because the interface residues are highly conserved in human and mouse IgG subclasses, electrostatic steering effects disclosed herein can be applied to human and mouse IgG1, IgG2, IgG3, and IgG4. This strategy can also be extended to modifying uncharged residues to charged residues at the CH3 domain interface.

In part, the disclosure provides desired pairing of asymmetric Fc-containing polypeptide chains using Fc sequences engineered to be complementary on the basis of charge pairing (electrostatic steering). One of a pair of Fc sequences with electrostatic complementarity can be arbitrarily fused to the TβRII or ActRIIB polypeptide of the construct, with or without an optional linker, to generate an ActRIIB:TβRII heteromultimer. This single chain can be coexpressed in a cell of choice along with the Fc sequence complementary to the first Fc to favor generation of the desired multichain construct (e.g., ActRIIB:TβRII heteromultimer). In this example based on electrostatic steering, SEQ ID NO: 68 [human G1Fc(E356K/D399K)] and SEQ ID NO: 69 [human G1Fc(K392D/K409D)] are examples of complementary Fc sequences in which the engineered amino acid substitutions are double underlined, and the TGFβ superfamily type I or type II receptor polypeptide of the construct can be fused to either SEQ ID NO: 68 or SEQ ID NO: 69, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 6) will generate complementary Fc pairs which may be used instead of the complementary hG1Fc pair below (SEQ ID NOs: 68 and 69).

| (SEQ ID NO: 68) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSRKEMTKNQ VSLTCLVKGF |
| 151 | YPSDIAVEWE SNGQPENNYK TTPPVLKSDG SFFLYSKLTV DKSRWQQGNV |
| 201 | FSCSVMHEAL HNHYTQKSLS LSPGK |
| (SEQ ID NO: 69) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYD TTPPVLDSDG SFFLYSDLTV DKSRWQQGNV |
| 201 | FSCSVMHEAL HNHYTQKSLS LSPGK |

In part, the disclosure provides desired pairing of asymmetric Fc-containing polypeptide chains using Fc sequences engineered for steric complementarity. In part, the disclosure provides knobs-into-holes pairing as an example of steric complementarity. One of a pair of Fc sequences with steric complementarity can be arbitrarily fused to the TβRII or ActRIIB polypeptide of the construct, with or without an optional linker, to generate an ActRIIB:TβRII heteromultimer. This single chain can be co-expressed in a cell of choice along with the Fc sequence complementary to the first Fc to favor generation of the desired multi-chain construct. In this example based on knobs-into-holes pairing, SEQ ID NO: 70 [human G1Fc(T144Y)] and SEQ ID NO: 71 [human G1Fc(Y185T)] are examples of complementary Fc sequences in which the engineered amino acid substitutions are double underlined, and the TβRII or ActRIIB polypeptide of the construct can be fused to either SEQ ID NO: 70 or SEQ ID NO: 71, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 6) will generate complementary Fc pairs which may be used instead of the complementary hG1Fc pair below (SEQ ID NOs: 70 and 71).

| | (SEQ ID NO: 70) |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLYCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |
| | (SEQ ID NO: 71) |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLTSKLTV DKSRWQQGNV |
| 201 | FSCSVMHEAL HNHYTQKSLS LSPGK |

An example of Fc complementarity based on knobs-into-holes pairing combined with an engineered disulfide bond is disclosed in SEQ ID NO: 72 [hG1Fc(S132C/T144W)] and SEQ ID NO: 73 [hG1Fc(Y127C/T144S/L146A/Y185V)]. The engineered amino acid substitutions in these sequences are double underlined, and the TGFβ superfamily type I or type II polypeptide of the construct can be fused to either SEQ ID NO: 72 or SEQ ID NO: 73, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 6) will generate complementary Fc pairs which may be used instead of the complementary hG1Fc pair below (SEQ ID NOs: 72 and 73).

| (SEQ ID NO: 72) | |
|---|---|
| 1 | THTCPPCPAP ELLGGPSVFL FPPKPKDTLM ISRTPEVTCV WDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PCREEMTKNQ VSLWCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |
| (SEQ ID NO: 73) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVCTLP PSREEMTKNQ VSLSCAVKGF |
| 151 | YPSDIAVEWE SNGQPENNYK TTPPVLDSDG SFFLVSKLTV DKSRWQQGNV |
| 201 | FSCSVMHEAL HNHYTQKSLS LSPGK |

In part, the disclosure provides desired pairing of asymmetric Fc-containing polypeptide chains using Fc sequences engineered to generate interdigitating β-strand segments of human IgG and IgA C_{H}3 domains. Such methods include the use of strand-exchange engineered domain (SEED) C_{H}3 heterodimers allowing the formation of SEEDbody fusion proteins [Davis et al. (2010) Protein Eng Design Sel 23:195-202]. One of a pair of Fc sequences with SEEDbody complementarity can be arbitrarily fused to the TβRII or ActIIB of the construct, with or without an optional linker, to generate a TβRII or ActRIIB fusion polypeptide. This single chain can be co-expressed in a cell of choice along with the Fc sequence complementary to the first Fc to favor generation of the desired multi-chain construct. In this example based on SEEDbody (Sb) pairing, SEQ ID NO: 74 [hG1Fc(Sb_{AG})] and SEQ ID NO: 75 [hG1Fc(Sb_{GA})] are examples of complementary IgG Fc sequences in which the engineered amino acid substitutions from IgA Fc are double underlined, and the TβRII or ActRIIB polypeptide of the construct can be fused to either SEQ ID NO: 74 or SEQ ID NO: 75, but not both. Given the high degree of amino acid sequence identity between native hG1Fc, native hG2Fc, native hG3Fc, and native hG4Fc, it can be appreciated that amino acid substitutions at corresponding positions in hG1Fc, hG2Fc, hG3Fc, or hG4Fc (see FIG. 6) will generate an Fc monomer which may be used in the complementary IgG-IgA pair below (SEQ ID NOs: 74 and 75).

| (SEQ ID NO: 74 | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PFRPEVHLLP PSREEMTKNQ VSLTCLARGF |
| 151 | YPKDIAVEWE SNGQPENNYK TTPSRQEPSQ GTTTFAVTSK LTVDKSRWQQ |
| 201 | GNVFSCSVMH EALHNHYTQK TISLSPGK |
| (SEQ ID NO: 75) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PPSEELALNE LVTLTCLVKG |
| 151 | FYPSDIAVEW ESNGQELPRE KYLTWAPVLD SDGSFFLYSI LRVAAEDWKK |
| 201 | GDTFSCSVMH EALHNHYTQK SLDRSPGK |

In part, the disclosure provides desired pairing of asymmetric Fc-containing polypeptide chains with a cleavable leucine zipper domain attached at the C-terminus of the Fc C_{H}3 domains. Attachment of a leucine zipper is sufficient to cause preferential assembly of heterodimeric antibody heavy chains [Wranik et al (2012) J Biol Chem 287:43331-43339]. As disclosed herein, one of a pair of Fc sequences attached to a leucine zipper-forming strand can be arbitrarily fused to the TβRII or ActRIIB polypeptide of the construct, with or without an optional linker, to generate a TβRII or ActRIIB fusion polypeptide. This single chain can be co-expressed in a cell of choice along with the Fc sequence attached to a complementary leucine zipper-forming strand to favor generation of the desired multi-chain construct. Proteolytic digestion of the construct with the bacterial endoproteinase Lys-C post purification can release the leucine zipper domain, resulting in an Fc construct whose structure is identical to that of native Fc. In this example based on leucine zipper pairing, SEQ ID NO: 76 [hG1Fc-Ap1 (acidic)] and SEQ ID NO: 77 [hG1Fc-Bp1 (basic)] are examples of complementary IgG Fc sequences in which the engineered complimentary leucine zipper sequences are underlined, and the TβRII or ActRIIB polypeptide of the construct can be fused to either SEQ ID NO: 76 or SEQ ID NO: 77, but not both. Given the high degree of amino acid sequence identity between native hG1Fc, native hG2Fc, native hG3Fc, and native hG4Fc, it can be appreciated that leucine zipper-forming sequences attached, with or without an optional linker, to hG1Fc, hG2Fc, hG3Fc, or hG4Fc (see FIG. 6) will generate an Fc monomer which may be used in the complementary leucine zipper-forming pair below (SEQ ID NOs: 76 and 77).

| (SEQ ID NO: 76) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGKGGSAQ LEKELQALEK ENAQLEWELQ |
| 251 | ALEKELAQGA T |
| (SEQ ID NO: 77) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGKGGSAQ LKKKLQALKK KNAQLKWKLQ |
| 251 | ALKKKLAQGA T |

In certain aspects, the disclosure relates to TβRII polypeptides (e.g., TβRII-Fc fusion proteins) comprising one or more amino acid modifications that alter the isoelectric point (pI) of the TβRII polypeptide and/or ActRIIB polypeptides (e.g., ActRIIB-Fc fusion proteins) comprising one or more amino acid modifications that alter the isoelectric point of the ActRIIB polypeptide. In some embodiments, one or more candidate domains that have a pI value higher than about 5.0, 5.5, 6.0, 6.5, 7.0, 7.5, 8.0, 8.5, or 9.0 are selected for construction of the full multidomain protein. In other embodiments, one or more candidate domains that have a pI value less than about 9.0, 8.5, 8.0, 7.5, 7.0, 6.5, 6.0, 5.5, or 5.0 are selected for construction of the full multidomain protein. It will be understood by one skilled in the art that a single protein will have multiple charge forms. Without wishing to be bound by any particular theory, the charge of a protein can be modified by a number of different mechanisms including but not limited to, amino acid substitution, cationization, deamination, carboxyl-terminal amino acid heterogeneity, phosphorylation and glycosylation.

The pI of a protein may be determined by a variety of methods including but not limited to, isoelectric focusing and various computer algorithms (see for example Bjellqvist et al., 1993, Electrophoresis 14:1023). In one embodiment, pI is determined using a Pharmacia Biotech Multiphor 2 electrophoresis system with a multi temp refrigerated bath recirculation unit and an EPS 3501 XL power supply. Pre-cast ampholine gels (e.g., Amersham Biosciences, pI range 2.5-10) are loaded with protein samples. Broad range pI marker standards (e.g., Amersham, pI range 3-10, 8.mu·L) are used to determine relative pI for the proteins. Electrophoresis may be performed, for example, at 1500 V, 50 mA for 105 minutes. The gel is fixed using, for example, a Sigma fixing solution (5×) diluted with purified water to 1× Staining is performed, for example, overnight at room temperature using Simply Blue stain (Invitrogen). Destaining is carried out, for example, with a solution that consisted of 25% ethanol, 8% acetic acid and 67% purified water. Isoelectric points are determined using, for example, a Bio-Rad Densitometer relative to calibration curves of the standards. The one or more metrics may further include metrics characterizing stability of the domain under one or more different conditions selected from the group consisting of different pH values, different temperatures, different shear stresses, and different freeze/thaw cycles.

In part, the disclosure provides desired pairing of asymmetric Fc-containing polypeptide chains by methods described above in combination with additional mutations in the Fc domain which facilitate purification of the desired heteromeric species. An example is complementarity of Fc domains based on knobs-into-holes pairing combined with an engineered disulfide bond, as disclosed in SEQ ID NOs: 72-73, plus additional substitution of two negatively charged amino acids (aspartic acid or glutamic acid) in one Fc-containing polypeptide chain and two positively charged amino acids (e.g., arginine) in the complementary Fc-containing polypeptide chain (SEQ ID NOs: 78-79). These four amino acid substitutions facilitate selective purification of the desired heteromeric fusion protein from a heterogeneous polypeptide mixture based on differences in isoelectric point or net molecular charge. The engineered amino acid substitutions in these sequences are double underlined below, and the TβRII or ActRIIB polypeptide of the construct can be fused to either SEQ ID NO: 78 or SEQ ID NO: 79, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 6) will generate complementary Fc pairs which may be used instead of the complementary hG1Fc pair below (SEQ ID NOs: 78-79).

| (SEQ ID NO: 78) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PCREEMTENQ VSLWCLVKGF |
| 151 | YPSDIAVEWE SNGQPENNYK TTPPVLDSDG SFFLYSKLTV DKSRWQQGNV |
| 201 | FSCSVMHEAL HNHYTQDSLS LSPGK |
| (SEQ ID NO: 79) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVCTLP PSREEMTKNQ VSLSCAVKGF |
| 151 | YPSDIAVEWE SRGQPENNYK TTPPVLDSRG SFFLVSKLTV DKSRWQQGNV |
| 201 | FSCSVMHEAL HNHYTQKSLS LSPGK |

Another example involves complementarity of Fc domains based on knobs-into-holes pairing combined with an engineered disulfide bond, as disclosed in SEQ ID NOs: 72-73, plus a histidine-to-arginine substitution at position 213 in one Fc-containing polypeptide chain (SEQ ID NO: 80). This substitution (denoted H435R in the numbering system of Kabat et al.) facilitates separation of desired heteromer from undesirable homodimer based on differences in affinity for protein A. The engineered amino acid substitution is indicated by double underline, and the TβRII or ActRIIB polypeptide of the construct can be fused to either SEQ ID NO: 80 or SEQ ID NO: 73, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see Figure: 6) will generate complementary Fc pairs which may be used instead of the complementary hG1Fc pair of SEQ ID NO: 80 (below) and SEQ ID NO: 73.

(SEQ ID NO: 80)

| | |
|---|---|
| 1 | THTCPPCPAP ELLGGPSVFL FPPKPKDTLM |
| | ISRTPEVTCV VVDVSHEDPE |
| 51 | VKFNWYVDGV EVHNAKTKPR EEQYNSTYRV |
| | VSVLTVLHQD WLNGKEYKCK |
| 101 | VSNKALPAPI EKTISKAKGQ PREPQVYTLP |
| | PCREEMTKNQ VSLWCLVKGF |
| 151 | YPSDIAVEWE SNGQPENNYK TTPPVLDSDG |
| | SFFLYSKLTV DKSRWQQGNV |
| 201 | FSCSVMHEAL HNRYTQKSLS LSPGK |

As described above, various methods are known in the art that increase desired pairing of Fc-containing fusion polypeptide chains in a single cell line to produce a preferred asymmetric fusion protein at acceptable yields [Klein et al (2012) mAbs 4:653-663; and Spiess et al (2015) Molecular Immunology 67(2A): 95-106]. In addition, ActRIIB:TβRII heteromultimers may be generated using a combination of heavy and light chain fusion proteins comprising either an TβRII or ActRIIB polypeptide. For example, in some embodiments, a TβRII polypeptide may be fused, with or without a linker domain, to an immunoglobulin heavy chain (IgG1, IgG2, IgG3, IgG4, IgM, IgA1, or IgA2) that comprises at least a portion of the C_{H}1 domain. Similarly, an ActRIIB polypeptide may be fused, with or without a linker domain, to an immunoglobulin light chain (kappa or lambda) that comprises at least a portion of the light chain constant domain (C_{L}). In alternative embodiments, an ActRIIB polypeptide may be fused, with or without a linker domain, to an immunoglobulin heavy chain (IgG1, IgG2, IgG3, IgG4, IgM, IgA1, or IgA2) that comprises at least a portion of the C_{H}1 domain, and an TβRII polypeptide may be fused, with or without a linker domain, to an immunoglobulin light chain (kappa or lambda) that comprises at least a portion of the light chain constant domain (C_{L}). This design takes advantage of the natural ability of the heavy chains to heterodimerize with light chains. In particular, heterodimerization of a heavy and light chain occurs between the C_{H}1 with the C_{L}, which is generally stabilized by covalent linking of the two domains via a disulfide bridge. Constructs employing the full-length heavy chain, or at least a portion of the heavy chain comprising the hinge region, could give rise to antibody-like molecules comprising two “light chains” and two “heavy chains”. See FIG. 10. A potential advantage of this design is that it may more closely mimic the naturally occurring TβRII-ligand-ActRIIB complex and may display higher affinity for the ligand than comparable single heterodimers. In some embodiments, this design may be modified by incorporating various heavy chain truncations including, for example, truncations that comprise the C_{H}1 domain and some or all of the hinge domain (giving rise to F(ab′)₂-like molecules) as well as truncations that only comprise the C_{H}1 domain or a fragment thereof (giving rise to Fab-like molecules). See FIG. 10G. Various methods for designing such heteromultimer constructs are described in US 2009/0010879, Klein et al [(2012) mAbs 4:653-663], and Spiess et al [(2015) Molecular Immunology 67(2A): 95-106] the contents of which are incorporated in their entirety herein.

In some embodiments, it is desirable to generate antibody-like ActRIIB:TβRII heterodimers comprising at least one branch of the complex comprising an TβRII-C_{L}:ActRIIB-C_{H}1 heterodimer pair and at least a second branch comprising an ActRIIB-C_{L}:TβRII-C_{H}1 heterodimer pair. See, e.g., FIG. 10B. Such heterodimer complexes can be generated, for example, using combinations of heavy chain and light chain asymmetrical pairing technologies [Spiess et al (2015) Molecular Immunology 67(2A): 95-106]. For example, in CrossMab technology, [Schaefer et al (2011). Proc. Natl. Acad. Sci. U.S.A. 108: 11187-11192] light chain mispairing is overcome using domain crossovers and heavy chains heterodimerized using knobs-into-holes [Merchant et al (1998) Nat. Biotechnol. 16: 677-681]. For the domain crossovers either the variable domains or the constant domains are swapped between light and heavy chains to create two asymmetric Fab arms that drive cognate light chain pairing while preserving the structural and functional integrity of the variable domain [Fenn et al (2013) PLoS ONE 8: e61953]. An alternative approach for overcoming light chain mispairing is designing heavy and light chains with orthogonal Fab inter-faces [Lewis (2014) Nat. Biotechnol. 32: 191-198]. This has been accomplished by computational modeling [Das et al (2008) Annu. Rev. Biochem.77: 363-382] in combination with X-ray crystallography to identify mutations at the V_{H}/V_{L} and C_{H}1/C_{L} interfaces. For the heterodimers generated using this methodology, it may be necessary to engineer mutations into both V_{H}/V_{L} and C_{H}1/C_{L} interfaces to minimize heavy/light chain mispairing. The designed orthogonal Fab interface may be used in conjunction with a heavy chain heterodimerization strategy to facilitate efficient IgG production in a single host cell. Electrostatic steering may also be used to generate orthogonal Fab interfaces to facilitate the construction of such heterodimers. Peptide linkers may be used to ensure cognate pairing of light and heavy chains in a format known as “LUZ-Y” [Wranik et al (2012) J. Biol. Chem. 287: 43331-43339], wherein heavy chain heterodimerization is accomplished using leucine zippers which may be subsequently removed by proteolysis in vitro.

In some embodiments, the disclosure provides for TβRII polypeptides fusion proteins, as well as ActRIIB:TβRII heteromultimers comprising the same, comprising an amino acid sequence that is at least 70%, 75%, 80%, 85%, 90%, 91%, 92%, 93%, 94%, 95%, 96%, 97%, 98%, or 99% identical to the amino acid sequence of any one of SEQ ID NOs: 11, 13, 15, 17, 18, 27, 85, 87, 91, and 93 or biologically active fragments thereof. In some embodiments, the TβRII polypeptides fusion proteins, as well as ActRIIB:TβRII heteromultimers comprising the same, comprise an amino acid sequence that is at least 70%, 75%, 80%, 85%, 90%, 91%, 92%, 93%, 94%, 95%, 96%, 97%, 98%, or 99% identical to the amino acid sequence of any one of SEQ ID NOs: 85, 87, 91, and 93, or biologically active fragments thereof. In some embodiments, the TβRII polypeptides fusion proteins, as well as ActRIIB:TβRII heteromultimers comprising the same, comprise an amino acid sequence that is at least 70%, 75%, 80%, 85%, 90%, 91%, 92%, 93%, 94%, 95%, 96%, 97%, 98%, or 99% identical to the amino acid sequence of any one of SEQ ID NO: 87, or a biologically active fragment thereof. In some embodiments, the TβRII polypeptides fusion protein, as well as ActRIIB:TβRII heteromultimers comprising the same, comprises an amino acid sequence that is at least 70%, 75%, 80%, 85%, 90%, 91%, 92%, 93%, 94%, 95%, 96%, 97%, 98%, or 99% identical to the amino acid sequence of SEQ ID NO: 93, or a biologically active fragment thereof.

In some embodiments, the disclosure provides for ActRIIB polypeptides fusion proteins, as well as ActRIIB:TβRII heteromultimers comprising the same, comprising an amino acid sequence that is at least 70%, 75%, 80%, 85%, 90%, 91%, 92%, 93%, 94%, 95%, 96%, 97%, 98%, or 99% identical to the amino acid sequence of any one of SEQ ID NOs: 51, 52, 54, 55, 82, 84, 88, 90, and 109 or biologically active fragments thereof. In some embodiments, the ActRIIB polypeptides fusion proteins, as well as ActRIIB:TβRII heteromultimers comprising the same, comprise an amino acid sequence that is at least 70%, 75%, 80%, 85%, 90%, 91%, 92%, 93%, 94%, 95%, 96%, 97%, 98%, or 99% identical to the amino acid sequence of any one of SEQ ID NOs: 82, 84, 88, and 90, or biologically active fragments thereof. In some embodiments, the ActRIIB polypeptides fusion proteins, as well as ActRIIB:TβRII heteromultimers comprising the same, comprise an amino acid sequence that is at least 70%, 75%, 80%, 85%, 90%, 91%, 92%, 93%, 94%, 95%, 96%, 97%, 98%, or 99% identical to the amino acid sequence of any one of SEQ ID NO: 84, or a biologically active fragment thereof. In some embodiments, the ActRIIB polypeptides fusion protein, as well as ActRIIB:TβRII heteromultimers comprising the same, comprises an amino acid sequence that is at least 70%, 75%, 80%, 85%, 90%, 91%, 92%, 93%, 94%, 95%, 96%, 97%, 98%, or 99% identical to the amino acid sequence of SEQ ID NO: 90, or a biologically active fragment thereof.

In some embodiments, the TβRII fusion proteins described herein have improved binding affinity for TGFβ1 and TGFβ3. In some embodiments, a TβRII fusion protein comprising a linker at least 10 amino acids in length (e.g., a fusion protein having the amino acid sequence of any one of SEQ ID NOs: 11, 13 and 15) has improved binding affinity for TGFβ1 and TGFβ3 as compared to a reference TβRII fusion protein (e.g., a TβRII fusion protein having the amino acid sequence of SEQ ID NO: 9). In some embodiments, the TβRII fusion protein binds to TGFβ1 with a K_{D} of less than 200 pM, less than 150 pM, less than 100 pM, less than 75 pM, less than 50 pM or less than 25 pM. In some embodiments, the fusion protein binds to TGFβ3 with a K_{D} of less than 75 pM, less than 70 pM, less than 60 pM, less than 50 pM, less than 40 pM, less than 35 pM, less than 25 pM, less than 15, less than 10, or less than 5 pM.

In some embodiments any of the TβRII polypeptides, as well as ActRIIB:TβRII heteromultimers comprising the same, disclosed herein inhibits one or more of activin (e.g., activin A, activin B, activin C, activin E, activin AC, activin AB, activin BC, activin AE, and activin BE), GDF8, GDF11, BMP10, TGFβ1, and TGFβ3 in a measurable assay. In some embodiments, the reporter gene assay is a CAGA reporter assay. In some embodiments, the CAGA assay is based on a human lung carcinoma cell line transfected with a pGL3(CAGA)12 reporter plasmid (Dennler et al, 1998, EMBO 17: 3091-3100) as well as a Renilla reporter plasmid (pRLCMV) to control for transfection efficiency. The CAGA motif is present in the promoters of TGFβ-responsive genes (for example, PAI-1), so this vector is of general use for factors signaling through SMAD2 and SMAD3. See, e.g., Example 2.

In some embodiments, any of the fusion polypeptides disclosed herein comprises the following components: a) any of the TβRII or ActRIIB polypeptides disclosed herein (“A”), b) any of the linkers disclosed herein (“B”), c) any of the heterologous portions disclosed herein (“C”), and optionally a linker (“X”). In such embodiments, the fusion polypeptide may be arranged in a manner as follows (N-terminus to C-terminus): A-B-C or C-B-A. In such embodiments, the fusion polypeptide may be arranged in a manner as follows (N-terminus to C-terminus): X-A-B-C or X-C-B-A. In some embodiments, the fusion polypeptide comprises each of A, B and C (and optionally a leader sequence such as the amino acid sequence of SEQ ID NO: 23), and comprises no more than 100, 90, 80, 70, 60, 50, 40, 30, 20, 10, 5, 4, 3, 2 or 1 additional amino acids (but which may include further post-translational modifications, such as PEGylation).

In some embodiments, the fusion polypeptide comprises a leader sequence (e.g., SEQ ID NO: 23) positioned in a manner as follows (N-terminus to C-terminus): X-A-B-C, and the fusion polypeptide comprises 1, 2, 3, 4, or 5 amino acids between X and A. In some embodiments, the fusion polypeptide comprises a leader sequence (e.g., SEQ ID NO: 23) positioned in a manner as follows (N-terminus to C-terminus): X-C-B-A, and the fusion polypeptide comprises 1, 2, 3, 4, or 5 amino acids between X and C. In some embodiments, the fusion polypeptide comprises a leader sequence (e.g., SEQ ID NO: 23) positioned in a manner as follows (N-terminus to C-terminus): X-A-B-C, and the fusion polypeptide comprises an alanine between X and A. In some embodiments, the fusion polypeptide comprises a leader sequence (e.g., SEQ ID NO: 23) positioned in a manner as follows (N-terminus to C-terminus): X-C-B-A, and the fusion polypeptide comprises an alanine between X and C. In some embodiments, the fusion polypeptide comprises a leader sequence (e.g., SEQ ID NO: 23) positioned in a manner as follows (N-terminus to C-terminus): X-A-B-C, and the fusion polypeptide comprises a glycine and an alanine between X and A. In some embodiments, the fusion polypeptide comprises a leader sequence (e.g., SEQ ID NO: 23) positioned in a manner as follows (N-terminus to C-terminus): X-C-B-A, and the fusion polypeptide comprises a glycine and an alanine between X and C. In some embodiments, the fusion polypeptide comprises a leader sequence (e.g., SEQ ID NO: 23) positioned in a manner as follows (N-terminus to C-terminus): X-A-B-C, and the fusion polypeptide comprises a threonine between X and A. In some embodiments, the fusion polypeptide comprises a leader sequence (e.g., SEQ ID NO: 23) positioned in a manner as follows (N-terminus to C-terminus): X-C-B-A, and the fusion polypeptide comprises a threonine between X and C.

In some embodiments, the TβRII fusion polypeptide, as well as ActRIIB:TβRII heteromultimers comprising the same, comprises an amino acid sequence that is at least 70%, 75%, 80%, 85%, 90%, 91%, 92%, 93%, 94%, 95%, 96%, 97%, 98%, or 99% identical to any of the TβRII polypeptide amino acid sequences disclosed herein (e.g., SEQ ID NO: 18 or 27), wherein the TβRII polypeptide portion of the fusion polypeptide comprises no more than 10, 9, 8, 7, 6, 5, 4, 3, 2 or 1 additional amino acids (but which may include further post-translational modifications, such as PEGylation and/or glycosylation). In some embodiments, the TβRII fusion polypeptide, as well as ActRIIB:TβRII heteromultimers comprising the same, comprises an amino acid sequence that is at least 70%, 75%, 80%, 85%, 90%, 91%, 92%, 93%, 94%, 95%, 96%, 97%, 98%, or 99% identical to any of the linker sequences disclosed herein (e.g., SEQ ID NO: 6), wherein the linker portion of the fusion polypeptide comprises no more than 5, 4, 3, 2 or 1 additional amino acids (but which may include further post-translational modifications, such as PEGylation and/or glycosylation). In some embodiments, the TβRII fusion polypeptide, as well as ActRIIB:TβRII heteromultimers comprising the same, comprises an amino acid sequence that is at least 70%, 75%, 80%, 85%, 90%, 91%, 92%, 93%, 94%, 95%, 96%, 97%, 98%, or 99% identical to any of the heterologous portion sequences disclosed herein (e.g., SEQ ID NOs: 68, 69, 72, or 73), wherein the heterologous portion of the fusion polypeptide comprises no more than 25, 20, 15, 10, 5, 4, 3, 2, or 1 additional amino acids (but which may include further post-translational modifications, such as PEGylation and/or glycosylation). In some embodiments, the TβRII fusion polypeptide, as well as ActRIIB:TβRII heteromultimers comprising the same, comprises any of the TβRII polypeptide amino acid sequences disclosed herein (e.g., SEQ ID NO: 18 or 27), wherein the TβRII polypeptide portion of the fusion polypeptide comprises no more than 10, 9, 8, 7, 6, 5, 4, 3, 2 or 1 additional amino acids (but which may include further post-translational modifications, such as PEGylation and/or glycosylation). In some embodiments, the TβRII fusion polypeptide, as well as ActRIIB:TβRII heteromultimers comprising the same, comprises any of the linker sequences disclosed herein (e.g., SEQ ID NO: 6), wherein the linker portion of the fusion polypeptide comprises no more than 5, 4, 3, 2 or 1 additional amino acids (but which may include further post-translational modifications, such as PEGylation and/or glycosylation). In some embodiments, the TβRII fusion polypeptide, as well as ActRIIB:TβRII heteromultimers comprising the same, comprises any of the heterologous portion sequences disclosed herein (e.g., SEQ ID NO: 68, 69, 72, or 73), wherein the heterologous portion of the fusion polypeptide comprises no more than 25, 20, 15, 10, 5, 4, 3, 2, or 1 additional amino acids (but which may include further post-translational modifications, such as PEGylation and/or glycosylation).

In some embodiments, the ActRIIB fusion polypeptide, as well as ActRIIB:TβRII heteromultimers comprising the same, comprises an amino acid sequence that is at least 70%, 75%, 80%, 85%, 90%, 91%, 92%, 93%, 94%, 95%, 96%, 97%, 98%, or 99% identical to any of the ActRIIB polypeptide amino acid sequences disclosed herein (e.g., SEQ ID NO: 51, 52, 54, 55, or 109), wherein the ActRIIB polypeptide portion of the fusion polypeptide comprises no more than 10, 9, 8, 7, 6, 5, 4, 3, 2 or 1 additional amino acids (but which may include further post-translational modifications, such as PEGylation and/or glycosylation). In some embodiments, the ActRIIB fusion polypeptide, as well as ActRIIB:TβRII heteromultimers comprising the same, comprises an amino acid sequence that is at least 70%, 75%, 80%, 85%, 90%, 91%, 92%, 93%, 94%, 95%, 96%, 97%, 98%, or 99% identical to any of the linker sequences disclosed herein (e.g., SEQ ID NO: 6), wherein the linker portion of the fusion polypeptide comprises no more than 5, 4, 3, 2 or 1 additional amino acids (but which may include further post-translational modifications, such as PEGylation and/or glycosylation). In some embodiments, the ActRIIB fusion polypeptide, as well as ActRIIB:TβRII heteromultimers comprising the same, comprises an amino acid sequence that is at least 70%, 75%, 80%, 85%, 90%, 91%, 92%, 93%, 94%, 95%, 96%, 97%, 98%, or 99% identical to any of the heterologous portion sequences disclosed herein (e.g., SEQ ID NOs: 68, 69, 72, or 73), wherein the heterologous portion of the fusion polypeptide comprises no more than 25, 20, 15, 10, 5, 4, 3, 2, or 1 additional amino acids (but which may include further post-translational modifications, such as PEGylation and/or glycosylation). In some embodiments, the ActRIIB fusion polypeptide, as well as ActRIIB:TβRII heteromultimers comprising the same, comprises any of the ActRIIB polypeptide amino acid sequences disclosed herein (e.g., SEQ ID NO: 51, 52, 54, 55, or 109), wherein the ActRIIB polypeptide portion of the fusion polypeptide comprises no more than 10, 9, 8, 7, 6, 5, 4, 3, 2 or 1 additional amino acids (but which may include further post-translational modifications, such as PEGylation and/or glycosylation). In some embodiments, the ActRIIB fusion polypeptide, as well as ActRIIB:TβRII heteromultimers comprising the same, comprises any of the linker sequences disclosed herein (e.g., SEQ ID NO: 6), wherein the linker portion of the fusion polypeptide comprises no more than 5, 4, 3, 2 or 1 additional amino acids (but which may include further post-translational modifications, such as PEGylation and/or glycosylation). In some embodiments, the ActRIIB fusion polypeptide, as well as ActRIIB:TβRII heteromultimers comprising the same, comprises any of the heterologous portion sequences disclosed herein (e.g., SEQ ID NO: 68, 69, 72, or 73), wherein the heterologous portion of the fusion polypeptide comprises no more than 25, 20, 15, 10, 5, 4, 3, 2, or 1 additional amino acids (but which may include further post-translational modifications, such as PEGylation and/or glycosylation).

In some embodiments, the disclosure provides for a TβRII fusion polypeptide, as well as ActRIIB:TβRII heteromultimers comprising the same, wherein the fusion polypeptide consists or consists essentially of (and not necessarily in the following order): a) an amino acid sequence that is at least 70%, 75%, 80%, 85%, 90%, 91%, 92%, 93%, 94%, 95%, 96%, 97%, 98%, or 99% identical to any of the TβRII polypeptide amino acid sequences disclosed herein (e.g., SEQ ID NO: 18 or 27), wherein the TβRII polypeptide portion of the fusion polypeptide comprises no more than 10, 9, 8, 7, 6, 5, 4, 3, 2 or 1 additional amino acids (but which may include further post-translational modifications, such as PEGylation and/or glycosylation); b) an amino acid sequence that is at least 70%, 75%, 80%, 85%, 90%, 91%, 92%, 93%, 94%, 95%, 96%, 97%, 98%, or 99% identical to any of the linker sequences disclosed herein (e.g., SEQ ID NO: 6), wherein the linker portion of the fusion polypeptide comprises no more than 5, 4, 3, 2 or 1 additional amino acids (but which may include further post-translational modifications, such as PEGylation and/or glycosylation); and c) an amino acid sequence that is at least 70%, 75%, 80%, 85%, 90%, 91%, 92%, 93%, 94%, 95%, 96%, 97%, 98%, or 99% identical to any of the heterologous portion sequences disclosed herein (e.g., SEQ ID NO: 68, 69, 72, or 73), wherein the heterologous portion of the fusion polypeptide comprises no more than 25, 20, 15, 10, 5, 4, 3, 2, or 1 additional amino acids (but which may include further post-translational modifications, such as PEGylation and/or glycosylation); and d) optionally a leader sequence (e.g., SEQ ID NO: 23). In some embodiments, the disclosure provides for a TβRII fusion polypeptide, as well as ActRIIB:TβRII heteromultimers comprising the same, wherein the fusion polypeptide consists or consists essentially of (and not necessarily in the following order): a) any of the TβRII polypeptide amino acid sequences disclosed herein (e.g., SEQ ID NO: 18 or 27), wherein the TβRII polypeptide portion of the fusion polypeptide comprises no more than 10, 9, 8, 7, 6, 5, 4, 3, 2 or 1 additional amino acids (but which may include further post-translational modifications, such as PEGylation and/or glycosylation); b) any of the linker sequences disclosed herein (e.g., SEQ ID NO: 6), wherein the linker portion of the fusion polypeptide comprises no more than 5, 4, 3, 2 or 1 additional amino acids (but which may include further post-translational modifications, such as PEGylation and/or glycosylation); and c) any of the heterologous portion sequences disclosed herein (e.g., SEQ ID NO: 68, 69, 72, 73), wherein the heterologous portion of the fusion polypeptide comprises no more than 25, 20, 15, 10, 5, 4, 3, 2, or 1 additional amino acids (but which may include further post-translational modifications, such as PEGylation and/or glycosylation); and d) optionally a leader sequence (e.g., SEQ ID NO: 23).

In some embodiments, the disclosure provides for a ActRIIB fusion polypeptide, as well as ActRIIB:TβRII heteromultimers comprising the same, wherein the fusion polypeptide consists or consists essentially of (and not necessarily in the following order): a) an amino acid sequence that is at least 70%, 75%, 80%, 85%, 90%, 91%, 92%, 93%, 94%, 95%, 96%, 97%, 98%, or 99% identical to any of the ActRIIB polypeptide amino acid sequences disclosed herein (e.g., SEQ ID NO: 51, 52, 54, 55, or 109), wherein the ActRIIB polypeptide portion of the fusion polypeptide comprises no more than 10, 9, 8, 7, 6, 5, 4, 3, 2 or 1 additional amino acids (but which may include further post-translational modifications, such as PEGylation and/or glycosylation); b) an amino acid sequence that is at least 70%, 75%, 80%, 85%, 90%, 91%, 92%, 93%, 94%, 95%, 96%, 97%, 98%, or 99% identical to any of the linker sequences disclosed herein (e.g., SEQ ID NO: 6), wherein the linker portion of the fusion polypeptide comprises no more than 5, 4, 3, 2 or 1 additional amino acids (but which may include further post-translational modifications, such as PEGylation and/or glycosylation); and c) an amino acid sequence that is at least 70%, 75%, 80%, 85%, 90%, 91%, 92%, 93%, 94%, 95%, 96%, 97%, 98%, or 99% identical to any of the heterologous portion sequences disclosed herein (e.g., SEQ ID NO: 68, 69, 72, or 73), wherein the heterologous portion of the fusion polypeptide comprises no more than 25, 20, 15, 10, 5, 4, 3, 2, or 1 additional amino acids (but which may include further post-translational modifications, such as PEGylation and/or glycosylation); and d) optionally a leader sequence (e.g., SEQ ID NO: 23). In some embodiments, the disclosure provides for a ActRIIB fusion polypeptide, as well as ActRIIB:TβRII heteromultimers comprising the same, wherein the fusion polypeptide consists or consists essentially of (and not necessarily in the following order): a) any of the ActRIIB polypeptide amino acid sequences disclosed herein (e.g., SEQ ID NO: 51, 52, 54, 55, or 109), wherein the ActRIIB polypeptide portion of the fusion polypeptide comprises no more than 10, 9, 8, 7, 6, 5, 4, 3, 2 or 1 additional amino acids (but which may include further post-translational modifications, such as PEGylation and/or glycosylation); b) any of the linker sequences disclosed herein (e.g., SEQ ID NO: 6), wherein the linker portion of the fusion polypeptide comprises no more than 5, 4, 3, 2 or 1 additional amino acids (but which may include further post-translational modifications, such as PEGylation and/or glycosylation); and c) any of the heterologous portion sequences disclosed herein (e.g., SEQ ID NO: 68, 69, 72, 73), wherein the heterologous portion of the fusion polypeptide comprises no more than 25, 20, 15, 10, 5, 4, 3, 2, or 1 additional amino acids (but which may include further post-translational modifications, such as PEGylation and/or glycosylation); and d) optionally a leader sequence (e.g., SEQ ID NO: 23).

In some embodiments, the disclosure provides for a TβRII fusion polypeptide, as well as ActRIIB:TβRII heteromultimers comprising the same, consisting of or consisting essentially of (and not necessarily in the following order): a) a TβRII polypeptide portion consisting of an amino acid sequence that is at least 70%, 75%, 80%, 85%, 90%, 91%, 92%, 93%, 94%, 95%, 96%, 97%, 98%, or 99% identical to the amino acid sequence of SEQ ID NO: 18 and no more than 10, 9, 8, 7, 6, 5, 4, 3, 2 or 1 additional amino acids (but which may include further post-translational modifications, such as PEGylation and/or glycosylation); b) a linker portion consisting of an amino acid sequence that is at least 70%, 75%, 80%, 85%, 90%, 91%, 92%, 93%, 94%, 95%, 96%, 97%, 98%, or 99% identical to the amino acid sequence of SEQ ID NO: 6 and no more than 5, 4, 3, 2 or 1 additional amino acids (but which may include further post-translational modifications, such as PEGylation and/or glycosylation); and c) a heterologous portion consisting of an amino acid sequence that is at least 70%, 75%, 80%, 85%, 90%, 91%, 92%, 93%, 94%, 95%, 96%, 97%, 98%, or 99% identical to the amino acid sequence of SEQ ID NO: 69 or 73 and no more than 25, 20, 15, 10, 5, 4, 3, 2, or 1 additional amino acids (but which may include further post-translational modifications, such as PEGylation and/or glycosylation); and d) optionally a leader sequence (e.g., SEQ ID NO: 23). In some embodiments, the disclosure provides for a TβRII fusion polypeptide, as well as ActRIIB:TβRII heteromultimers comprising the same, consisting or consisting essentially of (and not necessarily in the following order): a) a TβRII polypeptide portion consisting of the amino acid sequence of SEQ ID NO: 18 and no more than 10, 9, 8, 7, 6, 5, 4, 3, 2 or 1 additional amino acids (but which may include further post-translational modifications, such as PEGylation and/or glycosylation); b) a linker portion consisting of the amino acid sequence of SEQ ID NO: 6 and no more than 5, 4, 3, 2 or 1 additional amino acids (but which may include further post-translational modifications, such as PEGylation and glycosylation); and c) a heterologous portion consisting of the amino acid sequence of SEQ ID NO: 69 or 73 and no more than 25, 20, 15, 10, 5, 4, 3, 2, or 1 additional amino acids (but which may include further post-translational modifications, such as PEGylation); and d) optionally a leader sequence (e.g., SEQ ID NO: 23).

In some embodiments, the disclosure provides for an ActRIIB fusion polypeptide, as well as ActRIIB:TβRII heteromultimers comprising the same, consisting of or consisting essentially of (and not necessarily in the following order): a) a ActRIIB polypeptide portion consisting of an amino acid sequence that is at least 70%, 75%, 80%, 85%, 90%, 91%, 92%, 93%, 94%, 95%, 96%, 97%, 98%, or 99% identical to the amino acid sequence of SEQ ID NO: 51 and no more than 10, 9, 8, 7, 6, 5, 4, 3, 2 or 1 additional amino acids (but which may include further post-translational modifications, such as PEGylation and/or glycosylation); b) a linker portion consisting of an amino acid sequence that is at least 70%, 75%, 80%, 85%, 90%, 91%, 92%, 93%, 94%, 95%, 96%, 97%, 98%, or 99% identical to the amino acid sequence of SEQ ID NO: 6 and no more than 5, 4, 3, 2 or 1 additional amino acids (but which may include further post-translational modifications, such as PEGylation and/or glycosylation); and c) a heterologous portion consisting of an amino acid sequence that is at least 70%, 75%, 80%, 85%, 90%, 91%, 92%, 93%, 94%, 95%, 96%, 97%, 98%, or 99% identical to the amino acid sequence of SEQ ID NO: 68 or 72 and no more than 25, 20, 15, 10, 5, 4, 3, 2, or 1 additional amino acids (but which may include further post-translational modifications, such as PEGylation and/or glycosylation); and d) optionally a leader sequence (e.g., SEQ ID NO: 23). In some embodiments, the disclosure provides for a ActRIIB fusion polypeptide, as well as ActRIIB:TβRII heteromultimers comprising the same, consisting or consisting essentially of (and not necessarily in the following order): a) a ActRIIB polypeptide portion consisting of the amino acid sequence of SEQ ID NO: 51 and no more than 10, 9, 8, 7, 6, 5, 4, 3, 2 or 1 additional amino acids (but which may include further post-translational modifications, such as PEGylation and/or glycosylation); b) a linker portion consisting of the amino acid sequence of SEQ ID NO: 6 and no more than 5, 4, 3, 2 or 1 additional amino acids (but which may include further post-translational modifications, such as PEGylation and glycosylation); and c) a heterologous portion consisting of the amino acid sequence of SEQ ID NO: 68 or 72 and no more than 25, 20, 15, 10, 5, 4, 3, 2, or 1 additional amino acids (but which may include further post-translational modifications, such as PEGylation); and d) optionally a leader sequence (e.g., SEQ ID NO: 23).

In some embodiments, a heteromeric protein complex of the disclosure comprises an antigen-binding domain of antibody that binds to one or more of TGFβ1, TGFβ2, TGFβ3 and at least one ActRIIB polypeptide domain (e.g. a polypeptide that is at least 70%, 75%, 80%, 85%, 90%, 91%, 92%, 93%, 94%, 95%, 97%, 98%, 99% or 100% identical to an extracellular domain of an ActRIIB protein from humans or other species as such as those described herein, e.g., SEQ ID Nos: 51, 52, 54, 55, and 109). In some embodiments, the first ActRIIB polypeptide is part of a fusion polypeptide that comprises a first member of an interaction pair (“C₁”), and further comprises an additional first member of an interaction pair (“A₁”). In some embodiments, the second ActRIIB polypeptide is part of a fusion polypeptide that comprises a second member of an interaction pair (“B₁”). In some embodiments, the variable heavy chain (V_{H}) polypeptide is part of a fusion polypeptide that comprises a second member of an interaction pair (“C₂”), and further comprises a first member of an interaction pair (“A₂”). In some embodiments, the variable light chain (V_{L}) polypeptide is part of a fusion polypeptide that comprises a second member of an interaction pair (“B₂”). In some embodiments, in each fusion polypeptide, a linker may be positioned between the first or second ActRIIB polypeptide and the corresponding member of the interaction pair, between interaction pairs, and between the V_{H} and V_{L} polypeptides and a member of the interaction pair. In some embodiments, A₁ and A₂ may be the same or different; B₁ and B₂ may be the same or different, and C₁ and C₂ may be the same or different. Suitable interaction pairs included, for example, constant heavy chain and/or light chain immunoglobulin interaction pairs, truncations, and variants thereof as described herein [e.g., Spiess et al (2015) Molecular Immunology 67(2A): 95-106]. FIG. 11A is an example of a heterodimer comprising a first and second ActRIIB extracellular domain. FIG. 11B is an example of a heteromultimer comprising a single ActRIIB extracellular domain.

In some embodiments, the disclosure provides for a heteromultimer comprising an interaction pair, wherein one member of the interaction pair comprises a TGFβ-binding portion wherein the TGFβ-binding portion is an antibody or antigen-binding fragment thereof that binds any one or more of TGFβ1, TGFβ2, or TGFβ3; and wherein the second member of the interaction pair comprises an ActRIIB polypeptide portion that is at least 70%, 75%, 80%, 85%, 90%, 91%, 92%, 93%, 94%, 95%, 97%, 98%, 99% or 100% identical to any of the ActRIIB sequences disclosed herein (e.g., SEQ ID NO: 52). In some embodiments, the antibody or antigen-binding fragment thereof binds to TGFβ1 and TGFβ3 with significantly greater affinity than to TGFβ1. In some embodiments, the antibody or antigen-binding fragment thereof binds to TGFβ1 with significantly greater affinity than to TGFβ1 or TGFβ2. In some embodiments, the antibody or antigen-binding fragment thereof binds to TGFβ 1. In some embodiments, the antibody or antigen-binding fragment thereof does not bind to TGFβ2 or does not bind to TGFβ2 with appreciable affinity. In some embodiments, the antibody or antigen-binding fragment thereof does not bind to TGFβ2 or TGFβ3, or does not bind to TGFβ2 or TGFβ3 with appreciable affinity. In some embodiments, the second member comprises a dimer of any two ActRIIB polypeptide portions disclosed herein. In some embodiments, the ActRIIB polypeptide dimerizes with a TβRII polypeptide portion that is at least 70%, 75%, 80%, 85%, 90%, 91%, 92%, 93%, 94%, 95%, 97%, 98%, 99% or 100% identical to any of the TβRII sequences disclosed herein (e.g., to SEQ ID NO: 18). In some embodiments, the ActRIIB polypeptide portion is a monomeric or “single-arm” ActRIIB polypeptide portion. In some embodiments, the interaction pair comprises a heterologous moiety that facilitates the interaction. In some embodiments, the heterologous moiety is any of the Fc portions disclosed herein. In some embodiments, the ActRIIB polypeptide portion is fused to a first heterologous moiety (e.g., a first Fc portion) and the antibody or antigen-binding fragment thereof portion is fused to a second heterologous moiety (e.g., a first Fc portion). In some embodiments, the ActRIIB polypeptide portion is fused to the N-terminus of the first Fc portion, and the antibody or antigen-binding fragment thereof portion is fused to the N-terminus of the second Fc portion. In some embodiments, the ActRIIB polypeptide portion is fused to the N-terminus of the first Fc portion, and the antibody or antigen-binding fragment thereof portion is fused to the C-terminus of the second Fc portion. In some embodiments, the ActRIIB polypeptide portion is fused to the C-terminus of the first Fc portion, and the antibody or antigen-binding fragment thereof portion is fused to the N-terminus of the second Fc portion. In some embodiments, the ActRIIB polypeptide portion is fused to the N-terminus of the first Fc portion, and the antibody or antigen-binding fragment thereof portion is fused to the N-terminus of the second Fc portion; and the ActRIIB polypeptide portion is a heterodimer with any of the TβRII polypeptides disclosed herein. In some embodiments, the ActRIIB polypeptide portion is fused to the N-terminus of the first Fc portion, and the antibody or antigen-binding fragment thereof portion is fused to the C-terminus of the second Fc portion; and the ActRIIB polypeptide portion is a heterodimer with any of the TβRII polypeptides disclosed herein. In some embodiments, the ActRIIB polypeptide portion is fused to the C-terminus of the first Fc portion, and the antibody or antigen-binding fragment thereof portion is fused to the N-terminus of the second Fc portion; and the ActRIIB polypeptide portion is a heterodimer with any of the TβRII polypeptides disclosed herein. In embodiments comprising an ActRIIB polypeptide portion and a TβRII polypeptide, the ActRIIB polypeptide may be fused to the Fc portion, or the TβRII polypeptide may be fused to the Fc portion. In some embodiments, the VL portion of the antibody or antigen-binding fragment thereof is fused to the Fc portion, and in some embodiments, the VH of the antibody or antigen-binding fragment thereof is fused to the Fc portion. The disclosure contemplates linkers to facilitate the fusion between any of the components in the interaction pair. In some embodiments, the interaction pair comprises a second interaction pair that facilitates that interaction between the TβRII polypeptide and the ActRIIB polypeptide. FIG. 11A provides an illustrative example of an interaction pair comprising an ActRIIB polypeptide portion that is fused to the N-terminus of the first Fc portion, and the antibody or antigen-binding fragment thereof portion is fused to the N-terminus of the second Fc portion; and wherein the ActRIIB polypeptide portion is a heterodimer with a TβRII polypeptide.

In some embodiments, the disclosure provides for a fusion protein comprising any of the ActRIIB polypeptides disclosed herein (e.g., an ActRIIB polypeptide comprising an amino acid sequence that is at least 70%, 75%, 80%, 85%, 90%, 91%, 92%, 93%, 94%, 95%, 97%, 98%, 99% or 100% identical to the amino acid sequence of SEQ ID NO: 51 or 52) fused to any of the TβRII polypeptides disclosed herein (e.g., a TβRII polypeptide comprising an amino acid sequence that is at least 70%, 75%, 80%, 85%, 90%, 91%, 92%, 93%, 94%, 95%, 97%, 98%, 99% or 100% identical to the amino acid sequence of SEQ ID NO: 170). In some embodiments, the ActRIIB polypeptide portion is N-terminal to the TβRII polypeptide portion. In some embodiments, the ActRIIB polypeptide portion is C-terminal to the TβRII polypeptide portion. In some embodiments, the ActRIIB polypeptide portion of the fusion protein is fused directly to the TβRII polypeptide portion of the fusion protein. In some embodiments, a heterologous portion (e.g., any of the Fc portions disclosed herein) and/or one or more linker portions separate the ActRIIB and TβRII polypeptide portions in the fusion protein. In some embodiments, the heterologous portion is an Fc polypeptide portion comprising an amino acid sequence that is at least 70%, 75%, 80%, 85%, 90%, 91%, 92%, 93%, 94%, 95%, 97%, 98%, 99% or 100% identical to the amino acid sequence of SEQ ID NO: 163. In some embodiments, the heterologous portion is an Fc polypeptide portion comprising an amino acid sequence that is at least 70%, 75%, 80%, 85%, 90%, 91%, 92%, 93%, 94%, 95%, 97%, 98%, 99% or 100% identical to the amino acid sequence of SEQ ID NO: 72 (which may optionally lack the C-terminal lysine residue). In some embodiments, the heterologous portion is an Fc polypeptide portion comprising an amino acid sequence that is at least 70%, 75%, 80%, 85%, 90%, 91%, 92%, 93%, 94%, 95%, 97%, 98%, 99% or 100% identical to the amino acid sequence of SEQ ID NO: 73 (which may optionally lack the C-terminal lysine residue). In some embodiments, the TβRII polypeptide portion is fused to the Fc portion by means of a linker (e.g., any of the linkers disclosed herein). In some embodiments, the TβRII polypeptide portion is fused to the Fc portion by means of a glycine-serine-rich linker, such as a linker comprising an amino acid sequence that is at least 70%, 75%, 80%, 85%, 90%, 91%, 92%, 93%, 94%, 95%, 97%, 98%, 99% or 100% identical to the amino acid sequence of SEQ ID NO: 165. In some embodiments, the ActRIIB polypeptide portion is fused to the Fc portion by means of a linker (e.g., any of the linkers disclosed herein). In some embodiments, the ActRIIB polypeptide portion is fused to the Fc portion by means of a linker comprising a glycine linker, such as a linker comprising a GGG amino acid sequence. In some embodiments, the fusion protein comprises any of the signal sequences disclosed herein. In some embodiments, the signal sequence comprises an amino acid sequence that is at least 70%, 75%, 80%, 85%, 90%, 91%, 92%, 93%, 94%, 95%, 97%, 98%, 99% or 100% identical to the amino acid sequence of SEQ ID NO: 23. In some embodiments, the fusion protein comprises an amino acid sequence that is at least 70%, 75%, 80%, 85%, 90%, 91%, 92%, 93%, 94%, 95%, 97%, 98%, 99% or 100% identical to the amino acid sequence of SEQ ID NO: 183. In some embodiments, the fusion protein comprises an amino acid sequence that is at least 70%, 75%, 80%, 85%, 90%, 91%, 92%, 93%, 94%, 95%, 97%, 98%, 99% or 100% identical to the amino acid sequence of SEQ ID NO: 195.

(SEQ ID NO: 183)

| | |
|---|---|
| 1 | MDAMKRGLCC VLLLCGAVFV SPGASGRGEA |
| | ETRECIYYNA NWELERTNQS |
| 51 | GLERCEGEQD KRLHCYASWR NSSGTIELVK |
| | KGCWLDDFNC YDRQECVATE |
| 101 | ENPQVYFCCC EGNFCNERFT HLPEAGGPEV |
| | TYEPPPTAPT GGGTHTCPPC |
| 151 | PAPELLGGPS VFLFPPKPKD TLMISRTPEV |
| | TCVVVDVSHE DPEVKFNWYV |
| 201 | DGVEVHNAKT KPREEQYNST YRVVSVLTVL |
| | HQDWLNGKEY KCKVSNKALP |
| 251 | APIEKTISKA KGQPREPQVY TLPPSREEMT |
| | KNQVSLTCLV KGFYPSDIAV |
| 301 | EWESNGQPEN NYKTTPPVLD SDGSFFLYSK |
| | LTVDKSRWQQ GNVFSCSVMH |
| 351 | EALHNHYTQK SLSLSPGAGG GGSGGGGSGG |
| | GGSGGGGSGT IPPHVQKSDV |
| 401 | EMEAQKDEII CPSCNRTAHP LRHINNDMIV |
| | TDNNGAVKFP QLCKFCDVRF |
| 451 | STCDNQKSCM SNCSITSICE KPQEVCVAVW |
| | RKNDENITLE TVCHDPKLPY |
| 501 | HDFILEDAAS PKCIMKEKKK PGETFFMCSC |
| | SSDECNDNII FSEEYNTSNP |
| 551 | D |

(SEQ ID NO: 195)

GRGEA ETRECIYYNA NWELERTNQS
GLERCEGEQD KRLHCYASWR NSSGTIELVK
KGCWLDDFNC YDRQECVATE ENPQVYFCCC
EGNFCNERFT HLPEAGGPEV TYEPPPTAPT
GGGTHTCPPC PAPELLGGPS VFLFPPKPKD
TLMISRTPEV TCVVVDVSHE DPEVKFNWYV
DGVEVHNAKT KPREEQYNST YRVVSVLTVL
HQDWLNGKEY KCKVSNKALP APIEKTISKA
KGQPREPQVY TLPPSREEMT KNQVSLTCLV
KGFYPSDIAV EWESNGQPEN NYKTTPPVLD
SDGSFFLYSK LTVDKSRWQQ GNVFSCSVMH
EALHNHYTQK SLSLSPGAGG GGSGGGGSGG
GGSGGGGSGT IPPHVQKSDV EMEAQKDEII
CPSCNRTAHP LRHINNDMIV TDNNGAVKFP
QLCKFCDVRF STCDNQKSCM SNCSITSICE
KPQEVCVAVW RKNDENITLE TVCHDPKLPY
HDFILEDAAS PKCIMKEKKK PGETFFMCSC
SSDECNDNII FSEEYNTSNP D

In some embodiments, any of the ActRIIB and TβRII polypeptides disclosed herein are encoded by a nucleic acid comprising a nucleotide sequence that is at least 70%, 75%, 80%, 85%, 90%, 91%, 92%, 93%, 94%, 95%, 97%, 98%, 99% or 100% identical to the nucleotide sequence of SEQ ID NO: 186.

(SEQ ID NO: 186)

| | |
|---|---|
| 1 | ATGGATGCAA TGAAGAGAGG GCTCTGCTGT |
| | GTGCTGCTGC TGTGTGGAGC |
| 51 | AGTCTTCGTT TCGCCCGGCG CCAGCGGTAG |
| | GGGCGAAGCA GAAACCCGCG |
| 101 | AATGTATATA TTATAATGCT AATTGGGAAC |
| | TTGAAAGGAC AAATCAATCC |
| 151 | GGACTTGAAC GTTGTGAAGG GGAACAAGAT |
| | AAACGACTCC ATTGTTATGC |
| 201 | ATCATGGAGA AATTCTTCCG GAACTATTGA |
| | ATTGGTAAAG AAAGGATGTT |
| 251 | GGTTGGACGA TTTTAATTGT TACGACCGCC |
| | AAGAATGCGT TGCAACAGAA |
| 301 | GAAAATCCTC AAGTTTATTT CTGTTGTTGC |
| | GAGGGGAACT TTTGTAATGA |
| 351 | AAGGTTTACC CATCTCCCTG AAGCAGGCGG |
| | ACCTGAGGTG ACATATGAAC |
| 401 | CACCACCTAC TGCTCCAACC GGTGGCGGTA |
| | CCCATACCTG TCCGCCATGT |
| 451 | CCCGCTCCCG AGCTACTTGG CGGCCCCTCT |
| | GTATTCTTGT TTCCGCCTAA |
| 501 | GCCGAAAGAT ACTTTGATGA TTTCACGAAC |
| | TCCAGAAGTT ACCTGTGTAG |
| 551 | TAGTCGATGT TAGTCATGAA GATCCCGAAG |
| | TAAAATTTAA TTGGTATGTT |
| 601 | GATGGGGTAG AAGTTCACAA CGCTAAAACC |
| | AAACCTCGAG AAGAACAATA |
| 651 | TAATTCCACC TATCGCGTTG TTTCTGTGCT |
| | GACAGTGTTG CATCAAGATT |
| 701 | GGCTTAACGG GAAAGAATAT AAATGTAAAG |
| | TGTCTAATAA GGCTCTTCCT |
| 751 | GCTCCGATTG AAAAGACTAT TAGTAAGGCA |
| | AAGGGTCAAC CACGTGAGCC |
| 801 | CCAAGTATAT ACATTGCCGC CCAGTCGAGA |
| | AGAAATGACG AAGAATCAAG |
| 851 | TTTCTTTGAC TTGTCTCGTG AAGGGATTTT |
| | ACCCATCAGA TATTGCTGTC |
| 901 | GAATGGGAAT CTAACGGTCA ACCAGAAAAT |
| | AATTATAAAA CGACTCCACC |
| 951 | TGTCCTCGAT AGCGATGGAT CTTTCTTTCT |
| | GTACTCCAAA CTGACTGTTG |
| 1001 | ATAAATCCCG GTGGCAACAA GGTAATGTTT |
| | TCAGTTGTAG CGTTATGCAC |
| 1051 | GAAGCACTAC ATAATCATTA TACACAAAAG |
| | TCACTGTCTC TCAGTCCCGG |
| 1101 | AGCAGGCGGC GGTGGCTCAG GCGGTGGTGG |
| | TTCAGGCGGC GGCGGGTCAG |
| 1151 | GCGGTGGTGG GAGCGGGACT ATTCCCCCAC |
| | ATGTCCAAAA GTCAGACGTT |
| 1201 | GAGATGGAAG CTCAAAAGGA CGAGATAATA |
| | TGTCCTTCCT GCAACAGAAC |
| 1251 | CGCACACCCT CTCAGGCACA TAAACAATGA |
| | TATGATCGTG ACAGATAATA |
| 1301 | ATGGCGCTGT GAAATTCCCC CAGCTCTGCA |
| | AGTTCTGCGA CGTTCGCTTC |
| 1351 | AGCACTTGCG ATAATCAAAA GTCTTGTATG |
| | TCTAATTGTT CCATTACTAG |
| 1401 | CATTTGCGAG AAACCCCAAG AGGTGTGCGT |
| | CGCCGTCTGG CGGAAGAACG |
| 1451 | ATGAAAATAT TACCCTCGAA ACGGTGTGTC |
| | ACGATCCGAA ACTGCCATAT |
| 1501 | CACGATTTCA TCTTGGAAGA CGCAGCCTCA |
| | CCGAAATGTA TCATGAAAGA |
| 1551 | GAAGAAGAAA CCAGGGGAAA CCTTCTTTAT |
| | GTGCTCTTGC TCCAGCGACG |
| 1601 | AATGTAACGA TAATATTATT TTCAGTGAGG |
| | AGTACAATAC TTCTAACCCA |
| 1651 | GATTAG |

In some embodiments, any of the ActRIIB and TβRII polypeptide fusion proteins disclosed herein multimerize with another protein. In some embodiments, any of the ActRIIB and TβRII polypeptide fusion proteins disclosed herein homomultimerize (e.g., homodimerize). For example, in some embodiments, the disclosure contemplates a homomultimer comprising two or more fusion proteins comprising an amino acid sequence that is at least 70%, 75%, 80%, 85%, 90%, 91%, 92%, 93%, 94%, 95%, 97%, 98%, 99% or 100% identical to the amino acid sequence of SEQ ID NO: 183. In some embodiments, the disclosure contemplates a homomultimer comprising two or more fusion proteins comprising an amino acid sequence that is at least 70%, 75%, 80%, 85%, 90%, 91%, 92%, 93%, 94%, 95%, 97%, 98%, 99% or 100% identical to the amino acid sequence of SEQ ID NO: 195.

In some embodiments, any of the ActRIIB and TβRII polypeptide fusion proteins disclosed herein heteromultimerize with one or more different proteins/polypeptides. In some embodiments, any of the ActRIIB and TβRII polypeptide fusion proteins disclosed herein heteromultimerize with a protein/polypeptide comprising an ActRIIB polypeptide portion but lacking a TβRII polypeptide portion. In such embodiments, the resulting fusion protein would comprise two ActRIIB polypeptide portion “arms,” but a single TβRII polypeptide portion arm. In some embodiments, each unit of the heteromultimer comprises a member of an interaction pair. In some embodiments, the member of the interaction pair is any of the Fc portions disclosed herein. In some embodiments, the Fc portions have been modified to promote heteromultimer formation and/or to inhibit homomultimer formation. In some embodiments, the Fc portions have been modified to promote heterodimer formation and/or to inhibit homodimer formation. In some embodiments, the Fc portions have been modified to include any of the “knob-in-hole” mutations disclosed herein. In some embodiments, the heterologous portion is an Fc polypeptide portion comprising an amino acid sequence that is at least 70%, 75%, 80%, 85%, 90%, 91%, 92%, 93%, 94%, 95%, 97%, 98%, 99% or 100% identical the amino acid sequence of SEQ ID NO: 72 (which may optionally lack the C-terminal lysine residue). In some embodiments, the heterologous portion is an Fc polypeptide portion comprising an amino acid sequence that is at least 70%, 75%, 80%, 85%, 90%, 91%, 92%, 93%, 94%, 95%, 97%, 98%, 99% or 100% identical the amino acid sequence of SEQ ID NO: 73 (which may optionally lack the C-terminal lysine residue).

In some embodiments, any of the ActRIIB and TβRII polypeptide fusion proteins disclosed herein heteromultimerize with a protein/polypeptide comprising a TβRII polypeptide portion but lacking an ActRIIB polypeptide portion. In such embodiments, the resulting fusion protein would comprise two TβRII polypeptide portion “arms,” but a single ActRIIB polypeptide portion arm. In some embodiments, each unit of the heteromultimer comprises a member of an interaction pair. In some embodiments, the member of the interaction pair is any of the Fc portions disclosed herein. In some embodiments, the Fc portions have been modified to promote heteromultimer formation and/or to inhibit homomultimer formation. In some embodiments, the Fc portions have been modified to promote heterodimer formation and/or to inhibit homodimer formation. In some embodiments, the Fc portions have been modified to include any of the “knob-in-hole” mutations disclosed herein. In some embodiments, the ActRIIB and TβRII polypeptide fusion protein in such heteromultimers comprises an amino acid sequence that is at least 70%, 75%, 80%, 85%, 90%, 91%, 92%, 93%, 94%, 95%, 97%, 98%, 99% or 100% identical to the amino acid sequence of SEQ ID NO: 184. In some embodiments, the protein/polypeptide comprising the TβRII polypeptide portion but lacking the ActRIIB polypeptide portion comprises an amino acid sequence that is at least 70%, 75%, 80%, 85%, 90%, 91%, 92%, 93%, 94%, 95%, 97%, 98%, 99% or 100% identical to the amino acid sequence of SEQ ID NO: 185. In some embodiments, the heteromultimer is a heterodimer comprising a first fusion protein comprising the amino acid sequence of SEQ ID NO: 184 and a second fusion protein comprising the amino acid sequence of SEQ ID NO: 185. In some embodiments, the ActRIIB and TβRII polypeptide fusion protein in such heteromultimers comprises an amino acid sequence that is at least 70%, 75%, 80%, 85%, 90%, 91%, 92%, 93%, 94%, 95%, 97%, 98%, 99% or 100% identical to the amino acid sequence of SEQ ID NO: 196. In some embodiments, the protein/polypeptide comprising the TβRII polypeptide portion but lacking the ActRIIB polypeptide portion comprises an amino acid sequence that is at least 70%, 75%, 80%, 85%, 90%, 91%, 92%, 93%, 94%, 95%, 97%, 98%, 99% or 100% identical to the amino acid sequence of SEQ ID NO: 197. In some embodiments, the heteromultimer is a heterodimer comprising a first fusion protein comprising the amino acid sequence of SEQ ID NO: 196 and a second fusion protein comprising the amino acid sequence of SEQ ID NO: 197.

(SEQ ID NO: 184)

| | |
|---|---|
| 1 | MDAMKRGLCC VLLLCGAVFV SPGASGRGEA |
| | ETRECIYYNA NWELERTNQS |
| 51 | GLERCEGEQD KRLHCYASWR NSSGTIELVK |
| | KGCWLDDFNC YDRQECVATE |
| 101 | ENPQVYFCCC EGNFCNERFT HLPEAGGPEV |
| | TYEPPPTAPT GGGTHTCPPC |
| 151 | PAPELLGGPS VFLFPPKPKD TLMISRTPEV |
| | TCVVVDVSHE DPEVKFNWYV |
| 201 | DGVEVHNAKT KPREEQYNST YRVVSVLTVL |
| | HQDWLNGKEY KCKVSNKALP |
| 251 | APIEKTISKA KGQPREPQVY TLPPCREEMT |
| | KNQVSLWCLV KGFYPSDIAV |
| 301 | EWESNGQPEN NYKTTPPVLD SDGSFFLYSK |
| | LTVDKSRWQQ GNVFSCSVMH |
| 351 | EALHNHYTQK SLSLSPGAGG GGSGGGGSGG |
| | GGSGGGGSGT IPPHVQKSDV |
| 401 | EMEAQKDEII CPSCNRTAHP LRHINNDMIV |
| | TDNNGAVKFP QLCKFCDVRF |
| 451 | STCDNQKSCM SNCSITSICE KPQEVCVAVW |
| | RKNDENITLE TVCHDPKLPY |
| 501 | HDFILEDAAS PKCIMKEKKK PGETFFMCSC |
| | SSDECNDNII FSEEYNTSNP |
| 551 | D |

(SEQ ID NO: 185)

| | |
|---|---|
| 1 | MDAMKRGLCC VLLLCGAVFV SPGASNTKVD |
| | KRVTGGGTHT CPPCPAPELL |
| 51 | GGPSVFLFPP KPKDTLMISR TPEVTCVVVD |
| | VSHEDPEVKF NWYVDGVEVH |
| 101 | NAKTKPREEQ YNSTYRVVSV LTVLHQDWLN |
| | GKEYKCKVSN KALPAPIEKT |
| 151 | ISKAKGQPRE PQVCTLPPSR EEMTKNQVSL |
| | SCAVKGFYPS DIAVEWESNG |
| 201 | QPENNYKTTP PVLDSDGSFF LVSKLTVDKS |
| | RWQQGNVFSC SVMHEALHNH |
| 251 | YTQKSLSLSP GAGGGGSGGG GSGGGGSGGG |
| | GSGTIPPHVQ KSDVEMEAQK |
| 301 | DEIICPSCNR TAHPLRHINN DMIVTDNNGA |
| | VKFPQLCKFC DVRFSTCDNQ |
| 351 | KSCMSNCSIT SICEKPQEVC VAVWRKNDEN |
| | ITLETVCHDP KLPYHDFILE |
| 401 | DAASPKCIMK EKKKPGETFF MCSCSSDECN |
| | DNIIFSEEYN TSNPD |

(SEQ ID NO: 196)

GRGEA ETRECIYYNA NWELERTNQS
GLERCEGEQD KRLHCYASWR NSSGTIELVK
KGCWLDDFNC YDRQECVATE ENPQVYFCCC
EGNFCNERFT HLPEAGGPEV TYEPPPTAPT
GGGTHTCPPC PAPELLGGPS VFLFPPKPKD
TLMISRTPEV TCVVVDVSHE DPEVKFNWYV
DGVEVHNAKT KPREEQYNST YRVVSVLTVL
HQDWLNGKEY KCKVSNKALP APIEKTISKA
KGQPREPQVY TLPPCREEMT KNQVSLWCLV
KGFYPSDIAV EWESNGQPEN NYKTTPPVLD
SDGSFFLYSK LTVDKSRWQQ GNVFSCSVMH
EALHNHYTQK SLSLSPGAGG GGSGGGGSGG
GGSGGGGSGT IPPHVQKSDV EMEAQKDEII
CPSCNRTAHP LRHINNDMIV TDNNGAVKFP
QLCKFCDVRF STCDNQKSCM SNCSITSICE
KPQEVCVAVW RKNDENITLE TVCHDPKLPY
HDFILEDAAS PKCIMKEKKK PGETFFMCSC
SSDECNDNII FSEEYNTSNP D

(SEQ ID NO: 197)

NTKVD KRVTGGGTHT CPPCPAPELL
GGPSVFLFPP KPKDTLMISR TPEVTCVVVD
VSHEDPEVKF NWYVDGVEVH NAKTKPREEQ
YNSTYRVVSV LTVLHQDWLN GKEYKCKVSN
KALPAPIEKT ISKAKGQPRE PQVCTLPPSR
EEMTKNQVSL SCAVKGFYPS DIAVEWESNG
QPENNYKTTP PVLDSDGSFF LVSKLTVDKS
RWQQGNVFSC SVMHEALHNH YTQKSLSLSP
GAGGGGSGGG GSGGGGSGGG GSGTIPPHVQ
KSDVEMEAQK DEIICPSCNR TAHPLRHINN
DMIVTDNNGA VKFPQLCKFC DVRFSTCDNQ
KSCMSNCSIT SICEKPQEVC VAVWRKNDEN
ITLETVCHDP KLPYHDFILE DAASPKCIMK
EKKKPGETFF MCSCSSDECN DNIIFSEEYN
TSNPD

In some embodiments, any of the ActRIIB and TβRII polypeptides disclosed herein for use in any of the heteromultimers disclosed herein is encoded by a nucleic acid comprising a nucleotide sequence that is at least 70%, 75%, 80%, 85%, 90%, 91%, 92%, 93%, 94%, 95%, 97%, 98%, 99% or 100% identical to the nucleotide sequence of SEQ ID NO: 187. In some embodiments, any of the proteins comprising the TβRII polypeptide portion but lacking the ActRIIB polypeptide portion is encoded by a nucleotide sequence that is at least 70%, 75%, 80%, 85%, 90%, 91%, 92%, 93%, 94%, 95%, 97%, 98%, 99% or 100% identical to the nucleotide sequence of SEQ ID NO: 188.

(SEQ ID NO: 187)

| | |
|---|---|
| 1 | ATGGATGCAA TGAAGAGAGG GCTCTGCTGT |
| | GTGCTGCTGC TGTGTGGAGC |
| 51 | AGTCTTCGTT TCGCCCGGCG CCAGTGGCCG |
| | GGGTGAAGCC GAAACTCGCG |
| 101 | AATGTATTTA TTATAATGCT AATTGGGAAC |
| | TCGAACGTAC AAATCAATCT |
| 151 | GGGCTCGAAC GGTGTGAGGG TGAACAAGAT |
| | AAAAGACTCC ATTGTTATGC |
| 201 | TTCTTGGAGA AATTCTAGCG GAACAATTGA |
| | ACTGGTTAAG AAGGGTTGTT |
| 251 | GGCTGGACGA TTTTAATTGT TATGACCGCC |
| | AAGAATGCGT CGCAACAGAA |
| 301 | GAAAATCCTC AAGTTTACTT TTGTTGTTGC |
| | GAGGGTAACT TTTGTAATGA |
| 351 | AAGGTTTACG CACCTGCCTG AAGCAGGCGG |
| | GCCTGAGGTG ACATATGAAC |
| 401 | CGCCACCAAC CGCTCCAACC GGTGGCGGTA |
| | CCCATACCTG TCCACCATGT |
| 451 | CCTGCCCCAG AGCTGCTAGG TGGGCCAAGC |
| | GTGTTTCTGT TTCCACCTAA |
| 501 | GCCAAAAGAT ACTCTGATGA TATCTAGGAC |
| | TCCAGAAGTG ACCTGTGTCG |
| 551 | TTGTTGATGT TTCTCACGAA GATCCAGAAG |
| | TGAAATTTAA TTGGTATGTC |
| 601 | GATGGAGTTG AAGTCCACAA CGCTAAAACT |
| | AAACCAAGAG AGGAACAATA |
| 651 | TAATTCTACA TATAGGGTTG TGAGTGTGCT |
| | GACAGTGTTG CACCAGGATT |
| 701 | GGTTGAACGG TAAAGAATAT AAATGTAAAG |
| | TGTCTAATAA GGCTTTGCCC |
| 751 | GCTCCTATTG AAAAGACGAT AAGCAAGGCT |
| | AAGGGCCAAC CACGCGAGCC |
| 801 | TCAAGTCTAT ACACTTCCAC CCTGTAGGGA |
| | AGAAATGACC AAGAATCAAG |
| 851 | TGTCCTTGTG GTGTCTTGTT AAGGGGTTTT |
| | ACCCATCTGA TATTGCAGTC |
| 901 | GAATGGGAAT CAAACGGCCA ACCCGAAAAT |
| | AATTATAAAA CTACTCCGCC |
| 951 | AGTCTTGGAT TCTGATGGAA GCTTCTTCCT |
| | ATACTCAAAA CTAACTGTTG |
| 1001 | ATAAATCACG TTGGCAACAA GGAAATGTGT |
| | TTTCCTGTTC AGTCATGCAC |
| 1051 | GAAGCCCTGC ATAATCATTA TACTCAGAAA |
| | TCATTGAGTT TGTCACCAGG |
| 1101 | AGCTGGAGGA GGTGGAAGTG GTGGTGGTGG |
| | CTCTGGCGGC GGCGGCTCCG |
| 1151 | GCGGCGGTGG GTCAGGAACT ATACCCCCTC |
| | ATGTGCAAAA GTCCGATGTC |
| 1201 | GAGATGGAAG CTCAAAAGGA CGAGATTATT |
| | TGTCCTTCCT GCAACCGCAC |
| 1251 | GGCACACCCT CTCCGCCACA TCAACAATGA |
| | TATGATCGTG ACCGATAATA |
| 1301 | ATGGGGCCGT GAAATTCCCG CAGCTTTGCA |
| | AGTTCTGCGA CGTTCGTTTC |
| 1351 | TCTACTTGCG ATAATCAAAA GTCTTGTATG |
| | TCAAATTGTT CTATTACAAG |
| 1401 | CATTTGCGAA AAGCCTCAAG AGGTGTGCGT |
| | CGCAGTGTGG CGCAAGAACG |
| 1451 | ATGAAAATAT CACGCTTGAA ACTGTGTGTC |
| | ACGATCCGAA ACTTCCATAT |
| 1501 | CACGATTTCA TCCTAGAGGA CGCAGCAAGC |
| | CCCAAATGTA TCATGAAAGA |
| 1551 | GAAGAAGAAA CCCGGAGAAA CCTTCTTCAT |
| | GTGCTCATGC TCTTCCGACG |
| 1601 | AATGTAACGA TAATATTATA TTTAGCGAGG |
| | AGTACAATAC TTCAAACCCC |
| 1651 | GATTAG |

(SEQ ID NO: 188)

| | |
|---|---|
| 1 | ATGGATGCAA TGAAGAGAGG GCTCTGCTGT |
| | GTGCTGCTGC TGTGTGGAGC |
| 51 | AGTCTTCGTT TCGCCCGGCG CCAGCAACAC |
| | CAAGGTGGAC AAGAGAGTTA |
| 101 | CCGGTGGTGG AACTCACACA TGCCCACCGT |
| | GCCCAGCACC TGAACTCCTG |
| 151 | GGGGGACCGT CAGTCTTCCT CTTCCCCCCA |
| | AAACCCAAGG ACACCCTCAT |
| 201 | GATCTCCCGG ACCCCTGAGG TCACATGCGT |
| | GGTGGTGGAC GTGAGCCACG |
| 251 | AAGACCCTGA GGTCAAGTTC AACTGGTACG |
| | TGGACGGCGT GGAGGTGCAT |
| 301 | AATGCCAAGA CAAAGCCGCG GGAGGAGCAG |
| | TACAACAGCA CGTACCGTGT |
| 351 | GGTCAGCGTC CTCACCGTCC TGCACCAGGA |
| | CTGGCTGAAT GGCAAGGAGT |
| 401 | ACAAGTGCAA GGTCTCCAAC AAAGCCCTCC |
| | CAGCCCCCAT CGAGAAAACC |
| 451 | ATCTCCAAAG CCAAAGGGCA GCCCCGAGAA |
| | CCACAGGTGT GCACCCTGCC |
| 501 | CCCATCCCGG GAGGAGATGA CCAAGAACCA |
| | GGTCAGCCTG TCCTGCGCCG |
| 551 | TCAAAGGCTT CTATCCCAGC GACATCGCCG |
| | TGGAGTGGGA GAGCAATGGG |
| 601 | CAGCCGGAGA ACAACTACAA GACCACGCCT |
| | CCCGTGCTGG ACTCCGACGG |
| 651 | CTCCTTCTTC CTCGTGAGCA AGCTCACCGT |
| | GGACAAGAGC AGGTGGCAGC |
| 701 | AGGGGAACGT CTTCTCATGC TCCGTGATGC |
| | ATGAGGCTCT GCACAACCAC |
| 751 | TACACGCAGA AGAGCCTCTC CCTGTCTCCG |
| | GGTGCTGGTG GTGGAGGTTC |
| 801 | TGGAGGTGGA GGAAGTGGTG GAGGTGGTTC |
| | TGGAGGTGGT GGTTCCGGAA |
| 851 | CGATCCCACC GCACGTTCAG AAGTCGGATG |
| | TGGAAATGGA GGCCCAGAAA |
| 901 | GATGAAATCA TCTGCCCCAG CTGTAATAGG |
| | ACTGCCCATC CACTGAGACA |
| 951 | TATTAATAAC GACATGATAG TCACTGACAA |
| | CAACGGTGCA GTCAAGTTTC |
| 1001 | CACAACTGTG TAAATTTTGT GATGTGAGAT |
| | TTTCCACCTG TGACAACCAG |
| 1051 | AAATCCTGCA TGAGCAACTG CAGCATCACC |
| | TCCATCTGTG AGAAGCCACA |
| 1101 | GGAAGTCTGT GTGGCTGTAT GGAGAAAGAA |
| | TGACGAGAAC ATAACACTAG |
| 1151 | AGACAGTTTG CCATGACCCC AAGCTCCCCT |
| | ACCATGACTT TATTCTGGAA |
| 1201 | GATGCTGCTT CTCCAAAGTG CATTATGAAG |
| | GAAAAAAAAA AGCCTGGTGA |
| 1251 | GACTTTCTTC ATGTGTTCCT GTAGCTCTGA |
| | TGAGTGCAAT GACAACATCA |
| 1301 | TCTTCTCAGA AGAATATAAC ACCAGCAATC |
| | CTGACTGA |

In some embodiments, the heteromultimers disclosed herein do not bind with appreciable affinity to CD4, CD8, CD25, CTLA-4, IL-10, TGFβ Receptor, PD-1, PD-L1, PD-L2, RANK, RANKL, HER2/neu, EGFR1, CD20, VEGF, TNF-α, TNFR2, FoxP3, CD80, CD86, IFN-α, IFN-β, IFN-γ, GITR, 4-1BB, OX-40, TLR1-10, ErbB-1, HER1, ErbB-3/HER3, ErbB-4/HER4, IGFR, IGFBP, IGF-1R, PDGFR, FGFR, VEGFR, HGFR, TRK receptor, ephrin receptors, AXL receptors, LTK receptors, TIE receptors, angiopoietin1, 2, ROR receptor, DDR receptor, RET receptor, KLG receptor, RYK receptor, MuSK receptor, ILβR, IlαR, TNTRSF, TRAIL receptor, ARTC1, alpha-actinin-4, Bcr-abl, B-RAF, caspases, beta-catenin, fibronectin, GPNMB, GDP-L, LDLR, HLA-A2, MLA-A11, HSP70, KIAA205, MART2, MUM-1, 2, 3, PAP, neo-PAP, NFYC, OGT, OS-9, pml-RARalpha fusion protein, PRDX5, PTPRK, KRAS2, NRAS, HRAS, RBAF600, SIRT2. SNRPD1, SYT-SSX1 or -SSX2 fusion protein, Triosephosphate Isomerase, BAGE, BAGE-1. BAGE-2, 3, 4, 5, GAGE-1, 2, 3, 4, 5, 6, 7, 8, GnT-V, HERV-K MEL, KK-LC, KM-HN-1, LAGE, LAGE-1, CAMEL, MAGE-1, MAGE-A2, MAGE-A3, MAGE-A4, MAGE-AS, MAGE-A6, MAGE-A8, MAGE-A9, MAGE-A10. MAGE-A11, MAGE-A12, MAGE-3, MAGE-B1, MAGE-B2, MAGE-B5. MAGE-B6, MAGE-C1, MAGE-C2, mucin 1 (MUC1), MART-1/Melan-A (MLANA), gp100, gp100/Pme117 (S1LV), tyrosinase (TYR), TRP-1, HAGE, NA-88, NY-ESO-1, NY-ESO-1/LAGE-2, SAGE, Sp17. SSX-1, 2, 3, 4, TRP2-1NT2, carcino-embryonic antigen (CEA), Kallikfein 4, mammaglobm-A, OA1, prostate specific antigen (PSA), prostate specific membrane antigen, TRP-1/, 75. TRP-2, AIM-2. BING-4, CPSF, cyclin D1, Ep-CAM, EpbA3, FGF-5, gp250, iCE), AFP, M-CSF, mdm-2, MUCI, p53 (TP53), PBF, FRAME, PSMA, RAGE-1. RNF43, RU2AS, SOX10, STEAPI, survivin (BIRCS), hTERT, telomerase, WT1, SYCP1, BRDT, SPANX, XAGE, ADAM2, PAGE-5, LIP1, CTAGE-1, CSAGE, MMA1, CAGE, BORIS, HOM-TES-85, AF15q14, HCA66I, LDHC, MORC, SGY-1, SPO11, TPX1, NY-SAR-35, FTHLI7, NXF2 TDRD1, TEX 15, FATE, TPTE, estrogen receptors (ER), androgen receptors (AR), CD40, CD30, CD20, CD19, CD33, CD4, CD25, CD3, CA 72-4, CA 15-3, CA 27-29, CA 125, CA 19-9, beta-human chorionic gonadotropin, 1-2 microglobulin, squamous cell carcinoma antigen, neuron-specific enoJase, heat shock protein gp96, GM2, sargramostim, CTLA-4, 707-AP, ART-4, CAP-1, CLCA2, Cyp-B, HST-2, HPV proteins, EBV proteins, Hepatitis B or C virus proteins, and/or HIV proteins.

In some embodiments, the disclosure provides for a TβRII fusion polypeptide wherein the polypeptide does not comprise an additional ligand binding domain in addition to the TβRII domain in the same linear sequence. In some embodiments, the polypeptide comprises a linear amino acid sequence comprising a TβRII domain and a heterologous portion (e.g., an Fc portion), but the linear amino acid sequence does not comprise any additional ligand binding domains. In some embodiments, the polypeptide comprises a linear amino acid sequence comprising a TβRII domain and an Fc portion, but the linear amino acid sequence does not comprise any additional ligand binding domains. In some embodiments, the disclosure provides for a TβRII fusion polypeptide wherein the polypeptide does not comprise multiple ligand binding domains in a single linear amino acid sequence. In some embodiments, the disclosure provides for a TβRII fusion polypeptide wherein the polypeptide does not comprise more than one continuous linker sequence in a single linear amino acid sequence. In some embodiments, the polypeptide does not comprise multiple continuous glycine and/or serine linkers (e.g., a linker comprising (GGGGS)n, wherein n=>4) in a single linear amino acid sequence. In some embodiments, the disclosure provides for a TβRII fusion polypeptide wherein the heterologous portion is an Fc domain, and wherein only one continuous linker is covalently bound to the Fc domain. In some embodiments, the only one continuous linker comprises or consists of a (GGGGS)n linker, wherein n=>4.

### B. Alternative Multispecific Binders

In some embodiments, the disclosure provides for a multispecific binder of TGFβ-superfamily ligands. In some embodiments, the multispecific binder is capable of binding to a) at least one of TGFβ1 and TGFβ3, and b) at least one of activin A, activin B, activin AB, GDF11, and GDF8. In some embodiments, the multispecific binder comprises: a) a first portion that is capable of binding to TGFβ1 and/or TGFβ3; and b) a second portion that is capable of binding to at least one of activin A, activin B, activin AB, GDF11, and GDF8. In some embodiments, the multispecific binder comprises a TβRII polypeptide and a follistatin or a follistatin-like protein domain. In some embodiments, the multispecific binder comprises a TβRII polypeptide and an antibody or antigen-binding fragment, wherein the antibody or antigen-binding fragment is capable of binding to one or more of activin A, activin B, activin AB, GDF11, and/or GDF8. In particular embodiments, the multispecific binder comprises a TβRII polypeptide and an antibody or antigen-binding fragment, wherein the antibody or antigen-binding fragment is capable of binding to GDF8.

i. Follistatin and Follistatin-Like Polypeptides

In some embodiments, the disclosure provides for a multispecific binder comprising any of the TβRII polypeptides disclosed herein and a follistatin or follistatin-like polypeptide. As used herein, the term “follistatin” refers to a family of follistatin (FST) proteins and follistatin-related proteins, derived from any species. Follistatin is an autocrine glycoprotein that is expressed in nearly all tissues of higher animals. It was initially isolated from follicular fluid and was identified as a protein fraction that inhibited follicle-stimulating hormone (FSH) secretion from the anterior pituitary, and therefore was designated as FSH-suppressing protein (FSP). Subsequently, its primary function has been determined to be the binding and neutralization of members of the TGF-β superfamily including, for example, activin, a paracrine hormone that enhances secretion of FSH in the anterior pituitary.

The term “follistatin polypeptide” is used to refer to polypeptides comprising any naturally occurring polypeptide of the follistatin family as well as any variants thereof (including mutants, fragments, fusions, and peptidomimetic forms) that retain a useful activity, including, for example, ligand binding (e.g., myostatin (GDF8), GDF11, activin A, activin B) or heparin binding. For example, follistatin polypeptides include polypeptides comprising an amino acid sequence derived from the sequence of any known follistatin having a sequence at least about 80% identical to the sequence of a follistatin polypeptide, and preferably at least 85%, 90%, 95%, 97%, 99% or greater identity. The term “follistatin polypeptide” may refer to fusion proteins that comprise any of the polypeptides mentioned above along with a heterologous (non-follistatin) portion. An amino acid sequence is understood to be heterologous to follistatin if it is not uniquely found in the long (315 amino acid) form of human follistatin, represented by SEQ ID NO: 112. Many examples of heterologous portions are provided herein, and such heterologous portions may be immediately adjacent, by amino acid sequence, to the follistatin polypeptide portion of a fusion protein, or separated by intervening amino acid sequence, such as a linker or other sequence.

Follistatin is a single-chain polypeptide with a range of molecular weights from 31 to 49 kDa based on alternative mRNA splicing and variable glycosylation of the protein. The alternatively spliced mRNAs encode two proteins of 315 amino acids (i.e., FST315) and 288 amino acids (i.e., FST288); follistatin 315 can be further proteolytically degraded to follistatin 303 (FST303). Analysis of the amino acid sequence has revealed that the native human follistatin polypeptide comprises five domains (from the N-terminal side): a signal sequence peptide (amino acids 1-29 of SEQ ID NO: 110), an N-terminal domain (FSN) (amino acids 30-94 of SEQ ID NO: 110), follistatin domain I (FSDI) (amino acids 95-164 of SEQ ID NO: 110), follistatin domain II (FSDII) (amino acids (168-239 of SEQ ID NO: 110), and follistatin domain III (FSDIII) (amino acids 245-316 of SEQ ID NO: 110). See PNAS, U.S.A., 1988, Vol. 85, No 12, pp 4218-4222. In some embodiments, any of the follistatin polypeptides disclosed herein comprises any one or more of the follistatin polypeptide domains disclosed herein.

The human follistatin-288 (FST288) precursor has the following amino acid sequence, with the signal peptide indicated in bold, the N-terminal domain (FSN) indicated by single underlining, and the follistatin domains I-III (FSI, FSII, FSIII) indicated by double underlining.

(SEQ ID NO: 110)
MVRARHQPGGLCLLLLLLCQFMEDRSAQAGNCWLRQAKNGRCQVLYK
TELSKEECCSTGRLSTSWTEEDVNDNTLFKWMIENGGAPNCIPCKET
CENVDCGPGKKCRMNKKNKPRCVCAPDCSNITWKGPVCGLDGKTYRN
ECALLKARCKEQPELEVQYQGRCKKTCRDVECPGSSTCVVDQTNNAY
CVTCNRICPEPASSEQYLCGNDGVTYSSACHLRKATCLLGRSIGLAY
EGKCIKAKSCEDIQCTGGKKCLWDFKVGRGRCSLCDELCPDSKSDEP
VCASDNATYASECAMKEAACSSGVLLEVKHSGSCN

The processed (mature) human follistatin variant FST(288) has the following amino acid sequence with the N-terminal domain indicated by single underlining, and the follistatin domains I-III indicated by double underlining. Moreover, it will be appreciated that any of the initial amino acids G or N, prior to the first cysteine may be removed by processing or intentionally eliminated without any consequence, and polypeptides comprising such slightly smaller polypeptides are further included.

(SEQ ID NO: 111)
GNCWLRQAKNGRCQVLYKTELSKEECCSTGRLSTSWTEEDVNDN
TLFKWMIFNGGAPNCIPCKETCENVDCGPGKKCRMNKKNKPRCV
CAPDCSNITWKGPVCGLDGKTYRNECALLKARCKEQPELEVQYQ
GRCKKTCRDVFCPGSSTCVVDQTNNAYCVTCNRICPEPASSEQY
LCGNDGVTYSSACHLRKATCLLGRSIGLAYEGKCIKAKSCEDIQ
CTGGKKCLWDFKVGRGRCSLCDELCPDSKSDEPVCASDNATYAS
ECAMKEAACSSGVLLEVKHSGSCN

The human follistatin-315 (FST315) precursor has the following amino acid sequence, with the signal peptide indicated in bold, the N-terminal domain (FSN) indicated by single underlining, and the follistatin domains I-III (FSI, FSII, FSIII) indicated by double underlining (NCBI Accession Number AAH04107.1; 344 amino acids).

(SEQ ID NO: 112)
MVRARHQPGGLCLLLLLLCQFMEDR
SAQAGNQWLRQAKNGRCQVLYKTEL
SKEECCSTGRLSTSWTEEDVNDNTL
EKWMIENGGAPNCIPCKETCENVDC
GPGKKCRMNKKNKPRCVCAPDCSNI
TWKGPVCGLDGKTYRNECALLKARC
KEQPELEVQYQGRCKKTCRDVECPG
SSTCVVDQTNNAYCVTCNRICPEPA
SSEQYLCGNDGVTYSSACHLRKATC
LLGRSIGLAYEGKCIKAKSCEDIQC
TGGKKCLWDFKVGRGRCSLCDELCP
DSKSDEPVCASDNATYASECAMKEA
ACSSGVLLEVKHSGSCNSISEDTEE
EEEDEDQDYSFPISSILEW

The processed (mature) human FST(315) has the following amino acid sequence with the N-terminal domain indicated by single underlining, and the follistatin domains I-III indicated by double underlining. Moreover, it will be appreciated that any of the initial amino acids G or N, prior to the first cysteine may be removed by processing or intentionally eliminated without any consequence, and polypeptides comprising such slightly smaller polypeptides are further included.

(SEQ ID NO: 113)
GNCWLRQAKNGRCQVLYKTELSKEE
CCSTGRLSTSWTEEDVNDNTLFKWM
IFNGGAPNCIPCKETCENVDCGPGK
KCRMNKKNKPRCVCAPDCSNITWKG
PVCGLDGKTYRNECALLKARCKEQP
ELEVQYQGRCKKTCRDVECPGSSTC
VVDQTNNAYCVTCNRICPEPASSEQ
YLQGNDGVTYSSACHLRKATCLLGR
SIGLAYEGKCIKAKSCEDIQCTGGK
KCLWDEKVGRGRCSLCDELCPDSKS
DEPVCASDNATYASECAMKEAACSS
GVLLEVKHSGSCNSISEDTEEEEED
EDQDYSFPISSILEW

Follistatin proteins herein may be referred to as FST. If followed by a number, such as FST(288), this indicates that the protein is the 288 form of follistatin. If presented as FST(288)-Fc, this indicates a C-terminal Fc fusion to the FST(288), which may or may not include an intervening linker. The Fc in this instance may be any immunoglobulin Fc portion as that term is defined herein. If presented as FST(288)-IgG2, this indicates a C-terminal Fc fusion to the FST(288) of the Fc portion of human IgG2.

The term “biologically active”, in all its grammatical forms, when used in the context of a follistatin polypeptide or variant or fragment thereof, refers to a polypeptide with the ability to bind a ligand from at least one of the (1) activin or (2) bone morphogenic protein (BMP) class of ligands. In some embodiments, the “biologically active” follistatin is capable of binding to GDF8. In some embodiments, a biologically active polypeptide or fragment thereof inhibits the activity of a ligand from at least one of the (1) activin or (2) bone morphogenic protein (BMP) class of ligands. In some embodiments, a biologically active follistatin polypeptide or variant or fragment thereof inhibits GDF8, activin A and/or GDF-11 in a cell-based reporter gene assay with a lower IC₅₀ than the IC₅₀ of a follistatin polypeptide comprising the amino acid sequence of SEQ ID NO: 111. In some embodiments, a biologically active follistatin polypeptide or variant or fragment thereof inhibits GDF8, activin A and/or GDF-11 in a cell-based reporter gene assay with an equal IC₅₀ as compared to the IC₅₀ of a follistatin polypeptide comprising the amino acid sequence of SEQ ID NO: 111. In some embodiments, a biologically active follistatin polypeptide or variant or fragment thereof binds to one or more ligands selected from the group consisting of: GDF8 (myostatin), GDF11, activin A and activin B with a K_{D} less than 1 nM, 100 pM, 50 pM or 10 pM. In some embodiments, a biologically active follistatin polypeptide or variant or fragment thereof binds heparin with a greater affinity as compared to a follistatin polypeptide comprising the amino acid sequence of SEQ ID NO: 113. In some embodiments, a biologically active follistatin polypeptide or variant or fragment thereof binds heparin with an equal binding affinity to a follistatin polypeptide comprising the amino acid sequence of SEQ ID NO: 113. In some embodiments, the follistatin proteins are truncated forms exemplified by polypeptides comprising SEQ ID NO: 111, 116, 117, 118, 119, 120, 121, 122, 123, 124 or 125, and variants thereof. In some embodiments, any of the follistatin polypeptides, fragments, functional variants, and modified forms disclosed herein may have similar, the same or improved biological activities as compared to a wild-type follistatin polypeptide (e.g., a polypeptide having the amino acid sequence of SEQ ID NO: 111 or 113). For example, in some embodiments, a follistatin variant of the disclosure may bind to and inhibit function of a follistatin ligand (e.g., activin A, activin AB, activin B, and GDF8). In some embodiments, a follistatin polypeptide modulates growth of tissues, particularly muscle. Examples of follistatin polypeptides include polypeptides comprising, consisting essentially of or consisting of the amino acid sequences by any of SEQ ID NOs: 110-125, 135, 137-139, and 141-148 or biologically active fragments thereof, as well as polypeptides comprising, consisting essentially of or consisting of amino acid sequences that are at least 80%, 81%, 82%, 83%, 84%, 85%, 86%, 87%, 88%, 89%, 90%, 91%, 92%, 93%, 94%, 95%, 96%, 97%, 98%, 99% or 100% identical to an amino acid sequence of any of SEQ ID NOs: 110-125, 135, 137-139, and 141-148, or biologically active fragments thereof. In particular embodiments, the follistatin polypeptide comprises, consists or consists essentially of an amino acid sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to the amino acid sequence of SEQ ID NO: 111. Variations on these polypeptides may be prepared according to the following guidance. Unless stated otherwise, the numbering of amino acids in the follistatin polypeptides is based on the sequence of SEQ ID NO: 110, regardless of whether the native leader sequence is used. As described above, follistatin is characterized by three cysteine-rich regions (i.e., FS domains I-III) that are believed to mediate follistatin-ligand binding. Furthermore, researchers have demonstrated that polypeptide constructs comprising only one of the three FS-binding domains (e.g., FSDI) retains strong affinity towards certain follistatin-ligands (e.g., myostatin) and is biologically active in vivo. See Nakatani et al., The FASEB Journal, Vol. 22477-487 (2008). Therefore, variant follistatin polypeptides of the disclosure may comprise one or more active portions of a follistatin protein. For example, constructs of the disclosure may begin at a residue corresponding to amino acids 30-95 of SEQ ID NO: 112 and end at a position corresponding to amino acids 316-344 of SEQ ID NO: 112. Other examples include constructs that begin at a position from 30-95 of SEQ ID NO: 110 and end at a position corresponding to amino acids 164-167 or 238-244 of SEQ ID NO: 110. Others may include any of SEQ ID Nos. 116-125. Further examples include constructs that end at a position corresponding to an amino acid selected from the group consisting of the amino acid corresponding to amino acid 289, 290, 291, 292, 293, 294, 295, 296, 297, 298, 299, 300, 301, 302, 303, 304, and 305 of SEQ ID NO: 113. In some embodiments, follistatin polypeptides and constructs of the disclosure may comprise follistatin polypeptides which do not include residues corresponding to the amino acids selected from the group consisting of amino acids 289-315, 290-315, 291-315, 292-315, 293-315, 294-315, 295-315, 296-315, 297-315, 298-315, 299-315, 300-315, 301-315, 302-315, 303-315, 304-315, and 305-315 of SEQ ID NO: 113.

Follistatin polypeptides of the disclosure may include any naturally occurring domain of a follistatin protein as well as variants thereof (e.g., mutants, fragments, and peptidomimetic forms) that retain a useful activity. For example, it is well-known that FST(315) and FST(288) have high affinity for both activin (activin A and activin B) and myostatin (and the closely related GDF11) and that the follistatin domains (e.g., FSN and FSD I-III) are thought to be involved in the binding of such TGF-β ligands. However, it believed that each of these three domains may have a different affinity for these TGF-β ligands. For example, a recent study has demonstrated that polypeptide constructs comprising only the N-terminal domain (FSN) and two FSDI domains in tandem retained high affinity for myostatin, demonstrated little or no affinity for activin and promoted systemic muscle growth when introduced into a mouse by gene expression (Nakatani et al., The FASEB Journal, Vol. 22477-487 (2008)).

Additionally, the FSDI domain contains the heparin binding domain of human follistatin, which has the amino acid sequence of KKCRMNKKNKPR (SEQ ID NO: 114). This heparin binding domain can be represented as BBXBXXBBXBXB (SEQ ID NO: 115) wherein “B” means a basic amino acid, particularly lysine (K) or arginine (R). Accordingly, the present disclosure encompasses, in part, variant follistatin proteins that demonstrate selective binding and/or inhibition of a given TGF-β ligand relative to the naturally occurring FST protein (e.g., maintaining high-affinity for myostatin while having a significantly reduced affinity for activin).

In certain aspects, the disclosure includes polypeptides comprising the FSN domain, as set forth below, and, for example, one or more heterologous polypeptide, and moreover, it will be appreciated that any of the initial amino acids G or N, prior to the first cysteine may be deleted, as in the example shown below (SEQ ID NO:117).

(SEQ ID NO: 116)
GNCWLRQAKNGRCQVLYKTELSKEECCSTGRLS
TSWTEEDVNDNTLFKWMIFNGGAPNCIPCKET
(SEQ ID NO: 117)
CWLRQAKNGRCQVLYKTELSKEECCSTGRLST
SWTEEDVNDNTLFKWMIFNGGAPNCIPCKET

In certain aspects, the disclosure includes polypeptides comprising the FSDI domain which contains the minimal core activities of myostatin (and/or GDF11) binding along with heparin binding as set forth below, and, for example, one or more heterologous polypeptide.

(SEQ ID NO: 118)
CENVDCGPGKKCRMNKKNKPRCVCAPDCSNITWKG
PVCGLDGKTYRNECALLKARCKEQPELEVQYQGRC

An FSDI sequence may be advantageously maintained in structural context by expression as a polypeptide further comprising the FSN domain. Accordingly, the disclosure includes polypeptides comprising the FSN-FSDI sequence, as set forth below (SEQ ID NO: 119), and, for example, one or more heterologous polypeptide, and moreover, it will be appreciated that any of the initial amino acids G or N, prior to the first cysteine may be removed by processing or intentionally eliminated without any consequence, and polypeptides comprising such slightly smaller polypeptides are further included.

(SEQ ID NO: 119)
CWLRQAKNGRCQVLYKTELSKEECCSTG
RLSTSWTEEDVNDNTLFKWMIFNGGAPN
CIPCKETCENVDCGPGKKCRMNKKNKPR
CVCAPDCSNITWKGPVCGLDGKTYRNEC
ALLKARCKEQPELEVQYQGRC

As demonstrated by Nakani et al., an FSN-FSDI-FSDI construct is sufficient to confer systemic muscle growth when genetically expressed in a mouse, and accordingly the disclosure includes polypeptides comprising the amino acid sequences below and, for example, one or more heterologous polypeptide.

(SEQ ID NO: 120)
CWLRQAKNGRCQVLYKTELSKEECCSTGRLSTSWTEEDV
NDNTLFKWMIFNGGAPNCIPCKETCENVDCGPGKKCRMN
KKNKPRCVCAPDCSNITWKGPVCGLDGKTYRNECALLKA
RCKEQPELEVQYQGRCKKTCENVDCGPGKKCRMNKKNKP
RCVCAPDCSNITWKGPVCGLDGKTYRNECALLKARCKEQ
PELEVQYQGRC

The FSDI sequence confers myostatin and GDF11 binding. It has been demonstrated that activins, particularly activin A but also activin B, are also negative regulators of muscle, and therefore a follistatin polypeptide that inhibits both the myostatin/GDF11 group and the activin A/activin B group may provide a more potent muscle effect. Moreover, in view of the findings herein demonstrating the low systemic availability of certain follistatin polypeptides, particularly those comprising a heparin binding domain, and more particularly in a homodimeric form, such as an Fc fusion, safety concerns associated with the known effects of activin inhibition on the reproductive axis and other tissues are alleviated. Given that FSDII confers activin A and B binding, the disclosure provides polypeptides comprising FSDI and FSDII (SEQ ID NO: 121), as well as FSN-FSDI-FSDII constructs (SEQ ID NO: 122) and, for example, one or more heterologous polypeptide.

(SEQ ID NO: 121)
CENVDCGPGKKCRMNKKNKPRCVCAPDCSNITWKGPVCGL
DGKTYRNECALLKARCKEQPELEVQYQGRCKKTCRDVFCP
GSSTCVVDQTNNAYCVTCNRICPEPASSEQYLCGNDGVTY
SSACHLRKATCLLGRSIGLAYEGKC
(SEQ ID NO: 122)
CWLRQAKNGRCQVLYKTELSKEECCSTGRLSTSWTEEDVN
DNTLFKWMIFNGGAPNCIPCKETCENVDCGPGKKCRMNKK
NKPRCVCAPDCSNITWKGPVCGLDGKTYRNECALLKARCK
EQPELEVQYQGRCKKTCRDVFCPGSSTCVVDQTNNAYCVT
CNRICPEPASSEQYLCGNDGVTYSSACHLRKATCLLGRSI
GLAYEGKC

As described in the Examples, a follistatin polypeptide of 291 amino acids (representing a truncation of the naturally occurring FST-315) has advantageous properties. Accordingly, unprocessed (SEQ ID NO: 123) and mature FST(291) (SEQ ID NO: 124) polypeptides are included in the disclosure and may be combined with heterologous proteins. Moreover, it will be appreciated that any of the initial amino acids G or N, prior to the first cysteine may be removed by processing or intentionally eliminated without any consequence, and polypeptides comprising such slightly smaller polypeptides are further included, such as the example shown below (SEQ ID NO: 125).

(SEQ ID NO: 123)
MVRARHQPGGLCLLLLLLCQFMEDRSAQAGNCWLRQAKN
GRCQVLYKTELSKEECCSTGRLSTSWTEEDVNDNTLFKW
MIFNGGAPNCIPCKETCENVDCGPGKKCRMNKKNKPRCV
CAPDCSNITWKGPVCGLDGKTYRNECALLKARCKEQPEL
EVQYQGRCKKTCRDVFCPGSSTCVVDQTNNAYCVTCNRI
CPEPASSEQYLCGNDGVTYSSACHLRKATCLLGRSIGLA
YEGKCIKAKSCEDIQCTGGKKCLWDFKVGRGRCSLCDEL
CPDSKSDEPVCASDNATYASECAMKEAACSSGVLLEVKH
SGSCNSIS
(SEQ ID NO: 124)
GNCWLRQAKNGRCQVLYKTELSKEECCSTGRLSTSWTEE
DVNDNTLFKWMIFNGGAPNCIPCKETCENVDCGPGKKCR
MNKKNKPRCVCAPDCSNITWKGPVCGLDGKTYRNECALL
KARCKEQPELEVQYQGRCKKTCRDVFCPGSSTCVVDQTN
NAYCVTCNRICPEPASSEQYLCGNDGVTYSSACHLRKAT
CLLGRSIGLAYEGKCIKAKSCEDIQCTGGKKCLWDFKVG
RGRCSLODELCPDSKSDEPVCASDNATYASECAMKEAAC
SSGVLLEVKHSGSCNSIS
(SEQ ID NO: 125)
CWLRQAKNGRCQVLYKTELSKEECCSTGRLSTSWTEEDV
NDNTLFKWMIFNGGAPNCIPCKETCENVDCGPGKKCRMN
KKNKPRCVCAPDCSNITWKGPVCGLDGKTYRNECALLKA
RCKEQPELEVQYQGRCKKTCRDVFCPGSSTCVVDQTNNA
YCVTCNRICPEPASSEQYLCGNDGVTYSSACHLRKATCL
LGRSIGLAYEGKCIKAKSCEDIQCTGGKKCLWDFKVGRG
RCSLCDELCPDSKSDEPVCASDNATYASECAMKEAACSS
GVLLEVKHSGSCNSIS

In certain embodiments, the present invention relates to antagonizing a ligand of follistatin (also referred to as a follistatin ligand) with a subject follistatin polypeptide (e.g., an FST-IgG fusion polypeptide). Thus, compositions and methods of the present disclosure are useful for treating disorders associated with abnormal activity of one or more ligands of follistatin. Exemplary ligands of follistatin include some TGF-β family members, such as activin A, activin B, myostatin (GDF8) and GDF11.

The follistatin variations described herein may be combined in various ways with each other or with heterologous amino acid sequences. For example, variant follistatin proteins of the disclosure include polypeptides that comprise one or more FS domains selected from FSDI (amino acids 95-164 of SEQ ID NO: 110), FSDII (amino acids 168-239 of SEQ ID NO: 110), or FSDIII (amino acids 245-316 of SEQ ID NO: 110) as well as proteins that comprise one or more FS domains selected from a sequence at least 80%, 85%, 90%, 92%, 95%, 96%, 97%, 98% or 99% identical to FSDI (amino acids 95-164 of SEQ ID NO: 110), FSDII (amino acids 168-239 of SEQ ID NO: 110), or FSDIII (amino acids 245-316 of SEQ ID NO: 110). In some embodiments, any of the follistatin polypeptides disclosed herein comprises any of the FS domains disclosed herein. These FS domains may be combined in any order within a variant follistatin polypeptide of the disclosure provided that such recombinant proteins maintain the desired activity including, for example, follistatin ligand-binding activity (e.g., myostatin) and biological activity (e.g., inducing muscle mass and/or strength). Examples of such follistatin variant polypeptides include, for example, polypeptides having domain structures such as FSDI-FSDII-FSDIII, FSDI-FSDIII, FSDI-FSDI-FSDIII, FSDI-FSDII, FSDI-FSDI, FSN-FSDI-FSDII-FSDIII, FSN-FSDI-FSDII, FSN-FSDI-FSDI, FSN-FSDI-FSDIII, FSN-FSDI-FSDI-FSDIII, and polypeptides obtained by fusing other heterologous polypeptides to the N-termini or the C-termini of these polypeptides. These domains may be directly linked or liked via a linker polypeptide. Optionally, polypeptide linkers may be any sequence and may comprise 1-50, preferably 1-10, and more preferably 1-5 amino acids. In certain aspects, preferred linkers contain no cysteine amino acids.

As referenced herein, “follistatin variants” includes follistatin polypeptides that are fragments and/or mutants/modified polypeptides as compared to a reference wildtype follistatin protein (e.g., a follistatin protein having the amino acid sequence of any of SEQ ID NOs: 110-113). In some embodiments, follistatin variants of the disclosure have reduced or abolished binding affinity for one or more follistatin ligands as compared to a wildtype follistatin polypeptide (e.g., a polypeptide having the amino acid sequence of SEQ ID NO: 113). In certain aspects, the disclosure provides follistatin variants that have reduced or abolished binding affinity for activin as compared to a wildtype follistatin polypeptide (e.g., a polypeptide having the amino acid sequence of SEQ ID NO: 113). In certain aspects, the disclosure provides follistatin variants that have reduced or abolished binding affinity for activin but retain high affinity for myostatin as compared to a wildtype follistatin polypeptide (e.g., a polypeptide having the amino acid sequence of SEQ ID NO: 113). In certain aspects, the disclosure provides follistatin variants that have reduced or abolished binding affinity for GDF11 as compared to a wildtype follistatin polypeptide (e.g., a polypeptide having the amino acid sequence of SEQ ID NO: 113).

In some embodiments, follistatin fragments or variants of the disclosure have increased binding affinity for heparin. In some embodiments, follistatin fragments or variants of the disclosure have a binding affinity for heparin which is equivalent to the binding affinity of a follistatin polypeptide comprising SEQ ID NO: 111. In some embodiments, follistatin fragments or variants have a binding affinity for heparin that is at least 20%, 30%, 40%, 50%, 60%, 70%, 80%, 90% or 95% of the binding affinity for heparin of a follistatin polypeptide comprising SEQ ID NO: 111. In some embodiments, follistatin fragments or variants of the disclosure have a binding affinity for heparin which is greater than the binding affinity of a follistatin polypeptide comprising SEQ ID NO: 111. In some embodiments, follistatin fragments or variants of the disclosure have a binding affinity for heparin which is greater than the binding affinity of a follistatin polypeptide comprising SEQ ID NO: 113. In some embodiments, follistatin fragments or variants of the disclosure have an unmasked heparin binding domain. In some embodiments, follistatin fragments or variants of the disclosure comprise a heparin binding domain which comprises the endogenous follistatin heparin binding sequence. In some embodiments, follistatin fragments or variants of the disclosure comprise a heparin binding domain which comprises the endogenous follistatin heparin binding sequence (e.g., SEQ ID NO: 114). In some embodiments, follistatin fragments or variants of the disclosure comprise a heterologous heparin binding sequence.

In certain aspects, the disclosure provides follistatin fragments or variants that do not comprise a sequence corresponding to the FSDII domain or functionally active FSDII domain. For example, follistatin polypeptides of the disclosure may include a variant obtained through partial or complete deletion of the FSDII domain. In certain aspects, such follistatin variants include the deletion of one or more cysteine residues within the FSDII region or substitution with non-cysteine amino acids.

The follistatin proteins of the disclosure may comprise a signal sequence. The signal sequence can be a native signal sequence of a follistatin protein (e.g., amino acids 1-29 of SEQ ID NO: 110) or a signal sequence from another protein, such as tissue plasminogen activator (TPA) signal sequence or a honey bee melatin (HBM) signal sequence. In some embodiments, the signal sequence is removed during processing of the follistatin protein.

Further N-linked glycosylation sites (N-X-S/T) may be added to a follistatin polypeptide, and may increase the serum half-life of an FST-Fc fusion protein. N-X-S/T sequences may be generally introduced at positions outside the ligand-binding pocket. N-X-S/T sequences may be introduced into the linker between the follistatin sequence and the Fc or other fusion component. Such a site may be introduced with minimal effort by introducing an N in the correct position with respect to a pre-existing S or T, or by introducing an S or T at a position corresponding to a pre-existing N. Any S that is predicted to be glycosylated may be altered to a T without creating an immunogenic site, because of the protection afforded by the glycosylation. Likewise, any T that is predicted to be glycosylated may be altered to an S. Accordingly, a follistatin variant may include one or more additional, non-endogenous N-linked glycosylation consensus sequences.

In certain embodiments, the present disclosure contemplates making functional variants by modifying the structure of a follistatin polypeptide for such purposes as enhancing therapeutic efficacy, or stability (e.g., ex vivo shelf life and resistance to proteolytic degradation in vivo). Modified follistatin polypeptides can also be produced, for instance, by amino acid substitution, deletion, or addition. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a follistatin polypeptide results in a functional homolog can be readily determined by assessing the ability of the variant follistatin polypeptide to produce a response in cells in a fashion similar to the wild-type follistatin polypeptide, or to bind to one or more ligands, such as activin or myostatin in a fashion similar to wild-type follistatin.

In certain embodiments, the present invention contemplates specific mutations of the follistatin polypeptides so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine (where “X” is any amino acid) which is specifically recognized by appropriate cellular glycosylation enzymes. The alteration may also be made by the addition of, or substitution by, one or more serine or threonine residues to the sequence of the wild-type follistatin polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a follistatin polypeptide is by chemical or enzymatic coupling of glycosides to the follistatin polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. These methods are described in WO 87/05330 published Sep. 11, 1987, and in Aplin and Wriston (1981) CRC Crit. Rev. Biochem., pp. 259-306, incorporated by reference herein. Removal of one or more carbohydrate moieties present on an ActRIIB polypeptide may be accomplished chemically and/or enzymatically. Chemical deglycosylation may involve, for example, exposure of the follistatin polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Chemical deglycosylation is further described by Hakimuddin et al. (1987) Arch. Biochem. Biophys. 259:52 and by Edge et al. (1981) Anal. Biochem. 118:131. Enzymatic cleavage of carbohydrate moieties on follistatin polypeptides can be achieved by the use of a variety of endo- and exo-glycosidases as described by Thotakura et al. (1987) Meth. Enzymol. 138:350. The sequence of a follistatin polypeptide may be adjusted, as appropriate, depending on the type of expression system used, as mammalian, yeast, insect and plant cells may all introduce differing glycosylation patterns that can be affected by the amino acid sequence of the peptide. In some embodiments, follistatin proteins for use in humans will be expressed in a cell line (e.g., a mammalian cell line) that provides proper glycosylation, such as HEK293 or CHO cell lines, although other expression cell lines are expected to be useful as well.

This disclosure further contemplates a method of generating variants, particularly sets of combinatorial variants of an follistatin polypeptide, including, optionally, truncation variants; pools of combinatorial mutants are especially useful for identifying functional variant sequences. The purpose of screening such combinatorial libraries may be to generate, for example, follistatin polypeptide variants that have altered properties, such as altered pharmacokinetics, or altered ligand binding as compared to a wildtype follistatin polypeptide (e.g., a polypeptide having the amino acid sequence of SEQ ID NO: 111 or 113). A variety of screening assays are provided below, and such assays may be used to evaluate variants. For example, a follistatin polypeptide variant may be screened for its ability to bind to a follistatin ligand, and/or to prevent binding of a follistatin ligand to a follistatin polypeptide.

The activity of a follistatin polypeptide or its variants may also be tested in a cell-based or in vivo assay. For example, the effect of a follistatin polypeptide variant on the expression of genes involved in muscle production may be assessed. This may, as needed, be performed in the presence of one or more recombinant follistatin ligand proteins (e.g., activin A), and cells may be transfected so as to produce a follistatin polypeptide and/or variants thereof, and optionally, a follistatin ligand. Likewise, a follistatin polypeptide may be administered to a mouse or other animal, and one or more muscle properties, such as muscle mass or strength may be assessed. In some embodiments, any of the follistatin polypeptides disclosed herein may be administered to an animal model of muscle contractures, and the effects of the follistatin polypeptide on the animal model may be assessed (see, e.g., Example 8). Such assays are either described in the application or are well known and routine in the art. A responsive reporter gene may be used in such cell lines to monitor effects on downstream signaling.

Combinatorially-derived variants can be generated which have a selective potency relative to a naturally occurring follistatin polypeptide. Such variant proteins, when expressed from recombinant DNA constructs, can be used in gene therapy protocols. Likewise, mutagenesis can give rise to variants which have intracellular half-lives dramatically different than the corresponding a wild-type follistatin polypeptide. For example, the altered protein can be rendered either more stable or less stable to proteolytic degradation or other processes which result in destruction of, or otherwise inactivation of a native follistatin polypeptide. Such variants, and the genes which encode them, can be utilized to alter follistatin polypeptide levels by modulating the half-life of the follistatin polypeptides. For instance, a short half-life can give rise to more transient biological effects and, when part of an inducible expression system, can allow tighter control of recombinant follistatin polypeptide levels within the cell.

In certain embodiments, the follistatin polypeptides of the disclosure may further comprise post-translational modifications in addition to any that are naturally present in the follistatin polypeptides. Such modifications include, but are not limited to, acetylation, carboxylation, glycosylation, phosphorylation, lipidation, and acylation. As a result, the modified follistatin polypeptides may contain non-amino acid elements, such as polyethylene glycols, lipids, poly- or mono-saccharide, and phosphates. Effects of such non-amino acid elements on the functionality of a follistatin polypeptide may be tested as described herein for other follistatin polypeptide variants. When a follistatin polypeptide is produced in cells by cleaving a nascent form of the follistatin polypeptide, post-translational processing may also be important for correct folding and/or function of the protein. Different cells (such as CHO, HeLa, MDCK, 293, WI38, NIH-3T3 or HEK293) have specific cellular machinery and characteristic mechanisms for such post-translational activities and may be chosen to ensure the correct modification and processing of the follistatin polypeptides.

In certain aspects, functional variants or modified forms of the follistatin polypeptides include fusion proteins having at least a portion of a follistatin polypeptide and one or more fusion domains. Well known examples of such fusion domains include, but are not limited to, polyhistidine, Glu-Glu, glutathione S transferase (GST), thioredoxin, protein A, protein G, an immunoglobulin heavy chain constant region (e.g., an Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners. As another example, a fusion domain may be selected so as to facilitate detection of the follistatin polypeptides. Examples of such detection domains include the various fluorescent proteins (e.g., GFP) as well as “epitope tags,” which are usually short peptide sequences for which a specific antibody is available. Well known epitope tags for which specific monoclonal antibodies are readily available include FLAG, influenza virus haemagglutinin (HA), and c-myc tags. In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation. In certain preferred embodiments, a follistatin polypeptide is fused with a domain that stabilizes the follistatin polypeptide in vivo (a “stabilizer” domain). By “stabilizing” is meant anything that increases serum half-life, regardless of whether this is because of decreased destruction, decreased clearance by the kidney, or other pharmacokinetic effect. Fusions with the Fc portion of an immunoglobulin are known to confer desirable pharmacokinetic properties on a wide range of proteins. Likewise, fusions to human serum albumin can confer desirable properties. Other types of fusion domains that may be selected include multimerizing (e.g., dimerizing, tetramerizing) domains and functional domains (that confer an additional biological function, such as further stimulation of muscle growth).

As specific examples, the present disclosure provides fusion proteins comprising follistatin polypeptides fused to a polypeptide comprising a heterologous moiety/domain. In some embodiments, the heterologous moiety is serum albumin. In some embodiments, the heterologous moiety is a constant domain of an immunoglobulin, such as a CH1, CH2 or CH3 domain of an immunoglobulin or an Fc. Fc domains derived from human IgG1 and IgG2 are provided below (SEQ ID NO: 126 and SEQ ID NO: 127, respectively). As described herein, an IgG2, IgG4 or IgG2/4 Fc domain is particularly advantageous for fusion with follistatin polypeptides that retain heparin binding activity because these Fc species have reduced CDC and/or ADCC activity which may be harmful to the cells to which these heparin binding polypeptides may adhere. Other mutations are known that decrease either CDC or ADCC activity, and collectively, any of these variants are included in the disclosure and may be used as advantageous components of a follistatin fusion protein. In some embodiments, any of the follistatin polypeptides disclosed herein is conjugated to an Fc domain comprising an amino acid sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to the amino acid sequence of SEQ ID NO: 126, or fragments thereof. In some embodiments, any of the follistatin polypeptides disclosed herein is conjugated to an Fc domain comprising an amino acid sequence that is at least 81%, 82%, 83%, 84%, 85%, 86%, 87%, 88%, 89%, 90%, 91%, 92%, 93%, 94%, 95%, 96%, 97%, 98%, 99% or 100% identical to the amino acid sequence of SEQ ID NO: 127, or fragments thereof. Optionally, the Fc domain of SEQ ID NO: 126 (or variant or fragment thereof) has one or more mutations at residues such as Asp-265, Lys-322, and Asn-434 (numbered in accordance with the corresponding full-length IgG1). In certain cases, the mutant Fc domain having one or more of these mutations (e.g., Asp-265 mutation) has reduced ability of binding to the Fc receptor relative to a wildtype Fc domain. In other cases, the mutant Fc domain having one or more of these mutations (e.g., Asn-434 mutation) has increased ability of binding to the MHC class I-related Fc-receptor (FcRN) relative to a wildtype Fc domain.

Examples of human IgG1 and IgG2 amino acid sequences that may be employed are shown below:

IgG1
(SEQ ID NO: 126)
THTCPPCPAPELLGGPSVFLFPPKPKDTLMISRTPEVTCV
VVDVSHEDPEVKFNWYVDGVEVHNAKTKPREEQYNSTYRV
VSVLTVLHQDWLNGKEYKCKVSNKALPAPIEKTISKAKGQ
PREPQVYTLPPSREEMTKNQVSLTCLVKGFYPSDIAVEWE
SNGQPENNYKTTPPVLDSDGSFFLYSKLTVDKSRWQQGNV
ESCSVMHEALHNHYTQKSLSLSPGK
IgG2
(SEQ ID NO: 127)
VECPPCPAPPVAGPSVFLEPPKPKDTLMISRTPEVTCVV
VDVSHEDPEVQFNWYVDGVEVHNAKTKPREEQFNSTFRV
VSVLTVVHQDWLNGKEYKCKVSNKGLPAPIEKTISKTKG
QPREPQVYTLPPSREEMTKNQVSLTCLVKGFYPSDIAVE
WESNGQPENNYKTTPPMLDSDGSFFLYSKLTVDKSRWQQ
GNVFSCSVMHEALHNHYTQKSLSLSPGK

It is understood that different elements of the fusion proteins may be arranged in any manner that is consistent with the desired functionality. For example, a follistatin polypeptide may be placed C-terminal to a heterologous moiety/domain, or, alternatively, a heterologous moiety/domain may be placed C-terminal to a follistatin polypeptide. The follistatin polypeptide domain and the heterologous domain need not be adjacent in a fusion protein, and additional domains or amino acid sequences may be included C- or N-terminal to either domain or between the domains. In some embodiments, the follistatin polypeptide is conjugated directly to the heterologous moiety/domain. In other embodiments, the follistatin polypeptide is conjugated to the heterologous moiety/domain by means of a linker. In some embodiments, the linker is a glycine, threonine and/or serine rich linker. Other near neutral amino acids, such as, but not limited to, Asn, Pro and Ala, may also be used in the linker sequence. In some embodiments, the linker comprises various permutations of amino acid sequences containing Gly and Thr. In some embodiments, the linker comprises various permutations of amino acid sequences containing Gly and Ser. In some embodiments, the linker has a length of at least 3, 4, 5, 7, 10, 12, 15, 20, 21, 25, 30, 35, 40, 45 or 50 amino acids. In some embodiments, the linker comprises GlyGlyGly (GGG), or repetitions thereof. In some embodiments, the linker comprises the amino acid sequence of ThrGlyGlyGly (TGGG) (SEQ ID NO: 128) or repetitions thereof. In some embodiments, the linker is 1-5, 1-10 or 1-15 amino acids in length. In some embodiments, the linker consists of ThrGlyGlyGly (TGGG) (SEQ ID NO: 128). In some embodiments, the linker is greater than 10 amino acids in length. In some embodiments, the linker comprises between 10-100, 10-90, 10-80, 10-70, 10-60, 10-50, 10-40, 10-30, 10-20, 10-15 amino acids. In some embodiments, the linker comprises at least 10, 15, 20, 25, 30, 35, 40, 45, 50, 55, 60, 65, 70, 75, 80, 85, 90, or 95 amino acids. In some embodiments, the linker comprises an amino acid sequence that is at least 80%, 85%, 90%, 95%, 96%, 97%, 98%, 99% or 100% identical to GAPGGGGGAAAAAGGGGGGAP (SEQ ID NO: 129) or fragments thereof. In some embodiments, the linker comprises an amino acid sequence that is at least 80%, 85%, 90%, 95%, 96%, 97%, 98%, 99% or 100% identical to GAPGGGGGAAAAAGGGGGGAPGGGGGAAAAAGGGGGGAP (SEQ ID NO: 130), or fragments thereof. In some embodiments, the linker comprises a sequence at least 80%, 85%, 90%, 95%, 96%, 97%, 98%, 99% or 100% identical to GAPGGGGGAAAAAGGGGGGAPGGGGGAAAAAGGGGGGAPGGGGGAAAAAGGGGG GAP (SEQ ID NO: 131), or fragments thereof. In some embodiments, the linker comprises the amino acid sequence of ALEVLFQGP (SEQ ID NO: 132). In some embodiments, the linker does not consist of or comprise the amino acid sequence of any one of SEQ ID NOs: 129-132.

As used herein, the term “immunoglobulin Fc domain” or simply “Fc” is understood to mean the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In a preferred embodiment the immunoglobulin Fc region comprises at least an immunoglobulin hinge region a CH2 domain and a CH3 domain, and preferably lacks the CH1 domain. It is also understood that a follistatin polypeptide may comprise only a domain of an immunoglobulin, such as a CH1 domain, a CH2 domain or a CH3 domain. Many of these domains confer desirable pharmacokinetic properties as well as dimerization or higher order multimerization.

In one embodiment, the class of immunoglobulin from which the heavy chain constant region is derived is IgG (Igγ) (γ subclasses 1, 2, 3, or 4). Other classes of immunoglobulin, IgA (Iga), IgD (Igδ), IgE (Igε) and IgM (Igμ), may be used. The choice of appropriate immunoglobulin heavy chain constant region is discussed in detail in U.S. Pat. Nos. 5,541,087 and 5,726,044. The choice of particular immunoglobulin heavy chain constant region sequences from certain immunoglobulin classes and subclasses to achieve a particular result is considered to be within the level of skill in the art. In certain embodiments, the constant domain of an IgG immunoglobulin has reduced or no substantial ADCC and/or CDC activity relative to native human IgG1. The portion of the DNA construct encoding the immunoglobulin Fc region preferably comprises at least a portion of a hinge domain, and preferably at least a portion of a CH₃ domain of Fc gamma or the homologous domains in any of IgA, IgD, IgE, or IgM.

Furthermore, it is contemplated that substitution or deletion of amino acids within the immunoglobulin heavy chain constant regions may be useful in the practice of the methods and compositions disclosed herein. One example would be to introduce amino acid substitutions in the upper CH2 region to create an Fc variant with reduced affinity for Fc receptors (Cole et al. (1997) J. Immunol. 159:3613). Additionally, in many instances, the C-terminal lysine, or K, will be removed and thus any of the polypeptides described herein may omit the C-terminal K that is found in an Fc domain, such as those shown in SEQ ID NO: 126 or SEQ ID NO: 127.

In certain embodiments, the final (carboxy-terminal) lysine, or K, of the follistatin polypeptide is absent. For example, the protein may comprise an amino acid sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to the sequence of SEQ ID NOs: 144 or 145, wherein the final (carboxy-terminal) lysine (K) of SEQ ID NO: 144 or 145, respectively, is optionally absent.

In certain embodiments, the follistatin polypeptides of the present disclosure contain one or more modifications that are capable of stabilizing the follistatin polypeptides. For example, such modifications enhance the in vitro half-life of the follistatin polypeptides, enhance circulatory half-life of the follistatin polypeptides or reducing proteolytic degradation of the follistatin polypeptides. Such stabilizing modifications include, but are not limited to, fusion proteins (including, for example, fusion proteins comprising a follistatin polypeptide and a stabilizer domain), modifications of a glycosylation site (including, for example, addition of a glycosylation site to a follistatin polypeptide), and modifications of carbohydrate moiety (including, for example, removal of carbohydrate moieties from a follistatin polypeptide). In the case of fusion proteins, a follistatin polypeptide is fused to a stabilizer domain such as an IgG molecule (e.g., an Fc domain). As used herein, the term “stabilizer domain” not only refers to a fusion domain (e.g., Fc) as in the case of fusion proteins, but also includes nonproteinaceous modifications such as a carbohydrate moiety, or nonproteinaceous polymer, such as polyethylene glycol.

A representative follistatin-Fc fusion protein is FST(288)-IgG2 fusion has the unprocessed and mature amino acid sequences shown below.

Unprocessed FST(288)-IgG2
(SEQ ID NO: 135)
MVRARHQPGGLCLLLLLLCQFMEDRSAQAGNCWLRQAKNGRCQVLYK
TELSKEECCSTGRLSTSWTEEDVNDNTLFKWMIFNGGAPNCIPCKET
CENVDCGPGKKCRMNKKNKPRCVCAPDCSNITWKGPVCGLDGKTYRN
ECALLKARCKEQPELEVQYQGRCKKTCRDVFCPGSSTCVVDQTNNAY
CVTCNRICPEPASSEQYLCGNDGVTYSSACHLRKATCLLGRSIGLAY
EGKCIKAKSCEDIQCTGGKKCLWDFKVGRGRCSLCDELCPDSKSDEP
VCASDNATYASECAMKEAACSSGVLLEVKHSGSCNTGGGVECPPCPA
PPVAGPSVFLFPPKPKDTLMISRTPEVTCVVVDVSHEDPEVQFNWYV
DGVEVHNAKTKPREEQFNSTFRVVSVLTVVHQDWLNGKEYKCKVSNK
GLPAPIEKTISKTKGQPREPQVYTLPPSREEMTKNQVSLTCLVKGFY
PSDIAVEWESNGQPENNYKTTPPMLDSDGSFFLYSKLTVDKSRWQQG
NVFSCSVMHEALHNHYTQKSLSLSPGK

Which is encoded by the following nucleic
acid sequence
(SEQ ID NO: 136)
atggtccgcgcgaggcaccagccgggtgggctttgcctcctgctgct
gctgctctgccagttcatggaggaccgcagtgcccaggctgggaact
gctggctccgtcaagcgaagaacggccgctgccaggtcctgtacaag
accgaactgagcaaggaggagtgctgcagcaccggccggctgagcac
ctcgtggaccgaggaggacgtgaatgacaacacactcttcaagtgga
tgattttcaacgggggcgcccccaactgcatcccctgtaaagaaacg
tgtgagaacgtggactgtggacctgggaaaaaatgccgaatgaacaa
gaagaacaaaccccgctgcgtctgcgccccggattgttccaacatca
cctggaagggtccagtctgcgggctggatgggaaaacctaccgcaat
gaatgtgcactcctaaaggcaagatgtaaagagcagccagaactgga
agtccagtaccaaggcagatgtaaaaagacttgtcgggatgttttct
gtccaggcagctccacatgtgtggtggaccagaccaataatgcctac
tgtgtgacctgtaatcggatttgcccagagcctgcttcctctgagca
atatctctgtgggaatgatggagtcacctactccagtgcctgccacc
tgagaaaggctacctgcctgctgggcagatctattggattagcctat
gagggaaagtgtatcaaagcaaagtcctgtgaagatatccagtgcac
tggtgggaaaaaatgtttatgggatttcaaggttgggagaggccggt
gttccctctgtgatgagctgtgccctgacagtaagtcggatgagcct
gtctgtgccagtgacaatgccacttatgccagcgagtgtgccatgaa
ggaagctgcctgctcctcaggtgtgctactggaagtaaagcactccg
gatcttgcaacaccggtggtggagtcgagtgcccaccgtgcccagca
ccacctgtggcaggaccgtcagtcttcctcttccccccaaaacccaa
ggacaccctcatgatctcccggacccctgaggtcacgtgcgtggtgg
tggacgtgagccacgaagaccccgaggtccagttcaactggtacgtg
gacggcgtggaggtgcataatgccaagacaaagccacgggaggagca
gttcaacagcacgttccgtgtggtcagcgtcctcaccgtcgtgcacc
aggactggctgaacggcaaggagtacaagtgcaaggtctccaacaaa
ggcctcccagcccccatcgagaaaaccatctccaaaaccaaagggca
gccccgagaaccacaggtgtacaccctgcccccatcccgggaggaga
tgaccaagaaccaggtcagcctgacctgcctggtcaaaggcttctac
cccagcgacatcgccgtggagtgggagagcaatgggcagccggagaa
caactacaagaccacacctcccatgctggactccgacggctccttct
tcctctacagcaagctcaccgtggacaagagcaggtggcagcagggg
aacgtcttctcatgctccgtgatgcatgaggctctgcacaaccacta
cacgcagaagagcctctccctgtctccgggtaaatgagaattc
Mature FST(288)-IgG2
(SEQ ID NO: 137)
GNCWLRQAKNGRCQVLYKTELSKEECCSTGRLSTSWTEEDVNDNTLF
KWMIFNGGAPNCIPCKETCENVDCGPGKKCRMNKKNKPRCVCAPDCS
NITWKGPVCGLDGKTYRNECALLKARCKEQPELEVQYQGRCKKTCRD
VFCPGSSTCVVDQTNNAYCVTCNRICPEPASSEQYLCGNDGVTYSSA
CHLRKATCLLGRSIGLAYEGKCIKAKSCEDIQCTGGKKCLWDFKVGR
GRCSLCDELCPDSKSDEPVCASDNATYASECAMKEAACSSGVLLEVK
HSGSCNTGGGVECPPCPAPPVAGPSVFLFPPKPKDTLMISRTPEVTC
VVVDVSHEDPEVQFNWYVDGVEVHNAKTKPREEQFNSTFRVVSVLTV
VHQDWLNGKEYKCKVSNKGLPAPIEKTISKTKGQPREPQVYTLPPSR
EEMTKNQVSLTCLVKGFYPSDIAVEWESNGQPENNYKTTPPMLDSDG
SFFLYSKLTVDKSRWQQGNVFSCSVMHEALHNHYTQKSLSLSPGK

The initial “GN” sequence may be removed, yielding the following polypeptide. (SEQ ID NO: 68 )

(SEQ ID NO: 138)
CWLRQAKNGRCQVLYKTELSKEECCSTGRLSTSWTEEDVNDNTLFKWMIFN
GGAPNCIPCKETCENVDCGPGKKCRMNKKNKPRCVCAPDCSNITWKGPVCG
LDGKTYRNECALLKARCKEQPELEVQYQGRCKKTCRDVFCPGSSTCVVDQT
NNAYCVTCNRICPEPASSEQYLCGNDGVTYSSACHLRKATCLLGRSIGLAY
EGKCIKAKSCEDIQCTGGKKCLWDFKVGRGRCSLCDELCPDSKSDEPVCAS
DNATYASECAMKEAACSSGVLLEVKHSGSCNTGGGVECPPCPAPPVAGPSV
FLFPPKPKDTLMISRTPEVTCVVVDVSHEDPEVQFNWYVDGVEVHNAKTKP
REEQFNSTFRVVSVLTVVHQDWLNGKEYKCKVSNKGLPAPIEKTISKTKGQ
PREPQVYTLPPSREEMTKNQVSLTCLVKGFYPSDIAVEWESNGQPENNYKT
TPPMLDSDGSFFLYSKLTVDKSRWQQGNVFSCSVMHEALHNHYTQKSLSLS
PGK

A further representative follistatin-Fc fusion protein is FST(315)-IgG2 fusion, which has the unprocessed and mature amino acid sequences shown below.

Unprocessed FST(315)-IgG2
(SEQ ID NO: 139)
MVRARHQPGGLCLLLLLLCQFMEDRSAQAGNCWLRQAKNGRCQVLYKTELS
KEECCSTGRLSTSWTEEDVNDNTLFKWMIFNGGAPNCIPCKETCENVDCGP
GKKCRMNKKNKPRCVCAPDCSNITWKGPVCGLDGKTYRNECALLKARCKEQ
PELEVQYQGRCKKTCRDVFCPGSSTCVVDQTNNAYCVTCNRICPEPASSEQ
YLCGNDGVTYSSACHLRKATCLLGRSIGLAYEGKCIKAKSCEDIQCTGGKK
CLWDFKVGRGRCSLCDELCPDSKSDEPVCASDNATYASECAMKEAACSSGV
LLEVKHSGSCNSISEDTEEEEEDEDQDYSFPISSILEWTGGGVECPPCPAP
PVAGPSVFLFPPKPKDTLMISRTPEVTCVVVDVSHEDPEVQFNWYVDGVEV
HNAKTKPREEQFNSTFRVVSVLTVVHQDWLNGKEYKCKVSNKGLPAPIEKT
ISKTKGQPREPQVYTLPPSREEMTKNQVSLTCLVKGFYPSDIAVEWESNGQ
PENNYKTTPPMLDSDGSFFLYSKLTVDKSRWQQGNVFSCSVMHEALHNHYT
QKSLSLSPGK

Which is encoded by the following nucleic acid
sequence
(SEQ ID NO: 140)
atggtccgcgcgaggcaccagccgggtgggctttgcctcctgctgctgctg
ctctgccagttcatggaggaccgcagtgcccaggctgggaactgctggctc
cgtcaagcgaagaacggccgctgccaggtcctgtacaagaccgaactgagc
aaggaggagtgctgcagcaccggccggctgagcacctcgtggaccgaggag
gacgtgaatgacaacacactcttcaagtggatgattttcaacgggggtgcc
cccaactgcatcccctgtaaagaaacgtgtgagaacgtggactgtggacct
gggaaaaaatgccgaatgaacaagaagaacaaaccccgctgcgtctgcgcc
ccggattgttccaacatcacctggaagggtccagtctgcgggctggatggg
aaaacctaccgcaatgaatgtgcactcctaaaggcaagatgtaaagagcag
ccagaactggaagtccagtaccaaggcagatgtaaaaagacttgtcgggat
gttttctgtccaggcagctccacatgtgtggtggaccagaccaataatgcc
tactgtgtgacctgtaatcggatttgcccagagcctgcttcctctgagcaa
tatctctgtgggaatgatggagtcacctactccagtgcctgccacctgaga
aaggctacctgcctgctgggcagatctattggattagcctatgagggaaag
tgtatcaaagcaaagtcctgtgaagatatccagtgcactggtgggaaaaaa
tgtttatgggatttcaaggttgggagaggccggtgttccctctgtgatgag
ctgtgccctgacagtaagtcggatgagcctgtctgtgccagtgacaatgcc
acttatgccagcgagtgtgccatgaaggaagctgcctgctcctcaggtgtg
ctactggaagtaaagcactccggatcttgcaactccatttcggaagacacc
gaggaagaggaggaagatgaagaccaggactacagctttcctatatcttct
attctagagtggaccggtggtggagtcgagtgcccaccgtgcccagcacca
cctgtggcaggaccgtcagtcttcctcttccccccaaaacccaaggacacc
ctcatgatctcccggacccctgaggtcacgtgcgtggtggtggacgtgagc
cacgaagaccccgaggtccagttcaactggtacgtggacggcgtggaggtg
cataatgccaagacaaagccacgggaggagcagttcaacagcacgttccgt
gtggtcagcgtcctcaccgtcgtgcaccaggactggctgaacggcaaggag
tacaagtgcaaggtctccaacaaaggcctcccagcccccatcgagaaaacc
atctccaaaaccaaagggcagccccgagaaccacaggtgtacaccctgccc
ccatcccgggaggagatgaccaagaaccaggtcagcctgacctgcctggtc
aaaggcttctaccccagcgacatcgccgtggagtgggagagcaatgggcag
ccggagaacaactacaagaccacacctcccatgctggactccgacggctcc
ttcttcctctacagcaagctcaccgtggacaagagcaggtggcagcagggg
aacgtcttctcatgctccgtgatgcatgaggctctgcacaaccactacacg
cagaagagcctctccctgtctccgggtaaatgagaattc
Mature FST(315)-IgG2
(SEQ ID NO: 141)
GNCWLRQAKNGRCQVLYKTELSKEECCSTGRLSTSWTEEDVNDNTLFKWMI
FNGGAPNCIPCKETCENVDCGPGKKCRMNKKNKPRCVCAPDCSNITWKGPV
CGLDGKTYRNECALLKARCKEQPELEVQYQGRCKKTCRDVFCPGSSTCVVD
QTNNAYCVTCNRICPEPASSEQYLCGNDGVTYSSACHLRKATCLLGRSIGL
AYEGKCIKAKSCEDIQCTGGKKCLWDFKVGRGRCSLCDELCPDSKSDEPVC
ASDNATYASECAMKEAACSSGVLLEVKHSGSCNSISEDTEEEEEDEDQDYS
FPISSILEWTGGGVECPPCPAPPVAGPSVFLFPPKPKDTLMISRTPEVTCV
VVDVSHEDPEVQFNWYVDGVEVHNAKTKPREEQFNSTFRVVSVLTVVHQDW
LNGKEYKCKVSNKGLPAPIEKTISKTKGQPREPQVYTLPPSREEMTKNQVS
LTCLVKGFYPSDIAVEWESNGQPENNYKTTPPMLDSDGSFELYSKLTVDKS
RWQQGNVFSCSVMHEALHNHYTQKSLSLSPGK

The initial “GN” sequence may be removed, yielding the following polypeptide. (

(SEQ ID NO: 142)
CWLRQAKNGRCQVLYKTELSKEECCSTGRLSTSWTEEDVNDNTLFKWMIFN
GGAPNCIPCKETCENVDCGPGKKCRMNKKNKPRCVCAPDCSNITWKGPVCG
LDGKTYRNECALLKARCKEQPELEVQYQGRCKKTCRDVFCPGSSTCVVDQT
NNAYCVTCNRICPEPASSEQYLCGNDGVTYSSACHLRKATCLLGRSIGLAY
EGKCIKAKSCEDIQCTGGKKCLWDFKVGRGRCSLCDELCPDSKSDEPVCAS
DNATYASECAMKEAACSSGVLLEVKHSGSCNSISEDTEEEEEDEDQDYSFP
ISSILEWTGGGVECPPCPAPPVAGPSVFLFPPKPKDTLMISRTPEVTCVVV
DVSHEDPEVQFNWYVDGVEVHNAKTKPREEQFNSTFRVVSVLTVVHQDWLN
GKEYKCKVSNKGLPAPIEKTISKTKGQPREPQVYTLPPSREEMTKNQVSLT
CLVKGFYPSDIAVEWESNGQPENNYKTTPPMLDSDGSFFLYSKLTVDKSRW
QQGNVFSCSVMHEALHNHYTQKSLSLSPGK

A further representative follistatin-Fc fusion is the FST(291)-IgG1 fusion, which has the unprocessed and mature amino acid sequences shown below.

Unprocessed FST(291)-IgG1
(SEQ ID NO: 143)
MVRARHQPGGLCLLLLLLCQFMEDRSAQAGNCWLRQAKNGRCQVLYKTELS
KEECCSTGRLSTSWTEEDVNDNTLFKWMIFNGGAPNCIPCKETCENVDCGP
GKKCRMNKKNKPRCVCAPDCSNITWKGPVCGLDGKTYRNECALLKARCKEQ
PELEVQYQGRCKKTCRDVFCPGSSTCVVDQTNNAYCVTCNRICPEPASSEQ
YLCGNDGVTYSSACHLRKATCLLGRSIGLAYEGKCIKAKSCEDIQCTGGKK
CLWDFKVGRGRCSLCDELCPDSKSDEPVCASDNATYASECAMKEAACSSGV
LLEVKHSGSCNSISTGGGTHTCPPCPAPELLGGPSVFLFPPKPKDTLMISR
TPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYS
KLTVDKSRWQQGNVFSCSVMHEALHNHYTQKSLSLSPGK
Mature FST(291)-IgG1
(SEQ ID NO: 144)
GNCWLRQAKNGRCQVLYKTELSKEECCSTGRLSTSWTEEDVNDNTLFKWMI
FNGGAPNCIPCKETCENVDCGPGKKCRMNKKNKPRCVCAPDCSNITWKGPV
CGLDGKTYRNECALLKARCKEQPELEVQYQGRCKKTCRDVFCPGSSTCVVD
QTNNAYCVTCNRICPEPASSEQYLCGNDGVTYSSACHLRKATCLLGRSIGL
AYEGKCIKAKSCEDIQCTGGKKCLWDFKVGRGRCSLCDELCPDSKSDEPVC
ASDNATYASECAMKEAACSSGVLLEVKHSGSCNSISTGGGTHTCPPCPAPE
LLGGPSVFLEPPKPKDTLMISRTPEVTCVVVDVSHEDPEVKFNWYVDGVEV
HNAKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYT
QKSLSLSPGK

The initial “GN” sequence may be removed, yielding the following polypeptide.

(SEQ ID NO: 145)
CWLRQAKNGRCQVLYKTELSKEECCSTGRLSTSWTEEDVNDNTLFKWMIFN
GGAPNCIPCKETCENVDCGPGKKCRMNKKNKPRCVCAPDCSNITWKGPVCG
LDGKTYRNECALLKARCKEQPELEVQYQGRCKKTCRDVFCPGSSTCVVDQT
NNAYCVTCNRICPEPASSEQYLCGNDGVTYSSACHLRKATCLLGRSIGLAY
EGKCIKAKSCEDIQCTGGKKCLWDFKVGRGRCSLCDELCPDSKSDEPVCAS
DNATYASECAMKEAACSSGVLLEVKHSGSCNSISTGGGTHTCPPCPAPELL
GGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQK
SLSLSPGK

The FST(291)-IgG2 fusion has the unprocessed and mature amino acid sequences shown below.

Unprocessed FST(291)-IgG2
(SEQ ID NO: 146)
MVRARHQPGGLCLLLLLLCQFMEDRSAQAGNCWLRQAKNGRCQVLYKTELS
KEECCSTGRLSTSWTEEDVNDNTLFKWMIFNGGAPNCIPCKETCENVDCGP
GKKCRMNKKNKPRCVCAPDCSNITWKGPVCGLDGKTYRNECALLKARCKEQ
PELEVQYQGRCKKTCRDVFCPGSSTCVVDQTNNAYCVTCNRICPEPASSEQ
YLCGNDGVTYSSACHLRKATCLLGRSIGLAYEGKCIKAKSCEDIQCTGGKK
CLWDFKVGRGRCSLCDELCPDSKSDEPVCASDNATYASECAMKEAACSSGV
LLEVKHSGSCNSISTGGGVECPPCPAPPVAGPSVFLFPPKPKDTLMISRTP
EVTCVVVDVSHEDPEVQFNWYVDGVEVHNAKTKPREEQFNSTFRVVSVLTV
VHQDWLNGKEYKCKVSNKGLPAPIEKTISKTKGQPREPQVYTLPPSREEMT
KNQVSLTCLVKGFYPSDIAVEWESNGQPENNYKTTPPMLDSDGSFFLYSKL
TVDKSRWQQGNVFSCSVMHEALHNHYTQKSLSLSPGK
Mature FST(291)-IgG2
(SEQ ID NO: 147)
GNCWLRQAKNGRCQVLYKTELSKEECCSTGRLSTSWTEEDVNDNTLFKWMI
FNGGAPNCIPCKETCENVDCGPGKKCRMNKKNKPRCVCAPDCSNITWKGPV
CGLDGKTYRNECALLKARCKEQPELEVQYQGRCKKTCRDVFCPGSSTCVVD
QTNNAYCVTCNRICPEPASSEQYLCGNDGVTYSSACHLRKATCLLGRSIGL
AYEGKCIKAKSCEDIQCTGGKKCLWDFKVGRGRCSLCDELCPDSKSDEPVC
ASDNATYASECAMKEAACSSGVLLEVKHSGSCNSISTGGGVECPPCPAPPV
AGPSVFLEPPKPKDTLMISRTPEVTCVVVDVSHEDPEVQFNWYVDGVEVHN
AKTKPREEQFNSTFRVVSVLTVVHQDWLNGKEYKCKVSNKGLPAPIEKTIS
KTKGQPREPQVYTLPPSREEMTKNQVSLTCLVKGFYPSDIAVEWESNGQPE
NNYKTTPPMLDSDGSFFLYSKLTVDKSRWQQGNVFSCSVMHEALHNHYTQK
SLSLSPGK

The initial “GN” sequence may be removed, yielding the following polypeptide.

(SEQ ID NO: 148)
CWLRQAKNGRCQVLYKTELSKEECCSTGRLSTSWTEEDVNDNTLFKWMIFN
GGAPNCIPCKETCENVDCGPGKKCRMNKKNKPRCVCAPDCSNITWKGPVCG
LDGKTYRNECALLKARCKEQPELEVQYQGRCKKTCRDVFCPGSSTCVVDQT
NNAYCVTCNRICPEPASSEQYLCGNDGVTYSSACHLRKATCLLGRSIGLAY
EGKCIKAKSCEDIQCTGGKKCLWDFKVGRGRCSLCDELCPDSKSDEPVCAS
DNATYASECAMKEAACSSGVLLEVKHSGSCNSISTGGGVECPPCPAPPVAG
PSVFLFPPKPKDTLMISRTPEVTCVVVDVSHEDPEVQFNWYVDGVEVHNAK
TKPREEQFNSTFRVVSVLTVVHQDWLNGKEYKCKVSNKGLPAPIEKTISKT
KGQPREPQVYTLPPSREEMTKNQVSLTCLVKGFYPSDIAVEWESNGQPENN
YKTTPPMLDSDGSFFLYSKLTVDKSRWQQGNVFSCSVMHEALHNHYTQKSL
SLSPGK

In certain embodiments, the present invention makes available isolated and/or purified forms of the follistatin polypeptides, which are isolated from, or otherwise substantially free of, other proteins.

In certain embodiments, follistatin polypeptides (unmodified or modified) of the disclosure can be produced by a variety of art-known techniques. For example, such follistatin polypeptides can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). Alternatively, the follistatin polypeptides, fragments or variants thereof may be recombinantly produced using various expression systems (e.g., E. coli, Chinese Hamster Ovary cells, COS cells, baculovirus) as is well known in the art (also see below). In a further embodiment, the modified or unmodified follistatin polypeptides may be produced by digestion of naturally occurring or recombinantly produced full-length follistatin polypeptides by using, for example, a protease, e.g., trypsin, thermolysin, chymotrypsin, pepsin, or paired basic amino acid converting enzyme (PACE). Computer analysis (using a commercially available software, e.g., MacVector, Omega, PCGene, Molecular Simulation, Inc.) can be used to identify proteolytic cleavage sites. Alternatively, such follistatin polypeptides may be produced from naturally occurring or recombinantly produced full-length follistatin polypeptides such as standard techniques known in the art, such as by chemical cleavage (e.g., cyanogen bromide, hydroxylamine).

In some embodiments, any of the follistatin or follistatin-like polypeptides disclosed herein is conjugated to any of the TβRII polypeptides disclosed herein. In some embodiments, the follistatin or follistatin-like polypeptide is directly fused to the TβRII polypeptide. In some embodiments, the follistatin or follistatin-like polypeptide is fused to the TβRII polypeptide by means of a linker. In some embodiments, the multi-specific binder is laid out in a format similar to that shown in FIG. 15A. In some embodiments, the multi-specific binder comprises a follistatin amino acid sequence that is at least 80%, 85%, 90%, 91%, 92%, 93%, 94%, 95%, 96%, 97%, 98%, 99% or 100% identical to the amino acid sequence of SEQ ID NO: 111. In some embodiments, the multi-specific binder comprises a heterologous domain. In some embodiments, the heterologous domain is an Fc domain. In some embodiments, the multispecific binder comprises an amino acid sequence that is at least 80%, 85%, 90%, 91%, 92%, 93%, 94%, 95%, 96%, 97%, 98%, 99% or 100% identical to the amino acid sequence of SEQ ID NO: 163.

| (SEQ ID NO: 163) | |
|---|---|
| 1 | 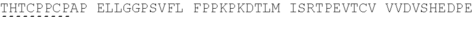 |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPG |

In some embodiments, the multispecific binder comprises a linker between the follistatin polypeptide portion and the Fc portion. In some embodiments, the linker comprises the amino acid sequence of GGG or the amino acid sequence of SEQ ID NO: 3.

In some embodiments, the multi-specific binder comprises an amino acid sequence that is at least 80%, 85%, 90%, 91%, 92%, 93%, 94%, 95%, 96%, 97%, 98%, 99% or 100% identical to the amino acid sequence of SEQ ID NO: 164.

(SEQ ID NO: 164)

| | |
|---|---|
| 1 | MVRARHQPGG LCLLLLLLCQ FMEDRSAQAG NCWLRQAKNG |
| | RCQVLYKTEL |
| 51 | SKEECCSTGR LSTSWTEEDV NDNTLFKWMI FNGGAPNCIP |
| | CKETCENVDC |
| 101 | GPGKKCRMNK KNKPRCVCAP DCSNITWKGP VCGLDGKTYR |
| | NECALLKARC |
| 151 | KEQPELEVQY QGRCKKTCRD VFCPGSSTCV VDQTNNAYCV |
| | TCNRICPEPA |
| 201 | SSEQYLCGND GVTYSSACHL RKATCLLGRS IGLAYEGKCI |
| | KAKSCEDIQC |
| 251 | TGGKKCLWDF KVGRGRCSLC DELCPDSKSD EPVCASDNAT |
| | YASECAMKEA |
| 301 | ACSSGVLLEV KHSGSCNTGG GTHTCPPCPA PELLGGPSVF |
| | LFPPKPKDTL |
| 351 | MISRTPEVTC VVVDVSHEDP EVKFNWYVDG VEVHNAKTKP |
| | REEQYNSTYR |
| 401 | VVSVLTVLHQ DWLNGKEYKC KVSNKALPAP IEKTISKAKG |
| | QPREPQVYTL |
| 451 | PPSREEMTKN QVSLTCLVKG FYPSDIAVEW ESNGQPENNY |
| | KTTPPVLDSD |
| 501 | GSFFLYSKLT VDKSRWQQGN VFSCSVMHEA LHNHYTQKSL |
| | SLSPGK |

In some embodiments, the multispecific binder comprises a TβRII polypeptide portion that is at least 80%, 85%, 90%, 91%, 92%, 93%, 94%, 95%, 96%, 97%, 98%, 99% or 100% identical to the amino acid sequence of SEQ ID NO: 170. In some embodiments, the TβRII polypeptide portion is fused to the follistatin polypeptide portion or the Fc portion by means of a linker. In some embodiments, the TβRII polypeptide portion is fused to the C-terminus of the Fc portion (e.g., the C-terminus of an amino acid sequence that is at least 80%, 85%, 90%, 91%, 92%, 93%, 94%, 95%, 96%, 97%, 98%, 99% or 100% identical to the amino acid sequence of SEQ ID NO: 164) by means of a linker. In some embodiments, the linker used to fuse the follistatin polypeptide portion or Fc portion to the TβRII polypeptide portion comprises an amino acid sequence that is at least 80%, 85%, 90%, 91%, 92%, 93%, 94%, 95%, 96%, 97%, 98%, 99% or 100% identical to the amino acid sequence of SEQ ID NO: 165.

(SEQ ID NO: 165)
AGGGGSGGGGSGGGGSGGGGSG

A representative nucleotide encoding a portion of a multispecific binder may comprise a nucleotide sequence that is at least 80%, 85%, 90%, 91%, 92%, 93%, 94%, 95%, 96%, 97%, 98%, 99% or 100% identical to the nucleotide sequence of SEQ ID NO: 166.

```
(SEQ ID NO: 166)
      1 ATGGTCCGCG CGAGGCACCA GCCGGGTGGG CTTTGCCTCC
          TGCTGCTGCT
    51 GCTCTGCCAG TTCATGGAGG ACCGCAGTGC CCAGGCTGGG
          AACTGCTGGC
  101 TCCGTCAAGC GAAGAACGGC CGCTGCCAGG TCCTGTACAA
          GACCGAACTG
  151 AGCAAGGAGG AGTGCTGCAG CACCGGCCGG CTGAGCACCT
          CGTGGACCGA
  201 GGAGGACGTG AATGACAACA CACTCTTCAA GTGGATGATT
          TTCAACGGGG
  251 GCGCCCCCAA CTGCATCCCC TGTAAAGAAA CGTGTGAGAA
          CGTGGACTGT
  301 GGACCTGGGA AAAAATGCCG AATGAACAAG AAGAACAAAC
          CCCGCTGCGT
  351 CTGCGCCCCG GATTGTTCCA ACATCACCTG GAAGGGTCCA
          GTCTGCGGGC
  401 TGGATGGGAA AACCTACCGC AATGAATGTG CACTCCTAAA
          GGCAAGATGT
  451 AAAGAGCAGC CAGAACTGGA AGTCCAGTAC CAAGGCAGAT
          GTAAAAAGAC
  501 TTGTCGGGAT GTTTTCTGTC CAGGCAGCTC CACATGTGTG
          GTGGACCAGA
  551 CCAATAATGC CTACTGTGTG ACCTGTAATC GGATTTGCCC
          AGAGCCTGCT
  601 TCCTCTGAGC AATATCTCTG TGGGAATGAT GGAGTCACCT
          ACTCCAGTGC
  651 CTGCCACCTG AGAAAGGCTA CCTGCCTGCT GGGCAGATCT
          ATTGGATTAG
  701 CCTATGAGGG AAAGTGTATC AAAGCAAAGT CCTGTGAAGA
          TATCCAGTGC
  751 ACTGGTGGGA AAAAATGTTT ATGGGATTTC AAGGTTGGGA
          GAGGCCGGTG
  801 TTCCCTCTGT GATGAGCTGT GCCCTGACAG TAAGTCGGAT
          GAGCCTGTCT
  851 GTGCCAGTGA CAATGCCACT TATGCCAGCG AGTGTGCCAT
          GAAGGAAGCT
  901 GCCTGCTCCT CAGGTGTGCT ACTGGAAGTA AAGCACTCCG
          GATCTTGCAA
  951 CACCGGTGGT GGAACTCACA CATGCCCACC GTGCCCAGCA
          CCTGAACTCC
1001 TGGGGGGACC GTCAGTCTTC CTCTTCCCCC CAAAACCCAA
          GGACACCCTC
1051 ATGATCTCCC GGACCCCTGA GGTCACATGC GTGGTGGTGG
          ACGTGAGCCA
1101 CGAAGACCCT GAGGTCAAGT TCAACTGGTA CGTGGACGGC
          GTGGAGGTGC
1151 ATAATGCCAA GACAAAGCCG CGGGAGGAGC AGTACAACAG
          CACGTACCGT
1201 GTGGTCAGCG TCCTCACCGT CCTGCACCAG GACTGGCTGA
          ATGGCAAGGA
1251 GTACAAGTGC AAGGTCTCCA ACAAAGCCCT CCCAGCCCCC
          ATCGAGAAAA
1301 CCATCTCCAA AGCCAAAGGG CAGCCCCGAG AACCACAGGT
          GTACACCCTG
1351 CCCCCATCCC GGGAGGAGAT GACCAAGAAC CAGGTCAGCC
          TGACCTGCCT
1401 GGTCAAAGGC TTCTATCCCA GCGACATCGC CGTGGAGTGG
          GAGAGCAATG
1451 GGCAGCCGGA GAACAACTAC AAGACCACGC CTCCCGTGCT
          GGACTCCGAC
1501 GGCTCCTTCT TCCTCTATAG CAAGCTCACC GTGGACAAGA
          GCAGGTGGCA
1551 GCAGGGGAAC GTCTTCTCAT GCTCCGTGAT GCATGAGGCT
          CTGCACAACC
1601 ACTACACGCA GAAGAGCCTC TCCCTGTCTC CGGGTAAATG
          A
```

In some embodiments, any of the multispecific binders disclosed herein comprise an amino acid sequence that is at least 80%, 85%, 90%, 91%, 92%, 93%, 94%, 95%, 96%, 97%, 98%, 99% or 100% identical to the amino acid sequence of SEQ ID NO: 180.

GNCWLRQAKNGRCQVLYKTELSKEECCSTGRLSTSWTEEDVND
NTLFKWMIFNGGAPNCIPCKETCENVDCGPGKKCRMNKKNKPR
CVCAPDCSNITWKGPVCGLDGKTYRNECALLKARCKEQPELEV
QYQGRCKKTCRDVFCPGSSTCVVDQTNNAYCVTCNRICPEPAS
SEQYLCGNDGVTYSSACHLRKATCLLGRSIGLAYEGKCIKAKS
CEDIQCTGGKKCLWDFKVGRGRCSLCDELCPDSKSDEPVCASD
NATYASECAMKEAACSSGVLLEVKHSGSCNTGGGTHTCPPCPA
PELLGGPSVFLFPPKPKDTLMISRTPEVTCVVVDVSHEDPEVK
FNWYVDGVEVHNAKTKPREEQYNSTYRVVSVLTVLHQDWLNGK
EYKCKVSNKALPAPIEKTISKAKGQPREPQVYTLPPSREEMTK
NQVSLTCLVKGFYPSDIAVEWESNGQPENNYKTTPPVLDSDGS
FFLYSKLTVDKSRWQQGNVFSCSVMHEALHNHYTQKSLSLSPG
KAGGGGSGGGGSGGGGSGGGGSGTIPPHVQKSDVEMEAQKDEI
ICPSCNRTAHPLRHINNDMIVTDNNGAVKFPQLCKFCDVRFST
CDNQKSCMSNCSITSICEKPQEVCVAVWRKNDENITLETVCHD
PKLPYHDFILEDAASPKCIMKEKKKPGETFFMCSCSSDECNDN
IIFSEEYNTSNPD

In some embodiments, any of the multispecific binders disclosed herein comprise an amino acid sequence that is at least 80%, 85%, 90%, 91%, 92%, 93%, 94%, 95%, 96%, 97%, 98%, 99% or 100% identical to the amino acid sequence of SEQ ID NO: 181.

GNCWLRQAKNGRCQVLYKTELSKEECCSTGRLSTSWTEEDVND
NTLFKWMIFNGGAPNCIPCKETCENVDCGPGKKCRMNKKNKPR
CVCAPDCSNITWKGPVCGLDGKTYRNECALLKARCKEQPELEV
QYQGRCKKTCRDVFCPGSSTCVVDQTNNAYCVTCNRICPEPAS
SEQYLCGNDGVTYSSACHLRKATCLLGRSIGLAYEGKCIKAKS
CEDIQCTGGKKCLWDFKVGRGRCSLCDELCPDSKSDEPVCASD
NATYASECAMKEAACSSGVLLEVKHSGSCNTGGGTHTCPPCPA
PELLGGPSVFLFPPKPKDTLMISRTPEVTCVVVDVSHEDPEVK
FNWYVDGVEVHNAKTKPREEQYNSTYRVVSVLTVLHQDWLNGK
EYKCKVSNKALPAPIEKTISKAKGQPREPQVYTLPPSREEMTK
NQVSLTCLVKGFYPSDIAVEWESNGQPENNYKTTPPVLDSDGS
FFLYSKLTVDKSRWQQGNVFSCSVMHEALHNHYTQKSLSLSPG
AGGGGSGGGGSGGGGSGGGGSGTIPPHVQKSDVEMEAQKDEII
CPSCNRTAHPLRHINNDMIVTDNNGAVKFPQLCKFCDVRFSTC
DNQKSCMSNCSITSICEKPQEVCVAVWRKNDENITLETVCHDP
KLPYHDFILEDAASPKCIMKEKKKPGETFFMCSCSSDECNDNI
IFSEEYNTSNPD

ii. Antibodies and Antigen-Binding Fragments Thereof

In some embodiments, the disclosure provides for a multispecific binder comprising any of the TβRII polypeptides disclosed herein and an antibody or antigen-binding fragment thereof. In some embodiments, the multispecific binder comprises a TβRII polypeptide and an antibody or antigen-binding fragment, wherein the antibody or antigen-binding fragment is capable of bining to one or more of activin A, activin B, activin AB, GDF11, and/or GDF8. In particular embodiments, the multispecific binder comprises a TβRII polypeptide and an antibody or antigen-binding fragment, wherein the antibody or antigen-binding fragment is capable of binding to GDF8.

As used herein, the term “antibody” (Ab), which is synonymous with the term “immunoglobulin” (Ig), means a tetramer comprising two heavy (H) chains (about 50-70 kDa) and two light (L) chains (about 25 kDa) inter-connected by disulfide bonds. There are two types of light chain: λ and K. In humans they are similar, but only one type is present in each antibody. Heavy chains are classified as mu, delta, gamma, alpha, or epsilon, and define the antibody's isotype as IgM, IgD, IgG, IgA, and IgE1 respectively. See generally, Fundamental Immunology Ch. 7 (Paul, W., ed., 2nd ed. Raven Press, N.Y. (1989)). Each heavy chain (herein sometimes referred to as H-chain or Hc) is comprised of a heavy chain variable domain (VH, or H-variable domain) and a heavy chain constant region (CH). The heavy chain constant region is comprised of three domains, CH1, CH2 and CH3. Each light chain (herein sometimes referred to as L-chain or Lc) is comprised of a light chain variable domain (VL, or L-variable domain) and a light chain constant region. The light chain constant region is comprised of one domain, CL. Within light and heavy chains, the variable and constant regions are joined by a “J” region of about 12 or more amino acids, with the heavy chain also including a “D” region of about 3 or more amino acids. The VH and VL regions can be further subdivided into regions of hypervariability, termed “complementarity determining regions” (CDR), interspersed with regions that are more conserved, termed “framework regions” (FR). Each VH and VL is composed of three CDRs (H-CDR herein designates a CDR from the heavy chain; and L-CDR herein designates a CDR from the light chain) and four FRs, arranged from amino-terminus to carboxyl-terminus in the following order: FR1, CDR1, FR2, CDR2, FR3, CDR3, FR4. In some embodiments, the assignment of amino acids to each domain is in accordance with the definitions of Kabat, Sequences of Proteins of Immunological Interest (National Institutes of Health, Bethesda, MD (1987 and 1991)), or Chothia & Lesk, J. MoI. Biol. 196:901-917 (1987); Chothia et al., Nature 342:878-883 (1989).

As used herein, the term “antigen-binding fragment” refers to one or more fragments of an antibody that retain the ability to specifically bind to an antigen. It has been shown that the antigen-binding function of an antibody can be performed by fragments of a full-length antibody. Non-limiting examples of binding fragments encompassed within the term “antigen-binding fragment” include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fab′ fragment which is obtained by cleaving a disulfide bond of the hinge region of the F(ab′)2; (iv) a Fd fragment consisting of the VH and CH1 domains; (v) a Fv fragment consisting of the VL and VH domains of a single arm of an antibody, (vi) a dAb fragment (Ward et al., (1989) Nature 341:544-546), which consists of a VH domain; (vii) an isolated complementarity determining region (CDR); and (viii) a dsFv, which consists of a VH::VL heterodimer stabilized by a disulfide bond. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al. Science 242:423-426 (1988) and Huston et al. Proc. Natl. Acad. Sci. USA 85:5879-5883 (1988)). Also within the scope of this disclosure are antigen-binding molecules comprising a VH and/or a VL, In the case of a VH, the molecule may also comprise one or more of a CH 1, hinge, CH2 or CH3 region. Such single chain antibodies are also intended to be encompassed within the term “antigen-binding fragment” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. Proc. Natl. Acad. Sci. USA 90:6444-6448 (1993); Poljak et al. Structure 2:1121-1123 (1994)).

As used herein, the term “antigen-binding fragment” also includes, e.g., single domain antibodies such as camelized single domain antibodies. See, e.g., Muyldermans et al. (2001) Trends Biochem Sci 26:230-235; Nuttall et al. (2000) Curr Pharm Biotech 1:253-263; Reichmann et al. (1999) J Immunol Meth 231:25-38; PCT application publication nos. WO 94/04678 and WO 94/25591; and U.S. Pat. No. 6,005,079, all of which are incorporated herein by reference in their entireties. In some embodiments, the disclosure provides single domain antibodies comprising two VH domains with modifications such that single domain antibodies are formed.

As used herein, the terms “anti-ligand antibody” or “anti-ligand antigen-binding fragment” and the like are used to reference an antibody that is capable of binding/targeting a TGF-β superfamily ligand (e.g., activin A, activin B, activin AB, nodal, GDF11, GDF8). In some embodiments, an anti-ligand antibody or antigen-binding fragment thereof is capable of binding/targeting GDF8. In some embodiments, the anti-ligand antibody or antigen-binding fragment thereof is multi-specific and is capable of binding/targeting multiple TGF-β superfamily ligands (e.g., more than one of activin A, activin B, activin AB, nodal, GDF11, GDF8).

As used herein, the term “epitope” or “antigenic determinant” refers to a site on an antigen (e.g., GDF8) to which an immunoglobulin or antibody specifically binds. An epitope can be formed both from contiguous amino acids or noncontiguous amino acids juxtaposed by tertiary folding of a protein. An epitope formed from contiguous amino acids is typically retained on exposure to denaturing solvents, whereas an epitope formed by tertiary folding is typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from, e.g., GDF8, are tested for reactivity with the given antibody or antigen-binding fragment. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

In some embodiments, any of the anti-ligand antibodies or antigen-binding fragments disclosed herein are capable of binding to a TGF-0 superfamily ligand (e.g., activin A, activin B, activin AB, nodal, GDF11, GDF8) in a manner such that the bound ligand is no longer capable of interacting with a receptor (e.g., ActRIIA, ActRIIB, ALK4, ALK7, BMPRII, ALK1, ALK2, ALK3, ALK6, and/or TGFβRII). In some embodiments, any of the anti-ligand antibodies or antigen-binding fragments disclosed herein are capable of binding to a TGF-β superfamily ligand (e.g., activin A, activin B, activin AB, nodal, GDF11, GDF8) in a manner such that the bound ligand is no longer capable of triggering any downstream signaling event. In some embodiments, any of the anti-ligand antibodies or antigen-binding fragments are capable of inhibiting SMAD2, SMAD3, SMAD1, SMAD5 and/or SMAD8 signaling in a cell. In some embodiments, any of the anti-ligand antibodies or antigen-binding fragments are capable of inhibiting SMAD2, SMAD3, SMAD1, SMAD5 and/or SMAD8 signaling in a cell by at least 5%, 10%, 15%, 20%, 25%, 30%, 35%, 40%, 45%, 50%, 55%, 60%, 70%, 75%, 80%, 85%, 90%, 95% or 100% as compared to the same cell type under essentially the same conditions in the absence of the antibody or antigen-binding fragement. In particular embodiments, any of the anti-ligand antibodies or antigen-binding fragments are capable of inhibiting SMAD2 and/or SMAD3 signaling in a cell. In particular embodiments, any of the anti-ligand antibodies or antigen-binding fragments are capable of inhibiting SMAD2 and/or SMAD3 signaling in a cell by at least 5%, 10%, 15%, 20%, 25%, 30%, 35%, 40%, 45%, 50%, 55%, 60%, 70%, 75%, 80%, 85%, 90%, 95% or 100% as compared to the same cell type under similar conditions in the absence of the antibody or antigen-binding fragement.

In some embodiments, the antibody or antigen-binding fragment is capable of binding to a GDF8 polypeptide. In some embodiments, the antibody or antigen-binding fragment is capable of binding to a GDF8 polypeptide having an amino acid sequence that is at least 80%, 85%, 90%, 91%, 92%, 93%, 94%, 95%, 96%, 97%, 98%, 99% or 100% identical to the amino acid sequence of SEQ ID NO: 149, or a functional fragment thereof. A functional fragment of GDF8 would be capable of binding one or more TGFβ-superfamily receptors (e.g., ActRIIA, ActRIIB, ALK4 and/or ALK7) and triggering downstream signaling of the receptor(s). In some embodiments, the antibody or antigen-binding fragment binds to the wrist region of GDF8 (see, e.g., Walker et al., 2017, BMC Biol., 15:19).

(GenBank Accesion No. NP_005250.1):
SEQ ID NO: 149
MQKLQLCVYIYLFMLIVAGPVDLNENSEQKENVEKEGLCNACTWRQNTKS
SRIEAIKIQILSKLRLETAPNISKDVIRQLLPKAPPLRELIDQYDVQRDD
SSDGSLEDDDYHATTETIITMPTESDFLMQVDGKPKCCFFKFSSKIQYNK
VVKAQLWIYLRPVETPTTVFVQILRLIKPMKDGTRYTGIRSLKLDMNPGT
GIWQSIDVKTVLQNWLKQPESNLGIEIKALDENGHDLAVTFPGPGEDGLN
PFLEVKVTDTPKRSRRDFGLDCDEHSTESRCCRYPLTVDFEAFGWDWIIA
PKRYKANYCSGECEFVFLQKYPHTHLVHQANPRGSAGPCCTPTKMSPINM
LYFNGKEQIIYGKIPAMVVDRCGCS

In some embodiments, the antibody or antigen-binding fragment is capable of binding to a GDF11 polypeptide. In some embodiments, the antibody or antigen-binding fragment is capable of binding to a GDF11 polypeptide having an amino acid sequence that is at least 80%, 85%, 90%, 91%, 92%, 93%, 94%, 95%, 96%, 97%, 98%, 99% or 100% identical to the amino acid sequence of SEQ ID NO: 150, or a functional fragment thereof. A functional fragment of GDF11 would be capable of binding one or more TGFβ-superfamily receptors (e.g., ActRIIA, ActRIIB, ALK4 and/or ALK7) and triggering downstream signaling of the receptor(s). In some embodiments, the antibody or antigen-binding fragment binds to the wrist region of GDF11 (see, e.g., Walker et al., 2017, BMC Biol., 15:19).

(GenBank Accesion No. NP_005802.1):
SEQ ID NO: 150
MVLAAPLLLGFLLLALELRPRGEAAEGPAAAAAAAAAAAAAGVGGERSSR
PAPSVAPEPDGCPVCVWRQHSRELRLESIKSQILSKLRLKEAPNISREVV
KQLLPKAPPLQQILDLHDFQGDALQPEDFLEEDEYHATTETVISMAQETD
PAVQTDGSPLCCHFHFSPKVMFTKVLKAQLWVYLRPVPRPATVYLQILRL
KPLTGEGTAGGGGGGRRHIRIRSLKIELHSRSGHWQSIDFKQVLHSWFRQ
PQSNWGIEINAFDPSGTDLAVTSLGPGAEGLHPFMELRVLENTKRSRRNL
GLDCDEHSSESRCCRYPLTVDFEAFGWDWIIAPKRYKANYCSGQCEYMFM
QKYPHTHLVQQANPRGSAGPCCTPTKMSPINMLYFNDKQQIIYGKIPGMV
VDRCGCS

In some embodiments, the antibody or antigen-binding fragment binds both GDF8 and GDF11. In some embodiments, the antibody or antigen-binding fragment binds an epitope common to both GDF8 and GDF11.

In some embodiments, the antibody or antigen-binding fragment comprises one or more of the CDRs selected from the group consisting of SEQ ID NOs: 151-162. In some embodiments, the antibody or antigen-binding fragment comprises one or more of the CDRs selected from the group consisting of SEQ ID NOs: 151-162, but with 1, 2, 3, 4, 5 or 6 conservative substitutions. In some embodiments, the antibody or antigen-binding fragment comprises a variable heavy chain (VH) and a variable light chain (VL), wherein the VH CDR1 comprises SEQ NO:151 or SEQ ID NO:157, wherein the VH CDR2 comprises SEQ ID NO:152 or SEQ ID NO:158, and wherein the VH CDR3 comprises SEQ ID NO:153 or SEQ ID NO:159; and wherein the VL CDR1 comprises SEQ ID NO:154 or SEQ ID NO:160, wherein the VL CDR2 comprises SEQ ID NO:155 or SEQ ID NO:161, and wherein the VL CDR3 comprises SEQ ID NO:156 or SEQ ID NO:162.

(VH CDR1)
SEQ ID NO: 151
SYWMQ
(VH CDR2)
SEQ ID NO: 152
AIYPGDGDTRYTQKFKG
(VH CDR3)
SEQ ID NO: 153
ARMGGYDRYYFDY
(VL CDR1)
SEQ ID NO: 154
KSSQSLLNSANQKNYLA
(VL CDR2)
SEQ ID NO: 155
FASTRES
(VL CDR3)
SEQ ID NO: 156
QQHYNTPLT
(VH CDR1)
SEQ ID NO: 157
GYTFTSYWMQ
(VH CDR2)
SEQ ID NO: 158
AIYPGDGDT
(VH CDR3)
SEQ ID NO: 159
ARMGGYDRYYFDY
(VL CDR1)
SEQ ID NO: 160
KSSQSLLNSANQKNYLA
(VL CDR2)
SEQ ID NO: 161
FASTRES
(VL CDR3)
SEQ ID NO: 162
QQHYNTPLT

In some embodiments, the antibody or antigen-binding fragment is a full-length antibody. In some embodiments, the antibody is a monoclonal antibody. In some embodiments, the antibody is a human antibody. In some embodiments, the antibody is a humanized antibody.

In some embodiments, the antibody or antigen-binding fragment is a chimeric antibody. In some embodiments, the antibody or antigen-binding fragment is a humanized antibody or antigen-binding fragment. In some embodiments, the antibody or antigen-binding fragment is an antigen-binding fragment. In some embodiments, the antigen-binding fragment is selected from the group consisting of a Fab fragment, a F(ab′)2 fragment, a Fab′ fragment, a dAb, or an scFv.

Antibodies that recognize the same or overlapping epitope as a known antibody or compete for binding with a known antibody can be identified using routine techniques. Such techniques include, for example, an immunoassay, which shows the ability of one antibody to block the binding of another antibody to a target antigen, i.e., a competitive binding assay. Competitive binding is determined in an assay in which the immunoglobulin under test inhibits specific binding of a reference antibody to a common antigen, such as GDF8. Numerous types of competitive binding assays are known, for example: solid phase direct or indirect radioimmunoassay(RIA), solid phase direct or indirect enzyme immunoassay(EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies:A Laboratory Manual, Cold Spring Harbor Press (1988)); solid phase direct label RIA using I-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)). Typically, such an assay involves the use of purified antigen bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least about 50-55%, 55-60%, 60-65%, 65-70%, 70-75% or more.

Other techniques include, for example, epitope mapping methods, such as, x-ray analyses of crystals of antigen: antibody complexes which provides atomic resolution of the epitope and mass spectrometry combined with hydrogen/deuterium (H/D) exchange which studies the conformation and dynamics of antigen: antibody interactions. Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. The peptides are then regarded as leads for the definition of the epitope corresponding to the antibody used to screen the peptide library. For epitope mapping, computational algorithms have also been developed which have been shown to map conformational discontinuous epitopes.

The disclosure also features methods for producing any of the anti-ligand antibodies or antigen-binding fragments thereof described herein. In some embodiments, methods for preparing an antibody described herein can include immunizing a subject (e.g., a non-human mammal) with an appropriate immunogen (e.g., GDF8). Suitable immunogens for generating any of the antibodies described herein are set forth herein. For example, to generate an antibody that binds to GDF8, a skilled artisan can immunize a suitable subject (e.g., a non-human mammal such as a rat, a mouse, a gerbil, a hamster, a dog, a cat, a pig, a goat, a horse, or a non-human primate) with human GDF8.

A suitable subject (e.g., a non-human mammal) can be immunized with the appropriate antigen along with subsequent booster immunizations a number of times sufficient to elicit the production of an antibody by the mammal. The immunogen can be administered to a subject (e.g., a non-human mammal) with an adjuvant. Adjuvants useful in producing an antibody in a subject include, but are not limited to, protein adjuvants; bacterial adjuvants, e.g., whole bacteria (BCG, Corynebacterium parvum or Salmonella minnesota) and bacterial components including cell wall skeleton, trehalose dimycolate, monophosphoryl lipid A, methanol extractable residue (VER) of tubercle bacillus, complete or incomplete Freund's adjuvant; viral adjuvants; chemical adjuvants, e.g., aluminum hydroxide, and iodoacetate and cholesteryl hemisuccinate. Other adjuvants that can be used in the methods for inducing an immune response include, e.g., cholera toxin and parapoxvirus proteins. See also Bieg et al. (1999) Autoimmunity 31(1):15-24.See also, e.g., Lodmell et al. (2000) Vaccine 18:1059-1066; Johnson et al. (1999) J Med Chem 42:4640-4649; Baldridge et al. (1999) Methods 19:103-107; and Gupta et al. (1995) Vaccine 13(14): 1263-1276.

In some embodiments, the methods include preparing a hybridoma cell line that secretes a monoclonal antibody that binds to the immunogen. For example, a suitable mammal such as a laboratory mouse is immunized with a ligand, e.g., GDF8, as described above. Antibody-producing cells (e.g., B cells of the spleen) of the immunized mammal can be isolated two to four days after at least one booster immunization of the immunogen and then grown briefly in culture before fusion with cells of a suitable myeloma cell line. The cells can be fused in the presence of a fusion promoter such as, e.g., vaccinia virus or polyethylene glycol. The hybrid cells obtained in the fusion are cloned, and cell clones secreting the desired antibodies are selected. For example, spleen cells of Balb/c mice immunized with a suitable immunogen can be fused with cells of the myeloma cell line PAI or the myeloma cell line Sp2/0-Ag 14. After the fusion, the cells are expanded in suitable culture medium, which is supplemented with a selection medium, for example HAT medium, at regular intervals in order to prevent normal myeloma cells from overgrowing the desired hybridoma cells. The obtained hybrid cells are then screened for secretion of the desired antibodies, e.g., an antibody that binds to a TGF-β superfamily ligand (e.g., GDF8) as described herein.

In some embodiments, a skilled artisan can identify an anti-ligand antibody from a non-immune biased library as described in, e.g., U.S. Pat. No. 6,300,064 (to Knappik et al.; Morphosys AG) and Schoonbroodt et al. (2005) Nucleic Acids Res 33(9):e81.

In some embodiments, the methods described herein can involve, or be used in conjunction with, e.g., phage display technologies, bacterial display, yeast surface display, eukaryotic viral display, mammalian cell display, and cell-free (e.g., ribosomal display) antibody screening techniques (see, e.g., Etz et al. (2001) J Bacteriol 183:6924-6935; Cornelis (2000) Curr Opin Biotechnol 11:450-454; Klemm et al. (2000) Microbiology 146:3025-3032; Kieke et al. (1997) Protein Eng 10:1303-1310; Yeung et al. (2002) BiotechnolProg 18:212-220; Boder et al. (2000) Methods Enzymology328:430-444; Grabherr et al. (2001) Comb Chem High Throughput Screen 4:185-192; Michael et al. (1995) Gene Ther 2:660-668; Pereboev et al. (2001) J Virol 75:7107-7113; Schaffitzel et al. (1999) J Immunol Methods 231:119-135; and Hanes et al. (2000) Nat Biotechnol 18:1287-1292).

Methods for identifying antibodies using various phage display methods are known in the art. In phage display methods, functional antibody domains are displayed on the surface of phage particles which carry the polynucleotide sequences encoding them. Such phage can be utilized to display antigen-binding domains of antibodies, such as Fab, Fv, or disulfide-bond stabilized Fv antibody fragments, expressed from a repertoire or combinatorial antibody library (e.g., human or murine). Phage used in these methods are typically filamentous phage such as fd and M13. The antigen binding domains are expressed as a recombinantly fused protein to any of the phage coat proteins pIII, pVIII, or pIX. See, e.g., Shi et al. (2010) JMB 397:385-396. Examples of phage display methods that can be used to make the immunoglobulins, or fragments thereof, described herein include those disclosed in Brinkman et al. (1995) J Immunol Methods 182:41-50; Ames et al. (1995) J Immunol Methods 184:177-186; Kettleborough et al. (1994) Eur J Immunol 24:952-958; Persic et al. (1997) Gene 187:9-18; Burton et al. (1994) Advances in Immunology 57:191-280; and PCT publication nos. WO 90/02809, WO 91/10737, WO 92/01047, WO 92/18619, WO 93/11236, WO 95/15982, and WO 95/20401. Suitable methods are also described in, e.g., U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

In some embodiments, the phage display antibody libraries can be generated using mRNA collected from B cells from the immunized mammals. For example, a splenic cell sample comprising B cells can be isolated from mice immunized with a TGF-β superfamily ligand (e.g., GDF8) as described above. mRNA can be isolated from the cells and converted to cDNA using standard molecular biology techniques. See, e.g., Sambrook et al. (1989) “Molecular Cloning: A Laboratory Manual, 2nd Edition,” Cold Spring Harbor Laboratory Press, Cold Spring Harbor, N.Y.; Harlow and Lane (1988), supra; Benny K. C. Lo (2004), supra; and Borrebaek (1995), supra. The cDNA coding for the variable regions of the heavy chain and light chain polypeptides of immunoglobulins are used to construct the phage display library. Methods for generating such a library are described in, e.g., Merz et al. (1995) J Neurosci Methods 62(1-2):213-9; Di Niro et al. (2005) Biochem J 388(Pt 3):889-894; and Engberg et al. (1995) Methods Mol Biol 51:355-376.

In some embodiments, a combination of selection and screening can be employed to identify an antibody of interest from, e.g., a population of hybridoma-derived antibodies or a phage display antibody library. Suitable methods are known in the art and are described in, e.g., Hoogenboom (1997) Trends in Biotechnology 15:62-70; Brinkman et al. (1995), supra; Ames et al. (1995), supra; Kettleborough et al. (1994), supra; Persic et al. (1997), supra; and Burton et al. (1994), supra. For example, a plurality of phagemid vectors, each encoding a fusion protein of a bacteriophage coat protein (e.g., pIII, pVIII, or pIX of M13 phage) and a different antigen-combining region are produced using standard molecular biology techniques and then introduced into a population of bacteria (e.g., E. coli). Expression of the bacteriophage in bacteria can, in some embodiments, require use of a helper phage. In some embodiments, no helper phage is required (see, e.g., Chasteen et al., (2006) Nucleic Acids Res 34(21):e145). Phage produced from the bacteria are recovered and then contacted to, e.g., a target antigen bound to a solid support (immobilized). Phage may also be contacted to antigen in solution, and the complex is subsequently bound to a solid support.

A subpopulation of antibodies screened using the above methods can be characterized for their specificity and binding affinity for a particular antigen (e.g., GDF8) using any immunological or biochemical based method known in the art. For example, specific binding of an antibody to a TGF-β superfamily ligand (e.g., GDF8), may be determined for example using immunological or biochemical based methods such as, but not limited to, an ELISA assay, SPR assays, immunoprecipitation assay, affinity chromatography, and equilibrium dialysis as described above. Immunoassays which can be used to analyze immunospecific binding and cross-reactivity of the antibodies include, but are not limited to, competitive and non-competitive assay systems using techniques such as Western blots, RIA, ELISA (enzyme linked immunosorbent assay), “sandwich” immunoassays, immunoprecipitation assays, immunodiffusion assays, agglutination assays, complement-fixation assays, immunoradiometric assays, fluorescent immunoassays, and protein A immunoassays. Such assays are routine and well known in the art.

In embodiments where the selected CDR amino acid sequences are short sequences (e.g., fewer than 10-15 amino acids in length), nucleic acids encoding the CDRs can be chemically synthesized as described in, e.g., Shiraishi et al. (2007) Nucleic Acids Symposium Series 51(1):129-130 and U.S. Pat. No. 6,995,259. For a given nucleic acid sequence encoding an acceptor antibody, the region of the nucleic acid sequence encoding the CDRs can be replaced with the chemically synthesized nucleic acids using standard molecular biology techniques. The 5′ and 3′ ends of the chemically synthesized nucleic acids can be synthesized to comprise sticky end restriction enzyme sites for use in cloning the nucleic acids into the nucleic acid encoding the variable region of the donor antibody.

In some embodiments, the anti-ligand antibodies described herein comprise an altered heavy chain constant region that has reduced (or no) effector function relative to its corresponding unaltered constant region. Effector functions involving the constant region of the anti-ligand antibody may be modulated by altering properties of the constant or Fc region. Altered effector functions include, for example, a modulation in one or more of the following activities: antibody-dependent cellular cytotoxicity (ADCC), complement-dependent cytotoxicity (CDC), apoptosis, binding to one or more Fc-receptors, and pro-inflammatory responses. Modulation refers to an increase, decrease, or elimination of an effector function activity exhibited by a subject antibody containing an altered constant region as compared to the activity of the unaltered form of the constant region. In particular embodiments, modulation includes situations in which an activity is abolished or completely absent.

An altered constant region with altered FcR binding affinity and/or ADCC activity and/or altered CDC activity is a polypeptide which has either an enhanced or diminished FcR binding activity and/or ADCC activity and/or CDC activity compared to the unaltered form of the constant region. An altered constant region which displays increased binding to an FcR binds at least one FcR with greater affinity than the unaltered polypeptide. An altered constant region which displays decreased binding to an FcR binds at least one FcR with lower affinity than the unaltered form of the constant region. Such variants which display decreased binding to an FcR may possess little or no appreciable binding to an FcR, e.g., 0 to 50% (e.g., less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1%) of the binding to the FcR as compared to the level of binding of a native sequence immunoglobulin constant or Fc region to the FcR. Similarly, an altered constant region that displays modulated ADCC and/or CDC activity may exhibit either increased or reduced ADCC and/or CDC activity compared to the unaltered constant region. For example, in some embodiments, the anti-ligand antibody comprises an altered constant region can exhibit approximately 0 to 50% (e.g., less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1%) of the ADCC and/or CDC activity of the unaltered form of the constant region. An anti-ligand antibody described herein comprising an altered constant region displaying reduced ADCC and/or CDC may exhibit reduced or no ADCC and/or CDC activity.

In some embodiments, an anti-ligand antibody or antigen-binding fragment described herein exhibits reduced or no effector function. In some embodiments, an anti-ligand antibody comprises a hybrid constant region, or a portion thereof, such as a G2/G4 hybrid constant region (see e.g., Burton et al. (1992) Adv Immun 51:1-18; Canfield et al. (1991) J Exp Med 173:1483-1491; and Mueller et al. (1997) Mol Immunol 34(6):441-452). See above.

In some embodiments, an anti-ligand antibody or antigen-binding fragment may contain an altered constant region exhibiting enhanced or reduced complement dependent cytotoxicity (CDC). Modulated CDC activity may be achieved by introducing one or more amino acid substitutions, insertions, or deletions in an Fc region of the antibody. See, e.g., U.S. Pat. No. 6,194,551. Alternatively or additionally, cysteine residue(s) may be introduced in the Fc region, thereby allowing interchain disulfide bond formation in this region. The homodimeric antibody thus generated may have improved or reduced internalization capability and/or increased or decreased complement-mediated cell killing. See, e.g., Caron et al. (1992) J Exp Med 176:1191-1195 and Shopes (1992) Immunol 148:2918-2922; PCT publication nos. WO 99/51642 and WO 94/29351; Duncan and Winter (1988) Nature 322:738-40; and U.S. Pat. Nos. 5,648,260 and 5,624,821.

The antibodies or antigen-binding fragments thereof described herein can be produced using a variety of techniques known in the art of molecular biology and protein chemistry. For example, a nucleic acid encoding one or both of the heavy and light chain polypeptides of an antibody can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system such that it can be maintained in two different organisms, for example in mammalian or insect cells for expression and in a prokaryotic host for cloning and amplification.

Several possible vector systems are available for the expression of cloned heavy chain and light chain polypeptides from nucleic acids in mammalian cells. One class of vectors relies upon the integration of the desired gene sequences into the host cell genome. Cells which have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet 1:327). The selectable marker gene can be either linked to the DNA gene sequences to be expressed, or introduced into the same cell by co-transfection (Wigler et al. (1979) Cell 16:77). A second class of vectors utilizes DNA elements which confer autonomously replicating capabilities to an extrachromosomal plasmid. These vectors can be derived from animal viruses, such as bovine papillomavirus (Sarver et al. (1982) Proc Natl Acad Sci USA, 79:7147), cytomegalovirus, polyoma virus (Deans et al. (1984) Proc Natl Acad Sci USA 81:1292), or SV40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, cationic liposomes, electroporation, viral infection, dextran-mediated transfection, polybrene-mediated transfection, protoplast fusion, and direct microinjection.

Appropriate host cells for the expression of antibodies or antigen-binding fragments thereof include yeast, bacteria, insect, plant, and mammalian cells. Of particular interest are bacteria such as E. coli, fungi such as Saccharomyces cerevisiae and Pichia pastoris, insect cells such as SF9, mammalian cell lines (e.g., human cell lines), as well as primary cell lines.

In some embodiments, an antibody or fragment thereof can be expressed in, and purified from, transgenic animals (e.g., transgenic mammals). For example, an antibody can be produced in transgenic non-human mammals (e.g., rodents) and isolated from milk as described in, e.g., Houdebine (2002) Curr Opin Biotechnol 13(6):625-629; van Kuik-Romeijn et al. (2000) Transgenic Res 9(2):155-159; and Pollock et al. (1999) J Immunol Methods 231(1-2):147-157.

The antibodies and fragments thereof can be produced from the cells by culturing a host cell transformed with the expression vector containing nucleic acid encoding the antibodies or fragments, under conditions, and for an amount of time, sufficient to allow expression of the proteins. Such conditions for protein expression will vary with the choice of the expression vector and the host cell, and will be easily ascertained by one skilled in the art through routine experimentation. For example, antibodies expressed in E. coli can be refolded from inclusion bodies (see, e.g., Hou et al. (1998) Cytokine 10:319-30). Bacterial expression systems and methods for their use are well known in the art (see Current Protocols in Molecular Biology, Wiley & Sons, and Molecular Cloning—A Laboratory Manual—3rd Ed., Cold Spring Harbor Laboratory Press, New York (2001)). The choice of codons, suitable expression vectors and suitable host cells will vary depending on a number of factors, and may be easily optimized as needed. An antibody (or fragment thereof) described herein can be expressed in mammalian cells or in other expression systems including but not limited to yeast, baculovirus, and in vitro expression systems (see, e.g., Kaszubska et al. (2000) Protein Expression and Purification 18:213-220).

Following expression, the antibodies and fragments thereof can be isolated. An antibody or fragment thereof can be isolated or purified in a variety of ways known to those skilled in the art depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) “Protein Purification, 3rd edition,” Springer-Verlag, New York City, New York. The degree of purification necessary will vary depending on the desired use. In some instances, no purification of the expressed antibody or fragments thereof will be necessary.

Methods for determining the yield or purity of a purified antibody or fragment thereof are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

The antibodies or antigen-binding fragments thereof can be modified following their expression and purification. The modifications can be covalent or non-covalent modifications. Such modifications can be introduced into the antibodies or fragments by, e.g., reacting targeted amino acid residues of the polypeptide with an organic derivatizing agent that is capable of reacting with selected side chains or terminal residues. Suitable sites for modification can be chosen using any of a variety of criteria including, e.g., structural analysis or amino acid sequence analysis of the antibodies or fragments.

In some embodiments, the antibodies or antigen-binding fragments thereof can be conjugated to a heterologous moiety. The heterologous moiety can be, e.g., a heterologous polypeptide, a therapeutic agent (e.g., a drug), or a detectable label such as, but not limited to, a radioactive label, an enzymatic label, a fluorescent label, a heavy metal label, a luminescent label, or an affinity tag such as biotin or streptavidin. Suitable heterologous polypeptides include, e.g., an antigenic tag (e.g., FLAG (DYKDDDDK (SEQ ID NO: 177)), polyhistidine (6-His; HHHHHH (SEQ ID NO: 178), hemagglutinin (HA; YPYDVPDYA (SEQ ID NO: 179)), glutathione-S-transferase(GST), or maltose-binding protein (MBP)) for use in purifying the antibodies or fragments. Heterologous polypeptides also include polypeptides (e.g., enzymes) that are useful as diagnostic or detectable markers, for example, luciferase, a fluorescent protein (e.g., green fluorescent protein (GFP)), or chloramphenicol acetyl transferase (CAT). Suitable radioactive labels include, e.g., 32P, 33P, 14C, 125I, 131I, 35S, and 3H. Suitable fluorescent labels include, without limitation, fluorescein, fluorescein isothiocyanate (FITC), green fluorescent protein (GFP), DyLight™ 488, phycoerythrin (PE), propidium iodide (PI), PerCP, PE-Alexa Fluor®700, Cy5, allophycocyanin, and Cy7. Luminescent labels include, e.g., any of a variety of luminescent lanthanide (e.g., europium or terbium) chelates. For example, suitable europium chelates include the europium chelate of diethylenetriaminepentaaceticacid (DTPA)or tetraazacyclododecane-1,4,7,10-tetraacetic acid (DOTA). Enzymatic labels include, e.g., alkaline phosphatase, CAT, luciferase, and horseradish peroxidase.

Two proteins (e.g., an antibody and a heterologous moiety) can be cross-linked using any of a number of known chemical cross linkers. Examples of such cross linkers are those which link two amino acid residues via a linkage that includes a “hindered” disulfide bond. In these linkages, a disulfide bond within the cross-linking unit is protected (by hindering groups on either side of the disulfide bond) from reduction by the action, for example, of reduced glutathione or the enzyme disulfide reductase. One suitable reagent, 4-succinimidyl oxycarbonyl-α-methyl-α(2-pyridyldithio) toluene (SMPT), forms such a linkage between two proteins utilizing a terminal lysine on one of the proteins and a terminal cysteine on the other. Heterobifunctional reagents that cross-link by a different coupling moiety on each protein can also be used. Other useful cross-linkers include, without limitation, reagents which link two amino groups (e.g., N-5-azido-2-nitrobenzoyloxysuccinimide), two sulfhydryl groups (e.g., 1,4-bis-maleimidobutane), an amino group and a sulfhydryl group (e.g., m-maleimido benzoyl-N-hydroxy succinimide ester), an amino group and a carboxyl group (e.g., 4-[p-azidosalicylamido]butylamine), and an amino group and a guanidinium group that is present in the side chain of arginine (e.g., p-azidophenyl glyoxal monohydrate).

In some embodiments, a radioactive label can be directly conjugated to the amino acid backbone of the antibody. Alternatively, the radioactive label can be included as partofalargermolecule(e.g., ¹²⁵Iinmeta-[¹²⁵I]iodophenyl-N-hydroxysuccinimide ([¹²⁵I]mIPNHS) which binds to free amino groups to form meta-iodophenyl (mIP) derivatives of relevant proteins (see, e.g., Rogers et al. (1997) J Nucl Med 38:1221-1229) or chelate (e.g., to DOTA or DTPA) which is in turn bound to the protein backbone. Methods of conjugating the radioactive labels or larger molecules/chelates containing them to the antibodies or antigen-binding fragments described herein are known in the art. Such methods involve incubating the proteins with the radioactive label under conditions (e.g., pH, salt concentration, and/or temperature) that facilitate binding of the radioactive label or chelate to the protein (see, e.g., U.S. Pat. No. 6,001,329).

Methods for conjugating a fluorescent label (sometimes referred to as a “fluorophore”) to a protein (e.g., an antibody) are known in the art of protein chemistry. For example, fluorophores can be conjugated to free amino groups (e.g., of lysines) or sulfhydryl groups (e.g., cysteines) of proteins using succinimidyl (NHS) ester or tetrafluorophenyl (TFP) ester moieties attached to the fluorophores. In some embodiments, the fluorophores can be conjugated to a heterobifunctional cross-linker moiety such as sulfo-SMCC. Suitable conjugation methods involve incubating an antibody protein, or fragment thereof, with the fluorophore under conditions that facilitate binding of the fluorophore to the protein. See, e.g., Welch and Redvanly (2003) “Handbook of Radiopharmaceuticals: Radiochemistry and Applications,” John Wiley and Sons (ISBN 0471495603).

In some embodiments, the antibodies or fragments can be modified, e.g., with a moiety that improves the stabilization and/or retention of the antibodies in circulation, e.g., in blood, serum, or other tissues. For example, the antibody or fragment can be PEGylated as described in, e.g., Lee et al. (1999) Bioconjug Chem 10(6): 973-8; Kinstler et al. (2002) Advanced Drug Deliveries Reviews 54:477-485; and Roberts et al. (2002) Advanced Drug Delivery Reviews 54:459-476 or HESylated (Fresenius Kabi, Germany; see, e.g., Pavisid et al. (2010) Int J Pharm 387(1-2):110-119). The stabilization moiety can improve the stability, or retention of, the antibody (or fragment) by at least about 1.5 (e.g., at least about 2, 5, 10, 15, 20, 25, 30, 40, or 50 or more) fold.

In some embodiments, the antibodies or antigen-binding fragments thereof described herein can be glycosylated. In some embodiments, an antibody or antigen-binding fragment thereof described herein can be subjected to enzymatic or chemical treatment, or produced from a cell, such that the antibody or fragment has reduced or absent glycosylation. Methods for producing antibodies with reduced glycosylation are known in the art and described in, e.g., U.S. Pat. No. 6,933,368; Wright et al. (1991) EMBO J 10(10):2717-2723; and Co et al. (1993) Mol Immunol 30:1361.

In some embodiments, any of the multispecific binders disclosed herein comprises any of the TβRII portions disclosed herein and any of the antibodies or antigen-binding fragment portions disclosed herein. In some embodiments, the multispecific binder has a structural layout similar to that illustrated in FIG. 15B. In some embodiments, the multispecific binder comprises an amino acid sequence that is at least 80%, 85%, 90%, 91%, 92%, 93%, 94%, 95%, 96%, 97%, 98%, 99% or 100% identical to the amino acid sequence of any one of or combination of SEQ ID NOs: 167-175. In some embodiments, the multispecific binder comprises an amino acid sequence that is at least 80%, 85%, 90%, 91%, 92%, 93%, 94%, 95%, 96%, 97%, 98%, 99% or 100% identical to the amino acid sequence of SEQ ID NO: 167. In some embodiments, the multispecific binder comprises an amino acid sequence that is at least 80%, 85%, 90%, 91%, 92%, 93%, 94%, 95%, 96%, 97%, 98%, 99% or 100% identical to the amino acid sequence of SEQ ID NO: 170. In some embodiments, the multispecific binder comprises an amino acid sequence that is at least 80%, 85%, 90%, 91%, 92%, 93%, 94%, 95%, 96%, 97%, 98%, 99% or 100% identical to the amino acid sequence of SEQ ID NO: 174. In particular embodiments, the multispecific binder comprises amino acid sequences that are at least 80%, 85%, 90%, 91%, 92%, 93%, 94%, 95%, 96%, 97%, 98%, 99% or 100% identical to the amino acid sequences of SEQ ID NOs: 167, 170 and 174. In further embodiments, the multispecific binder comprises amino acid sequences that are at least 80%, 85%, 90%, 91%, 92%, 93%, 94%, 95%, 96%, 97%, 98%, 99% or 100% identical to the amino acid sequences of SEQ ID NOs: 172 and 182. In some embodiments, the polypeptides in the multispecific binder comprise a leader/signal sequence (e.g., a signal sequence having an amino acid sequence that is at least 80%, 85%, 90%, 91%, 92%, 93%, 94%, 95%, 96%, 97%, 98%, 99% or 100% identical to the amino acid sequence of SEQ ID NO: 176).

SEQ ID NO: 167-(Variable Heavy Chain)
QVQLQQSGAELARPGASVKLSCKASGYTFTSYWMQWVKQRPGQGLEWIGA
IYPGDGDTRYTQKFKGKATLTADKSSSTAYMQLSSLASEDSAVYYCARMG
GYDRYYFDYWGQGTTLTVSS
SEQ ID NO: 168-(Constant region delta K)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPG
SEQ ID NO: 169-(Linker)
GGGSGGGGSGGGGSGGGGSG
SEQ ID NO: 170-(TβRII)
TIPPHVQKSDVEMEAQKDEIICPSCNRTAHPLRHINNDMIVTDNNGAVKF
PQLCKFCDVRFSTCDNQKSCMSNCSITSICEKPQEVCVAVWRKNDENITL
ETVCHDPKLPYHDFILEDAASPKCIMKEKKKPGETFFMCSCSSDECNDNI
IFSEEYNTSNPD
SEQ ID NO: 171-(Constant Region of Heavy chain +
TβRII)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGTGGGGSGGGGSGGGGSGGGGS
TIPPHVQKSDVEMEAQKDEIICPSCNRTAHPLRHINNDMIVTDNNGAVKF
PQLCKFCDVRFSTCDNQKSCMSNCSITSICEKPQEVCVAVWRKNDENITL
ETVCHDPKLPYHDFILEDAASPKCIMKEKKKPGETFFMCSCSSDECNDNI
IFSEEYNTSNPD
SEQ ID NO: 172-(Heavy chain + TβRII)
QVQLQQSGAELARPGASVKLSCKASGYTFTSYWMQWVKQRPGQGLEWIGA
IYPGDGDTRYTQKFKGKATLTADKSSSTAYMQLSSLASEDSAVYYCARMG
GYDRYYFDYWGQGTTLTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGTG
GGGSGGGGSGGGGSGGGGSTIPPHVQKSDVEMEAQKDEIICPSCNRTAHP
LRHINNDMIVTDNNGAVKFPQLCKFCDVRFSTCDNQKSCMSNCSITSICE
KPQEVCVAVWRKNDENITLETVCHDPKLPYHDFILEDAASPKCIMKEKKK
PGETFFMCSCSSDECNDNIIFSEEYNTSNPD
SEQ ID NO: 173-(Heavy chain + TβRII + leader)
MGWSCIMLFLAATATGAHSQVQLQQSGAELARPGASVKLSCKASGYTFTS
YWMQWVKQRPGQGLEWIGAIYPGDGDTRYTQKFKGKATLTADKSSSTAYM
QLSSLASEDSAVYYCARMGGYDRYYFDYWGQGTTLTVSSASTKGPSVFPL
APSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPP
CPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVM
HEALHNHYTQKSLSLSPGTGGGGSGGGGSGGGGSGGGGSTIPPHVQKSDV
EMEAQKDEIICPSCNRTAHPLRHINNDMIVTDNNGAVKFPQLCKFCDVRF
STCDNQKSCMSNCSITSICEKPQEVCVAVWRKNDENITLETVCHDPKLPY
HDFILEDAASPKCIMKEKKKPGETFFMCSCSSDECNDNIIFSEEYNTSNP
D
SEQ ID NO: 174-(Variable Light Chain)
DIVMTQSPSSLAMSVGQKVTMSCKSSQSLLNSANQKNYLAWYQQKPGQSP
KLLVYFASTRESGVPDRFIGSGSGTDFTLTISSVQAEDLADYFCQQHYNT
PLTFGAGTKLELK
SEQ ID NO: 175-(Constant Kappa LC)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC
SEQ ID NO: 182: (Light Chain)
DIVMTQSPSSLAMSVGQKVTMSCKSSQSLLNSANQKNYLAWYQQKPGQSP
KLLVYFASTRESGVPDRFIGSGSGTDFTLTISSVQAEDLADYFCQQHYNT
PLTFGAGTKLELKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC
SEQ ID NO: 176-(Leader Sequence)
MGWSCIMLFLAATATGAHS

### 3. Nucleic Acids and Methods of Manufacture

In certain embodiments, the present disclosure makes available isolated and/or purified forms of polypeptides of any of the binders and/or multispecific binders of TGFβ-superfamily ligands disclosed herein (e.g., TβRII or ActRIIB polypeptides as well as ActRIIB:TβRII heteromultimers comprising the same), which are isolated from, or otherwise substantially free of (e.g., at least 80%, 90%, 95%, 96%, 97%, 98%, or 99% free of), other proteins and/or other polypeptide species. Polypeptides will generally be produced by expression from recombinant nucleic acids.

In certain embodiments, the disclosure includes nucleic acids encoding soluble polypeptides of any of the binders or multispecific binders of TGFβ-superfamily ligands disclosed herein (e.g., TβRII or ActRIIB polypeptides as well as ActRIIB:TβRII heteromultimers comprising the same) comprising the coding sequence for an extracellular portion of a protein (e.g., a TβRII and/or ActRIIB protein). In further embodiments, this disclosure also pertains to a host cell comprising such nucleic acids. The host cell may be any prokaryotic or eukaryotic cell. For example, a polypeptide of the present disclosure may be expressed in bacterial cells such as E. coli, insect cells (e.g., using a baculovirus expression system), yeast, or mammalian cells. Other suitable host cells are known to those skilled in the art. Accordingly, some embodiments of the present disclosure further pertain to methods of producing the polypeptides.

In certain aspects, the disclosure provides isolated and/or recombinant nucleic acids encoding any of the polypeptides of any of the binders or multispecific binders of TGFβ-superfamily ligands disclosed herein (e.g., TβRII or ActRIIB polypeptides as well as ActRIIB:TβRII heteromultimers comprising the same), including fragments, functional variants and fusion proteins disclosed herein. SEQ ID NOs: 8, 10, 12, 14, 16, 46, 47, 56, 57, 83, 86, 89, and 92 encode TβRII or ActRIIB polypeptides as well as variants thereof comprising an extracellular domain fused to an IgG Fc domain. Other nucleotide sequences of the disclosure include SEQ ID NOs: 136, 140, and 166. The subject nucleic acids may be single-stranded or double stranded. Such nucleic acids may be DNA or RNA molecules. These nucleic acids may be used, for example, in methods for making polypeptides or as direct therapeutic agents (e.g., in an antisense, RNAi or gene therapy approach).

In certain aspects, the subject nucleic acids encoding polypeptides are further understood to include nucleic acids that are variants of SEQ ID NOs: 8, 10, 12, 14, 16, 46, 47, 56, 57, 83, 86, 89, 92, 136, 140, and 166. Variant nucleotide sequences include sequences that differ by one or more nucleotide substitutions, additions or deletions, such as allelic variants.

In certain embodiments, the disclosure provides isolated or recombinant nucleic acid sequences that are at least 70%, 75%, 80%, 85%, 90%, 91%, 92%, 93%, 94%, 95%, 96%, 97%, 98%, 99% or 100% identical to SEQ ID NOs: 8, 10, 12, 14, 16, 46, 47, 56, 57, 83, 86, 89, 92, 136, 140, and 166. In particular embodiments, the disclosure provides isolated or recombinant nucleic acid sequences that are at least 70%, 75%, 80%, 85%, 90%, 91%, 92%, 93%, 94%, 95%, 96%, 97%, 98%, 99% or 100% identical to SEQ ID NOs: 83, 86, 89, 92, 136, 140, and 166 or fragments thereof. One of ordinary skill in the art will appreciate that nucleic acid sequences complementary to SEQ ID NOs: 8, 10, 12, 14, 16, 46, 47, 56, 57, 83, 86, 89, 92, 136, 140, and 166 and variants of SEQ ID NOs: 8, 10, 12, 14, 16, 46, 47, 56, 57, 83, 86, 89, 92, 136, 140, and 166 are also within the scope of this disclosure. In further embodiments, the nucleic acid sequences of the disclosure can be isolated, recombinant, and/or fused with a heterologous nucleotide sequence, or in a DNA library.

In other embodiments, nucleic acids of the disclosure also include nucleotide sequences that hybridize under highly stringent conditions to the nucleotide sequences designated in SEQ ID NOs: 8, 10, 12, 14, 16, 46, 47, 56, 57, 83, 86, 89, 92, 136, 140, and 166 complement sequences of SEQ ID NOs: 8, 10, 12, 14, 16, 46, 47, 56, 57, 83, 86, 89, 92, 136, 140, and 166 or fragments thereof. As discussed above, one of ordinary skill in the art will understand readily that appropriate stringency conditions which promote DNA hybridization can be varied. For example, one could perform the hybridization at 6.0×sodium chloride/sodium citrate (SSC) at about 45□C, followed by a wash of 2.0×SSC at 50□C. For example, the salt concentration in the wash step can be selected from a low stringency of about 2.0×SSC at 50□C to a high stringency of about 0.2×SSC at 50□C. In addition, the temperature in the wash step can be increased from low stringency conditions at room temperature, about 22□C, to high stringency conditions at about 65□C. Both temperature and salt may be varied, or temperature or salt concentration may be held constant while the other variable is changed. In some embodiments, the disclosure provides nucleic acids which hybridize under low stringency conditions of 6×SSC at room temperature followed by a wash at 2×SSC at room temperature.

Isolated nucleic acids which differ from the nucleic acids as set forth in SEQ ID NOs: 8, 10, 12, 14, 16, 46, 47, 56, 57, 83, 86, 89, 92, 136, 140, and 166 due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

It will be appreciated by one of skill in the art that corresponding variants based on the long isoform of TβRII will include nucleotide sequences encoding the 25-amino acid insertion along with a conservative Val-Ile substitution at the flanking position C-terminal to the insertion. It will also be appreciated that corresponding variants based on either the long (A) or short (B) isoforms of TβRII will include variant nucleotide sequences comprising an insertion of 108 nucleotides, encoding a 36-amino-acid insertion (SEQ ID NO: 41), at the same location described for naturally occurring TβRII isoform C.

In certain embodiments, the recombinant nucleic acids of the disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used.

In certain aspects disclosed herein, the subject nucleic acid is provided in an expression vector comprising a nucleotide sequence encoding a polypeptide of any of the binders or multispecific binders of TGFβ-superfamily ligands disclosed herein (e.g., TβRII or ActRIIB polypeptides as well as ActRIIB:TβRII heteromultimers comprising the same) and operably linked to at least one regulatory sequence. Regulatory sequences are art-recognized and are selected to direct expression of the T polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, CA (1990). For instance, any of a wide variety of expression control sequences that control the expression of a DNA sequence when operatively linked to it may be used in these vectors to express DNA sequences encoding a polypeptide of any of the binders or multispecific binders of TGFβ-superfamily ligands disclosed herein (e.g., TβRII or ActRIIB polypeptides as well as ActRIIB:TβRII heteromultimers comprising the same). Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid included in the disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant polypeptide of any of the binders or multispecific binders of TGFβ-superfamily ligands disclosed herein (e.g., TβRII or ActRIIB polypeptides as well as ActRIIB:TβRII heteromultimers comprising the same) include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning A Laboratory Manual, 3rd Ed., ed. by Sambrook, Fritsch and Maniatis (Cold Spring Harbor Laboratory Press, 2001). In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In certain embodiments, a vector will be designed for production of the subject polypeptides of any of the binders or multispecific binders of TGFβ-superfamily ligands disclosed herein (e.g., TβRII or ActRIIB polypeptides as well as ActRIIB:TβRII heteromultimers comprising the same) in CHO cells, such as a Pcmv-Script vector (Stratagene, La Jolla, Calif.), pcDN4 vectors (Invitrogen, Carlsbad, Calif.) and pCI-neo vectors (Promega, Madison, Wisc.). In a preferred embodiment, a vector will be designed for production of the subject polypeptides in HEK-293 cells. As will be apparent, the subject gene constructs can be used to cause expression of the subject polypeptides in cells propagated in culture, e.g., to produce proteins, including fusion proteins or variant proteins, for purification.

This disclosure also pertains to a host cell transfected with a recombinant gene including a coding sequence (e.g., SEQ ID NOs: 8, 10, 12, 14, 16, 46, 47, 56, 57, 83, 86, 89, 92, 136, 140 and 166) for one or more of the subject polypeptides of any of the binders or multispecific binders of TGFβ-superfamily ligands disclosed herein (e.g., TβRII or ActRIIB polypeptides as well as ActRIIB:TβRII heteromultimers comprising the same). The host cell may be any prokaryotic or eukaryotic cell. For example, a ActRIIB:TβRII protein disclosed herein may be expressed in bacterial cells such as E. coli, insect cells (e.g., using a baculovirus expression system), yeast, or mammalian cells. Other suitable host cells are known to those skilled in the art.

Accordingly, the present disclosure further pertains to methods of producing the subject polypeptides of any of the binders or multispecific binders of TGFβ-superfamily ligands disclosed herein (e.g., TβRII or ActRIIB polypeptides as well as ActRIIB:TβRII heteromultimers comprising the same). For example, a host cell transfected with an expression vector encoding a polypeptide can be cultured under appropriate conditions to allow expression of the polypeptide to occur. The polypeptide may be secreted and isolated from a mixture of cells and medium containing the polypeptide. Alternatively, the polypeptide may be retained cytoplasmically or in a membrane fraction and the cells harvested, lysed and the protein isolated. A cell culture includes host cells, and media. Suitable media for cell culture are well known in the art. The subject polypeptides can be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of the polypeptides and affinity purification with an agent that binds to a domain fused to the polypeptide (e.g., a protein A column may be used to purify an Fc fusion). In a preferred embodiment, the polypeptide is a fusion protein containing a domain which facilitates its purification. As an example, purification may be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

In another embodiment, a fusion gene coding for a purification leader sequence, such as a poly-(His)/enterokinase cleavage site sequence at the N-terminus of the desired portion of the recombinant polypeptide of any of the binders or multispecific binders of TGFβ-superfamily ligands disclosed herein (e.g., TβRII or ActRIIB polypeptides as well as ActRIIB:TβRII heteromultimers comprising the same), can allow purification of the expressed fusion protein by affinity chromatography using a Ni²⁺ metal resin. The purification leader sequence can then be subsequently removed by treatment with enterokinase to provide the purified polypeptide (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

### 4. Screening Assays

In certain aspects, the present invention relates to the use of a binder or multispecific binder of a TGFβ-superfamily ligand, such as an ActRIIB:TβRII heteromultimer (e.g., soluble ActRIIB:TβRII heterodimers) to identify compounds (agents) which are agonist or antagonists of the ActRIIB and TβRII signaling pathways. Compounds identified through this screening can be tested to assess their ability to modulate, for example, TGFβ1, TGFβ3, activin, GDF11, GDF8, and/or BMP10 signaling activity in vitro. Optionally, these compounds can further be tested in animal models to assess their ability to modulate tissue growth in vivo.

There are numerous approaches to screening for therapeutic agents for modulating tissue growth by targeting TGFβ1, TGFβ3, activin, GDF11, GDF8, and/or BMP10 polypeptides. In certain embodiments, high-throughput screening of compounds can be carried out to identify agents that perturb ActRIIB- or TβRII-mediated cell signaling. In certain embodiments, the assay is carried out to screen and identify compounds that specifically inhibit or reduce binding of a TβRII or ActRIIB polypeptide to TGFβ1, TGFβ3, activin, GDF11, GDF8, and/or BMP10. Alternatively, the assay can be used to identify compounds that enhance binding of a TβRII or ActRIIB polypeptide to TGFβ1, TGFβ3, activin, GDF11, GDF8, and/or BMP10. In a further embodiment, the compounds can be identified by their ability to interact with a TGFβ1, TGFβ3, activin, GDF11, GDF8, BMP10, ActRIIB, or TβRII polypeptide.

A variety of assay formats will suffice and, in light of the present disclosure, those not expressly described herein will nevertheless be comprehended by one of ordinary skill in the art. As described herein, the test compounds (agents) of the invention may be created by any combinatorial chemical method. Alternatively, the subject compounds may be naturally occurring biomolecules synthesized in vivo or in vitro. Compounds (agents) to be tested for their ability to act as modulators of tissue growth can be produced, for example, by bacteria, yeast, plants or other organisms (e.g., natural products), produced chemically (e.g., small molecules, including peptidomimetics), or produced recombinantly. Test compounds contemplated by the present invention include non-peptidyl organic molecules, peptides, polypeptides, peptidomimetics, sugars, hormones, and nucleic acid molecules. In a specific embodiment, the test agent is a small organic molecule having a molecular weight of less than about 2,000 daltons.

The test compounds of the invention can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be optionally derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S transferase (GST), photoactivatible crosslinkers or any combinations thereof.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an alteration of binding affinity between a TβRII or ActRIIB polypeptide and TGFβ1, TGFβ3, activin, GDF11, GDF8, and/or BMP10 polypeptides.

Merely to illustrate, in an exemplary screening assay of the present invention, the compound of interest is contacted with an isolated and purified ActRIIB:TβRII heteromultimer which is ordinarily capable of binding to TGFβ1 and activin A. To the mixture of the compound and ActRIIB:TβRII heteromultimer is then added a composition containing a ActRIIB:TβRII-binding ligand (e.g., TGFβ1 or activin A). Detection and quantification of ActRIIB:TβRII/TGFβ1 or ActRIIB:TβRII/activin A complexes provides a means for determining the compound's efficacy at inhibiting (or potentiating) complex formation between the ActRIIB:TβRII protein and TGFβ1 or activin A. The efficacy of the compound can be assessed by generating dose response curves from data obtained using various concentrations of the test compound. Moreover, a control assay can also be performed to provide a baseline for comparison. For example, in a control assay, isolated and a purified TGFβ1 or activin A is added to a composition containing the ActRIIB:TβRII heteromultimer, and the formation of ActRIIB:TβRII/TGFβ1 or ActRIIB:TβRII/activin A complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

Complex formation between any of the binders or multispecific binders of TGFβ-superfamily ligands disclosed herein (e.g., an the ActRIIB:TβRII heteromultimer) and TGFβ1, GDF8 and/or activin A may be detected by a variety of techniques. For instance, modulation of the formation of complexes can be quantitated using, for example, detectably labeled proteins such as radiolabeled (e.g., ³²P, ³⁵S, ¹⁴C or ³H), fluorescently labeled (e.g., FITC), or enzymatically labeled ActRIIB:TβRII heteromultimer or TGFβ1 or activin A, by immunoassay, or by chromatographic detection.

In certain embodiments, the present invention contemplates the use of fluorescence polarization assays and fluorescence resonance energy transfer (FRET) assays in measuring, either directly or indirectly, the degree of interaction between a ActRIIB:TβRII heteromultimer and its binding protein. Further, other modes of detection, such as those based on optical waveguides (PCT Publication WO 96/26432 and U.S. Pat. No. 5,677,196), surface plasmon resonance (SPR), surface charge sensors, and surface force sensors, are compatible with many embodiments of the invention.

Moreover, the present invention contemplates the use of an interaction trap assay, also known as the “two hybrid assay,” for identifying agents that disrupt or potentiate interaction between any of the binders or multispecific binders of TGFβ-superfamily ligands disclosed herein (e.g., an ActRIIB:TβRII heteromultimer) and its binding protein. See for example, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, the present invention contemplates the use of reverse two hybrid systems to identify compounds (e.g., small molecules or peptides) that dissociate interactions between a ActRIIB:TβRII heteromultimer and its binding protein. See for example, Vidal and Legrain, (1999) Nucleic Acids Res 27:919-29; Vidal and Legrain, (1999) Trends Biotechnol 17:374-81; and U.S. Pat. Nos. 5,525,490; 5,955,280; and 5,965,368.

In certain embodiments, the subject compounds are identified by their ability to interact with any of the binders or multispecific binders of TGFβ-superfamily ligands disclosed herein (e.g., anActRIIB:TβRII heteromultimer) or TGFβ1, TGFβ3, activin, GDF11, GDF8, and/or BMP10 polypeptide of the invention. The interaction between the compound and the binder/multispecific binder or TGFβ 1, TGFβ3, activin, GDF11, GDF8, and/or BMP10 polypeptide may be covalent or non-covalent. For example, such interaction can be identified at the protein level using in vitro biochemical methods, including photo-crosslinking, radiolabeled ligand binding, and affinity chromatography (Jakoby W B et al., 1974, Methods in Enzymology 46: 1). In certain cases, the compounds may be screened in a mechanism based assay, such as an assay to detect compounds which bind to a TGFβ1, TGFβ3, activin, GDF11, GDF8, and/or BMP10 polypeptide or binder/multispecific binder (e.g., ActRIIB:TβRII heteromultimer). This may include a solid-phase or fluid-phase binding event. Alternatively, the gene encoding a TGFβ1, TGFβ3, activin, GDF11, GDF8, and/or BMP10 polypeptide or binder/multispecific binder (e.g., ActRIIB:TβRII heteromultimer) can be transfected with a reporter system (e.g., β-galactosidase, luciferase, or green fluorescent protein) into a cell and screened against the library preferably by a high-throughput screening or with individual members of the library. Other mechanism-based binding assays may be used, for example, binding assays which detect changes in free energy. Binding assays can be performed with the target fixed to a well, bead or chip or captured by an immobilized antibody or resolved by capillary electrophoresis. The bound compounds may be detected usually using colorimetric or fluorescence or surface plasmon resonance.

In certain aspects, the present invention provides methods and agents for modulating (stimulating or inhibiting) TGFβ1-, TGFβ3-, activin-, GDF11-, GDF8-, BMP10-mediated cell signaling. Therefore, any compound identified can be tested in whole cells or tissues, in vitro or in vivo, to confirm their ability to modulate TGFβ1, TGFβ3, activin, GDF11, GDF8, and/or BMP10 signaling. Various methods known in the art can be utilized for this purpose.

### 5. Exemplary Therapeutic Uses

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The terms “treatment”, “treating”, “alleviation” and the like are used herein to generally mean obtaining a desired pharmacologic and/or physiologic effect, and may also be used to refer to improving, alleviating, and/or decreasing the severity of one or more symptoms of a condition being treated. The effect may be prophylactic in terms of completely or partially delaying the onset or recurrence of a disease, condition, or symptoms thereof, and/or may be therapeutic in terms of a partial or complete cure for a disease or condition and/or adverse effect attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms).

The terms “patient”, “subject”, or “individual” are used interchangeably herein and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human.

As used herein, “combination”, “in combination with”, “conjoint administration” and the like refers to any form of administration such that the second therapy is still effective in the body (e.g., the two compounds are simultaneously effective in the patient, which may include synergistic effects of the two compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, an individual who receives such treatment can benefit from a combined effect of different therapies. One or more of any of the binders or multispecific binders of TGFβ-superfamily ligands disclosed herein (e.g. an ActRIIB:TβRII heteromultimer) can be administered concurrently with, prior to, or subsequent to, one or more other additional agents or supportive therapies. In general, each therapeutic agent will be administered at a dose and/or on a time schedule determined for that particular agent. The particular combination to employ in a regimen will take into account compatibility of the antagonist of the present disclosure with the therapy and/or the desired therapeutic effect to be achieved.

Naturally occurring ActRIIB and TβRII receptor-ligand complexes play essential roles in tissue growth as well as early developmental processes such as the correct formation of various structures or in one or more post-developmental capacities. Thus, ActRIIB:TβRII-associated conditions or disorders include, but are not limited to, abnormal tissue growth and developmental defects. In addition, ActRIIB:TβRII-associated conditions or disorders include, but are not limited to, conditions or disorders of red blood cell formation (e.g., anemia), pulmonary conditions or diseases, kidney conditions or diseases, muscle conditions or diseases, and tumors or cancers.

In part, the disclosure provides methods of treating ActRIIB:TβRII-associated conditions or diseases by administering to a patient in need thereof an effective amount of an ActRIIB:TβRII heteromultimer. For example, in some embodiments, the methods relate to preventing or reducing the severity and/or duration of an ActRIIB:TβRII-associated condition or diseases in a patient in need thereof by administering an effective amount of an of any of the binders/multispecific binders of TGFβ-superfamily ligands disclosed herein (e.g. an ActRIIB:TβRII heteromultimer). Optionally, such methods further include administering of any of the binders/multispecific binders of TGFβ-superfamily ligands disclosed herein (e.g. an ActRIIB:TβRII heteromultimer) in combination with one or more additional active agents or supportive therapies for treating an ActRIIB:TβRII-associated condition or diseases.

In certain embodiments, any of the binders or multispecific binders of TGFβ-superfamily ligands disclosed herein (e.g. a multispecific binder comprising: i) a TβRII portion and ii) either a follistatin or follistatin-like polypeptide portion or an anti-GDF8 antibody or antigen-binding portion) may be used as part of a treatment for a muscular disorder. In some embodiments, the muscular disorder is associated with muscle wasting or muscle loss. In some embodiments, the muscular disorder is associated with muscle fibrosis. In some embodiments, the muscular disorder is associated with both muscle wasting/loss and muscle fibrosis. In some embodiments, any of the binders or multispecific binders disclosed herein treats both the muscle wasting/loss and the muscle fibrosis in the subject.

In certain aspects, the disclosure related to methods of treating a muscle-related diseases or condition (e.g., muscular dystrophy, muscle atrophy, muscle wasting syndrome, sarcopenia, cachexia, musculodegenerative disorders, neuromuscular disorders, and ALS) by administering to a patient in need thereof an effective amount of any of the binders or multispecific binders of TGFβ-superfamily ligands disclosed herein (e.g. an ActRIIB:TβRII heteromultimer). In particular, any of the binders or multispecific binders of TGFβ-superfamily ligands disclosed herein (e.g. an ActRIIB:TβRII heteromultimer) may be used to treat muscle-related diseases or conditions that are associated with muscle weakness and/or loss. In some embodiments, such methods further include administering the ActRIIB:TβRII heteromultimer in combination with one or more additional active agents or supportive therapies for treating a muscle-related diseases or conditions.

In some embodiments, any of the binders or multispecific binders of TGFβ-superfamily ligands disclosed herein (e.g. an ActRIIB:TβRII heteromultimer) may be used as part of a treatment for a muscular dystrophy. The term “muscular dystrophy” refers to a group of degenerative muscle diseases characterized by gradual weakening and deterioration of skeletal muscles and sometimes the heart and respiratory muscles. Muscular dystrophies are genetic disorders characterized by progressive muscle wasting and weakness that begin with microscopic changes in the muscle. As muscles degenerate over time, the person's muscle strength declines. Exemplary muscular dystrophies that can be treated with a regimen including the subject ActRIIB:TβRII heteromultimer include: Duchenne muscular dystrophy (DMD), Becker muscular dystrophy (BMD), Emery-Dreifuss muscular dystrophy (EDMD), limb-girdle muscular dystrophy (LGMD), facioscapulohumeral muscular dystrophy (FSH or FSHD) (also known as Landouzy-Dejerine), myotonic dystrophy (MMD; also known as Steinert's Disease), oculopharyngeal muscular dystrophy (OPMD), distal muscular dystrophy (DD), congenital muscular dystrophy (CMD).

Duchenne muscular dystrophy (DMD) was first described by the French neurologist Guillaume Benjamin Amand Duchenne in the 1860s. Becker muscular dystrophy (BMD) is named after the German doctor Peter Emil Becker, who first described this variant of DMD in the 1950s. DMD is one of the most frequent inherited diseases in males, affecting one in 3,500 boys. DMD occurs when the dystrophin gene, located on the short arm of the X chromosome, is defective. Since males only carry one copy of the X chromosome, they only have one copy of the dystrophin gene. Without the dystrophin protein, muscle is easily damaged during cycles of contraction and relaxation. While early in the disease muscle compensates by regeneration, later on muscle progenitor cells cannot keep up with the ongoing damage and healthy muscle is replaced by non-functional fibro-fatty tissue.

In certain embodiments, any of the binders or multispecific binders of TGFβ-superfamily ligands disclosed herein (e.g. a multispecific binder comprising: i) a TβRII portion and ii) either a follistatin or follistatin-like polypeptide portion or an anti-GDF8 antibody or antigen-binding portion) may be used as part of a treatment for a muscular dystrophy.

In DMD patients, muscle becomes injured, therefore muscle attempts to regenerate and recover. Muscle has the ability to regenerate after injury. Two processes are involved in skeletal muscle regeneration: myolysis and muscle reconstruction. Myolysis involves the degradation of muscle fibers followed by infiltration of inflammatory cells to the injury site. The muscle reconstruction involves the activation of muscle stem cells and satellite cells, to proliferate and to differentiate into myoblasts to form myofibers. Differentiating myoblasts synthesize extracellular matrix (ECM) with the goal to reconstruct the proper muscle, however, muscle regeneration often results in accumulation of ECM that develops into fibrosis (Delaney, K., et al., 2017, Cell Biology International, 41(7), 706-715.). TGFβ signaling has been found to negatively regulate skeletal muscle and muscle mass development. During embryonic development, TGFβ1 is expressed during myogenesis, and is correlated with the fiber-type composition of the surrounding myotubes (McLennan, 1993, Developmental Dynamics, 197(4):281-290). In mature adult muscle, TGFβ has been shown to negatively regulate skeletal muscle regeneration by inhibiting or influencing a dormant population of satellite cells, as well as decreasing myofiber fusion and expression of muscle-specific genes, such as MyoD and myogenin via the SMAD 3 signaling pathway (Allen, 1987, Journal of Cellular Physiology, 133(3):567-72; and Liu, 2001, Genes & Development, 15(22):2950-66). This inhibition can also lead to muscle atrophy (Narola, 2013, PLoS One, 8(11):E79356). In mouse models studying muscle loss and age, TGFβ1 and TGFβ3 have been found to be correlated with age related muscle loss. When TGFβ is suppressed there is improvement in muscle growth, implying that high levels of TGFβ contribute to muscle loss (Beggs, 2004, Aging Cell, 3(6):353-361). Upregulation of TGFβ superfamily signaling has been shown to play a key role in DMD pathology. Higher levels of TGFβ1, TGFβ type I and TGFβ type II receptors is associated with the severity of DMD phenotype in mdx mice (Zhou, 2006). In DMD patients, mRNA profiling studies using DMD muscle tissue from patients at various disease stages have shown increased levels of TGFβ-1 signaling (Chen, 2005). Another study also show that upregulated TGFβ1 expression in skeletal muscle of DMD patients correlates between fibrotic pathology and clinical severity (Song, 2017, Experimental and Therapeutic Medicine, 13(4):1209-1214). One characteristic in DMD pathology is connective tissue proliferation in muscles, which leads to irreversible tissue damage of tissue muscle organization in dystrophic muscles. This characteristic also correlates with the observation that TGFβ-1 upregulation triggers extracellular matrix formation (Bernasconi, 1995, Journal of Clinical Investigation, 96(2):1137-44). For example, TGFβ1 regulates the extracellular matrix by increasing fibroblasts production and specific extracellular matrix proteins, type I and III collagen (Gumucio, 2015, Exercise and Sport Sciences Reviews, 43(2):93-99). Moreover, TGFβ1 stimulates muscle derived stem cells (MDSC) to differentiate into myofibroblasts, which then causes extracellular matrix overproduction and inhibits matrix degradation, ultimately resulting in muscle fibrosis (Li, 2004). The expression of TGFβ1 in muscle cells in vitro and in vivo show that TGFβ1 stimulates fibrotic cascades (Li, 2004, American Journal of Pathology, 164(3):1007-1019; Serrano, 2010, Experimental Cell Research, 316(18):3050-3058). These studies all demonstrate the role of TGFβ-1 in DMD, especially in connective tissue proliferation that results in muscle fibrosis. More evidence supports the role of TGFβ in DMD pathology. Macrophage is the source to produce TGFβ1, which contributes to fibrosis by activating fibroblasts that produce collagen and other factors that create the extracellular matrix (Wynn, 2007, Journal of Pathology, 214(2):199-210). In DMD, inflammatory response is also prevalent with the recruitment of macrophages, T-cells, neutrophils, mast cells, and eosinophils. Although there is recruitment of various inflammatory cell populations, DMD models show an exceptionally high number of macrophages (Villalta, 2010, Human Molecular Genetics, 20(4):790-805). When depleting macrophages in mdx models, myopathy decreased; therefore macrophages may play a role in muscle pathology (Tidball, 2010, American Journal of Physiology-Regulatory Integrative and Comparative Physi, 298(5):R1173-R1187).

In some embodiments, any of the binders or multispecific binders disclosed herein (e.g. a multispecific binder comprising: i) a TβRII portion and ii) either a follistatin or follistatin-like polypeptide portion or an anti-GDF8 antibody or antigen-binding portion) may be used to treat a disorder associated with both muscle wasting/muscle loss and muscle fibrosis. In some embodiments, the binder/multispecific binder is capable of both increasing muscle mass and decreasing muscle fibrosis in a subject having a muscle disorder (e.g., DMD).

BMD results from different mutations in the dystrophin gene. BMD patients have some dystrophin, but it is either of insufficient quantity or poor quality. The presence of some dystrophin protects the muscles of patients with BMD from degenerating as severely or as quickly as those of patients with DMD.

Studies in animals indicate that inhibition of the GDF8 signaling pathway may effectively treat various aspects of disease in DMD and BMD patients (Bogdanovich et al., 2002, Nature 420:418-421; Pistilli et al., 2011, Am J Pathol 178:1287-1297). Thus, ActRIIB:TβRII heteromultimers of the disclosure may act as GDF8 inhibitors (antagonists), and constitute an alternative means of blocking signaling by GDF8 and/or related TGFβ superfamily ligands in vivo in DMD and BMD patients.

Similarly, ActRIIB:TβRII heteromultimers of the disclosure may provide an effective means to increase muscle mass in other disease conditions that are in need of muscle growth. For example, amyotrophic lateral sclerosis (ALS), also called Lou Gehrig's disease or motor neuron disease, is a chronic, progressive, and incurable CNS disorder that attacks motor neurons, which are components of the central nervous system required for initiation of skeletal muscle contraction. In ALS, motor neurons deteriorate and eventually die, and though a person's brain normally remains fully functioning and alert, initiation of muscle contraction is blocked at the spinal level. Individuals who develop ALS are typically between 40 and 70 years old, and the first motor neurons to degenerate are those innervating the arms or legs. Patients with ALS may have trouble walking, may drop things, fall, slur their speech, and laugh or cry uncontrollably. As the disease progresses, muscles in the limbs begin to atrophy from disuse. Muscle weakness becomes debilitating, and patients eventually require a wheel chair or become confined to bed. Most ALS patients die from respiratory failure or from complications of ventilator assistance like pneumonia 3-5 years from disease onset.

In some embodiments, Charcot-Marie-Tooth Disease (CMT) may be treated by administration of an ActRIIB:TβRII heteromultimer described herein. CMT is a group of inherited disorders affecting the peripheral nerves and resulting in progressive, and often local, muscle weakness and degeneration. Aspects of the disease that may be treated include foot deformity (very high arched feet); foot drop (inability to hold foot horizontal); “Slapping” gait (feet slap on the floor when walking because of foot drop); loss of muscle in the lower legs; numbness in the feet; difficulty with balance; or weakness in the arms and hands.

In some embodiments, muscle of patients with a variety of systemic muscle disorder may be treated with an ActRIIB:TβRII heteromultimer disclosed herein including, for example, including: Lambert-Eaton Myasthenic Syndrome (LEMS); Metabolic Dystrophies; Spinal Muscular Atrophy (SMA); Dermatomyositis (DM); Distal Muscular Dystrophy (DD); Emery-Dreifuss Muscular Dystrophy (EDMD); Endocrine Myopathies; Friedreich's Ataxia (FA); Inherited Myopathies; Mitochondrial Myopathy; Myasthenia Gravis (MG); Polymyositis (PM).

In some embodiments, muscles of patients with a post-surgical or disuse atrophy of one or more muscles may be treating with any of the binders or multispecific binders of TGFβ-superfamily ligands disclosed herein (e.g. an ActRIIB: TβRII heteromultimer) including atrophy after: Hip Fracture; Total Hip Arthroplasty (THA); Total Knee Arthroplasty (TKA) or Rotator Cuff surgery.

In some embodiments, muscles of patients suffering from a variety of other diseases that cause muscle loss or weakening may be treated with any of the binders or multispecific binders of TGFβ-superfamily ligands disclosed herein (e.g. an ActRIIB:TβRII heteromultimer), including muscles of patients with the following diseases: sarcopenia, cachexia, various types of cancer, including lung, colon and ovarian cancer, patients on long term ventilation assistance, diabetes, chronic obstructive pulmonary disorder, renal failure, cardiac failure, trauma and disorders of the peripheral nerves.

Promotion of increased muscle mass by any of the binders or multispecific binders of TGFβ-superfamily ligands disclosed herein (e.g. an ActRIIB:TβRII heteromultimer) might also benefit those suffering from muscle wasting diseases. Gonzalez-Cadavid et al. (supra) reported that GDF8 expression correlates inversely with fat-free mass in humans and that increased expression of the GDF8 gene is associated with weight loss in men with AIDS wasting syndrome. By inhibiting the function of GDF8 in AIDS patients, at least certain symptoms of AIDS may be alleviated, if not completely eliminated, thus significantly improving quality of life in AIDS patients.

Cancer anorexia-cachexia syndrome is among the most debilitating and life-threatening aspects of cancer. This syndrome is a common feature of many types of cancer—present in approximately 80% of cancer patients at death—and is responsible not only for a poor quality of life and poor response to chemotherapy but also a shorter survival time than is found in patients with comparable tumors but without weight loss. Cachexia is typically suspected in patients with cancer if an involuntary weight loss of greater than five percent of premorbid weight occurs within a six-month period. Associated with anorexia, wasting of fat and muscle tissue, and psychological distress, cachexia arises from a complex interaction between the cancer and the host. Cancer cachexia affects cytokine production, release of lipid-mobilizing and proteolysis-inducing factors, and alterations in intermediary metabolism. Although anorexia is common, a decreased food intake alone is unable to account for the changes in body composition seen in cancer patients, and increasing nutrient intake is unable to reverse the wasting syndrome. Currently, there is no treatment to control or reverse the cachexic process. Since systemic overexpression of GDF8 in adult mice was found to induce profound muscle and fat loss analogous to that seen in human cachexia syndromes (Zimmers et al., supra), the subject ActRIIB:TβRII heteromultimer may be beneficially used to prevent, treat, or alleviate the symptoms of the cachexia syndrome, where muscle growth is desired.

In some embodiments, the present disclosure relates to methods of treating pulmonary hypertension (e.g., pulmonary arterial hypertension) comprising administering to a patient in need thereof an effective amount of any of the binders or multispecific binders of TGFβ-superfamily ligands disclosed herein (e.g. an ActRIIB:TβRII heteromultimer). For example, in some embodiments, the disclosure related to methods of preventing or reducing the severity or progression rate of one or more complications of pulmonary hypertension (e.g., smooth muscle and/or endothelial cell proliferation in the pulmonary artery, angiogenesis in the pulmonary artery, dyspnea, chest pain, pulmonary vascular remodeling, right ventricular hypertrophy, and pulmonary fibrosis) comprising administering to a patient in need thereof an effective amount of any of the binders or multispecific binders of TGFβ-superfamily ligands disclosed herein (e.g. an ActRIIB:TβRII heteromultimer). Optionally, methods disclosed herein for treating pulmonary hypertension may further comprise administering to the patient one or more supportive therapies or additional active agents for treating pulmonary hypertension. For example, the patient also may be administered one or more supportive therapies or active agents selected from the group consisting of: prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1-->3)-beta-D-glucopyranosyl]oleanolic acid; 3-O-[beta-D-glucopyranosyl-(1-->2)-beta-D-glucopyranosyl]oleanolic acid; 3-O-[beta-D-glucopyranosyl-(1-->3)-beta-D-glucopyranosyl]oleanolic acid 28-O-beta-D-glucopyranosyl ester; 3-O-[beta-D-glucopyranosyl-(1-->2)-beta-D-glucopyranosyl]oleanolic acid 28-O-beta-D-glucopyranosyl ester; 3-O-[a-L-rhamnopyranosyl-(1-->3)-beta-D-glucuronopyranosyl]oleanolic acid; 3-O-[alpha-L-rhamnopyranosyl-(1-->3)-beta-D-glucuronopyranosyl]oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1); oleanolic acid 3-O-β-D-glucopyranosyl (1→3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2,3]oleanolate) lung and/or heart transplantation.

Pulmonary hypertension (PH) has been previously classified as primary (idiopathic) or secondary. Recently, the World Health Organization (WHO) has classified pulmonary hypertension into five groups: Group 1: pulmonary arterial hypertension (PAH); Group 2: pulmonary hypertension with left heart disease; Group 3: pulmonary hypertension with lung disease and/or hypoxemia; Group 4: pulmonary hypertension due to chronic thrombotic and/or embolic disease; and Group 5: miscellaneous conditions (e.g., sarcoidosis, histiocytosis X, lymphangiomatosis and compression of pulmonary vessels). See, for example, Rubin (2004) Chest 126:7-10. In some embodiments, the methods disclosed herein relate treating PH designated as any one of Group 1-5 by the WHO. In some embodiments, the methods relate to treating PAH.

Pulmonary arterial hypertension is a serious, progressive and life-threatening disease of the pulmonary vasculature, characterized by profound vasoconstriction and an abnormal proliferation of smooth muscle cells in the walls of the pulmonary arteries. Severe constriction of the blood vessels in the lungs leads to very high pulmonary arterial pressures. These high pressures make it difficult for the heart to pump blood through the lungs to be oxygenated. Patients with PAH suffer from extreme shortness of breath as the heart struggles to pump against these high pressures. Patients with PAH typically develop significant increases in pulmonary vascular resistance (PVR) and sustained elevations in pulmonary artery pressure (PAP), which ultimately lead to right ventricular failure and death. Patients diagnosed with PAH have a poor prognosis and equally compromised quality of life, with a mean life expectancy of 2 to 5 years from the time of diagnosis if untreated.

A variety of factors contribute to the pathogenesis of pulmonary hypertension including proliferation of pulmonary cells which can contribute to vascular remodeling (i.e., hyperplasia). For example, pulmonary vascular remodeling occurs primarily by proliferation of arterial endothelial cells and smooth muscle cells of patients with pulmonary hypertension. Overexpression of various cytokines is believed to promote pulmonary hypertension. Further, it has been found that pulmonary hypertension may rise from the hyperproliferation of pulmonary arterial smooth cells and pulmonary endothelial cells. Still further, advanced PAH may be characterized by muscularization of distal pulmonary arterioles, concentric intimal thickening, and obstruction of the vascular lumen by proliferating endothelial cells. Pietra et al., J. Am. Coll. Cardiol., 43:255-325 (2004).

In some embodiments, the present disclosure relates to methods of treating an interstitial lung disease (e.g., idiopathic pulmonary fibrosis) comprising administering to a patient in need thereof an effective amount of any of the binders or multispecific binders of TGFβ-superfamily ligands disclosed herein (e.g. an ActRIIB:TβRII heteromultimer). In some embodiments, the interstitial lung disease is pulmonary fibrosis. In some embodiments, the interstitial lung disease is caused by any one of the following: silicosis, asbestosis, berylliosis, hypersensitivity pneumonitis, drug use (e.g., antibiotics, chemotherapeutic drugs, antiarrhythmic agents, statins), systemic sclerosis, polymyositis, dermatomyositis, systemic lupus erythematosus, rheumatoid arthritis, an infection (e.g., atypical pneumonia, pneumocystis pneumonia, tuberculosis, Chlamydia trachomatis, and/or respiratory syncytial virus), lymphangitic carcinomatosis, cigarette smoking, or developmental disorders. In some embodiments, the interstitial lung disease is idiopathic (e.g., sarcoidosis, idiopathic pulmonary fibrosis, Hamman-Rich syndrome, and/or antisynthetase syndrome). In particular embodiments, the interstitial lung disease is idiopathic pulmonary fibrosis. In some embodiments, the treatment for idiopathic pulmonary fibrosis is administered in combination with an additional therapeutic agent. In some embodiments, the additional therapeutic agent is selected from the group consisting of: pirfenidone, N-acetylcysteine, prednisone, azathioprine, nintedanib, derivatives thereof and combinations thereof.

In some embodiments, the disclosure relates to methods of treating a fibrotic or sclerotic disease, disorder or condition. As used herein, the terms “fibrotic disorder”, “fibrotic condition,” and “fibrotic disease,” are used interchangeably to refer to a disorder, condition or disease characterized by fibrosis. Examples of fibrotic disorders include, but are not limited to lupus, sclerotic disorders (e.g., scleroderma, atherosclerosis, and systemic scleroisis including, e.g., diffuse systemic sclerosis and progressive systemic sclerosis), vascular fibrosis, pancreatic fibrosis, liver fibrosis (e.g., cirrhosis), renal fibrosis, musculoskeletal fibrosis, cardiac fibrosis (e.g., endomyocardial fibrosis, idiopathic myocardiopathy), skin fibrosis (e.g., scleroderma, post-traumatic, operative cutaneous scarring, keloids and cutaneous keloid formation), eye fibrosis (e.g., glaucoma, sclerosis of the eyes, conjunctival and corneal scarring, and pterygium), myelofibrosis, chronic graft-versus-host disease, Peyronie's disease, post-cystoscopic urethral stenosis, idiopathic and pharmacologically induced retroperitoneal fibrosis, mediastinal fibrosis, proliferative fibrosis, neoplastic fibrosis, Dupuytren's disease, strictures, neural scarring, dermal scarring, idiopathic pulmonary fibrosis and radiation induced fibrosis.

In some embodiments, the present disclosure relates to methods of treating a tumor or cancer comprising administering to a patient in need thereof an effective amount of any of the binders or multispecific binders of TGFβ-superfamily ligands disclosed herein (e.g. an ActRIIB:TβRII heteromultimer). For example, in some embodiments, the disclosure related to methods of preventing or reducing the severity or progression rate of one or more complications of a tumor or cancer. Optionally, methods disclosed herein for treating a tumor or cancer may further comprise administering to the patient one or more supportive therapies or additional active agents for treating the tumor or cancer. In addition, any of the binders or multispecific binders of TGFβ-superfamily ligands disclosed herein (e.g. an ActRIIB:TβRII heteromultimer) may be used to treat malignant or premalignant conditions and to prevent progression to a neoplastic or malignant state, including but not limited to those disorders described herein. Such uses are indicated in conditions known or suspected of preceding progression to neoplasia or cancer, in particular, where non-neoplastic cell growth consisting of hyperplasia, metaplasia, or most particularly, dysplasia has occurred.

In general, “tumors” refers to benign and malignant cancers as well as dormant tumors. In general, “cancer” refers to primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). Metastasis can be local or distant. Metastases are most often detected through the sole or combined use of magnetic resonance imaging (MRI) scans, computed tomography (CT) scans, blood and platelet counts, liver function studies, chest X-rays, bone scans in addition to the monitoring of specific symptoms, and combinations thereof.

In some embodiments, any of the binders or multispecific binders of TGFβ-superfamily ligands disclosed herein (e.g. an ActRIIB:TβRII heteromultimer) may be used in the treatment of various forms of cancer, including, but not limited to, cancer of the bladder, breast, colon, kidney, liver, lung, ovary, cervix, pancreas, rectum, prostate, stomach, epidermis; a hematopoietic tumor of lymphoid or myeloid lineage; a tumor of mesenchymal origin such as a fibrosarcoma or rhabdomyosarcoma; other tumor types such as melanoma, teratocarci-noma, neuroblastoma, glioma, adenocarcinoma and non-small lung cell carcinoma. Examples of cancers include, but are not limited to, carcinoma, lymphoma, glioblastoma, melanoma, sarcoma, and leukemia, myeloma, or lymphoid malignancies. More particular examples of such cancers are noted below and include: squamous cell cancer (e.g., epithelial squamous cell cancer), Ewing sarcoma, Wilms tumor, astrocytomas, lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma multiforme, cervical cancer, ovarian cancer, liver cancer, hepatoma, hepatocellular carcinoma, neuroendocrine tumors, medullary thyroid cancer, differentiated thyroid carcinoma, breast cancer, ovarian cancer, colon cancer, rectal cancer, endometrial cancer or uterine carcinoma, salivary gland carcinoma, kidney or renal cancer, prostate cancer, vulvar cancer, anal carcinoma, penile carcinoma, as well as head-and-neck cancer.

Other examples of cancers or malignancies include, but are not limited to: acute childhood Other examples of cancers or malignancies include, but are not limited to: acute childhood lymphoblastic leukemia, acute lymphoblastic leukemia, acute lymphocytic leukemia, acute myeloid leukemia, adrenocortical carcinoma, adult (primary) hepatocellular cancer, adult (primary) liver cancer, adult acute lymphocytic leukemia, adult acute myeloid leukemia, adult Hodgkin's lymphoma, adult lymphocytic leukemia, adult non-Hodgkin's lymphoma, adult primary liver cancer, adult soft tissue sarcoma, AIDs-related lymphoma, AIDs-related malignancies, anal cancer, astrocytoma, bile duct cancer, bone cancer, brain stem glioma, brain tumors, breast cancer, cancer of the renal pelvis and ureter, central nervous system (primary) lymphoma, central nervous system lymphoma, cerebellar astrocytoma, cerebral astrocytoma, cervical cancer, childhood (primary) hepatocellular cancer, childhood (primary) liver cancer, childhood acute lymphoblastic leukemia, childhood acute myeloid leukemia, childhood brain stem glioma, childhood cerebellar astrocytoma, childhood cerebral astrocytoma, childhood extracranial germ cell tumors, childhood Hodgkin's disease, childhood Hodgkin's lymphoma, childhood hypothalamic and visual pathway glioma, childhood lymphoblastic leukemia, childhood medulloblastoma, childhood non-Hodgkin's lymphoma, childhood pineal and supratentorial primitive neuroectodermal tumors, childhood primary liver cancer, childhood rhabdomyosarcoma, childhood soft tissue sarcoma, childhood visual pathway and hypothalamic glioma, chronic lymphocytic leukemia, chronic myelogenous leukemia, colon cancer, cutaneous t-cell lymphoma, endocrine pancreas islet cell carcinoma, endometrial cancer, ependymoma, epithelial cancer, esophageal cancer, Ewing's sarcoma and related tumors, exocrine pancreatic cancer, extracranial germ cell tumor, extragonadal germ cell tumor, extrahepatic bile duct cancer, eye cancer, female breast cancer, Gaucher's disease, gallbladder cancer, gastric cancer, gastrointestinal carcinoid tumor, gastrointestinal tumors, germ cell tumors, gestational trophoblastic tumor, hairy cell leukemia, head and neck cancer, hepatocellular cancer, Hodgkin's lymphoma, hypergammaglobulinemia, hypopharyngeal cancer, intestinal cancers, intraocular melanoma, islet cell carcinoma, islet cell pancreatic cancer, Kaposi's sarcoma, kidney cancer, laryngeal cancer, lip and oral cavity cancer, liver cancer, lung cancer, lymphoproliferative disorders, macroglobulinemia, male breast cancer, malignant mesothelioma, malignant thymoma, medulloblastoma, melanoma, mesothelioma, metastatic occult primary squamous neck cancer, metastatic primary squamous neck cancer, metastatic squamous neck cancer, multiple myeloma, multiple myeloma/plasma cell neoplasm, myelodysplastic syndrome, myelogenous leukemia, myeloid leukemia, myeloproliferative disorders, nasal cavity and paranasal sinus cancer, nasopharyngeal cancer, neuroblastoma, non-Hodgkin's lymphoma, nonmelanoma skin cancer, non-small cell lung cancer, occult primary metastatic squamous neck cancer, oropharyngeal cancer, osteo-/malignant fibrous sarcoma, osteosarcoma/malignant fibrous histiocytoma, osteosarcoma/malignant fibrous histiocytoma of bone, ovarian epithelial cancer, ovarian germ cell tumor, ovarian low malignant potential tumor, pancreatic cancer, paraproteinemias, parathyroid cancer, penile cancer, pheochromocytoma, pituitary tumor, primary central nervous system lymphoma, primary liver cancer, prostate cancer, rectal cancer, renal cell cancer, renal pelvis and ureter cancer, retinoblastoma, rhabdomyosarcoma, salivary gland cancer, sarcoidosis sarcomas, Sezary syndrome, skin cancer, small cell lung cancer, small intestine cancer, soft tissue sarcoma, squamous neck cancer, stomach cancer, supratentorial primitive neuroectodermal and pineal tumors, t-cell lymphoma, testicular cancer, thymoma, thyroid cancer, transitional cell cancer of the renal pelvis and ureter, transitional renal pelvis and ureter cancer, trophoblastic tumors, ureter and renal pelvis cell cancer, urethral cancer, uterine cancer, uterine sarcoma, vaginal cancer, visual pathway and hypothalamic glioma, vulvar cancer, Waldenstrom's macroglobulinemia, Wilms' tumor, and any other hyperproliferative disease, besides neoplasia, located in an organ system listed above.

Dysplasia is frequently a forerunner of cancer, and is found mainly in the epithelia. It is the most disorderly form of non-neoplastic cell growth, involving a loss in individual cell uniformity and in the architectural orientation of cells. Dysplasia characteristically occurs where there exists chronic irritation or inflammation. In some embodiments, an ActRIIB:TβRII heteromultimer may be used to treat a dysplastic disorders. Dysplastic disorders include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, opthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders which may be treated with an ActRIIB:TβRII heteromultimer include, but are not limited to, benign dysproliferative disorders (e.g., benign tumors, fibrocystic conditions, tissue hypertrophy, intestinal polyps or adenomas, and esophageal dysplasia), leukoplakia, keratoses, Bowen's disease, Farner's Skin, solar cheilitis, and solar keratosis.

Additional hyperproliferative diseases, disorders, and/or conditions which may be treated with an ActRIIB:TβRII heteromultimer, include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, epithelial carcinoma, glioma, astrocytoma, medulloblastoma, craniopharyngioma, ependymoma, pinealoma, emangioblastoma, acoustic neuroma, oligodendroglioma, meningioma, melanoma, neuroblastoma, and retinoblastoma.

In certain aspects, therapeutic cancer agents such as cytotoxic agents, anti-angiogenic agents, pro-apoptotic agents, immunomodulator agents, antibiotics, hormones, hormone antagonists, chemokines, drugs, prodrugs, toxins, enzymes or other active agents may be used in combination with any of the binders or multispecific binders of TGFβ-superfamily ligands disclosed herein (e.g. an ActRIIB:TβRII heteromultimer). Drugs of use may possess a pharmaceutical property selected from, for example: antimitotic, anti-kinase, alkylating, antimetabolite, antibiotic, alkaloid, anti-angiogenic, pro-apoptotic agents, and combinations thereof.

For example, anti-PD1 antibodies have been used for treatment of melanoma, non-small-cell lung cancer, bladder cancer, prostate cancer, colorectal cancer, head and neck cancer, triple-negative breast cancer, leukemia, lymphoma and renal cell cancer (Topalian et al., 2012, N Engl J Med 366:2443-54; Lipson et al., 2013, Clin Cancer Res 19:462-8; Berger et al., 2008, Clin Cancer Res 14:3044-51; Gildener-Leapman et al., 2013, Oral Oncol 49:1089-96; Menzies & Long, 2013, Ther Adv Med Oncol 5:278-85). Exemplary anti-PD1 antibodies include lambrolizumab (MK-3475, MERCK), nivolumab (BMS-936558, Bristol-Myers Squibb), AMP-224 (Merck), and pidilizumab (CT-011, Curetech Ltd.). Anti-PD1 antibodies are commercially available, for example from ABCAM (AB137132), Biolegend (EH12.2H7, RMP1-14) and Affymetrix Ebioscience (J105, J116, MIH4).

Anti-CTL4A antibodies have been used in clinical trials for treatment of melanoma, prostate cancer, small cell lung cancer, non-small cell lung cancer (Robert & Ghiringhelli, 2009, Oncologist 14:848-61; Ott et al., 2013, Clin Cancer Res 19:5300; Weber, 2007, Oncologist 12:864-72; Wada et al., 2013, J Transl Med 11:89). Exemplary anti-CTLA4 antibodies include ipilimumab (Bristol-Myers Squibb) and tremelimumab (Pfizer). Anti-PD1 antibodies are commercially available, for example from ABCAM (AB134090), Sino Biological Inc. (11159-H03H, 11159-H08H), and Thermo Scientific Pierce (PA5-29572, PA5-23967, PA5-26465, MA1-12205, MA1-35914). Ipilimumab has recently received FDA approval for treatment of metastatic melanoma (Wada et al., 2013, J Transl Med 11:89).

Although checkpoint inhibitor against CTLA4, PD1 and PD-L1 are the most clinically advanced, other potential checkpoint antigens are known and may be used as the target of therapeutic inhibitors in combination with any of the binders or multispecific binders of TGFβ-superfamily ligands disclosed herein (e.g. an ActRIIB:TβRII heteromultimer), such as LAG3, B7-H3, B7-H4 and TIM3 (Pardoll, 2012, Nature Reviews Cancer 12:252-264). These and other known agents that stimulate immune response to tumors and/or pathogens may be used in combination with ActRIIB:TβRII heteromultimers alone or in further combination with an interferon, such as interferon-α, and/or an antibody-drug conjugate for improved cancer therapy. Other known co-stimulatory pathway modulators that may be used in combination include, but are not limited to, agatolimod, belatacept, blinatumomab, CD40 ligand, anti-B7-1 antibody, anti-B7-2 antibody, anti-B7-H4 antibody, AG4263, eritoran, anti-OX40 antibody, ISF-154, and SGN-70; B7-1, B7-2, ICAM-1, ICAM-2, ICAM-3, CD48, LFA-3, CD30 ligand, CD40 ligand, heat stable antigen, B7h, OX40 ligand, LIGHT, CD70 and CD24.

In some embodiments, the present disclosure relates to methods of treating a kidney (renal) disease or condition comprising administering to a patient in need thereof an effective amount of any of the binders or multispecific binders of TGFβ-superfamily ligands disclosed herein (e.g. an ActRIIB:TβRII heteromultimer). For example, in some embodiments, the disclosure relates to methods of preventing or reducing the severity or progression rate of one or more complications of a kidney disease or condition. Optionally, methods disclosed herein for treating a kidney disease or condition may further comprise administering to the patient one or more supportive therapies or additional active agents for treating the kidney disease or condition. For example, the patient also may be administered one or more supportive therapies or active agents selected from the group consisting of: angiotensin-converting enzyme (ACE) inhibitors, angiotensin II receptor blockers, water pills, statins, erythropoietin, diuretics, calcium and/or vitamin D supplement, a phosphate binder, calcium, glucose or sodium polystyrene sulfonate (e.g., Kayexalate, Kionex), by hemodialysis and/or peritoneal dialysis, Lasix® (furosemide), Demadex® (torsemide), Edecrin® (ethacrynic acid), and sodium edecrin.

The kidneys maintain many features of the blood, including volume, pH balance, electrolyte concentrations, and blood pressure, as well as bearing responsibility for toxin and waste filtration. These functions depend upon the intricate structure of the kidney nephrons, constant flow of blood through the various capillaries of the kidney, and the regulation of the kidney by signals from the rest of the body, including endocrine hormones. Problems with kidney function manifest by direct mechanisms (e.g., genetic defects, infection, or toxin exposure) and by indirect mechanisms progressively proceeding from long term stressors like hypertrophy and hyperfiltration (themselves often a result of more direct insults to kidney function). Due to the central role of the kidney in blood maintenance and waste secretion, kidney-associated disease manifestations are many and varied; they can be reviewed in Harrison's Principles of Internal Medicine, 18^{th} edition, McGraw Hill, N.Y., Part 13, Chp 277-289.

Therefore, methods of this disclosure can be applied to various kidney-associated diseases or conditions. As used herein, kidney-associated disease or condition can refer to any disease, disorder, or condition that affects the kidneys or the renal system. Examples of kidney-associated diseases or conditions include, but are not limited to, chronic kidney diseases (or failure), acute kidney diseases (or failure), primary kidney diseases, non-diabetic kidney diseases, glomerulonephritis, interstitial nephritis, diabetic kidney diseases, diabetic chronic kidney disease, diabetic nephropathy, glomerulosclerosis, rapid progressive glomerulonephritis, renal fibrosis, Alport syndrome, IDDM nephritis, mesangial proliferative glomerulonephritis, membranoproliferative glomerulonephritis, crescentic glomerulonephritis, renal interstitial fibrosis, focal segmental glomerulosclerosis, membranous nephropathy, minimal change disease, pauci-immune rapid progressive glomerulonephritis, IgA nephropathy, polycystic kidney disease, Dent's disease, nephrocytinosis, Heymann nephritis, polycystic kidney disease (e.g., autosomal dominant (adult) polycystic kidney disease and autosomal recessive (childhood) polycystic kidney disease), acute kidney injury, nephrotic syndrome, renal ischemia, podocyte diseases or disorders, proteinuria, glomerular diseases, membranous glomerulonephritis, focal segmental glomerulonephritis, pre-eclampsia, eclampsia, kidney lesions, collagen vascular diseases, benign orthostatic (postural) proteinuria, IgM nephropathy, membranous nephropathy, sarcoidosis, diabetes mellitus, kidney damage due to drugs, Fabry's disease, aminoaciduria, Fanconi syndrome, hypertensive nephrosclerosis, interstitial nephritis, acute interstitial nephritis, Sickle cell disease, hemoglobinuria, myoglobinuria, Wegener's Granulomatosis, Glycogen Storage Disease Type 1, chronic kidney disease, chronic renal failure, low Glomerular Filtration Rate (GFR), nephroangiosclerosis, lupus nephritis, ANCA-positive pauci-immune crescentic glomerulonephritis, chronic allograft nephropathy, nephrotoxicity, renal toxicity, kidney necrosis, kidney damage, glomerular and tubular injury, kidney dysfunction, nephritic syndrome, acute renal failure, chronic renal failure, proximal tubal dysfunction, acute kidney transplant rejection, chronic kidney transplant rejection, non-IgA mesangioproliferative glomerulonephritis, postinfectious glomerulonephritis, vasculitides with renal involvement of any kind, any hereditary renal disease, any interstitial nephritis, renal transplant failure, kidney cancer, kidney disease associated with other conditions (e.g., hypertension, diabetes, and autoimmune disease), Dent's disease, nephrocytinosis, Heymann nephritis, a primary kidney disease, a collapsing glomerulopathy, a dense deposit disease, a cryoglobulinemia-associated glomerulonephritis, an Henoch-Schonlein disease, a postinfectious glomerulonephritis, a bacterial endocarditis, a microscopic polyangitis, a Churg-Strauss syndrome, an anti-GBM-antibody mediated glomerulonephritis, amyloidosis, a monoclonal immunoglobulin deposition disease, a fibrillary glomerulonephritis, an immunotactoid glomerulopathy, ischemic tubular injury, a medication-induced tubulo-interstitial nephritis, a toxic tubulo-interstitial nephritis, an infectious tubulo-interstitial nephritis, a bacterial pyelonephritis, a viral infectious tubulo-interstitial nephritis which results from a polyomavirus infection or an HIV infection, a metabolic-induced tubulo-interstitial disease, a mixed connective disease, a cast nephropathy, a crystal nephropathy which may results from urate or oxalate or drug-induced crystal deposition, an acute cellular tubulo-interstitial allograft rejection, a tumoral infiltrative disease which results from a lymphoma or a post-transplant lymphoproliferative disease, an obstructive disease of the kidney, vascular disease, a thrombotic microangiopathy, a nephroangiosclerosis, an atheroembolic disease, a mixed connective tissue disease, a polyarteritis nodosa, a calcineurin-inhibitor induced-vascular disease, an acute cellular vascular allograft rejection, an acute humoral allograft rejection, early renal function decline (ERFD), end stage renal disease (ESRD), renal vein thrombosis, acute tubular necrosis, acute interstitial nephritis, established chronic kidney disease, renal artery stenosis, ischemic nephropathy, uremia, drug and toxin-induced chronic tubulointerstitial nephritis, reflux nephropathy, kidney stones, Goodpasture's syndrome, normocytic normochromic anemia, renal anemia, diabetic chronic kidney disease, IgG4-related disease, von Hippel-Lindau syndrome, tuberous sclerosis, nephronophthisis, medullary cystic kidney disease, renal cell carcinoma, adenocarcinoma, nephroblastoma, lymphoma, leukemia, hyposialylation disorder, chronic cyclosporine nephropathy, renal reperfusion injury, renal dysplasia, azotemia, bilateral arterial occlusion, acute uric acid nephropathy, hypovolemia, acute bilateral obstructive uropathy, hypercalcemic nephropathy, hemolytic uremic syndrome, acute urinary retention, malignant nephrosclerosis, postpartum glomerulosclerosis, scleroderma, non-Goodpasture's anti-GBM disease, microscopic polyarteritis nodosa, allergic granulomatosis, acute radiation nephritis, post-streptococcal glomerulonephritis, Waldenstrom's macroglobulinemia, analgesic nephropathy, arteriovenous fistula, arteriovenous graft, dialysis, ectopic kidney, medullary sponge kidney, renal osteodystrophy, solitary kidney, hydronephrosis, microalbuminuria, uremia, haematuria, hyperlipidemia, hypoalbuminaemia, lipiduria, acidosis, edma, tubulointerstitial renal fibrosis, hypertensive sclerosis, juxtaglomerular cell tumor, Fraser syndrome, Horseshoe kidney, renal tubular dysgenesis, hypokalemia, hypomagnesemia, hypercalcemia, hypophosphatemia, uromodulin-associated kidney disease, Nail-patella syndrome, lithium nephrotoxity, TNF-alpha nephrotoxicity, honeybee resin related renal failure, sugarcane harvesting acute renal failure, complete LCAT deficiency, Fraley syndrome, Page kidney, reflux nephropathy, Bardet-Biedl syndrome, collagenofibrotic glomerulopathy, Dent disease, Denys-Drash syndrome, congenital nephrotic syndrome, immunotactoid glomerulopathy, fibronextin glomerulopathy, Galloway Mowat syndrome, lipoprotein glomerulopathy, MesoAmerican nephropathy, beta-thalassemia renal disease, haemolytic uraemic syndrome, Henoch-Schonlein-Purpura disease, retroperitoneal fibrosis, polyarteritis nodose, cardiorenal syndrome, medullary kidney disease, renal artery stenosis, uromodulin kidney disease, and hyperkalemia.

In some embodiments, any of the binders or multispecific binders of TGFβ-superfamily ligands disclosed herein (e.g. an ActRIIB:TβRII heteromultimer) may be used to treat chronic kidney disease, optionally in combination with one or more supportive therapies for treating chronic kidney disease. Chronic kidney disease (CKD), also known as chronic renal disease, is a progressive loss in renal function over a period of months or years. The symptoms of worsening kidney function may include feeling generally unwell and experiencing a reduced appetite. Often, chronic kidney disease is diagnosed as a result of screening of people known to be at risk of kidney problems, such as those with high blood pressure or diabetes and those with a blood relative with CKD. This disease may also be identified when it leads to one of its recognized complications, such as cardiovascular disease, anemia, or pericarditis. Recent professional guidelines classify the severity of CKD in five stages, with stage 1 being the mildest and usually causing few symptoms and stage 5 being a severe illness with poor life expectancy if untreated. Stage 5 CKD is often called end-stage kidney disease, end-stage renal disease, or end-stage kidney failure, and is largely synonymous with the now outdated terms chronic renal failure or chronic kidney failure; and usually means the patient requires renal replacement therapy, which may involve a form of dialysis, but ideally constitutes a kidney transplant. CKD is initially without specific symptoms and is generally only detected as an increase in serum creatinine or protein in the urine. As the kidney function decreases, various symptoms may manifest as described below. Blood pressure may be increased due to fluid overload and production of vasoactive hormones created by the kidney via the renin-angiotensin system, increasing one's risk of developing hypertension and/or suffering from congestive heart failure. Urea may accumulate, leading to azotemia and ultimately uremia (symptoms ranging from lethargy to pericarditis and encephalopathy). Due to its high systemic circulation, urea is excreted in eccrine sweat at high concentrations and crystallizes on skin as the sweat evaporates (“uremic frost”). Potassium may accumulate in the blood (hyperkalemia with a range of symptoms including malaise and potentially fatal cardiac arrhythmias). Hyperkalemia usually does not develop until the glomerular filtration rate falls to less than 20-25 ml/min/1.73 m², at which point the kidneys have decreased ability to excrete potassium. Hyperkalemia in CKD can be exacerbated by acidemia (which leads to extracellular shift of potassium) and from lack of insulin. Erythropoietin synthesis may be decreased causing anemia. Fluid volume overload symptoms may occur, ranging from mild edema to life-threatening pulmonary edema. Hyperphosphatemia, due to reduced phosphate excretion, may occur generally following the decrease in glomerular filtration. Hyperphosphatemia is associated with increased cardiovascular risk, being a direct stimulus to vascular calcification. Hypocalcemia may manifest, which is generally caused by stimulation of fibroblast growth factor-23. Osteocytes are responsible for the increased production of FGF23, which is a potent inhibitor of the enzyme 1-alpha-hydroxylase (responsible for the conversion of 25-hydroxycholecalciferol into 1,25-dihydroxyvitamin D3). Later, this progresses to secondary hyperparathyroidism, renal osteodystrophy, and vascular calcification that further impairs cardiac function. Metabolic acidosis (due to accumulation of sulfates, phosphates, uric acid etc.) may occur and cause altered enzyme activity by excess acid acting on enzymes; and also increased excitability of cardiac and neuronal membranes by the promotion of hyperkalemia due to excess acid (acidemia). Acidosis is also due to decreased capacity to generate enough ammonia from the cells of the proximal tubule. Iron deficiency anemia, which increases in prevalence as kidney function decreases, is especially prevalent in those requiring haemodialysis. It is multifactoral in cause, but includes increased inflammation, reduction in erythropoietin, and hyperuricemia leading to bone marrow suppression. People with CKD suffer from accelerated atherosclerosis and are more likely to develop cardiovascular disease than the general population. Patients afflicted with CKD and cardiovascular disease tend to have significantly worse prognoses than those suffering only from the latter.

In another embodiment, any of the binders or multispecific binders of TGFβ-superfamily ligands disclosed herein (e.g. an ActRIIB:TβRII heteromultimer) may be used in patients with chronic kidney disease mineral bone disorder (CKD-MBD), a broad syndrome of interrelated skeletal, cardiovascular, and mineral-metabolic disorders arising from kidney disease. CKD-MBD encompasses various skeletal pathologies often referred to as renal osteodystrophy (ROD), which is a preferred embodiment for treatment with, an activin and/or GDF antagonist, or combinations of such antagonists. Depending on the relative contribution of different pathogenic factors, ROD is manifested as diverse pathologic patterns of bone remodeling (Hruska et al., 2008, Chronic kidney disease mineral bone disorder (CKD-MBD); in Rosen et al. (ed) Primer on the Metabolic Bone Diseases and Disorders of Mineral Metabolism, 7th ed. American Society for Bone and Mineral Research, Washington D.C., pp 343-349). At one end of the spectrum is ROD with uremic osteodystrophy and low bone turnover, characterized by a low number of active remodeling sites, profoundly suppressed bone formation, and low bone resorption. At the other extreme is ROD with hyperparathyroidism, high bone turnover, and osteitis fibrosa. Given that any of the binders or multispecific binders of TGFβ-superfamily ligands disclosed herein (e.g. an ActRIIB:TβRII heteromultimer) may exert both anabolic and antiresorptive effects, these agents may be useful in patients across the ROD pathology spectrum.

In some embodiments, the present disclosure relates to methods of increasing red blood cell levels in a patient comprising administering to a patient in need thereof (e.g., for treating anemia or disease or condition associated with anemia) an effective amount of any of the binders or multispecific binders of TGFβ-superfamily ligands disclosed herein (e.g. an ActRIIB:TβRII heteromultimer). For example, in some embodiments, the disclosure relates to methods of preventing or reducing the severity or progression rate of one or more complications of anemia. Optionally, methods disclosed herein for treating anemia or disease or condition associated with anemia may further comprise administering to the patient one or more supportive therapies or additional active agents for treating anemia or disease or condition associated with anemia.

In some embodiments, any of the binders or multispecific binders of TGFβ-superfamily ligands disclosed herein (e.g. an ActRIIB:TβRII heteromultimer) may be used to increase red blood cell, hemoglobin or reticulocyte levels in healthy individuals, and such multispecific binders may be used in selected patient populations. Examples of appropriate patient populations include those with undesirably low red blood cell or hemoglobin levels, such as patients having an anemia, and those that are at risk for developing undesirably low red blood cell or hemoglobin levels, such as those patients that are about to undergo major surgery or other procedures that may result in substantial blood loss. In one embodiment, a patient with adequate red blood cell levels is treated with any of the binders or multispecific binders of TGFβ-superfamily ligands disclosed herein (e.g. an ActRIIB:TβRII heteromultimer) to increase red blood cell levels, and then blood is drawn and stored for later use in transfusions.

In some embodiments, any of the binders or multispecific binders of TGFβ-superfamily ligands disclosed herein (e.g. an ActRIIB:TβRII heteromultimer) may be used to increase red blood cell levels in patients having an anemia. When observing hemoglobin levels in humans, a level of less than normal for the appropriate age and gender category may be indicative of anemia, although individual variations are taken into account. For example, a hemoglobin level of 12 g/dl is generally considered the lower limit of normal in the general adult population. Potential causes include blood-loss, nutritional deficits, medication reaction, various problems with the bone marrow and many diseases. More particularly, anemia has been associated with a variety of disorders that include, for example, chronic renal failure, myelodysplastic syndrome, rheumatoid arthritis, bone marrow transplantation. Anemia may also be associated with the following conditions: solid tumors (e.g. breast cancer, lung cancer, colon cancer); tumors of the lymphatic system (e.g. chronic lymphocyte leukemia, non-Hodgkins and Hodgkins lymphomas); tumors of the hematopoietic system (e.g. leukemia, myelodysplastic syndrome, multiple myeloma); radiation therapy; chemotherapy (e.g. platinum containing regimens); inflammatory and autoimmune diseases, including, but not limited to, rheumatoid arthritis, other inflammatory arthritides, systemic lupus erythematosis (SLE), acute or chronic skin diseases (e.g. psoriasis), inflammatory bowel disease (e.g. Crohn's disease and ulcerative colitis); acute or chronic renal disease or failure including idiopathic or congenital conditions; acute or chronic liver disease; acute or chronic bleeding; situations where transfusion of red blood cells is not possible due to patient allo- or auto-antibodies and/or for religious reasons (e.g. some Jehovah's Witnesses); infections (e.g. malaria, osteomyelitis); hemoglobinopathies, including, for example, sickle cell disease, thalassemias; drug use or abuse, e.g. alcohol misuse; pediatric patients with anemia from any cause to avoid transfusion; and elderly patients or patients with underlying cardiopulmonary disease with anemia who cannot receive transfusions due to concerns about circulatory overload.

In some embodiments, any of the binders or multispecific binders of TGFβ-superfamily ligands disclosed herein (e.g. an ActRIIB:TβRII heteromultimer) may be used for treating ineffective erythropoiesis. Originally distinguished from aplastic anemia, hemorrhage, or peripheral hemolysis on the basis of ferrokinetic studies (Ricketts et al., 1978, Clin Nucl Med 3:159-164), ineffective erythropoiesis describes a diverse group of anemias in which production of mature RBCs is less than would be expected given the number of erythroid precursors (erythroblasts) present in the bone marrow (Tanno et al., 2010, Adv Hematol 2010:358283). In such anemias, tissue hypoxia persists despite elevated erythropoietin levels due to ineffective production of mature RBCs. A vicious cycle eventually develops in which elevated erythropoietin levels drive massive expansion of erythroblasts, potentially leading to splenomegaly (spleen enlargement) due to extramedullary erythropoiesis (Aizawa et al, 2003, Am J Hematol 74:68-72), erythroblast-induced bone pathology (Di Matteo et al, 2008, J Biol Regul Homeost Agents 22:211-216), and tissue iron overload, even in the absence of therapeutic RBC transfusions (Pippard et al, 1979, Lancet 2:819-821). Thus, by boosting erythropoietic effectiveness, any of the binders or multispecific binders of TGFβ-superfamily ligands disclosed herein (e.g. an ActRIIB: TβRII heteromultimer) may break the aforementioned cycle and may alleviate not only the underlying anemia but also the associated complications of elevated erythropoietin levels, splenomegaly, bone pathology, and tissue iron overload. In some embodiments, any of the binders or multispecific binders of TGFβ-superfamily ligands disclosed herein (e.g. an ActRIIB:TβRII heteromultimer) may be used to treat ineffective erythropoiesis, including anemia and elevated EPO levels, as well as complications such as splenomegaly, erythroblast-induced bone pathology, and iron overload, and their attendant pathologies. With splenomegaly, such pathologies include thoracic or abdominal pain and reticuloendothelial hyperplasia. Extramedullary hematopoiesis can occur not only in the spleen but potentially in other tissues in the form of extramedullary hematopoietic pseudotumors (Musallam et al., 2012, Cold Spring Harb Perspect Med 2:a013482). With erythroblast-induced bone pathology, attendant pathologies include low bone mineral density, osteoporosis, and bone pain (Haidar et al., 2011, Bone 48:425-432). With iron overload, attendant pathologies include hepcidin suppression and hyperabsorption of dietary iron (Musallam et al., 2012, Blood Rev 26(Suppl 1):S16-S19), multiple endocrinopathies and liver fibrosis/cirrhosis (Galanello et al., 2010, Orphanet J Rare Dis 5:11), and iron-overload cardiomyopathy (Lekawanvijit et al., 2009, Can J Cardiol 25:213-218).

In some embodiments, any of the binders or multispecific binders of TGFβ-superfamily ligands disclosed herein (e.g. an ActRIIB:TβRII heteromultimer) may be used for treating thalassemia. The most common causes of ineffective erythropoiesis are the thalassemia syndromes, hereditary hemoglobinopathies in which imbalances in the production of intact alpha- and beta-hemoglobin chains lead to increased apoptosis during erythroblast maturation (Schrier, 2002, Curr Opin Hematol 9:123-126). Thalassemias are collectively among the most frequent genetic disorders worldwide, with changing epidemiologic patterns predicted to contribute to a growing public health problem in both the U.S. and globally (Vichinsky, 2005, Ann NY Acad Sci 1054:18-24). Thalassemia syndromes are named according to their severity. Thus, α-thalassemias include α-thalassemia minor (also known as α-thalassemia trait; two affected α-globin genes), hemoglobin H disease (three affected α-globin genes), and α-thalassemia major (also known as hydrops fetalis; four affected α-globin genes). β-Thalassemias include β-thalassemia minor (also known as β-thalassemia trait; one affected β-globin gene), β-thalassemia intermedia (two affected β-globin genes), hemoglobin E thalassemia (two affected β-globin genes), and β-thalassemia major (also known as Cooley's anemia; two affected β-globin genes resulting in a complete absence of β-globin protein). β-Thalassemia impacts multiple organs, is associated with considerable morbidity and mortality, and currently requires life-long care. Although life expectancy in patients with β-thalassemia has increased in recent years due to use of regular blood transfusions in combination with iron chelation, iron overload resulting both from transfusions and from excessive gastrointestinal absorption of iron can cause serious complications such as heart disease, thrombosis, hypogonadism, hypothyroidism, diabetes, osteoporosis, and osteopenia (Rund et al, 2005, N Engl J Med 353:1135-1146).

In some embodiments, any of the binders or multispecific binders of TGFβ-superfamily ligands disclosed herein (e.g. an ActRIIB:TβRII heteromultimer) can be used for treating diseases of ineffective erythropoiesis other than thalassemia syndromes. Such disorders include siderblastic anemia (inherited or acquired); dyserythropoietic anemia (Types I and II); sickle cell anemia (sickle cell disease); hereditary spherocytosis; pyruvate kinase deficiency; megaloblastic anemias, potentially caused by conditions such as folate deficiency (due to congenital diseases, decreased intake, or increased requirements), cobalamin deficiency (due to congenital diseases, pernicious anemia, impaired absorption, pancreatic insufficiency, or decreased intake), certain drugs, or unexplained causes (congenital dyserythropoietic anema, refractory megaloblastic anemia, or erythroleukemia); myelophthisic anemias, including myelofibrosis (myeloid metaplasia) and myelophthisis; congenital erythropoietic porphyria; and lead poisoning.

In some embodiments, any of the binders or multispecific binders of TGFβ-superfamily ligands disclosed herein (e.g. an ActRIIB:TβRII heteromultimer) may be used for treating myelodysplastic syndrome (MDS). MDS is a diverse collection of hematological conditions characterized by ineffective production of myeloid blood cells and risk of transformation to acute myelogenous leukemia. In MDS patients, blood stem cells do not mature into healthy red blood cells, white blood cells, or platelets. MDS disorders include, for example, refractory anemia, refractory anemia with ringed sideroblasts, refractory anemia with excess blasts, refractory anemia with excess blasts in transformation, refractory cytopenia with multilineage dysplasia, and myelodysplastic syndrome associated with an isolated 5q chromosome abnormality. As these disorders manifest as irreversible defects in both quantity and quality of hematopoietic cells, most MDS patients are afflicted with chronic anemia. Therefore, MDS patients eventually require blood transfusions and/or treatment with growth factors (e.g., erythropoietin or G-CSF) to increase red blood cell levels. However, many MDS patients develop side-effect due to frequency of such therapies. For example, patients who receive frequent red blood cell transfusion can have tissue and organ damage from the buildup of extra iron. In some embodiments, patient suffering from MDS may be treated using a combination of any of the binders or multispecific binders of TGFβ-superfamily ligands disclosed herein (e.g. an ActRIIB:TβRII heteromultimer) and one or more additional therapeutic agents for treating MDS including, for example, thalidomide, lenalidomide, azacitadine, decitabine, erythropoietins, deferoxamine, antihymocyte globulin, filgrastrim (G-CSF) and an erythropoietin signaling pathway agonist.

In some embodiments, any of the binders or multispecific binders of TGFβ-superfamily ligands disclosed herein (e.g. an ActRIIB:TβRII heteromultimer) may be used for treating anemias of hypoproliferative bone marrow, which are typically associated with little change in red blood cell (RBC) morphology. Hypoproliferative anemias include: 1) anemia of chronic disease, 2) anemia of kidney disease, and 3) anemia associated with hypometabolic states. In each of these types, endogenous erythropoietin levels are inappropriately low for the degree of anemia observed. Other hypoproliferative anemias include: 4) early-stage iron-deficient anemia, and 5) anemia caused by damage to the bone marrow. In these types, endogenous erythropoietin levels are appropriately elevated for the degree of anemia observed.

In some embodiments, any of the binders or multispecific binders of TGFβ-superfamily ligands disclosed herein (e.g. an ActRIIB:TβRII heteromultimer) may be used to treat anemia associated with chronic disease. The most common type is anemia of chronic disease, which encompasses inflammation, infection, tissue injury, and conditions such as cancer, and is distinguished by both low erythropoietin levels and an inadequate response to erythropoietin in the bone marrow (Adamson, 2008, Harrison's Principles of Internal Medicine, 17th ed.; McGraw Hill, New York, pp 628-634). Many factors can contribute to cancer-related anemia. Some are associated with the disease process itself and the generation of inflamatory cytokines such as interleukin-1, interferon-gamma, and tumor necrosis factor (Bron et al., 2001, Semin Oncol 28(Suppl 8):1-6). Among its effects, inflammation induces the key iron-regulatory peptide hepcidin, thereby inhibiting iron export from macrophages and generally limiting iron availability for erythropoiesis (Ganz, 2007, J Am Soc Nephrol 18:394-400). Blood loss through various routes can also contribute to cancer-related anemia. The prevalence of anemia due to cancer progression varies with cancer type, ranging from 5% in prostate cancer up to 90% in multiple myeloma. Cancer-related anemia has profound consequences for patients, including fatigue and reduced quality of life, reduced treatment efficacy, and increased mortality.

Chronic kidney disease is associated with hypoproliferative anemia that varies in severity Many conditions resulting in a hypometabolic rate can produce a mild-to-moderate hypoproliferative anemia. Among such conditions are endocrine deficiency states. For example, anemia can occur in Addison's disease, hypothyroidism, hyperparathyroidism, or males who are castrated or treated with estrogen. Mild-to-moderate anemia can also occur with reduced dietary intake of protein, a condition particularly prevalent in the elderly. Finally, anemia can develop in patients with chronic liver disease arising from nearly any cause (Adamson, 2008, Harrison's Principles of Internal Medicine, 17th ed.; McGraw Hill, New York, pp 628-634).

In some embodiments, any of the binders or multispecific binders of TGFβ-superfamily ligands disclosed herein (e.g. an ActRIIB:TβRII heteromultimer) may be used to treat anemia resulting from acute blood loss. Anemia resulting from acute blood loss of sufficient volume, such as from trauma or postpartum hemorrhage, is known as acute post-hemorrhagic anemia. Acute blood loss initially causes hypovolemia without anemia since there is proportional depletion of RBCs along with other blood constituents. However, hypovolemia will rapidly trigger physiologic mechanisms that shift fluid from the extravascular to the vascular compartment, which results in hemodilution and anemia. If chronic, blood loss gradually depletes body iron stores and eventually leads to iron deficiency.

In some embodiments, any of the binders or multispecific binders of TGFβ-superfamily ligands disclosed herein (e.g. an ActRIIB:TβRII heteromultimer) may be used to treat iron-deficiency anemias. Iron-deficiency anemia is the final stage in a graded progression of increasing iron deficiency which includes negative iron balance and iron-deficient erythropoiesis as intermediate stages. Iron deficiency can result from increased iron demand, decreased iron intake, or increased iron loss, as exemplified in conditions such as pregnancy, inadequate diet, intestinal malabsorption, acute or chronic inflammation, and acute or chronic blood loss. With mild-to-moderate anemia of this type, the bone marrow remains hypoproliferative, and RBC morphology is largely normal; however, even mild anemia can result in some microcytic hypochromic RBCs, and the transition to severe iron-deficient anemia is accompanied by hyperproliferation of the bone marrow and increasingly prevalent microcytic and hypochromic RBCs (Adamson, 2008, Harrison's Principles of Internal Medicine, 17th ed.; McGraw Hill, New York, pp 628-634). Appropriate therapy for iron-deficiency anemia depends on its cause and severity, with oral iron preparations, parenteral iron formulations, and RBC transfusion as major conventional options.

In some embodiments, any of the binders or multispecific binders of TGFβ-superfamily ligands disclosed herein (e.g. an ActRIIB:TβRII heteromultimer) may be used to treat hypoproliferative anemia. Hypoproliferative anemias can result from primary dysfunction or failure of the bone marrow, instead of dysfunction secondary to inflammation, infection, or cancer progression. Prominent examples would be myelosuppression caused by cancer chemotherapeutic drugs or cancer radiation therapy. A broad review of clinical trials found that mild anemia can occur in 100% of patients after chemotherapy, while more severe anemia can occur in up to 80% of such patients (Groopman et al., 1999, J Natl Cancer Inst 91:1616-1634). Myelosuppressive drugs include: 1) alkylating agents such as nitrogen mustards (e.g., melphalan) and nitrosoureas (e.g., streptozocin); 2) antimetabolites such as folic acid antagonists (e.g., methotrexate), purine analogs (e.g., thioguanine), and pyrimidine analogs (e.g., gemcitabine); 3) cytotoxic antibiotics such as anthracyclines (e.g., doxorubicin); 4) kinase inhibitors (e.g., gefitinib); 5) mitotic inhibitors such as taxanes (e.g., paclitaxel) and vinca alkaloids (e.g., vinorelbine); 6) monoclonal antibodies (e.g., rituximab); and 7) topoisomerase inhibitors (e.g., topotecan and etoposide).

In some embodiments, any of the binders or multispecific binders of TGFβ-superfamily ligands disclosed herein (e.g. an ActRIIB:TβRII heteromultimer) may be used for treating anemias of disordered RBC maturation, which are characterized in part by undersized (microcytic), oversized (macrocytic), misshapen, or abnormally colored (hypochromic) RBCs.

In some embodiments, any of the binders or multispecific binders of TGFβ-superfamily ligands disclosed herein (e.g. an ActRIIB:TβRII heteromultimer) may be used in combination with supportive therapies for treating anemia or diseases associated with anemia. Such therapies include transfusion with either red blood cells or whole blood to treat anemia. In chronic or hereditary anemias, normal mechanisms for iron homeostasis are overwhelmed by repeated transfusions, eventually leading to toxic and potentially fatal accumulation of iron in vital tissues such as heart, liver, and endocrine glands. Thus, supportive therapies for patients chronically afflicted with anemia, particularly ineffective erythropoiesis, also include treatment with one or more iron-chelating molecules to promote iron excretion in the urine and/or stool and thereby prevent, or reverse, tissue iron overload (Hershko, 2006, Haematologica 91:1307-1312; Cao et al, 2011, Pediatr Rep 3(2):e17). Effective iron-chelating agents must be able to selectively bind and neutralize ferric iron, the oxidized form of non-transferrin bound iron which likely accounts for most iron toxicity through catalytic production of hydroxyl radicals and oxidation products (Esposito et al, 2003, Blood 102:2670-2677). These agents are structurally diverse, but all possess oxygen or nitrogen donor atoms able to form neutralizing octahedral coordination complexes with individual iron atoms in stoichiometries of 1:1 (hexadentate agents), 2:1 (tridentate), or 3:1 (bidentate) (Kalinowski et al, 2005, Pharmacol Rev 57:547-583). Effective iron-chelating agents also are relatively low molecular weight (less than 700 daltons), with solubility in both water and lipid to enable access to affected tissues. Specific examples of iron-chelating molecules are deferoxamine, a hexadentate agent of bacterial origin requiring daily parenteral administration, and the orally active synthetic agents deferiprone (bidentate) and deferasirox (tridentate). Combination therapy consisting of same-day administration of two iron-chelating agents shows promise in patients unresponsive to chelation monotherapy and also in overcoming issues of poor patient compliance with dereroxamine alone (Cao et al, 2011, Pediatr Rep 3(2):e17; Galanello et al, 2010, Ann NY Acad Sci 1202:79-86).

In certain embodiments, any of the binders or multispecific binders of TGFβ-superfamily ligands disclosed herein (e.g. an ActRIIB:TβRII heteromultimer) may be used in combination with hepcidin agonists for treating anemia, particularly anemias associated with ineffective erythropoiesis. A circulating polypeptide produced mainly in the liver, hepcidin is considered a master regulator of iron metabolism by virtue of its ability to induce the degradation of ferroportin, an iron-export protein localized on absorptive enterocytes, hepatocytes, and macrophages. Broadly speaking, hepcidin reduces availability of extracellular iron, so hepcidin agonists may be beneficial in the treatment of ineffective erythropoiesis (Nemeth, 2010, Adv Hematol 2010:750643). This view is supported by beneficial effects of increased hepcidin expression in a mouse model of β-thalassemia (Gardenghi et al, 2010, J Clin Invest 120:4466-4477).

In some embodiments, any of the binders or multispecific binders of TGFβ-superfamily ligands disclosed herein (e.g. an ActRIIB:TβRII heteromultimer) may be used in combination with EPO receptor activators to achieve an increase in red blood cells at lower dose ranges. This may be beneficial in reducing the known off-target effects and risks associated with high doses of EPO receptor activators. In certain embodiments, the present invention provides methods of treating or preventing anemia in an individual in need thereof by administering to the individual a therapeutically effective amount of an any of the binders or multispecific binders of TGFβ-superfamily ligands disclosed herein (e.g. an ActRIIB:TβRII heteromultimer)or a combination (or concomitant therapy) of a multispecific binder and a EPO receptor activator.

Any of the binders or multispecific binders of TGFβ-superfamily ligands disclosed herein (e.g. an ActRIIB:TβRII heteromultimer) may be used in combination with EPO receptor activators to reduce the required dose of these activators in patients that are susceptible to adverse effects of EPO. The primary adverse effects of EPO are an excessive increase in the hematocrit or hemoglobin levels and polycythemia. Elevated hematocrit levels can lead to hypertension (more particularly aggravation of hypertension) and vascular thrombosis. Other adverse effects of EPO which have been reported, some of which related to hypertension, are headaches, influenza-like syndrome, obstruction of shunts, myocardial infarctions and cerebral convulsions due to thrombosis, hypertensive encephalopathy, and red cell blood cell applasia (Singibarti, (1994) J. Clin Investig 72(suppl 6), S36-S43; Horl et al. (2000) Nephrol Dial Transplant 15(suppl 4), 51-56; Delanty et al. (1997) Neurology 49, 686-689; Bunn (2002) N Engl J Med 346(7), 522-523).

In some embodiments, patients may be treated with dosing regimen of any of the binders or multispecific binders of TGFβ-superfamily ligands disclosed herein (e.g. an ActRIIB:TβRII heteromultimer) intended to restore the patient to a target hemoglobin level, usually between about 10 g/dl and about 12.5 g/dl, and typically about 11.0 g/dl (see also Jacobs et al. (2000) Nephrol Dial Transplant 15, 15-19), although lower target levels may cause fewer cardiovascular side effects. Alternatively, hematocrit levels (percentage of the volume of a blood sample occupied by the cells) can be used as a measure for the condition of red blood cells. Hematocrit levels for healthy individuals range from 41 to 51% for adult males and from 35 to 45% for adult females. Target hematocrit levels are usually around 30-33%. Moreover, hemoglobin/hematocrit levels vary from person to person. Thus, optimally, the target hemoglobin/hematocrit level can be individualized for each patient.

In certain embodiments, the present invention provides methods for managing a patient that has been treated with, or is a candidate to be treated with, any of the binders or multispecific binders of TGFβ-superfamily ligands disclosed herein (e.g. an ActRIIB:TβRII heteromultimer) by measuring one or more hematologic parameters in the patient. The hematologic parameters may be used to evaluate appropriate dosing for a patient who is a candidate to be treated with any of the binders or multispecific binders of TGFβ-superfamily ligands disclosed herein (e.g. an ActRIIB:TβRII heteromultimer), to monitor the hematologic parameters during treatment with a binder/multispecific binder, to evaluate whether to adjust the dosage during treatment with a multispecific binder, and/or to evaluate an appropriate maintenance dose of a binder/multispecific binder. If one or more of the hematologic parameters are outside the normal level, dosing with a binder/multispecific binder may be reduced, delayed or terminated.

Hematologic parameters that may be measured in accordance with the methods provided herein include, for example, red blood cell levels, blood pressure, iron stores, and other agents found in bodily fluids that correlate with increased red blood cell levels, using art recognized methods. Such parameters may be determined using a blood sample from a patient. Increases in red blood cell levels, hemoglobin levels, and/or hematocrit levels may cause increases in blood pressure.

In one embodiment, if one or more hematologic parameters are outside the normal range, or on the high side of normal, in a patient who is a candidate to be treated with any of the binders or multispecific binders of TGFβ-superfamily ligands disclosed herein (e.g. an ActRIIB:TβRII heteromultimer) then onset of administration of the binder or multispecific binder may be delayed until the hematologic parameters have returned to a normal or acceptable level either naturally or via therapeutic intervention. For example, if a candidate patient is hypertensive or prehypertensive, then the patient may be treated with a blood pressure lowering agent in order to reduce the patient's blood pressure. Any blood pressure lowering agent appropriate for the individual patient's condition may be used including, for example, diuretics, adrenergic inhibitors (including alpha blockers and beta blockers), vasodilators, calcium channel blockers, angiotensin-converting enzyme (ACE) inhibitors, or angiotensin II receptor blockers. Blood pressure may alternatively be treated using a diet and exercise regimen. Similarly, if a candidate patient has iron stores that are lower than normal, or on the low side of normal, then the patient may be treated with an appropriate regimen of diet and/or iron supplements until the patient's iron stores have returned to a normal or acceptable level. For patients having higher than normal red blood cell levels and/or hemoglobin levels, then administration of the binder or multispecific binder may be delayed until the levels have returned to a normal or acceptable level.

In certain embodiments, if one or more hematologic parameters are outside the normal range, or on the high side of normal, in a patient who is a candidate to be treated with any of the binders or multispecific binders of TGFβ-superfamily ligands disclosed herein (e.g. an ActRIIB:TβRII heteromultimer) then the onset of administration may be not be delayed. However, the dosage amount or frequency of dosing of the binder or multispecific binder may be set at an amount that would reduce the risk of an unacceptable increase in the hematologic parameters arising upon administration of the binder or multispecific binder. Alternatively, a therapeutic regimen may be developed for the patient that combines any of the binders or multispecific binders of TGFβ-superfamily ligands disclosed herein (e.g. an ActRIIB:TβRII heteromultimer) with a therapeutic agent that addresses the undesirable level of the hematologic parameter. For example, if the patient has elevated blood pressure, then a therapeutic regimen involving administration of any of the binders or multispecific binders of TGFβ-superfamily ligands disclosed herein (e.g. an ActRIIB:TβRII heteromultimer) and a blood pressure lowering agent may be designed. For a patient having lower than desired iron stores, a therapeutic regimen of an any of the binders or multispecific binders of TGFβ-superfamily ligands disclosed herein (e.g. an ActRIIB:TβRII heteromultimer) and iron supplementation may be developed.

In one embodiment, baseline parameter(s) for one or more hematologic parameters may be established for a patient who is a candidate to be treated with any of the binders or multispecific binders of TGFβ-superfamily ligands disclosed herein (e.g. an ActRIIB:TβRII heteromultimer) and an appropriate dosing regimen establish for that patient based on the baseline value(s). Alternatively, established baseline parameters based on a patient's medical history could be used to inform an appropriate binder or multispecific binder dosing regimen for a patient. For example, if a healthy patient has an established baseline blood pressure reading that is above the defined normal range it may not be necessary to bring the patient's blood pressure into the range that is considered normal for the general population prior to treatment with the binder or multispecific binder. A patient's baseline values for one or more hematologic parameters prior to treatment with any of the binders or multispecific binders of TGFβ-superfamily ligands disclosed herein (e.g. an ActRIIB:TβRII heteromultimer) may also be used as the relevant comparative values for monitoring any changes to the hematologic parameters during treatment with the binder or multispecific binder.

In certain embodiments, one or more hematologic parameters are measured in patients who are being treated with any of the binders or multispecific binders of TGFβ-superfamily ligands disclosed herein (e.g. an ActRIIB:TβRII heteromultimer). The hematologic parameters may be used to monitor the patient during treatment and permit adjustment or termination of the dosing with the binder multispecific binder or additional dosing with another therapeutic agent. For example, if administration of the binder or multispecific binder results in an increase in blood pressure, red blood cell level, or hemoglobin level, or a reduction in iron stores, then the dose of the binder or multispecific binder may be reduced in amount or frequency in order to decrease the effects of the binder or multispecific binder on the one or more hematologic parameters. If administration of a binder or multispecific binder results in a change in one or more hematologic parameters that is adverse to the patient, then the dosing of the binder or multispecific binder may be terminated either temporarily, until the hematologic parameter(s) return to an acceptable level, or permanently. Similarly, if one or more hematologic parameters are not brought within an acceptable range after reducing the dose or frequency of administration of the binder or multispecific binder then the dosing may be terminated. As an alternative, or in addition to, reducing or terminating the dosing with the binder or multispecific binder, the patient may be dosed with an additional therapeutic agent that addresses the undesirable level in the hematologic parameter(s), such as, for example, a blood pressure lowering agent or an iron supplement. For example, if a patient being treated with a binder or multispecific binder has elevated blood pressure, then dosing with the binder or multispecific binder may continue at the same level and a blood pressure lowering agent is added to the treatment regimen, dosing with the multispecific binder may be reduce (e.g., in amount and/or frequency) and a blood pressure lowering agent is added to the treatment regimen, or dosing with the binder or multispecific binder may be terminated.

### 6. Pharmaceutical Compositions

The therapeutic agents described herein (e.g., ActRIIB:TβRII heteromultimer) may be formulated into pharmaceutical compositions. Pharmaceutical compositions for use in accordance with the present disclosure may be formulated in conventional manner using one or more physiologically acceptable carriers or excipients. Such formulations will generally be substantially pyrogen-free, in compliance with most regulatory requirements.

In certain embodiments, the therapeutic method of the disclosure includes administering the composition systemically, or locally as an implant or device. When administered, the therapeutic composition for use in this disclosure is in a pyrogen-free, physiologically acceptable form. Therapeutically useful agents other than the binder or multispecific binder which may also optionally be included in the composition as described above, may be administered simultaneously or sequentially with the subject compounds in the methods disclosed herein.

Typically, protein therapeutic agents disclosed herein will be administered parentally, and particularly intravenously or subcutaneously. Pharmaceutical compositions suitable for parenteral administration may comprise one or more of any of the binders or multispecific binders of TGFβ-superfamily ligands disclosed herein (e.g. an ActRIIB:TβRII heteromultimer) in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compositions and formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration

Further, the composition may be encapsulated or injected in a form for delivery to a target tissue site. In certain embodiments, compositions of the present invention may include a matrix capable of delivering one or more therapeutic compounds (e.g., ActRIIB:TβRII heteromultimer) to a target tissue site, providing a structure for the developing tissue and optimally capable of being resorbed into the body. For example, the matrix may provide slow release of any of the binders or multispecific binders of TGFβ-superfamily ligands disclosed herein (e.g. an ActRIIB:TβRII heteromultimer). Such matrices may be formed of materials presently in use for other implanted medical applications.

The choice of matrix material is based on biocompatibility, biodegradability, mechanical properties, cosmetic appearance and interface properties. The particular application of the subject compositions will define the appropriate formulation. Potential matrices for the compositions may be biodegradable and chemically defined calcium sulfate, tricalcium phosphate, hydroxyapatite, polylactic acid and polyanhydrides. Other potential materials are biodegradable and biologically well defined, such as bone or dermal collagen. Further matrices are comprised of pure proteins or extracellular matrix components. Other potential matrices are non-biodegradable and chemically defined, such as sintered hydroxyapatite, bioglass, aluminates, or other ceramics. Matrices may be comprised of combinations of any of the above-mentioned types of material, such as polylactic acid and hydroxyapatite or collagen and tricalcium phosphate. The bioceramics may be altered in composition, such as in calcium-aluminate-phosphate and processing to alter pore size, particle size, particle shape, and biodegradability.

In certain embodiments, methods of the invention can be administered for orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

The compositions of the invention may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

It is understood that the dosage regimen will be determined by the attending physician considering various factors which modify the action of the subject compounds of the invention (e.g., ActRIIB:TβRII heteromultimer). The various factors include, but are not limited to, the patient's age, sex, and diet, the severity disease, time of administration, and other clinical factors. Optionally, the dosage may vary with the type of matrix used in the reconstitution and the types of compounds in the composition. The addition of other known growth factors to the final composition, may also affect the dosage. Progress can be monitored by periodic assessment of bone growth and/or repair, for example, X-rays (including DEXA), histomorphometric determinations, and tetracycline labeling.

In certain embodiments, the present invention also provides gene therapy for the in vivo production of any of the binders or multispecific binders of TGFβ-superfamily ligands disclosed herein (e.g. an ActRIIB:TβRII heteromultimer). Such therapy would achieve its therapeutic effect by introduction of the binder multispecific binder polynucleotide sequences into cells or tissues having the disorders as listed above. Delivery of binder or multispecific binder polynucleotide sequences can be achieved using a recombinant expression vector such as a chimeric virus or a colloidal dispersion system. Preferred for therapeutic delivery of binder or multispecific binder polynucleotide sequences is the use of targeted liposomes.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing the binder or multispecific binder polynucleotide. In a preferred embodiment, the vector is targeted to bone or cartilage.

Alternatively, tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for binder or multispecific binder polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. RNA, DNA and intact virions can be encapsulated within the aqueous interior and be delivered to cells in a biologically active form (see e.g., Fraley, et al., Trends Biochem. Sci., 6:77, 1981). Methods for efficient gene transfer using a liposome vehicle, are known in the art, see e.g., Mannino, et al., Biotechniques, 6:682, 1988. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

The disclosure provides formulations that may be varied to include acids and bases to adjust the pH; and buffering agents to keep the pH within a narrow range.

### EXEMPLIFICATION

The invention now being generally described, it will be more readily understood by reference to the following examples, which are included merely for purposes of illustration of certain embodiments of the present invention, and are not intended to limit the invention.

### Example 1. Generation of TβRII Receptor Fusion Protein Variants

### TβRII ECD Variants

TβRII fusion proteins comprising a soluble extracellular portion of human TβRII and a human Fc portion were generated. For each fusion protein, a TβRII amino acid sequence having the amino acid sequence of SEQ ID NO: 18 was fused to an IgG Fc portion having the amino acid sequence of SEQ ID NO: 49 by means of one of several different linkers. Each of the fusion proteins also included a TPA leader sequence having the amino acid sequence of SEQ ID NO: 23 (below).

Tissue plasminogen activator (TPA): MDAMKRGLCCVLLLCGAVFVSP (SEQ ID NO: 23)

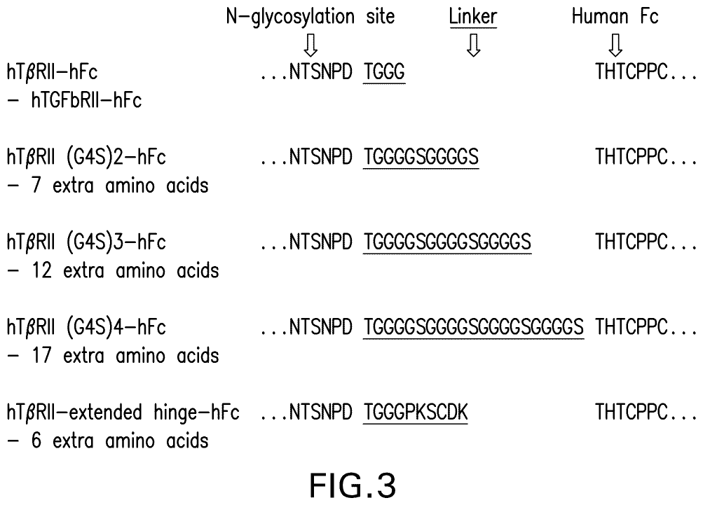

An illustration summary of several of the constructs designed is provided as FIG. 3. A table detailing the sequences for the different constructs tested in the Exemplification section is provided below:

| | Construct | |
|---|---|---|
| Construct | Amino Acid | |
| Name | Sequence | Linker Sequence |
| hTβRII-hFc | SEQ ID NO: 9 | TGGG (SEQ ID NO: 3) |
| hTβRII (G4S)2- | SEQ ID NO: 15 | TGGGGSGGGGS |
| hFc | | (SEQ ID NO: 4) |
| hTβRII (G4S)3- | SEQ ID NO: 11 | TGGGGSGGGGSGGGGS |
| hFc | | (SEQ ID NO: 5) |
| hTβRII (G4S)4- | SEQ ID NO: 13 | TGGGGSGGGGSGGGGSGGGGS |
| hFc | | (SEQ ID NO: 6) |
| hTβRII | SEQ ID NO: 17 | TGGGPKSCDK |
| extended hinge- | | (SEQ ID NO: 7) |
| hFc | | |
| hTβRII (G4S)5- | SEQ ID NO: 44 | TGGGGSGGGGSGGGGSGGGGS |
| hFc | | GGGGS (SEQ ID NO: 25) |
| hTβRII (G4S)6- | SEQ ID NO: 45 | TGGGGSGGGGSGGGGSGGGGS |
| hFc | | GGGGSGGGGS |
| | | (SEQ ID NO: 26) |

The amino acid sequences for the construct components and each of the constructs, along with the nucleic acid sequence used to express these constructs, are provided below.

TβRII-Portion: Amino Acid Sequence
(SEQ ID NO: 18)

| | |
|---|---|
| 1 | TIPPHVQKSD VEMEAQKDEI ICPSCNRTAH PLRHINNDMI |
| | VTDNNGAVKF |
| 51 | PQLCKFCDVR FSTCDNQKSC MSNCSITSIC EKPQEVCVAV |
| | WRKNDENITL |
| 101 | ETVCHDPKLP YHDFILEDAA SPKCIMKEKK KPGETFFMCS |
| | CSSDECNDNI |
| 151 | IFSEEYNTSN PD |

Fc Portion: Amino Acid Sequence
(SEQ ID NO: 49)

| | |
|---|---|
| 1 | THTCPPCPAP ELLGGPSVFL FPPKPKDTLM ISRTPEVTCV |
| | VVDVSHEDPE |
| 51 | VKFNWYVDGV EVHNAKTKPR EEQYNSTYRV VSVLTVLHQD |
| | WLNGKEYKCK |
| 101 | VSNKALPAPI EKTISKAKGQ PREPQVYTLP PSREEMTKNQ |
| | VSLTCLVKGF |
| 151 | YPSDIAVEWE SNGQPENNYK TTPPVLDSDG SFFLYSKLTV |
| | DKSRWQQGNV |
| 201 | FSCSVMHEAL HNHYTQKSLS LSPGK |

hTβRII-hFc: Nucleic Acid Sequence
(SEQ ID NO: 8)

| | |
|---|---|
| 1 | ATGGATGCAA TGAAGAGAGG GCTCTGCTGT GTGCTGCTGC |
| | TGTGTGGAGC |
| 51 | AGTCTTCGTT TCGCCCGGCG CCACGATCCC ACCGCACGTT |
| | CAGAAGTCGG |
| 101 | ATGTGGAAAT GGAGGCCCAG AAAGATGAAA TCATCTGCCC |
| | CAGCTGTAAT |
| 151 | AGGACTGCCC ATCCACTGAG ACATATTAAT AACGACATGA |
| | TAGTCACTGA |
| 201 | CAACAACGGT GCAGTCAAGT TTCCACAACT GTGTAAATTT |
| | TGTGATGTGA |
| 251 | GATTTTCCAC CTGTGACAAC CAGAAATCCT GCATGAGCAA |
| | CTGCAGCATC |
| 301 | ACCTCCATCT GTGAGAAGCC ACAGGAAGTC TGTGTGGCTG |
| | TATGGAGAAA |
| 351 | GAATGACGAG AACATAACAC TAGAGACAGT TTGCCATGAC |
| | CCCAAGCTCC |
| 401 | CCTACCATGA CTTTATTCTG GAAGATGCTG CTTCTCCAAA |
| | GTGCATTATG |
| 451 | AAGGAAAAAA AAAAGCCTGG TGAGACTTTC TTCATGTGTT |
| | CCTGTAGCTC |
| 501 | TGATGAGTGC AATGACAACA TCATCTTCTC AGAAGAATAT |
| | AACACCAGCA |
| 551 | ATCCTGACAC CGGTGGTGGA ACTCACACAT GCCCACCGTG |
| | CCCAGCACCT |
| 601 | GAACTCCTGG GGGGACCGTC AGTCTTCCTC TTCCCCCCAA |
| | AACCCAAGGA |
| 651 | CACCCTCATG ATCTCCCGGA CCCCTGAGGT CACATGCGTG |
| | GTGGTGGACG |
| 701 | TGAGCCACGA AGACCCTGAG GTCAAGTTCA ACTGGTACGT |
| | GGACGGCGTG |
| 751 | GAGGTGCATA ATGCCAAGAC AAAGCCGCGG GAGGAGCAGT |
| | ACAACAGCAC |
| 801 | GTACCGTGTG GTCAGCGTCC TCACCGTCCT GCACCAGGAC |
| | TGGCTGAATG |
| 851 | GCAAGGAGTA CAAGTGCAAG GTCTCCAACA AAGCCCTCCC |
| | AGCCCCCATC |
| 901 | GAGAAAACCA TCTCCAAAGC CAAAGGGCAG CCCCGAGAAC |
| | CACAGGTGTA |
| 951 | CACCCTGCCC CCATCCCGGG AGGAGATGAC CAAGAACCAG |
| | GTCAGCCTGA |
| 1001 | CCTGCCTGGT CAAAGGCTTC TATCCCAGCG ACATCGCCGT |
| | GGAGTGGGAG |
| 1051 | AGCAATGGGC AGCCGGAGAA CAACTACAAG ACCACGCCTC |
| | CCGTGCTGGA |
| 1101 | CTCCGACGGC TCCTTCTTCC TCTATAGCAA GCTCACCGTG |
| | GACAAGAGCA |
| 1151 | GGTGGCAGCA GGGGAACGTC TTCTCATGCT CCGTGATGCA |
| | TGAGGCTCTG |
| 1201 | CACAACCACT ACACGCAGAA GAGCCTCTCC CTGTCTCCGG |
| | GTAAATGA |

hTβRII-hFc: Amino Acid Sequence
(SEQ ID NO: 9)

| | |
|---|---|
| 1 | MDAMKRGLCC VLLLCGAVFV SPGATIPPHV QKSDVEMEAQ |
| | KDEITCPSCN |
| 51 | RTAHPLRHIN NDMIVTDNNG AVKFPQLCKF CDVRFSTCDN |
| | QKSCMSNCSI |
| 101 | TSICEKPQEV CVAVWRKNDE NITLETVCHD PKLPYHDFIL |
| | EDAASPKCIM |
| 151 | KEKKKPGETF FMCSCSSDEC NDNIIFSEEY NTSNPDTGGG |
| | THTCPPCPAP |
| 201 | ELLGGPSVFL FPPKPKDTLM ISRTPEVTCV VVDVSHEDPE |
| | VKFNWYVDGV |
| 251 | EVHNAKTKPR EEQYNSTYRV VSVLTVLHQD WLNGKEYKCK |
| | VSNKALPAPI |
| 301 | EKTISKAKGQ PREPQVYTLP PSREEMTKNQ VSLTCLVKGF |
| | YPSDIAVEWE |
| 351 | SNGQPENNYK TTPPVLDSDG SFFLYSKLTV DKSRWQQGNV |
| | FSCSVMHEAL |
| 401 | HNHYTQKSLS LSPGK |

hTβRII (G4S)3-hFc: Nucleic Acid Sequence
(SEQ ID NO: 10)

| | |
|---|---|
| 1 | ATGGATGCAA TGAAGAGAGG GCTCTGCTGT GTGCTGCTGC |
| | TGTGTGGAGC |
| 51 | AGTCTTCGTT TCGCCCGGCG CCACGATCCC ACCGCACGTT |
| | CAGAAGTCGG |
| 101 | ATGTGGAAAT GGAGGCCCAG AAAGATGAAA TCATCTGCCC |
| | CAGCTGTAAT |
| 151 | AGGACTGCCC ATCCACTGAG ACATATTAAT AACGACATGA |
| | TAGTCACTGA |
| 201 | CAACAACGGT GCAGTCAAGT TTCCACAACT GTGTAAATTT |
| | TGTGATGTGA |
| 251 | GATTTTCCAC CTGTGACAAC CAGAAATCCT GCATGAGCAA |
| | CTGCAGCATC |
| 301 | ACCTCCATCT GTGAGAAGCC ACAGGAAGTC TGTGTGGCTG |
| | TATGGAGAAA |
| 351 | GAATGACGAG AACATAACAC TAGAGACAGT TTGCCATGAC |
| | CCCAAGCTCC |
| 401 | CCTACCATGA CTTTATTCTG GAAGATGCTG CTTCTCCAAA |
| | GTGCATTATG |
| 451 | AAGGAAAAAA AAAAGCCTGG TGAGACTTTC TTCATGTGTT |
| | CCTGTAGCTC |
| 501 | TGATGAGTGC AATGACAACA TCATCTTCTC AGAAGAATAT |
| | AACACCAGCA |
| 551 | ATCCTGACAC CGGTGGTGGA GGAAGTGGTG GAGGTGGTTC |
| | TGGAGGTGGT |
| 601 | GGAAGTACTC ACACATGCCC ACCGTGCCCA GCACCTGAAC |
| | TCCTGGGGGG |
| 651 | ACCGTCAGTC TTCCTCTTCC CCCCAAAACC CAAGGACACC |
| | CTCATGATCT |
| 701 | CCCGGACCCC TGAGGTCACA TGCGTGGTGG TGGACGTGAG |
| | CCACGAAGAC |
| 751 | CCTGAGGTCA AGTTCAACTG GTACGTGGAC GGCGTGGAGG |
| | TGCATAATGC |
| 801 | CAAGACAAAG CCGCGGGAGG AGCAGTACAA CAGCACGTAC |
| | CGTGTGGTCA |
| 851 | GCGTCCTCAC CGTCCTGCAC CAGGACTGGC TGAATGGCAA |
| | GGAGTACAAG |
| 901 | TGCAAGGTCT CCAACAAAGC CCTCCCAGCC CCCATCGAGA |
| | AAACCATCTC |
| 951 | CAAAGCCAAA GGGCAGCCCC GAGAACCACA GGTGTACACC |
| | CTGCCCCCAT |
| 1001 | CCCGGGAGGA GATGACCAAG AACCAGGTCA GCCTGACCTG |
| | CCTGGTCAAA |
| 1051 | GGCTTCTATC CCAGCGACAT CGCCGTGGAG TGGGAGAGCA |
| | ATGGGCAGCC |
| 1101 | GGAGAACAAC TACAAGACCA CGCCTCCCGT GCTGGACTCC |
| | GACGGCTCCT |
| 1151 | TCTTCCTCTA TAGCAAGCTC ACCGTGGACA AGAGCAGGTG |
| | GCAGCAGGGG |
| 1201 | AACGTCTTCT CATGCTCCGT GATGCATGAG GCTCTGCACA |
| | ACCACTACAC |
| 1251 | GCAGAAGAGC CTCTCCCTGT CTCCGGGTAA ATGA |

hTβRII (G4S)3-hFc: Amino Acid Sequence
(SEQ ID NO: 11)

| | |
|---|---|
| 1 | MDAMKRGLCC VLLLCGAVFV SPGATIPPHV QKSDVEMEAQ |
| | KDEITCPSCN |
| 51 | RTAHPLRHIN NDMIVTDNNG AVKFPQLCKF CDVRFSTCDN |
| | QKSCMSNCSI |
| 101 | TSICEKPQEV CVAVWRKNDE NITLETVCHD PKLPYHDFIL |
| | EDAASPKCIM |
| 151 | KEKKKPGETF FMCSCSSDEC NDNIIFSEEY NTSNPDTGGG |
| | GSGGGGSGGG |
| 201 | GSTHTCPPCP APELLGGPSV FLFPPKPKDT LMISRTPEVT |
| | CVVVDVSHED |
| 251 | PEVKFNWYVD GVEVHNAKTK PREEQYNSTY RVVSVLTVLH |
| | QDWLNGKEYK |
| 301 | CKVSNKALPA PIEKTISKAK GQPREPQVYT LPPSREEMTK |
| | NQVSLTCLVK |
| 351 | GFYPSDIAVE WESNGQPENN YKTTPPVLDS DGSFFLYSKL |
| | TVDKSRWQQG |
| 401 | NVFSCSVMHE ALHNHYTQKS LSLSPGK |

hTβRII (G4S)4-hFc: Nucleic Acid Sequence
(SEQ ID NO: 12)

| | |
|---|---|
| 1 | ATGGATGCAA TGAAGAGAGG GCTCTGCTGT GTGCTGCTGC |
| | TGTGTGGAGC |
| 51 | AGTCTTCGTT TCGCCCGGCG CCACGATCCC ACCGCACGTT |
| | CAGAAGTCGG |
| 101 | ATGTGGAAAT GGAGGCCCAG AAAGATGAAA TCATCTGCCC |
| | CAGCTGTAAT |
| 151 | AGGACTGCCC ATCCACTGAG ACATATTAAT AACGACATGA |
| | TAGTCACTGA |
| 201 | CAACAACGGT GCAGTCAAGT TTCCACAACT GTGTAAATTT |
| | TGTGATGTGA |
| 251 | GATTTTCCAC CTGTGACAAC CAGAAATCCT GCATGAGCAA |
| | CTGCAGCATC |
| 301 | ACCTCCATCT GTGAGAAGCC ACAGGAAGTC TGTGTGGCTG |
| | TATGGAGAAA |
| 351 | GAATGACGAG AACATAACAC TAGAGACAGT TTGCCATGAC |
| | CCCAAGCTCC |
| 401 | CCTACCATGA CTTTATTCTG GAAGATGCTG CTTCTCCAAA |
| | GTGCATTATG |
| 451 | AAGGAAAAAA AAAAGCCTGG TGAGACTTTC TTCATGTGTT |
| | CCTGTAGCTC |
| 501 | TGATGAGTGC AATGACAACA TCATCTTCTC AGAAGAATAT |
| | AACACCAGCA |
| 551 | ATCCTGACAC CGGTGGTGGA GGTTCTGGAG GTGGAGGAAG |
| | TGGTGGAGGT |
| 601 | GGTTCTGGAG GTGGTGGAAG TACTCACACA TGCCCACCGT |
| | GCCCAGCACC |
| 651 | TGAACTCCTG GGGGGACCGT CAGTCTTCCT CTTCCCCCCA |
| | AAACCCAAGG |
| 701 | ACACCCTCAT GATCTCCCGG ACCCCTGAGG TCACATGCGT |
| | GGTGGTGGAC |
| 751 | GTGAGCCACG AAGACCCTGA GGTCAAGTTC AACTGGTACG |
| | TGGACGGCGT |
| 801 | GGAGGTGCAT AATGCCAAGA CAAAGCCGCG GGAGGAGCAG |
| | TACAACAGCA |
| 851 | CGTACCGTGT GGTCAGCGTC CTCACCGTCC TGCACCAGGA |
| | CTGGCTGAAT |
| 901 | GGCAAGGAGT ACAAGTGCAA GGTCTCCAAC AAAGCCCTCC |
| | CAGCCCCCAT |
| 951 | CGAGAAAACC ATCTCCAAAG CCAAAGGGCA GCCCCGAGAA |
| | CCACAGGTGT |
| 1001 | ACACCCTGCC CCCATCCCGG GAGGAGATGA CCAAGAACCA |
| | GGTCAGCCTG |
| 1051 | ACCTGCCTGG TCAAAGGCTT CTATCCCAGC GACATCGCCG |
| | TGGAGTGGGA |
| 1101 | GAGCAATGGG CAGCCGGAGA ACAACTACAA GACCACGCCT |
| | CCCGTGCTGG |
| 1151 | ACTCCGACGG CTCCTTCTTC CTCTATAGCA AGCTCACCGT |
| | GGACAAGAGC |
| 1201 | AGGTGGCAGC AGGGGAACGT CTTCTCATGC TCCGTGATGC |
| | ATGAGGCTCT |
| 1251 | GCACAACCAC TACACGCAGA AGAGCCTCTC CCTGTCTCCG |
| | GGTAAATGA |

hTβRII (G4S)4-hFc: Amino Acid Sequence
(SEQ ID NO: 13)

| | |
|---|---|
| 1 | MDAMKRGLCC VLLLCGAVFV SPGATIPPHV QKSDVEMEAQ |
| | KDEIICPSCN |
| 51 | RTAHPLRHIN NDMIVTDNNG AVKFPQLCKF CDVRFSTCDN |
| | QKSCMSNCSI |
| 101 | TSICEKPQEV CVAVWRKNDE NITLETVCHD PKLPYHDFIL |
| | EDAASPKCIM |
| 151 | KEKKKPGETF FMCSCSSDEC NDNIIFSEEY NTSNPDTGGG |
| | GSGGGGSGGG |
| 201 | GSGGGGSTHT CPPCPAPELL GGPSVFLFPP KPKDTLMISR |
| | TPEVTCVVVD |
| 251 | VSHEDPEVKF NWYVDGVEVH NAKTKPREEQ YNSTYRVVSV |
| | LTVLHQDWLN |
| 301 | GKEYKCKVSN KALPAPIEKT ISKAKGQPRE PQVYTLPPSR |
| | EEMTKNQVSL |
| 351 | TCLVKGFYPS DIAVEWESNG QPENNYKTTP PVLDSDGSFF |
| | LYSKLTVDKS |
| 401 | RWQQGNVFSC SVMHEALHNH YTQKSLSLSP GK |

hTβII (G4S)4-hFc: Amino Acid Sequence lacking
leader sequence

| | |
|---|---|
| | (SEQ ID NO: 94) |
| 1 | GATIPPHVQK SDVEMEAQKD EIICPSCNRT AHPLRHINND |
| | MIVTDNNGAV |
| 51 | KFPQLCKFCD VRFSTCDNQK SCMSNCSITS ICEKPQEVCV |
| | AVWRKNDENI |
| 101 | TLETVCHDPK LPYHDFILED AASPKCIMKE KKKPGETFFM |
| | CSCSSDECND |
| 151 | NIIFSEEYNT SNPDTGGGGS GGGGSGGGGS GGGGSTHTCP |
| | PCPAPELLGG |
| 201 | PSVFLFPPKP KDTLMISRTP EVTCVVVDVS HEDPEVKFNW |
| | YVDGVEVHNA |
| 251 | KTKPREEQYN STYRVVSVLT VLHQDWLNGK EYKCKVSNKA |
| | LPAPIEKTIS |
| 301 | KAKGQPREPQ VYTLPPSREE MTKNQVSLTC LVKGFYPSDI |
| | AVEWESNGQP |
| 351 | ENNYKTTPPV LDSDGSFFLY SKLTVDKSRW QQGNVFSCSV |
| | MHEALHNHYT |
| 401 | QKSLSLSPGK |

hTβRII (G4S)4-hFc: Amino Acid Sequence lacking
leader sequence and lacking glycine prior to
hTβRII portion
(SEQ ID NO: 95)

| | |
|---|---|
| 1 | ATIPPHVQKS DVEMEAQKDE IICPSCNRTA HPLRHINNDM |
| | IVTDNNGAVK |
| 51 | FPQLCKFCDV RFSTCDNQKS CMSNCSITSI CEKPQEVCVA |
| | VWRKNDENIT |
| 101 | LETVCHDPKL PYHDFILEDA ASPKCIMKEK KKPGETFFMC |
| | SCSSDECNDN |
| 151 | IIFSEEYNTS NPDTGGGGSG GGGSGGGGSG GGGSTHTCPP |
| | CPAPELLGGP |
| 201 | SVFLFPPKPK DTLMISRTPE VTCVVVDVSH EDPEVKFNWY |
| | VDGVEVHNAK |
| 251 | TKPREEQYNS TYRVVSVLTV LHQDWLNGKE YKCKVSNKAL |
| | PAPIEKTISK |
| 301 | AKGQPREPQV YTLPPSREEM TKNQVSLTCL VKGFYPSDIA |
| | VEWESNGQPE |
| 351 | NNYKTTPPVL DSDGSFFLYS KLTVDKSRWQ QGNVFSCSVM |
| | HEALHNHYTQ |
| 401 | KSLSLSPGK |

hTβRII (G4S)4-hFc: Amino Acid Sequence lacking
leader sequence and lacking glycine and alanine
prior to hTβRII portion
(SEQ ID NO: 96)

| | |
|---|---|
| 1 | TIPPHVQKSD VEMEAQKDEI ICPSCNRTAH PLRHINNDMI |
| | VTDNNGAVKF |
| 51 | PQLCKFCDVR FSTCDNQKSC MSNCSITSIC EKPQEVCVAV |
| | WRKNDENITL |
| 101 | ETVCHDPKLP YHDFILEDAA SPKCIMKEKK KPGETFFMCS |
| | CSSDECNDNI |
| 151 | IFSEEYNTSN PDTGGGGSGG GGSGGGGSGG GGSTHTCPPC |
| | PAPELLGGPS |
| 201 | VFLFPPKPKD TLMISRTPEV TCVVVDVSHE DPEVKFNWYV |
| | DGVEVHNAKT |
| 251 | KPREEQYNST YRVVSVLTVL HQDWLNGKEY KCKVSNKALP |
| | APIEKTISKA |
| 301 | KGQPREPQVY TLPPSREEMT KNQVSLTCLV KGFYPSDIAV |
| | EWESNGQPEN |
| 351 | NYKTTPPVLD SDGSFFLYSK LTVDKSRWQQ GNVFSCSVMH |
| | EALHNHYTQK |
| 401 | SLSLSPGK |

hTβRII (G4S)4-hFc: Amino Acid Sequence lacking
leader sequence and lacking glycine, alanine, and
threonine prior to hTβRII portion
(SEQ ID NO: 97)

| | |
|---|---|
| 1 | IPPHVQKSDV EMEAQKDEII CPSCNRTAHP LRHINNDMIV |
| | TDNNGAVKFP |
| 51 | QLCKFCDVRF STCDNQKSCM SNCSITSICE KPQEVCVAVW |
| | RKNDENITLE |
| 101 | TVCHDPKLPY HDFILEDAAS PKCIMKEKKK PGETFFMCSC |
| | SSDECNDNII |
| 151 | FSEEYNTSNP DTGGGGSGGG GSGGGGSGGG GSTHTCPPCP |
| | APELLGGPSV |
| 201 | FLFPPKPKDT LMISRTPEVT CVVVDVSHED PEVKFNWYVD |
| | GVEVHNAKTK |
| 251 | PREEQYNSTY RVVSVLTVLH QDWLNGKEYK CKVSNKALPA |
| | PIEKTISKAK |
| 301 | GQPREPQVYT LPPSREEMTK NQVSLTCLVK GFYPSDIAVE |
| | WESNGQPENN |
| 351 | YKTTPPVLDS DGSFFLYSKL TVDKSRWQQG NVFSCSVMHE |
| | ALHNHYTQKS |
| 401 | LSLSPGK |

hTβRII (G4S)4-hFc: Amino Acid Sequence lacking
leader sequence and lacking glycine, alanine,
threonine, and isoleucine prior to hTβRII portion
(SEQ ID NO: 98)

| | |
|---|---|
| 1 | PPHVQKSDVE MEAQKDEIIC PSCNRTAHPL RHINNDMIVT |
| | DNNGAVKFPQ |
| 51 | LCKFCDVRFS TCDNQKSCMS NCSITSICEK PQEVCVAVWR |
| | KNDENITLET |
| 101 | VCHDPKLPYH DFILEDAASP KCIMKEKKKP GETFFMCSCS |
| | SDECNDNIIF |
| 151 | SEEYNTSNPD TGGGGSGGGG SGGGGSGGGG STHTCPPCPA |
| | PELLGGPSVF |
| 201 | LFPPKPKDTL MISRTPEVTC VVVDVSHEDP EVKFNWYVDG |
| | VEVHNAKTKP |
| 251 | REEQYNSTYR VVSVLTVLHQ DWLNGKEYKC KVSNKALPAP |
| | IEKTISKAKG |
| 301 | QPREPQVYTL PPSREEMTKN QVSLTCLVKG FYPSDIAVEW |
| | ESNGQPENNY |
| 351 | KTTPPVLDSD GSFFLYSKLT VDKSRWQQGN VFSCSVMHEA |
| | LHNHYTQKSL |
| 401 | SLSPGK |

hTβRII (G4S)4-hFc: Amino Acid Sequence lacking
leader sequence and lacking glycine, alanine,
threonine, isoleucine, and proline prior to
hTβRII portion
(SEQ ID NO: 99)

| | |
|---|---|
| 1 | PHVQKSDVEM EAQKDEIICP SCNRTAHPLR HINNDMIVTD |
| | NNGAVKFPQL |
| 51 | CKFCDVRFST CDNQKSCMSN CSITSICEKP QEVCVAVWRK |
| | NDENITLETV |
| 101 | CHDPKLPYHD FILEDAASPK CIMKEKKKPG ETFFMCSCSS |
| | DECNDNIIFS |
| 151 | EEYNTSNPDT GGGGSGGGGS GGGGSGGGGS THTCPPCPAP |
| | ELLGGPSVFL |
| 201 | FPPKPKDTLM ISRTPEVTCV VVDVSHEDPE VKFNWYVDGV |
| | EVHNAKTKPR |
| 251 | EEQYNSTYRV VSVLTVLHQD WLNGKEYKCK VSNKALPAPI |
| | EKTISKAKGQ |
| 301 | PREPQVYTLP PSREEMTKNQ VSLTCLVKGF YPSDIAVEWE |
| | SNGQPENNYK |
| 351 | TTPPVLDSDG SFFLYSKLTV DKSRWQQGNV FSCSVMHEAL |
| | HNHYTQKSLS |
| 401 | LSPGK |

hTβRII (G4S)4-hFc: Amino Acid Sequence lacking
leader sequence and lacking glycine, alanine,
threonine, isoleucine, proline, and proline prior
to hTβRII portion
(SEQ ID NO: 100)

| | |
|---|---|
| 1 | HVQKSDVEME AQKDEIICPS CNRTAHPLRH INNDMIVTDN |
| | NGAVKFPQLC |
| 51 | KFCDVRFSTC DNQKSCMSNC SITSICEKPQ EVCVAVWRKN |
| | DENITLETVC |
| 101 | HDPKLPYHDF ILEDAASPKC IMKEKKKPGE TFFMCSCSSD |
| | ECNDNIIFSE |
| 151 | EYNTSNPDTG GGGSGGGGSG GGGSGGGGST HTCPPCPAPE |
| | LLGGPSVFLF |
| 201 | PPKPKDTLMI SRTPEVTCVV VDVSHEDPEV KFNWYVDGVE |
| | VHNAKTKPRE |
| 251 | EQYNSTYRVV SVLTVLHQDW LNGKEYKCKV SNKALPAPIE |
| | KTISKAKGQP |
| 301 | REPQVYTLPP SREEMTKNQV SLTCLVKGFY PSDIAVEWES |
| | NGQPENNYKT |
| 351 | TPPVLDSDGS FFLYSKLTVD KSRWQQGNVF SCSVMHEALH |
| | NHYTQKSLSL |
| 401 | SPGK |

hTβRII (G4S)2-hFc: Nucleic Acid Sequence
(SEQ ID NO: 14)

| | |
|---|---|
| 1 | ATGGATGCAA TGAAGAGAGG GCTCTGCTGT GTGCTGCTGC |
| | TGTGTGGAGC |
| 51 | AGTCTTCGTT TCGCCCGGCG CCACGATCCC ACCGCACGTT |
| | CAGAAGTCGG |
| 101 | ATGTGGAAAT GGAGGCCCAG AAAGATGAAA TCATCTGCCC |
| | CAGCTGTAAT |
| 151 | AGGACTGCCC ATCCACTGAG ACATATTAAT AACGACATGA |
| | TAGTCACTGA |
| 201 | CAACAACGGT GCAGTCAAGT TTCCACAACT GTGTAAATTT |
| | TGTGATGTGA |
| 251 | GATTTTCCAC CTGTGACAAC CAGAAATCCT GCATGAGCAA |
| | CTGCAGCATC |
| 301 | ACCTCCATCT GTGAGAAGCC ACAGGAAGTC TGTGTGGCTG |
| | TATGGAGAAA |
| 351 | GAATGACGAG AACATAACAC TAGAGACAGT TTGCCATGAC |
| | CCCAAGCTCC |
| 401 | CCTACCATGA CTTTATTCTG GAAGATGCTG CTTCTCCAAA |
| | GTGCATTATG |
| 451 | AAGGAAAAAA AAAAGCCTGG TGAGACTTTC TTCATGTGTT |
| | CCTGTAGCTC |
| 501 | TGATGAGTGC AATGACAACA TCATCTTCTC AGAAGAATAT |
| | AACACCAGCA |
| 551 | ATCCTGACAC CGGTGGAGGT GGTTCTGGAG GTGGTGGAAG |
| | TACTCACACA |
| 601 | TGCCCACCGT GCCCAGCACC TGAACTCCTG GGGGGACCGT |
| | CAGTCTTCCT |
| 651 | CTTCCCCCCA AAACCCAAGG ACACCCTCAT GATCTCCCGG |
| | ACCCCTGAGG |
| 701 | TCACATGCGT GGTGGTGGAC GTGAGCCACG AAGACCCTGA |
| | GGTCAAGTTC |
| 751 | AACTGGTACG TGGACGGCGT GGAGGTGCAT AATGCCAAGA |
| | CAAAGCCGCG |
| 801 | GGAGGAGCAG TACAACAGCA CGTACCGTGT GGTCAGCGTC |
| | CTCACCGTCC |
| 851 | TGCACCAGGA CTGGCTGAAT GGCAAGGAGT ACAAGTGCAA |
| | GGTCTCCAAC |
| 901 | AAAGCCCTCC CAGCCCCCAT CGAGAAAACC ATCTCCAAAG |
| | CCAAAGGGCA |
| 951 | GCCCCGAGAA CCACAGGTGT ACACCCTGCC CCCATCCCGG |
| | GAGGAGATGA |
| 1001 | CCAAGAACCA GGTCAGCCTG ACCTGCCTGG TCAAAGGCTT |
| | CTATCCCAGC |
| 1051 | GACATCGCCG TGGAGTGGGA GAGCAATGGG CAGCCGGAGA |
| | ACAACTACAA |
| 1101 | GACCACGCCT CCCGTGCTGG ACTCCGACGG CTCCTTCTTC |
| | CTCTATAGCA |
| 1151 | AGCTCACCGT GGACAAGAGC AGGTGGCAGC AGGGGAACGT |
| | CTTCTCATGC |
| 1201 | TCCGTGATGC ATGAGGCTCT GCACAACCAC TACACGCAGA |
| | AGAGCCTCTC |
| 1251 | CCTGTCTCCG GGTAAATGA |

hTβRII (G4S)2-hFc: Amino Acid Sequence
(SEQ ID NO: 15)

| | |
|---|---|
| 1 | MDAMKRGLCC VLLLCGAVFV SPGATIPPHV QKSDVEMEAQ |
| | KDEIICPSCN |
| 51 | RTAHPLRHIN NDMIVTDNNG AVKFPQLCKF CDVRFSTCDN |
| | QKSCMSNCSI |
| 101 | TSICEKPQEV CVAVWRKNDE NITLETVCHD PKLPYHDFIL |
| | EDAASPKCIM |
| 151 | KEKKKPGETF FMCSCSSDEC NDNIIFSEEY NTSNPDTGGG |
| | GSGGGGSTHT |
| 201 | CPPCPAPELL GGPSVFLFPP KPKDTLMISR TPEVTCVVVD |
| | VSHEDPEVKF |
| 251 | NWYVDGVEVH NAKTKPREEQ YNSTYRVVSV LTVLHQDWLN |
| | GKEYKCKVSN |
| 301 | KALPAPIEKT ISKAKGQPRE PQVYTLPPSR EEMTKNQVSL |
| | TCLVKGFYPS |
| 351 | DIAVEWESNG QPENNYKTTP PVLDSDGSFF LYSKLTVDKS |
| | RWQQGNVFSC |
| 401 | SVMHEALHNH YTQKSLSLSP GK |

hTβRII extended hinge-hFc: Nucleic Acid Sequence
(SEQ ID NO: 16)

| | |
|---|---|
| 1 | ATGGATGCAA TGAAGAGAGG GCTCTGCTGT GTGCTGCTGC |
| | TGTGTGGAGC |
| 51 | AGTCTTCGTT TCGCCCGGCG CCACGATCCC ACCGCACGTT |
| | CAGAAGTCGG |
| 101 | ATGTGGAAAT GGAGGCCCAG AAAGATGAAA TCATCTGCCC |
| | CAGCTGTAAT |
| 151 | AGGACTGCCC ATCCACTGAG ACATATTAAT AACGACATGA |
| | TAGTCACTGA |
| 201 | CAACAACGGT GCAGTCAAGT TTCCACAACT GTGTAAATTT |
| | TGTGATGTGA |
| 251 | GATTTTCCAC CTGTGACAAC CAGAAATCCT GCATGAGCAA |
| | CTGCAGCATC |
| 301 | ACCTCCATCT GTGAGAAGCC ACAGGAAGTC TGTGTGGCTG |
| | TATGGAGAAA |
| 351 | GAATGACGAG AACATAACAC TAGAGACAGT TTGCCATGAC |
| | CCCAAGCTCC |
| 401 | CCTACCATGA CTTTATTCTG GAAGATGCTG CTTCTCCAAA |
| | GTGCATTATG |
| 451 | AAGGAAAAAA AAAAGCCTGG TGAGACTTTC TTCATGTGTT |
| | CCTGTAGCTC |
| 501 | TGATGAGTGC AATGACAACA TCATCTTCTC AGAAGAATAT |
| | AACACCAGCA |
| 551 | ATCCTGACAC CGGTGGTGGA CCCAAATCTT GTGACAAAAC |
| | TCACACATGC |
| 601 | CCACCGTGCC CAGCACCTGA ACTCCTGGGG GGACCGTCAG |
| | TCTTCCTCTT |
| 651 | CCCCCCAAAA CCCAAGGACA CCCTCATGAT CTCCCGGACC |
| | CCTGAGGTCA |
| 701 | CATGCGTGGT GGTGGACGTG AGCCACGAAG ACCCTGAGGT |
| | CAAGTTCAAC |
| 751 | TGGTACGTGG ACGGCGTGGA GGTGCATAAT GCCAAGACAA |
| | AGCCGCGGGA |
| 801 | GGAGCAGTAC AACAGCACGT ACCGTGTGGT CAGCGTCCTC |
| | ACCGTCCTGC |
| 851 | ACCAGGACTG GCTGAATGGC AAGGAGTACA AGTGCAAGGT |
| | CTCCAACAAA |
| 901 | GCCCTCCCAG CCCCCATCGA GAAAACCATC TCCAAAGCCA |
| | AAGGGCAGCC |
| 951 | CCGAGAACCA CAGGTGTACA CCCTGCCCCC ATCCCGGGAG |
| | GAGATGACCA |
| 1001 | AGAACCAGGT CAGCCTGACC TGCCTGGTCA AAGGCTTCTA |
| | TCCCAGCGAC |
| 1051 | ATCGCCGTGG AGTGGGAGAG CAATGGGCAG CCGGAGAACA |
| | ACTACAAGAC |
| 1101 | CACGCCTCCC GTGCTGGACT CCGACGGCTC CTTCTTCCTC |
| | TATAGCAAGC |
| 1151 | TCACCGTGGA CAAGAGCAGG TGGCAGCAGG GGAACGTCTT |
| | CTCATGCTCC |
| 1201 | GTGATGCATG AGGCTCTGCA CAACCACTAC ACGCAGAAGA |
| | GCCTCTCCCT |
| 1251 | GTCCCCGGGT AAATGA |

hTβRII extended hinge-hFc: Amino Acid Sequence
(SEQ ID NO: 17)

| | |
|---|---|
| 1 | MDAMKRGLCC VLLLCGAVFV SPGATIPPHV QKSDVEMEAQ |
| | KDEIICPSCN |
| 51 | RTAHPLRHIN NDMIVTDNNG AVKFPQLCKF CDVRFSTCDN |
| | QKSCMSNCSI |
| 101 | TSICEKPQEV CVAVWRKNDE NITLETVCHD PKLPYHDFIL |
| | EDAASPKCIM |
| 151 | KEKKKPGETF FMCSCSSDEC NDNIIFSEEY NTSNPDTGGG |
| | PKSCDKTHTC |
| 201 | PPCPAPELLG GPSVFLFPPK PKDTLMISRT PEVTCVVVDV |
| | SHEDPEVKFN |
| 251 | WYVDGVEVHN AKTKPREEQY NSTYRVVSVL TVLHQDWLNG |
| | KEYKCKVSNK |
| 301 | ALPAPIEKTI SKAKGQPREP QVYTLPPSRE EMTKNQVSLT |
| | CLVKGFYPSD |
| 351 | IAVEWESNGQ PENNYKTTPP VLDSDGSFFL YSKLTVDKSR |
| | WQQGNVFSCS |
| 401 | VMHEALHNHY TQKSLSLSPG K |

hTβRII (G4S)5-hFc: Amino Acid Sequence
(SEQ ID NO: 44)

| | |
|---|---|
| 1 | MDAMKRGLCC VLLLCGAVFV SPGATIPPHV QKSDVEMEAQ |
| | KDEIICPSCN |
| 51 | RTAHPLRHIN NDMIVTDNNG AVKFPQLCKF CDVRFSTCDN |
| | QKSCMSNCSI |
| 101 | TSICEKPQEV CVAVWRKNDE NITLETVCHD PKLPYHDFIL |
| | EDAASPKCIM |
| 151 | KEKKKPGETF FMCSCSSDEC NDNIIFSEEY NTSNPDTGGG |
| | GSGGGGSGGG |
| 201 | GSGGGGSGGG GSTHTCPPCP APELLGGPSV FLFPPKPKDT |
| | LMISRTPEVT |
| 251 | CVVVDVSHED PEVKFNWYVD GVEVHNAKTK PREEQYNSTY |
| | RVVSVLTVLH |
| 301 | QDWLNGKEYK CKVSNKALPA PIEKTISKAK GQPREPQVYT |
| | LPPSREEMTK |
| 351 | NQVSLTCLVK GFYPSDIAVE WESNGQPENN YKTTPPVLDS |
| | DGSFFLYSKL |
| 401 | TVDKSRWQQG NVFSCSVMHE ALHNHYTQKS LSLSPGK |

hTβRII (G4S)6-hFc: Amino Acid Sequence
(SEQ ID NO: 45)

| | |
|---|---|
| 1 | MDAMKRGLCC VLLLCGAVFV SPGATIPPHV QKSDVEMEAQ |
| | KDEIICPSCN |
| 51 | RTAHPLRHIN NDMIVTDNNG AVKFPQLCKF CDVRFSTCDN |
| | QKSCMSNCSI |
| 101 | TSICEKPQEV CVAVWRKNDE NITLETVCHD PKLPYHDFIL |
| | EDAASPKCIM |
| 151 | KEKKKPGETF FMCSCSSDEC NDNIIFSEEY NTSNPDTGGG |
| | GSGGGGSGGG |
| 201 | GSGGGGSGGG GSGGGGSTHT CPPCPAPELL GGPSVFLFPP |
| | KPKDTLMISR |
| 251 | TPEVTCVVVD VSHEDPEVKF NWYVDGVEVH NAKTKPREEQ |
| | YNSTYRVVSV |
| 301 | LTVLHQDWLN GKEYKCKVSN KALPAPIEKT ISKAKGQPRE |
| | PQVYTLPPSR |
| 351 | EEMTKNQVSL TCLVKGFYPS DIAVEWESNG QPENNYKTTP |
| | PVLDSDGSFF |
| 401 | LYSKLTVDKS RWQQGNVFSC SVMHEALHNH YTQKSLSLSP |
| | GK |

hTβRII (G4S)5-hFc: Nucleotide Sequence
(SEQ ID NO: 46)

| | |
|---|---|
| 1 | ATGGATGCAA TGAAGAGAGG GCTCTGCTGT GTGCTGCTGC |
| | TGTGTGGAGC |
| 51 | AGTCTTCGTT TCGCCCGGCG CCACGATCCC ACCGCACGTT |
| | CAGAAGTCGG |
| 101 | ATGTGGAAAT GGAGGCCCAG AAAGATGAAA TCATCTGCCC |
| | CAGCTGTAAT |
| 151 | AGGACTGCCC ATCCACTGAG ACATATTAAT AACGACATGA |
| | TAGTCACTGA |
| 201 | CAACAACGGT GCAGTCAAGT TTCCACAACT GTGTAAATTT |
| | TGTGATGTGA |
| 251 | GATTTTCCAC CTGTGACAAC CAGAAATCCT GCATGAGCAA |
| | CTGCAGCATC |
| 301 | ACCTCCATCT GTGAGAAGCC ACAGGAAGTC TGTGTGGCTG |
| | TATGGAGAAA |
| 351 | GAATGACGAG AACATAACAC TAGAGACAGT TTGCCATGAC |
| | CCCAAGCTCC |
| 401 | CCTACCATGA CTTTATTCTG GAAGATGCTG CTTCTCCAAA |
| | GTGCATTATG |
| 451 | AAGGAAAAAA AAAAGCCTGG TGAGACTTTC TTCATGTGTT |
| | CCTGTAGCTC |
| 501 | TGATGAGTGC AATGACAACA TCATCTTCTC AGAAGAATAT |
| | AACACCAGCA |
| 551 | ATCCTGACAC CGGTGGAGGA GGTTCTGGTG GTGGAGGTTC |
| | TGGAGGTGGA |
| 601 | GGAAGTGGTG GAGGTGGTTC TGGAGGTGGT GGAAGTACTC |
| | ACACATGCCC |
| 651 | ACCGTGCCCA GCACCTGAAC TCCTGGGGGG ACCGTCAGTC |
| | TTCCTCTTCC |
| 701 | CCCCAAAACC CAAGGACACC CTCATGATCT CCCGGACCCC |
| | TGAGGTCACA |
| 751 | TGCGTGGTGG TGGACGTGAG CCACGAAGAC CCTGAGGTCA |
| | AGTTCAACTG |
| 801 | GTACGTGGAC GGCGTGGAGG TGCATAATGC CAAGACAAAG |
| | CCGCGGGAGG |
| 851 | AGCAGTACAA CAGCACGTAC CGTGTGGTCA GCGTCCTCAC |
| | CGTCCTGCAC |
| 901 | CAGGACTGGC TGAATGGCAA GGAGTACAAG TGCAAGGTCT |
| | CCAACAAAGC |
| 951 | CCTCCCAGCC CCCATCGAGA AAACCATCTC CAAAGCCAAA |
| | GGGCAGCCCC |
| 1001 | GAGAACCACA GGTGTACACC CTGCCCCCAT CCCGGGAGGA |
| | GATGACCAAG |
| 1051 | AACCAGGTCA GCCTGACCTG CCTGGTCAAA GGCTTCTATC |
| | CCAGCGACAT |
| 1101 | CGCCGTGGAG TGGGAGAGCA ATGGGCAGCC GGAGAACAAC |
| | TACAAGACCA |
| 1151 | CGCCTCCCGT GCTGGACTCC GACGGCTCCT TCTTCCTCTA |
| | TAGCAAGCTC |
| 1201 | ACCGTGGACA AGAGCAGGTG GCAGCAGGGG AACGTCTTCT |
| | CATGCTCCGT |
| 1251 | GATGCATGAG GCTCTGCACA ACCACTACAC GCAGAAGAGC |
| | CTCTCCCTGT |
| 1301 | CTCCGGGTAA ATGA |

hTβRII (G4S)6-hFc: Nucleotide Sequence
(SEQ ID NO: 47)

| | |
|---|---|
| 1 | ATGGATGCAA TGAAGAGAGG GCTCTGCTGT GTGCTGCTGC |
| | TGTGTGGAGC |
| 51 | AGTCTTCGTT TCGCCCGGCG CCACGATCCC ACCGCACGTT |
| | CAGAAGTCGG |
| 101 | ATGTGGAAAT GGAGGCCCAG AAAGATGAAA TCATCTGCCC |
| | CAGCTGTAAT |
| 151 | AGGACTGCCC ATCCACTGAG ACATATTAAT AACGACATGA |
| | TAGTCACTGA |
| 201 | CAACAACGGT GCAGTCAAGT TTCCACAACT GTGTAAATTT |
| | TGTGATGTGA |
| 251 | GATTTTCCAC CTGTGACAAC CAGAAATCCT GCATGAGCAA |
| | CTGCAGCATC |
| 301 | ACCTCCATCT GTGAGAAGCC ACAGGAAGTC TGTGTGGCTG |
| | TATGGAGAAA |
| 351 | GAATGACGAG AACATAACAC TAGAGACAGT TTGCCATGAC |
| | CCCAAGCTCC |
| 401 | CCTACCATGA CTTTATTCTG GAAGATGCTG CTTCTCCAAA |
| | GTGCATTATG |
| 451 | AAGGAAAAAA AAAAGCCTGG TGAGACTTTC TTCATGTGTT |
| | CCTGTAGCTC |
| 501 | TGATGAGTGC AATGACAACA TCATCTTCTC AGAAGAATAT |
| | AACACCAGCA |
| 551 | ATCCTGACAC CGGTGGAGGT GGAAGTGGTG GAGGAGGTTC |
| | TGGTGGTGGA |
| 601 | GGTTCTGGAG GTGGAGGAAG TGGTGGAGGT GGTTCTGGAG |
| | GTGGTGGAAG |
| 651 | TACTCACACA TGCCCACCGT GCCCAGCACC TGAACTCCTG |
| | GGGGGACCGT |
| 701 | CAGTCTTCCT CTTCCCCCCA AAACCCAAGG ACACCCTCAT |
| | GATCTCCCGG |
| 751 | ACCCCTGAGG TCACATGCGT GGTGGTGGAC GTGAGCCACG |
| | AAGACCCTGA |
| 801 | GGTCAAGTTC AACTGGTACG TGGACGGCGT GGAGGTGCAT |
| | AATGCCAAGA |
| 851 | CAAAGCCGCG GGAGGAGCAG TACAACAGCA CGTACCGTGT |
| | GGTCAGCGTC |
| 901 | CTCACCGTCC TGCACCAGGA CTGGCTGAAT GGCAAGGAGT |
| | ACAAGTGCAA |
| 951 | GGTCTCCAAC AAAGCCCTCC CAGCCCCCAT CGAGAAAACC |
| | ATCTCCAAAG |
| 1001 | CCAAAGGGCA GCCCCGAGAA CCACAGGTGT ACACCCTGCC |
| | CCCATCCCGG |
| 1051 | GAGGAGATGA CCAAGAACCA GGTCAGCCTG ACCTGCCTGG |
| | TCAAAGGCTT |
| 1101 | CTATCCCAGC GACATCGCCG TGGAGTGGGA GAGCAATGGG |
| | CAGCCGGAGA |
| 1151 | ACAACTACAA GACCACGCCT CCCGTGCTGG ACTCCGACGG |
| | CTCCTTCTTC |
| 1201 | CTCTATAGCA AGCTCACCGT GGACAAGAGC AGGTGGCAGC |
| | AGGGGAACGT |
| 1251 | CTTCTCATGC TCCGTGATGC ATGAGGCTCT GCACAACCAC |
| | TACACGCAGA |
| 1301 | AGAGCCTCTC CCTGTCTCCG GGTAAATGA |

The various constructs were successfully expressed in CHO cells and were purified to a high degree of purity as determined by analytical size-exclusion chromatography and SDS-PAGE. The hTβRII (G4S)2-hFc, hTβRII (G4S)3-hFc, hTβRII (G4S)4-hFc, hTβRII (G4S)5-hFc and hTβRII (G4S)6-hFc proteins displayed similarly strong stability as determined by SDS-PAGE analysis when maintained in PBS for 13 days at 37° C. The hTβRII (G4S)2-hFc, hTβRII (G4S)3-hFc, hTβRII (G4S)4-hFc proteins were also maintained in rat, mouse or human serum and displayed similarly strong stability.

### TβRII ECD Variants

In addition to the TβRII domains included in the fusion proteins described above (e.g., SEQ ID NO: 18), the disclosure also contemplates fusion proteins comprising alternative TβRII domains. For example, the fusion protein may comprise the wild-type hTβRIIₛₕₒᵣₜ(23-159) sequence shown below (SEQ ID NO: 27) or any of the other TβRII polypeptides disclosed below:

(SEQ ID NO: 27)

| | |
|---|---|
| 1 | TIPPHVQKSV NNDMIVTDNN GAVKFPQLCK FCDVRFSTCD |
| | NQKSCMSNCS |
| 51 | ITSICEKPQE VCVAVWRKND ENITLETVCH DPKLPYHDFI |
| | LEDAASPKCI |
| 101 | MKEKKKPGET FFMCSCSSDE CNDNIIFSEE YNTSNPD |

(1) The hTβRIIₛₕₒᵣₜ(23-159/D110K) amino acid sequence shown below (SEQ ID NO: 36), in which the substituted residue is underlined.

(SEQ ID NO: 36)

| | |
|---|---|
| 1 | TIPPHVQKSV NNDMIVTDNN GAVKFPQLCK FCDVRFSTCD |
| | NQKSCMSNCS |
| 51 | ITSICEKPQE VCVAVWRKND ENITLETVCH DPKLPYHKFI |
| | LEDAASPKCI |
| 101 | MKEKKKPGET FFMCSCSSDE CNDNIIFSEE YNTSNPD |

(2) The N-terminally truncated hTβRIIₛₕₒᵣₜ(29-159) amino acid sequence shown below (SEQ ID NO: 28).

(SEQ ID NO: 28)

| | |
|---|---|
| 1 | QKSVNNDMIV TDNNGAVKFP QLCKFCDVRF |
| | STCDNQKSCM SNCSITSICE |
| 51 | KPQEVCVAVW RKNDENITLE TVCHDPKLPY |
| | HDFILEDAAS PKCIMKEKKK |
| 101 | PGETFFMCSC SSDECNDNII FSEEYNTSNP D |

(3) The N-terminally truncated hTβRIIₛₕₒᵣₜ(35-159) amino acid sequence shown below (SEQ ID NO: 29).

(SEQ ID NO: 29)

| | |
|---|---|
| 1 | DMIVTDNNGA VKFPQLCKFC DVRFSTCDNQ |
| | KSCMSNCSIT SICEKPQEVC |
| 51 | VAVWRKNDEN ITLETVCHDP KLPYHDFILE |
| | DAASPKCIMK EKKKPGETFF |
| 101 | MCSCSSDECN DNIIFSEEYN TSNPD |

(4) The C-terminally truncated hTβRIIₛₕₒᵣₜ(23-153) amino acid sequence shown below (SEQ ID NO: 30).

(SEQ ID NO: 30)

| | |
|---|---|
| 1 | TIPPHVQKSV NNDMIVTDNN GAVKFPQLCK |
| | FCDVRFSTCD NQKSCMSNCS |
| 51 | ITSICEKPQE VCVAVWRKND ENITLETVCH |
| | DPKLPYHDFI LEDAASPKCI |
| 101 | MKEKKKPGET FFMCSCSSDE CNDNIIFSEE Y |

(5) The C-terminally truncated hTβRIIₛₕₒᵣₜ(23-153/N70D) amino acid sequence shown below (SEQ ID NO: 38), in which the substituted residue is underlined.

(SEQ ID NO: 38)

| | |
|---|---|
| 1 | TIPPHVQKSV NNDMIVTDNN GAVKFPQLCK FCDVRFSTCD |
| | NQKSCMSDCS |
| 51 | ITSICEKPQE VCVAVWRKND ENITLETVCH DPKLPYHDFI |
| | LEDAASPKCI |
| 101 | MKEKKKPGET FFMCSCSSDE CNDNIIFSEE Y |

Applicants also envision five corresponding variants (SEQ ID NOs: 37, 33, 34, 39) based on the wild-type hTβRII_{long}(23-184) sequence shown above and below (SEQ ID NO: 20), in which the 25 amino-acid insertion is underlined. Note that splicing results in a conservative amino acid substitution (Val→Ile) at the flanking position C-terminal to the insertion.

(SEQ ID NO: 20)

| | |
|---|---|
| 1 | TIPPHVQKSD VEMEAQKDEI ICPSCNRTAH PLRHINNDMI |
| | VTDNNGAVKF |
| 51 | PQLCKFCDVR FSTCDNQKSC MSNCSITSIC EKPQEVCVAV |
| | WRKNDENITL |
| 101 | ETVCHDPKLP YHDFILEDAA SPKCIMKEKK KPGETFFMCS |
| | CSSDECNDNI |
| 151 | IFSEEYNTSN PD |

(1) The hTβRII_{long}(23-184/D135K) amino acid sequence shown below (SEQ ID NO: 37), in which the substituted residue is double underlined.

(SEQ ID NO: 37)

| | |
|---|---|
| 1 | TIPPHVQKSD VEMEAQKDEI ICPSCNRTAH PLRHINNDMI |
| | VTDNNGAVKF |
| 51 | PQLCKFCDVR FSTCDNQKSC MSNCSITSIC EKPQEVCVAV |
| | WRKNDENITL |
| 101 | ETVCHDPKLP YHKFILEDAA SPKCIMKEKK KPGETFFMCS |
| | CSSDECNDNI |
| 151 | IFSEEYNTSN PD |

(2) The N-terminally truncated hTβRII_{long}(29-184) amino acid sequence shown below (SEQ ID NO: 33).

(SEQ ID NO: 33)

| | |
|---|---|
| 1 | QKSDVEMEAQ KDEIICPSCN RTAHPLRHIN NDMIVTDNNG |
| | AVKFPQLCKF |
| 51 | CDVRFSTCDN QKSCMSNCSI TSICEKPQEV CVAVWRKNDE |
| | NITLETVCHD |
| 101 | PKLPYHDFIL EDAASPKCIM KEKKKPGETF FMCSCSSDEC |
| | NDNIIFSEEY |
| 151 | NTSNPD |

(3) The N-terminally truncated hTβRII_{long}(60-184) amino acid sequence shown below (same as SEQ ID NO: 29).

| | |
|---|---|
| 1 | DMIVTDNNGA VKFPQLCKFC DVRFSTCDNQ KSCMSNCSIT |
| | SICEKPQEVC |
| 51 | VAVWRKNDEN ITLETVCHDP KLPYHDFILE DAASPKCIMK |
| | EKKKPGETFF |
| 101 | MCSCSSDECN DNIIFSEEYN TSNPD (same as SEQ ID |
| | NO: 29) |

(4) The C-terminally truncated hTβRII_{long}(23-178) amino acid sequence shown below (SEQ ID NO: 34).

(SEQ ID NO: 34)

| | |
|---|---|
| 1 | TIPPHVQKSD VEMEAQKDEI ICPSCNRTAH PLRHINNDMI |
| | VTDNNGAVKF |
| 51 | PQLCKFCDVR FSTCDNQKSC MSNCSITSIC EKPQEVCVAV |
| | WRKNDENITL |
| 101 | ETVCHDPKLP YHDFILEDAA SPKCIMKEKK KPGETFFMCS |
| | CSSDECNDNI |
| 151 | IFSEEY |

(5) The C-terminally truncated hTβRII_{long}(23-178/N95D) amino acid sequence shown below (SEQ ID NO: 39), in which the substituted residue is double underlined.

(SEQ ID NO: 39)

| | |
|---|---|
| 1 | TIPPHVQKSD VEMEAQKDEI ICPSCNRTAH PLRHINNDMI |
| | VTDNNGAVKF |
| 51 | PQLCKFCDVR FSTCDNQKSC MSDCSITSIC EKPQEVCVAV |
| | WRKNDENITL |
| 101 | ETVCHDPKLP YHDFILEDAA SPKCIMKEKK KPGETFFMCS |
| | CSSDECNDNI |
| 151 | IFSEEY |

Additional TβRII ECD variants include:
(A) The N- and C-terminally truncated hTβRIIₛₕₒᵣₜ(35-153) or hTβRII_{long}(60-178) amino acid sequence shown below (SEQ ID NO: 32).

(SEQ ID NO: 32)

| | |
|---|---|
| 1 | DMIVTDNNGA VKFPQLCKFC DVRFSTCDNQ KSCMSNCSIT |
| | SICEKPQEVC |
| 51 | VAVWRKNDEN ITLETVCHDP KLPYHDFILE DAASPKCIMK |
| | EKKKPGETFF |
| 101 | MCSCSSDECN DNIIFSEEY |

(B) The N- and C-terminally truncated hTβRIIₛₕₒᵣₜ(29-153) amino acid sequence shown below (SEQ ID NO: 31).

(SEQ ID NO: 31)

| | |
|---|---|
| 1 | QKSVNNDMIV TDNNGAVKFP QLCKFCDVRF STCDNQKSCM |
| | SNCSITSICE |
| 51 | KPQEVCVAVW RKNDENITLE TVCHDPKLPY HDFILEDAAS |
| | PKCIMKEKKK |
| 101 | PGETFFMCSC SSDECNDNII FSEEY |

(C) The N- and C-terminally truncated hTβRII_{long}(29-178) amino acid sequence shown below (SEQ ID NO: 35).

(SEQ ID NO: 35)

| | |
|---|---|
| 1 | QKSDVEMEAQ KDEIICPSCN RTAHPLRHIN NDMIVTDNNG |
| | AVKFPQLCKF |
| 51 | CDVRFSTCDN QKSCMSNCSI TSICEKPQEV CVAVWRKNDE |
| | NITLETVCHD |
| 101 | PKLPYHDFIL EDAASPKCIM KEKKKPGETF FMCSCSSDEC |
| | NDNIIFSEEY |

Any of the above variants (SEQ ID NOs: 36, 28, 29, 30, 38, 37, 33, 34, 39, 32, 31, and 35) could incorporate an insertion of 36 amino acids (SEQ ID NO: 41) between the pair of glutamate residues (positions 151 and 152 of SEQ ID NO: 1, or positions 176 and 177 of SEQ ID NO: 2) located near the C-terminus of the hTβRII ECD, as occurs naturally in the hTβRII isoform C (Konrad et al., BMC Genomics 8:318, 2007).
GRCKIRHIGS NNRLQRSTCQ NTGWESAHVM KTPGFR (SEQ ID NO: 41)

As an example, the paired glutamate residues flanking the optional insertion site are denoted below (underlined) for the hTβRIIₛₕₒᵣₜ(29-159) variant (SEQ ID NO: 28).

(SEQ ID NO: 28)

| | |
|---|---|
| 1 | QKSVNNDMIV TDNNGAVKFP QLCKFCDVRF STCDNQKSCM |
| | SNCSITSICE |
| 51 | KPQEVCVAVW RKNDENITLE TVCHDPKLPY HDFILEDAAS |
| | PKCIMKEKKK |
| 101 | PGETFFMCSC SSDECNDNII FSEEYNTSNP D |

### Fc Domain Variants

While the constructs described above were generated with an Fc domain having the amino acid sequence of SEQ ID NO: 49, the disclosure contemplates hTβRII-hFc fusion proteins comprising alternative Fc domains, including a human IgG2 Fc domain (SEQ ID NO: 42, below) or full-length human IgG1 Fc (hG1Fc) (SEQ ID NO: 43, below). Optionally, a polypeptide unrelated to an Fc domain could be attached in lace of the Fc domain.

(SEQ ID NO: 42)

| | |
|---|---|
| 1 | VECPPCPAPP VAGPSVFLFP PKPKDTLMIS RTPEVTCVVV |
| | DVSHEDPEVQ |
| 51 | FNWYVDGVEV HNAKTKPREE QFNSTFRVVS VLTVVHQDWL |
| | NGKEYKCKVS |
| 101 | NKGLPAPIEK TISKTKGQPR EPQVYTLPPS REEMTKNQVS |
| | LTCLVKGFYP |
| 151 | SDIAVEWESN GQPENNYKTT PPMLDSDGSF FLYSKLTVDK |
| | SRWQQGNVFS |
| 201 | CSVMHEALHN HYTQKSLSLS PGK |

(SEQ ID NO: 43)

| | |
|---|---|
| 1 | GGPKSCDKTH TCPPCPAPEL LGGPSVFLFP PKPKDTLMIS |
| | RTPEVTCVVV |
| 51 | DVSHEDPEVK FNWYVDGVEV HNAKTKPREE QYNSTYRVVS |
| | VLTVLHQDWL |
| 101 | NGKEYKCKVS NKALPAPIEK TISKAKGQPR EPQVYTLPPS |
| | REEMTKNQVS |
| 151 | LTCLVKGFYP SDIAVEWESN GQPENNYKTT PPVLDSDGSF |
| | FLYSKLTVDK |
| 201 | SRWQQGNVFS CSVMHEALHN HYTQKSLSLS PGK |

### Leader Sequence Variants

While the generated constructs described above included the TPA leader sequence, alternative leader sequences may be used, such as the native leader sequence (SEQ ID NO: 22-below) or the honey bee melittin (SEQ ID NO: 24-below) leader sequences.

Native:
(SEQ ID NO: 22)
MGRGLLRGLWPLHIVLWTRIAS
Honey bee melittin (HBML):
(SEQ ID NO: 24)
MKFLVNVALVFMVVYISYIYA

### Example 2. Differential Ligand Inhibition by Receptor Fusion Protein Variants in Cell-Based Assay

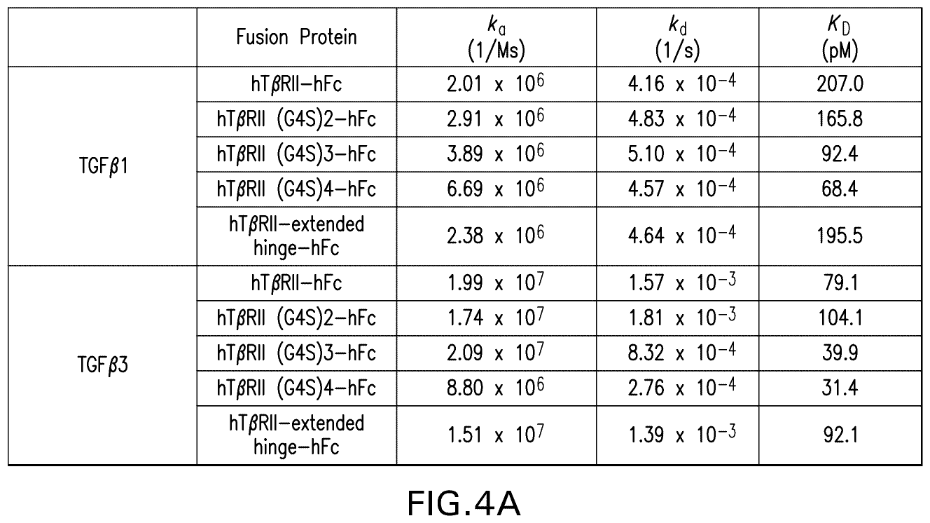

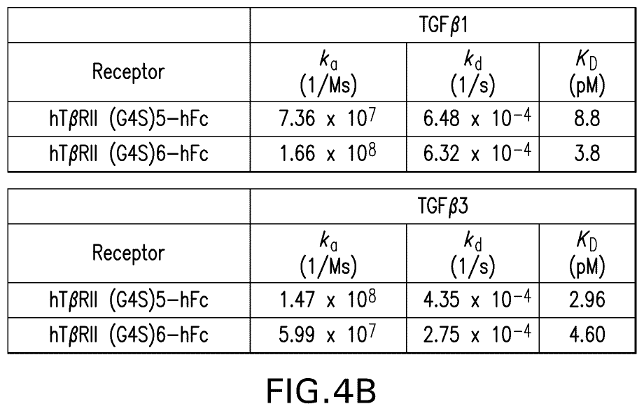

Affinities of TGFβ1, TGFβ2 and TGFβ3 for hTβRII (G4S)2-hFc; hTβRII (G4S)3-hFc; hTβRII (G4S)4-hFc; hTβRII-hFc; and hTβRII extended hinge-hFc proteins were evaluated in vitro with a Biacore™ instrument, and the results are summarized in FIGS. 4A and 4B. Each of the fusion proteins was capable of binding TGFβ1 and TGFβ3 with high affinity, but the constructs having linker lengths longer than or equal to (G4S)4 were surprisingly capable of binding to both TGFβ1 and TGFβ3 with higher affinity than constructs having linker lengths shorter than (G4S)4. Binding between TGFβ2 and any of the constructs was low or transient. Deglycosylation of the constructs did not change binding.

A reporter gene assay in A549 cells was used to determine the ability of hTβRII-hFc variants to inhibit activity of TGFβ1, TGFβ2 and TGFβ3. This assay is based on a human lung carcinoma cell line transfected with a pGL3(CAGA) 12 reporter plasmid (Dennler et al, 1998, EMBO 17: 3091-3100) as well as a Renilla reporter plasmid (pRLCMV) to control for transfection efficiency. The CAGA motif is present in the promoters of TGFβ-responsive genes (for example, PAI-1), so this vector is of general use for factors signaling through SMAD2 and SMAD3.

On the first day of the assay, A549 cells (ATCC®: CCL-185™) were distributed in 48-well plates. On the second day, a solution containing pGL3(CAGA)12, pRLCMV, X-tremeGENE 9 (Roche Applied Science), and OptiMEM (Invitrogen) was preincubated, then added to Eagle's minimum essential medium (EMEM, ATCC®) supplemented with 0.1% BSA, which was applied to the plated cells for incubation overnight at 37° C., 5% CO₂. On the third day, medium was removed, and cells were incubated overnight at 37° C., 5% CO₂ with a mixture of ligands and inhibitors prepared as described below.

Serial dilutions of test articles were made in a 48-well plate in assay buffer (EMEM+0.1% BSA). An equal volume of assay buffer containing the test ligand was added to obtain a final ligand concentration equal to the EC50 determined previously. Human TGFβ1, human TGFβ2, and human TGFβ3 were obtained from PeproTech. Test solutions were incubated at 37° C. for 30 minutes, then a portion of the mixture was added to all wells. After incubation with test solutions overnight, cells were rinsed with phosphate-buffered saline, then lysed with passive lysis buffer (Promega E1941) and stored overnight at −70° C. On the fourth and final day, plates were warmed to room temperature with gentle shaking. Cell lysates were transferred in duplicate to a chemiluminescence plate (96-well) and analyzed in a luminometer with reagents from a Dual-Luciferase Reporter Assay system (Promega E1980) to determine normalized luciferase activity.

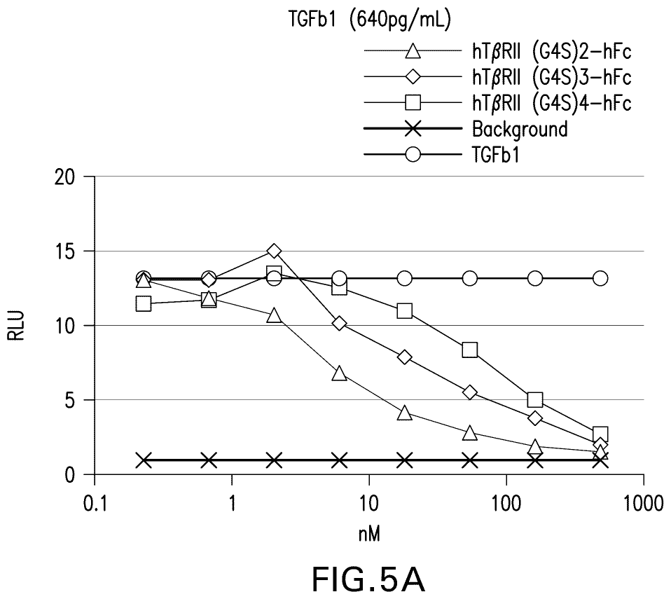

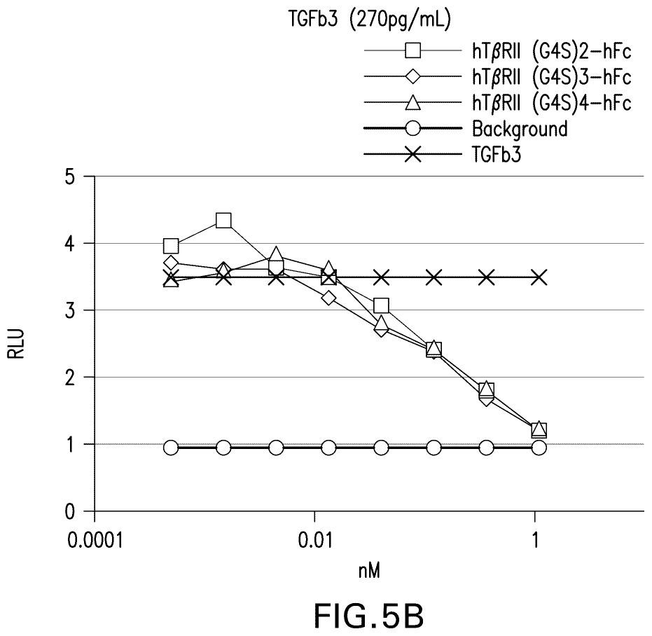

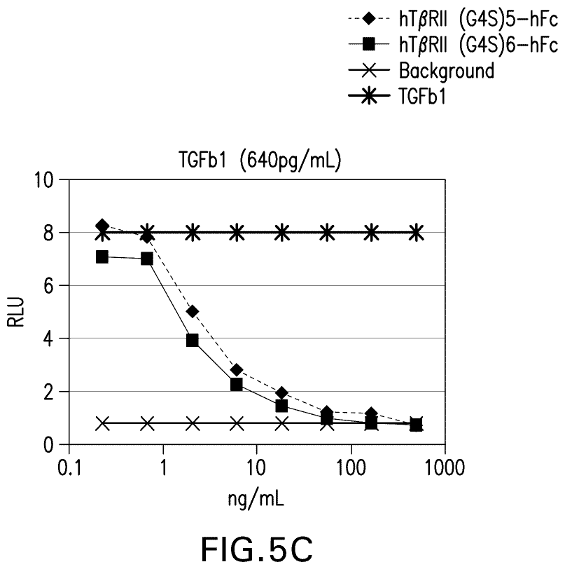

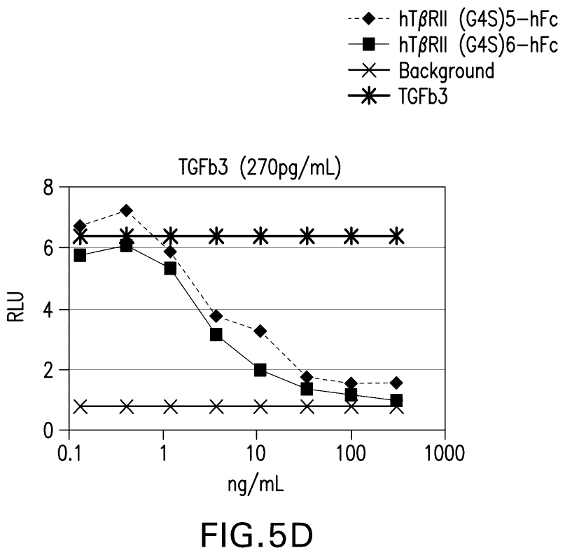

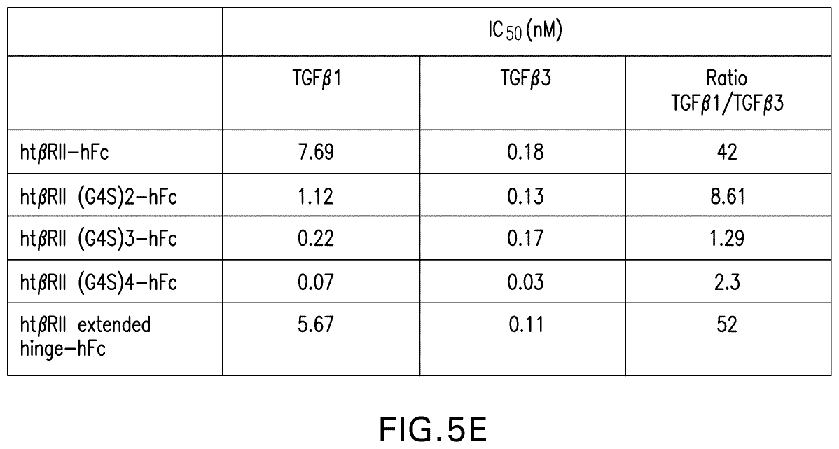

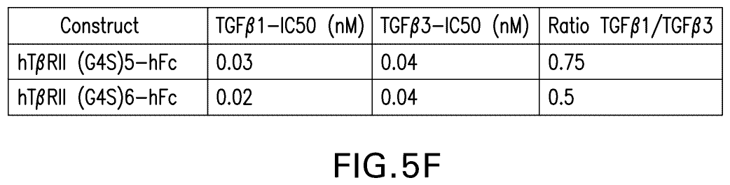

As illustrated in FIGS. 5A-5F, the hTβRII (G4S)2-hFc; hTβRII (G4S)3-hFc; hTβRII (G4S)4-hFc; hTβRII (G4S)5-hFc; hTβRII (G4S)6-hFc; hTβRII-hFc; and hTβRII extended hinge-hFc proteins all were capable of inhibiting both TGFβ1 and TGFβ3. Interestingly, while there was a correlation between improved TGFβ1 and TGFβ3 inhibition and linker length for the the hTβRII (G4S)2-hFc; hTβRII (G4S)3-hFc and hTβRII (G4S)4-hFc constructs (FIG. 5E), this improvement trend appeared to have plateaued for hTβRII (G4S)5-hFc and hTβRII (G4S)6-hFc constructs (FIG. 5F).

### Example 3. Generation of an ActRIIB:TβRII Heterodimer

Soluble ActRIIB-Fc:TβRII-Fc heteromeric complexes comprising the extracellular domains of human ActRIIB and human TβRII, which are each separately fused to an Fc domain with a linker positioned between the extracellular domain and the Fc domain, were constructed. The individual constructs are referred to as ActRIIB-Fc fusion polypeptide and TβRII-Fc fusion polypeptide, respectively, and the sequences for each are provided below.

A methodology for promoting formation of ActRIIB-Fc:TβRII-Fc heteromeric complexes, as opposed to ActRIIB-Fc or TβRII-Fc homodimeric complexes, is to introduce alterations in the amino acid sequence of the Fc domains to guide the formation of asymmetric heteromeric complexes. Many different approaches to making asymmetric interaction pairs using Fc domains are described in this disclosure.

In one approach, illustrated in the ActRIIB-Fc and TβRII-Fc polypeptide sequences of SEQ ID NOs: 82, 84, 85 and 87, respectively, one Fc domain is altered to introduce cationic amino acids at the interaction face, while the other Fc domain is altered to introduce anionic amino acids at the interaction face. ActRIIB-Fc fusion polypeptide and TβRII-Fc fusion polypeptide each employ the tissue plasminogen activator (TPA) leader: MDAMKRGLCCVLLLCGAVFVSP (SEQ ID NO: 23) and a (G4S)4 linker positioned between the ActRIIB or TβRII extracellular portion and the modified Fc portion.

The ActRIIB-Fc polypeptide sequence (SEQ ID NO: 82) is shown below:

(SEQ ID NO: 82)

| | |
|---|---|
| 1 | MDAMKRGLCC VLLLCGAVFV SPGASGRGEA ETRECIYYNA |
| | NWELERTNQS |
| 51 | GLERCEGEQD KRLHCYASWR NSSGTIELVK KGCWLDDFNC |
| | YDRQECVATE |
| 101 | ENPQVYFCCC EGNFCNERFT HLPEAGGPEV TYEPPPTAPT |
| | GGGGSGGGGS |
| 151 | GGGGSGGGGS THTCPPCPAP ELLGGPSVFL FPPKPKDTLM |
| | ISRTPEVTCV |
| 201 | VVDVSHEDPE VKFNWYVDGV EVHNAKTKPR EEQYNSTYRV |
| | VSVLTVLHQD |
| 251 | WLNGKEYKCK VSNKALPAPI EKTISKAKGQ PREPQVYTLP |
| | PSRKEMTKNQ |
| 301 | VSLTCLVKGF YPSDIAVEWE SNGQPENNYK TTPPVLKSDG |
| | SFFLYSKLTV |
| 351 | DKSRWQQGNV FSCSVMHEAL HNHYTQKSLS LSPGK |

The leader (signal) sequence and linker are underlined. To promote formation of ActRIIB-Fc:TβRII-Fc heterodimer rather than either of the possible homodimeric complexes, two amino acid substitutions (replacing acidic amino acids with lysine) can be introduced into the Fc domain of the ActRIIB fusion protein as indicated by double underline above. The amino acid sequence of SEQ ID NO: 82 may optionally be provided with lysine (K) removed from the C-terminus.

This ActRIIB-Fc fusion protein is encoded by the following nucleic acid sequence (SEQ ID NO: 83):

| (SEQ ID NO: 83) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CTTCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAG GTTCTGGAGG TGGAGGAAGT |
| 451 | GGTGGAGGTG GTTCTGGAGG TGGTGGAAGT ACTCACACAT GCCCACCGTG |
| 501 | CCCAGCACCT GAACTCCTGG GGGGACCGTC AGTCTTCCTC TTCCCCCCAA |
| 551 | AACCCAAGGA CACCCTCATG ATCTCCCGGA CCCCTGAGGT CACATGCGTG |
| 601 | GTGGTGGACG TGAGCCACGA AGACCCTGAG GTCAAGTTCA ACTGGTACGT |
| 651 | GGACGGCGTG GAGGTGCATA ATGCCAAGAC AAAGCCGCGG GAGGAGCAGT |
| 701 | ACAACAGCAC GTACCGTGTG GTCAGCGTCC TCACCGTCCT GCACCAGGAC |
| 751 | TGGCTGAATG GCAAGGAGTA CAAGTGCAAG GTCTCCAACA AAGCCCTCCC |
| 801 | AGCCCCCATC GAGAAAACCA TCTCCAAAGC CAAAGGGCAG CCCCGAGAAC |
| 851 | CACAGGTGTA CACCCTGCCC CCATCCCGGA AGGAGATGAC CAAGAACCAG |
| 901 | GTCAGCCTGA CCTGCCTGGT CAAAGGCTTC TATCCCAGCG ACATCGCCGT |
| 951 | GGAGTGGGAG AGCAATGGGC AGCCGGAGAA CAACTACAAG ACCACGCCTC |
| 1001 | CCGTGCTGAA GTCCGACGGC TCCTTCTTCC TCTATAGCAA GCTCACCGTG |
| 1051 | GACAAGAGCA GGTGGCAGCA GGGGAACGTC TTCTCATGCT CCGTGATGCA |
| 1101 | TGAGGCTCTG CACAACCACT ACACGCAGAA GAGCCTCTCC CTGTCTCCGG |
| 1151 | GTAAATGA |

The processed ActRIIB-Fc fusion polypeptide (SEQ ID NO: 84) is as follows, and may optionally be provided with lysine (K) removed from the C-terminus.

(SEQ ID NO: 84)

| | |
|---|---|
| 1 | GRGEAETREC IYYNANWELE RTNQSGLERC EGEQDKRLHC |
| | YASWRNSSGT IELVKKGCWL |
| 61 | DDFNCYDRQE CVATEENPQV YFCCCEGNFC NERFTHLPEA |
| | GGPEVTYEPP PTAPTGGGGS |
| 121 | GGGGSGGGGS GGGGSTHTCP PCPAPELLGG PSVFLFPPKP |
| | KDTLMISRTP EVTCVVVDVS |
| 181 | HEDPEVKFNW YVDGVEVHNA KTKPREEQYN STYRVVSVLT |
| | VLHQDWLNGK EYKCKVSNKA |
| 241 | LPAPIEKTIS KAKGQPREPQ VYTLPPSRKE MTKNQVSLTC |
| | LVKGFYPSDI AVEWESNGQP |
| 301 | ENNYKTTPPV LKSDGSFFLY SKLTVDKSRW QQGNVFSCSV |
| | MHEALHNHYT QKSLSLSPGK |

The complementary form of TβRII-Fc fusion polypeptide (SEQ ID NO: 85) is as follows:

(SEQ ID NO: 85)

| | |
|---|---|
| 1 | MDAMKRGLCC VLLLCGAVFV SPGATIPPHV QKSDVEMEAQ |
| | KDEIICPSCN |
| 51 | RTAHPLRHIN NDMIVTDNNG AVKFPQLCKF CDVRFSTCDN |
| | QKSCMSNCSI |
| 101 | TSICEKPQEV CVAVWRKNDE NITLETVCHD PKLPYHDFIL |
| | EDAASPKCIM |
| 151 | KEKKKPGETF FMCSCSSDEC NDNIIFSEEY NTSNPDTGGG |
| | GSGGGGSGGG |
| 201 | GSGGGGSTHT CPPCPAPELL GGPSVFLFPP KPKDTLMISR |
| | TPEVTCVVVD |
| 251 | VSHEDPEVKF NWYVDGVEVH NAKTKPREEQ YNSTYRVVSV |
| | LTVLHQDWLN |
| 301 | GKEYKCKVSN KALPAPIEKT ISKAKGQPRE PQVYTLPPSR |
| | EEMTKNQVSL |
| 351 | TCLVKGFYPS DIAVEWESNG QPENNY [symbol] TTP PVLDSDGSFF |
| | LYS [symbol] LTVDKS |
| 401 | RWQQGNVFSC SVMHEALHNH YTQKSLSLSP GK |

The leader sequence and linker are underlined. To guide heterodimer formation with the ActRIIB-Fc fusion polypeptide of SEQ ID NOs: 82 and 84 above, two amino acid substitutions (replacing lysines with aspartic acids) can be introduced into the Fc domain of the TβRII-Fc fusion polypeptide as indicated by double underline above. The amino acid sequence of SEQ ID NO: 85 may optionally be provided with lysine (K) added at the C-terminus.

This TβRII-Fc fusion protein is encoded by the following nucleic acid (SEQ ID NO: 86):

| (SEQ ID NO: 86) | |
|---|---|
| 1 | ATGGATGCGA TGAAACGCGG CCTGTGCTGC GTGCTGCTGC TGTGCGGCGC |
| 51 | GGTGTTTGTG AGCCCGGGCG CCACCATTCC GCCGCATGTG CAGAAAAGCG |
| 101 | ATGTGGAAAT GGAAGCGCAG AAAGATGAAA TTATTTGCCC GAGCTGCAAC |
| 151 | CGCACCGCGC ATCCGCTGCG CCATATTAAC AACGATATGA TTGTGACCGA |
| 201 | TAACAACGGC GCGGTGAAAT TTCCGCAGCT GTGCAAATTT TGCGATGTGC |
| 251 | GCTTTAGCAC CTGCGATAAC CAGAAAAGCT GCATGAGCAA CTGCAGCATT |
| 301 | ACCAGCATTT GCGAAAAACC GCAGGAAGTG TGCGTGGCGG TGTGGCGCAA |
| 351 | AAACGATGAA AACATTACCC TGGAAACCGT GTGCCATGAT CCGAAACTGC |
| 401 | CGTATCATGA TTTTATTCTG GAAGATGCGG CGAGCCCGAA ATGCATTATG |
| 451 | AAAGAAAAAA AAAAACCGGG CGAAACCTTT TTTATGTGCA GCTGCAGCAG |
| 501 | CGATGAATGC AACGATAACA TTATTTTTAG CGAAGAATAT AACACCAGCA |
| 551 | ACCCGGATAC CGGTGGCGGC GGCAGCGGCG GCGGCGGCAG CGGCGGCGGC |
| 601 | GGCAGCGGCG GCGGCGGCAG CACCCATACC TGCCCGCCGT GCCCGGCGCC |
| 651 | GGAACTGCTG GGCGGCCCGA GCGTGTTTCT GTTTCCGCCG AAACCGAAAG |
| 701 | ATACCCTGAT GATTAGCCGC ACCCCGGAAG TGACCTGCGT GGTGGTGGAT |
| 751 | GTGAGCCATG AAGATCCGGA AGTGAAATTT AACTGGTATG TGGATGGCGT |
| 801 | GGAAGTGCAT AACGCGAAAA CCAAACCGCG CGAAGAACAG TATAACAGCA |
| 851 | CCTATCGCGT GGTGAGCGTG CTGACCGTGC TGCATCAGGA TTGGCTGAAC |
| 901 | GGCAAAGAAT ATAAATGCAA AGTGAGCAAC AAAGCGCTGC CGGCGCCGAT |
| 951 | TGAAAAAACC ATTAGCAAAG CGAAAGGCCA GCCGCGCGAA CCGCAGGTGT |
| 1001 | ATACCCTGCC GCCGAGCCGC GAAGAAATGA CCAAAAACCA GGTGAGCCTG |
| 1051 | ACCTGCCTGG TGAAAGGCTT TTATCCGAGC GATATTGCGG TGGAATGGGA |
| 1101 | AAGCAACGGC CAGCCGGAAA ACAACTATGA TACCACCCCG CCGGTGCTGG |
| 1151 | ATAGCGATGG CAGCTTTTTT CTGTATAGCG ATCTGACCGT GGATAAAAGC |
| 1201 | CGCTGGCAGC AGGGCAACGT GTTTAGCTGC AGCGTGATGC ATGAAGCGCT |
| 1251 | GCATAACCAT TATACCCAGA AAAGCCTGAG CCTGAGCCCG GGCGATGATG |
| 1301 | ATGATAAAGC GCATCATCAT CATCATCATT AA |

The processed TβRII-Fc fusion protein sequence (SEQ ID NO: 87) is as follows and may optionally be provided with lysine (K) added at the C-terminus.

(SEQ ID NO: 87)

| | |
|---|---|
| 1 | TIPPHVQKSD VEMEAQKDEI ICPSCNRTAH PLRHINNDMI |
| | VTDNNGAVKF PQLCKFCDVR |
| 61 | FSTCDNQKSC MSNCSITSIC EKPQEVCVAV WRKNDENITL |
| | ETVCHDPKLP YHDFILEDAA |
| 121 | SPKCIMKEKK KPGETFFMCS CSSDECNDNI IFSEEYNTSN |
| | PDTGGGGSGG GGSGGGGSGG |
| 181 | GGSTHTCPPC PAPELLGGPS VFLFPPKPKD TLMISRTPEV |
| | TCVVVDVSHE DPEVKFNWYV |
| 241 | DGVEVHNAKT KPREEQYNST YRVVSVLTVL HQDWLNGKEY |
| | KCKVSNKALP APIEKTISKA |
| 301 | KGQPREPQVY TLPPSREEMT KNQVSLTCLV KGFYPSDIAV |
| | EWESNGQPEN NYDTTPPVLD |
| 361 | SDGSFFLYSD LTVDKSRWQQ GNVFSCSVMH EALHNHYTQK |
| | SLSLSPGK |

The ActRIIB-Fc and TβRII-Fc proteins of SEQ ID NO: 84 and SEQ ID NO: 87, respectively, may be co-expressed and purified from a CHO cell line, to give rise to a heteromeric complex comprising ActRIIB-Fc:TβRII-Fc.

In another approach to promote the formation of heteromultimer complexes using asymmetric Fc fusion proteins the Fc domains are altered to introduce complementary hydrophobic interactions and an additional intermolecular disulfide bond as illustrated in the ActRIIB-Fc and TβRII-Fc polypeptide sequences of SEQ ID NOs: 88-90 and 91-93, respectively. The ActRIIB-Fc fusion polypeptide and TβRII-Fc fusion polypeptide each employ the tissue plasminogen activator (TPA) leader.

The ActRIIB-Fc polypeptide sequence (SEQ ID NO: 88) is shown below:

(SEQ ID NO: 88)

| | |
|---|---|
| 1 | MDAMKRGLCC VLLLCGAVFV SPGASGRGEA ETRECIYYNA |
| | NWELERTNQS |
| 51 | GLERCEGEQD KRLHCYASWR NSSGTIELVK KGCWLDDFNC |
| | YDRQECVATE |
| 101 | ENPQVYFCCC EGNFCNERFT HLPEAGGPEV TYEPPPTAPT |
| | GGGGSGGGGS |
| 151 | GGGGSGGGGS THTCPPCPAP ELLGGPSVFL FPPKPKDTLM |
| | ISRTPEVTCV |
| 201 | VVDVSHEDPE VKFNWYVDGV EVHNAKTKPR EEQYNSTYRV |
| | VSVLTVLHQD |
| 251 | WLNGKEYKCK VSNKALPAPI EKTISKAKGQ PREPQVYTLP |
| | PCREEMTKNQ |
| 301 | VSLWCLVKGF YPSDIAVEWE SNGQPENNYK TTPPVLDSDG |
| | SFFLYSKLTV |
| 351 | DKSRWQQGNV FSCSVMHEAL HNHYTQKSLS LSPGK |

The leader (signal) sequence and linker are underlined. To promote formation of the ActRIIB-Fc:TβRII-Fc heterodimer rather than either of the possible homodimeric complexes, two amino acid substitutions (replacing a serine with a cysteine and a threonine with a trytophan) can be introduced into the Fc domain of the fusion protein as indicated by double underline above. The amino acid sequence of SEQ ID NO: 88 may optionally be provided with lysine (K) removed from the C-terminus.

This ActRIIB-Fc fusion protein is encoded by the following nucleic acid sequence (SEQ ID NO: 89):

(SEQ ID NO: 89)

| | |
|---|---|
| 1 | ATGGATGCAA TGAAGAGAGG GCTCTGCTGT GTGCTGCTGC |
| | TGTGTGGAGC |
| 51 | AGTCTTCGTT TCGCCCGGCG CCTCTGGGCG TGGGGAGGCT |
| | GAGACACGGG |
| 101 | AGTGCATCTA CTACAACGCC AACTGGGAGC TGGAGCGCAC |
| | CAACCAGAGC |
| 151 | GGCCTGGAGC GCTGCGAAGG CGAGCAGGAC AAGCGGCTGC |
| | ACTGCTACGC |
| 201 | CTCCTGGCGC AACAGCTCTG GCACCATCGA GCTCGTGAAG |
| | AAGGGCTGCT |
| 251 | GGCTAGATGA CTTCAACTGC TACGATAGGC AGGAGTGTGT |
| | GGCCACTGAG |
| 301 | GAGAACCCCC AGGTGTACTT CTGCTGCTGT GAAGGCAACT |
| | TCTGCAACGA |
| 351 | GCGCTTCACT CATTTGCCAG AGGCTGGGGG CCCGGAAGTC |
| | ACGTACGAGC |
| 401 | CACCCCCGAC AGCCCCCACC GGTGGTGGAG GTTCTGGAGG |
| | TGGAGGAAGT |
| 451 | GGTGGAGGTG GTTCTGGAGG TGGTGGAAGT ACTCACACAT |
| | GCCCACCGTG |
| 501 | CCCAGCACCT GAACTCCTGG GGGGGCCGTC AGTCTTCCTC |
| | TTCCCCCCAA |
| 551 | AACCCAAGGA CACCCTCATG ATCTCCCGGA CCCCTGAGGT |
| | CACATGCGTG |
| 601 | GTGGTGGACG TGAGCCACGA AGACCCTGAG GTCAAGTTCA |
| | ACTGGTACGT |
| 651 | GGACGGCGTG GAGGTGCATA ATGCCAAGAC AAAGCCGCGG |
| | GAGGAGCAGT |
| 701 | ACAACAGCAC GTACCGTGTG GTCAGCGTCC TCACCGTCCT |
| | GCACCAGGAC |
| 751 | TGGCTGAATG GCAAGGAGTA CAAGTGCAAG GTCTCCAACA |
| | AAGCCCTCCC |
| 801 | AGCCCCCATC GAGAAAACCA TCTCCAAAGC CAAAGGGCAG |
| | CCCCGAGAAC |
| 851 | CACAGGTGTA CACCCTGCCC CCATGCCGGG AGGAGATGAC |
| | CAAGAACCAG |
| 901 | GTCAGCCTGT GGTGCCTGGT CAAAGGCTTC TATCCCAGCG |
| | ACATCGCCGT |
| 951 | GGAGTGGGAG AGCAATGGGC AGCCGGAGAA CAACTACAAG |
| | ACCACGCCTC |
| 1001 | CCGTGCTGGA CTCCGACGGC TCCTTCTTCC TCTATAGCAA |
| | GCTCACCGTG |
| 1051 | GACAAGAGCA GGTGGCAGCA GGGGAACGTC TTCTCATGCT |
| | CCGTGATGCA |
| 1101 | TGAGGCTCTG CACAACCACT ACACGCAGAA GAGCCTCTCC |
| | CTGTCTCCGG |
| 1151 | GTAAATGA |

The processed ActRIIB-Fc fusion polypeptide is as follows:

(SEQ ID NO: 90)

| | |
|---|---|
| 1 | GRGEAETREC IYYNANWELE RTNQSGLERC EGEQDKRLHC |
| | YASWRNSSGT IELVKKGCWL |
| 61 | DDFNCYDRQE CVATEENPQV YFCCCEGNFC NERFTHLPEA |
| | GGPEVTYEPP PTAPTGGGGS |
| 121 | GGGGSGGGGS GGGGSTHTCP PCPAPELLGG PSVFLFPPKP |
| | KDTLMISRTP EVTCVVVDVS |
| 181 | HEDPEVKFNW YVDGVEVHNA KTKPREEQYN STYRVVSVLT |
| | VLHQDWLNGK EYKCKVSNKA |
| 241 | LPAPIEKTIS KAKGQPREPQ VYTLPPCREE MTKNQVSLWC |
| | LVKGFYPSDI AVEWESNGQP |
| 301 | ENNYKTTPPV LDSDGSFFLY SKLTVDKSRW QQGNVFSCSV |
| | MHEALHNHYT QKSLSLSPGK |

The complementary form of TβRII-Fc fusion polypeptide (SEQ ID NO: 91) is as follows and may optionally be provided with lysine (K) removed from the C-terminus.

(SEQ ID NO: 91)

| | |
|---|---|
| 1 | MDAMKRGLCC VLLLCGAVFV SPGATIPPHV QKSDVEMEAQ |
| | KDEIICPSCN |
| 51 | RTAHPLRHIN NDMIVTDNNG AVKFPQLCKF CDVRFSTCDN |
| | QKSCMSNCSI |
| 101 | TSICEKPQEV CVAVWRKNDE NITLETVCHD PKLPYHDFIL |
| | EDAASPKCIM |
| 151 | KEKKKPGETF FMCSCSSDEC NDNIIFSEEY NTSNPDTGGG |
| | GSGGGGSGGG |
| 201 | GSGGGGSTHT CPPCPAPELL GGPSVFLFPP KPKDTLMISR |
| | TPEVTCVVVD |
| 251 | VSHEDPEVKF NWYVDGVEVH NAKTKPREEQ YNSTYRVVSV |
| | LTVLHQDWLN |
| 301 | GKEYKCKVSN KALPAPIEKT ISKAKGQPRE PQVCTLPPSR |
| | EEMTKNQVSL |
| 351 | SCAVKGFYPS DIAVEWESNG QPENNYKTTP PVLDSDGSFF |
| | LVSKLTVDKS |
| 401 | RWQQGNVFSC SVMHEALHNH YTQKSLSLSP GK |

The leader sequence and the linker are underlined. To guide heterodimer formation with the ActRIIB-Fc fusion polypeptide of SEQ ID NOs: 88 and 91 above, four amino acid substitutions can be introduced into the Fc domain of the TβRII fusion polypeptide as indicated by double underline above. The amino acid sequence of SEQ ID NO: 91 may optionally be provided with lysine (K) removed from the C-terminus.

This A TβRII-Fc fusion protein is encoded by the following nucleic acid sequence (SEQ ID NO: 92):

(SEQ ID NO: 92)

| | |
|---|---|
| 1 | ATGGATGCAA TGAAGAGAGG GCTCTGCTGT GTGCTGCTGC |
| | TGTGTGGAGC |
| 51 | AGTCTTCGTT TCGCCCGGCG CCACGATCCC ACCGCACGTT |
| | CAGAAGTCGG |
| 101 | ATGTGGAAAT GGAGGCCCAG AAAGATGAAA TCATCTGCCC |
| | CAGCTGTAAT |
| 151 | AGGACTGCCC ATCCACTGAG ACATATTAAT AACGACATGA |
| | TAGTCACTGA |
| 201 | CAACAACGGT GCAGTCAAGT TTCCACAACT GTGTAAATTT |
| | TGTGATGTGA |
| 251 | GATTTTCCAC CTGTGACAAC CAGAAATCCT GCATGAGCAA |
| | CTGCAGCATC |
| 301 | ACCTCCATCT GTGAGAAGCC ACAGGAAGTC TGTGTGGCTG |
| | TATGGAGAAA |
| 351 | GAATGACGAG AACATAACAC TAGAGACAGT TTGCCATGAC |
| | CCCAAGCTCC |
| 401 | CCTACCATGA CTTTATTCTG GAAGATGCTG CTTCTCCAAA |
| | GTGCATTATG |
| 451 | AAGGAAAAAA AAAAGCCTGG TGAGACTTTC TTCATGTGTT |
| | CCTGTAGCTC |
| 501 | TGATGAGTGC AATGACAACA TCATCTTCTC AGAAGAATAT |
| | AACACCAGCA |
| 551 | ATCCTGACAC CGGTGGTGGA GGTTCTGGAG GTGGAGGAAG |
| | TGGTGGAGGT |
| 601 | GGTTCTGGAG GTGGTGGAAG TACTCACACA TGCCCACCGT |
| | GCCCAGCACC |
| 651 | TGAACTCCTG GGGGGACCGT CAGTCTTCCT CTTCCCCCCA |
| | AAACCCAAGG |
| 701 | ACACCCTCAT GATCTCCCGG ACCCCTGAGG TCACATGCGT |
| | GGTGGTGGAC |
| 751 | GTGAGCCACG AAGACCCTGA GGTCAAGTTC AACTGGTACG |
| | TGGACGGCGT |
| 801 | GGAGGTGCAT AATGCCAAGA CAAAGCCGCG GGAGGAGCAG |
| | TACAACAGCA |
| 851 | CGTACCGTGT GGTCAGCGTC CTCACCGTCC TGCACCAGGA |
| | CTGGCTGAAT |
| 901 | GGCAAGGAGT ACAAGTGCAA GGTCTCCAAC AAAGCCCTCC |
| | CAGCCCCCAT |
| 951 | CGAGAAAACC ATCTCCAAAG CCAAAGGGCA GCCCCGAGAA |
| | CCACAGGTGT |
| 1001 | GCACCCTGCC CCCATCCCGG GAGGAGATGA CCAAGAACCA |
| | GGTCAGCCTG |
| 1051 | TCCTGCGCCG TCAAAGGCTT CTATCCCAGC GACATCGCCG |
| | TGGAGTGGGA |
| 1101 | GAGCAATGGG CAGCCGGAGA ACAACTACAA GACCACGCCT |
| | CCCGTGCTGG |
| 1151 | ACTCCGACGG CTCCTTCTTC CTCGTGAGCA AGCTCACCGT |
| | GGACAAGAGC |
| 1201 | AGGTGGCAGC AGGGGAACGT CTTCTCATGC TCCGTGATGC |
| | ATGAGGCTCT |
| 1251 | GCACAACCAC TACACGCAGA AGAGCCTCTC CCTGTCTCCG |
| | GGTAAATGA |

A processed TβRII-Fc fusion protein sequence is as follows and may optionally be provided with lysine (K) removed from the C-terminus.

(SEQ ID NO: 93)

| | |
|---|---|
| 1 | TIPPHVQKSD VEMEAQKDEI ICPSCNRTAH PLRHINNDMI |
| | VTDNNGAVKF PQLCKFCDVR |
| 61 | FSTCDNQKSC MSNCSITSIC EKPQEVCVAV WRKNDENITL |
| | ETVCHDPKLP YHDFILEDAA |
| 121 | SPKCIMKEKK KPGETFFMCS CSSDECNDNI IFSEEYNTSN |
| | PDTGGGGSGG GGSGGGGSGG |
| 181 | GGSTHTCPPC PAPELLGGPS VFLFPPKPKD TLMISRTPEV |
| | TCVVVDVSHE DPEVKFNWYV |
| 241 | DGVEVHNAKT KPREEQYNST YRVVSVLTVL HQDWLNGKEY |
| | KCKVSNKALP APIEKTISKA |
| 301 | KGQPREPQVC TLPPSREEMT KNQVSLSCAV KGFYPSDIAV |
| | EWESNGQPEN NYKTTPPVLD |
| 361 | SDGSFFLVSK LTVDKSRWQQ GNVFSCSVMH EALHNHYTQK |
| | SLSLSPGK |

ActRIIB-Fc and TβRII-Fc proteins of SEQ ID NO: 90 and SEQ ID NO: 93, respectively, may be co-expressed and purified from a CHO cell line, to give rise to a heteromeric complex comprising ActRIIB-Fc:TβRII-Fc.

In order to compare the activity of the ActRIIB-Fc:TβRII-Fc heterodimers, ActRIIB-Fc and TβRII-Fc homodimers were generated, which each comprise either the ActRIIB or TβRII extracellular domains as present in any one of SEQ ID NO: 82, 84, 85, 87, 88, 90, 91, or 93; an unmodified hG1Fc domain (promotes homodimer formation); and a (G4S)4 linker positioned between the ActRIIB or TβRII extracellular portion and the unmodified Fc portion. Both of these homodimers were expressed using the TPA leader sequence of SEQ ID NO: 23.

Purification of various heterodimer and homodimers described above could be achieved by a series of column chromatography steps, including, for example, three or more of the following, in any order: protein A chromatography, Q sepharose chromatography, phenylsepharose chromatography, size exclusion chromatography and epitope-based affinity chromatography (e.g., with an antibody or functionally equivalent ligand directed against an epitope on TβRII or ActRIIB), and multimodal chromatography (e.g., with resin containing both electrostatic and hydrophobic ligands). The purification could be completed with viral filtration and buffer exchange.

### Example 4. Differential Ligand Inhibition by Receptor Fusion Protein Variants in Cell-Based Assay

A reporter gene assay in A549 cells was used to determine the ability of an ActRIIB-Fc:TβRII-Fc heterodimer to inhibit activity of TGFβ1, TGFβ2, TGFβ3, activin A, activin B, GDF11, GDF8, BMP9, and BMP10 and compared to the inhibiting activity of an ActRIIB-Fc homodimer and TβRII-Fc homodimer, which are all described above in Example 3. This assay is based on a human lung carcinoma cell line transfected with a pGL3(CAGA)12 reporter plasmid (Dennler et al, 1998, EMBO 17: 3091-3100) as well as a Renilla reporter plasmid (pRLCMV) to control for transfection efficiency. The CAGA motif is present in the promoters of TGFβ-responsive genes (for example, PAI-1), so this vector is of general use for factors signaling through SMAD2 and SMAD3.

On the first day of the assay, A549 cells (ATCC®: CCL-185™) were distributed in 48-well plates. On the second day, a solution containing pGL3(CAGA)12, pRLCMV, X-tremeGENE 9 (Roche Applied Science), and OptiMEM (Invitrogen) was preincubated, then added to Eagle's minimum essential medium (EMEM, ATCC®) supplemented with 0.1% BSA, which was applied to the plated cells for incubation overnight at 37° C., 5% CO₂. On the third day, medium was removed, and cells were incubated overnight at 37° C., 5% CO₂ with a mixture of ligands and inhibitors prepared as described below.

Serial dilutions of test articles were made in a 48-well plate in assay buffer (EMEM+0.1% BSA). An equal volume of assay buffer containing the test ligand was added to obtain a final ligand concentration equal to the EC50 determined previously. Test solutions were incubated at 37° C. for 30 minutes, then a portion of the mixture was added to all wells. After incubation with test solutions overnight, cells were rinsed with phosphate-buffered saline, then lysed with passive lysis buffer (Promega E1941) and stored overnight at −70° C. On the fourth and final day, plates were warmed to room temperature with gentle shaking. Cell lysates were transferred in duplicate to a chemiluminescence plate (96-well) and analyzed in a luminometer with reagents from a Dual-Luciferase Reporter Assay system (Promega E1980) to determine normalized luciferase activity.

As illustrated in FIG. 12, the TβRII-Fc homodimer was capable of inhibiting TGFβ 1 and TGFβ3 in this cell-based assay but did not inhibit TGFβ2, activin A, activin B, GDF11, GDF8, BMP9, or BMP10. In contrast, the ActRIIB-Fc homodimer was capable of inhibiting activin A, activin B, GDF11, GDF8, BMP9, and BMP10 but did not inhibit TGFβ1, TGFβ2, or TGFβ3. The ActRIIB-Fc:TβRII-Fc heterodimer was capable of inhibiting TGFβ1, TGFβ3, activin A, activin B, GDF11, GDF8, and BMP10 but did not inhibit BMP9 or TGFβ2. These data demonstrate that ActRIIB-Fc:TβRII-Fc heterodimers retain many of potent inhibitor characteristics of ActRIIB-Fc and TβRII-Fc homodimers and thus represent an interesting class of ligand traps that are uniquely capable of affecting two distinct groups of Smad 2/3-related TGFβ superfamily ligands (i.e., the TGFβs and activin/GDFs). Moreover, the ActRIIB-Fc:TβRII-Fc heterodimer did not inhibit BMP9, and thus with respect to ActRIIB-associated ligands, ActRIIB-Fc:TβRII-Fc is a more selective antagonist than an ActRIIB homodimer. Accordingly, ActRIIB-Fc:TβRII-Fc heterodimers will be more useful than ActRIIB homodimer in certain applications where such selective antagonism is desired in combination with inhibition of TGFβ1 and TGFβ3.

### Example 5. Synthesis of Alternative Multispecific Binders

Multispecific binders capable of binding to both GDF8 and TGFβ via follistatin-TGFβRII or αGDF8-TGFβRII will be generated.

A fusion protein follistatin-TGFβRII consisting of the binding domains of follistatin and TGFBRII (referred to in this example as FS288.Fc (G4S)4-TGFβBRII) will be designed to be in a single bifunctional construct. The design of FS288.Fc (G4S)4-TGFβRII consists of the follistatin native signal peptide, followed by the follistatin-288 isoform (SEQ ID NO: 111), a TGGG linker to the Fc IgG2 domain (SEQ ID NO: 163) which includes the hinge region that contains two disulfide bonds, a (G4S)4 linker (SEQ ID NO: 165) and then TGFβRII ECD (SEQ ID NO: 170) (FIG. 15A). In some embodiments, the FS288.Fc (G4S)4-TGFβRII will comprise the amino acid sequence of SEQ ID NO: 180 or 181.

A second protein will be developed that will include the TGFBRII portion and an antigen-binding fragment portion capable of binding GDF8 (referred to in this example as αGDF8-hIgG1-(G4S)4-TGFβRII). αGDF8-hIgG1-(G4S)4-TGFBRII will be made by the assembly of two protein sub-molecules. One contains the sequence of the αGDF8 antibody variable heavy chain region (SEQ ID NO: 167), followed by the sequences of human IgG1 constant region (SEQ ID NO: 168) which includes the hinge region with two disulfide bonds, a (G4S)4 linker (SEQ ID NO: 169) and then TGFβRII ECD (SEQ ID NO: 170) (FIG. 15B). The second protein sub-molecule contains the sequence of the αGDF8 antibody variable light chain region (SEQ ID NO: 174), followed by sequence of the human kappa constant light chain region (SEQ ID NO: 175). The signal peptide (SEQ ID NO: 176) is used so that all the proteins expressed will be secreted outside of the cells and into the condition media. The DNA constructs of those two new molecules will either be synthesized or cloned by using existing constructs. In some embodiments, the αGDF8-hIgG1-(G4S)4-TGFBRII will comprise the amino acid sequences of SEQ ID NOs: 172 and 182.

A small scale expression of the newly constructed plasmids will be performed in HEK293FT cells to verify if the constructs are made and if cells can express the molecule properly before investing materials and time into larger scale transfections. HEK293FT cells exhibit high transfection efficiency and protein production and will be used for small scale expression experiments. The protein production and quality will be checked via Western Blot assay and detecting with an anti-IgG Fc antibody.

After verifying the expression of these two new molecules in HEK293FT cells, all the studied molecules will be expressed in EXPI-CHO cells. Chinese hamster ovary (CHO) cells, an epithelial cell line derived from the ovary of the Chinese hamster, are the most commonly used mammalian hosts for industrial production of recombinant proteins. EXPI-CHO is a subtype of CHO cells. Conditioned media will be harvested and purified via mAb Protein A drip column. mAb columns made of Protein A bind to the heavy chain constant region (Fc) of IgG proteins, therefore this type of purification can yield high purified Fc-fusion protein in single step. After collecting the purified protein, the studied proteins will be used for ELISA analysis and Reporter Gene Assay (RGA) to characterize their binding to ligands and their activity to alter ligand-mediated signaling pathways in cell based assays.

ELISA is used to detect and quantify proteins via high specific protein to protein interactions. The plate will be coated with various ligands: GDF8, GDF11, Activin A, and Activin B to bind FS288; GDF11 and GDF8 to bind αGDF8 antibody; TGFβ1, TGFβ2, TGFβ3 to bind TGFβRII. FS288.Fc (G4S)4-TGFβBRII, αGDF8-hIgG1-(G4S)4-TGFβRII and their control molecule, αGDF8 antibody, FS288.Fc, and TGFβRII.Fc will be applied to the plate coated with ligands to determine binding. Finally anti-IgG Fe antibody will be used to detect and amplify the binding signals. As a negative control, wells coated with proteins of interest without ligands will be used to determine if there is any background signal because of non-specific binding.

RGA is a cell based assay used to characterize the activation of certain signaling pathways. The RGA assay may be used to investigate the activation of TGFβ SMAD 2/3 signaling activity using a reporter gene plasmid that contains CAGA12 as the response element and firefly luciferase as the reporter gene. Another plasmid containing CMV-renilla luciferase will be used as a transfection control. Upon SMAD 2/3-related ligands binding to cell surface receptors, intracellular SMAD signaling complexes will translocate to the nucleus and bind to CAGA12 and stimulate promoter activation and transcription of luciferase. When the studied molecules are hypothesized to trap specific ligands, the activation of CAGA12 mediated luciferase activity decreases accordingly.

It is expected that FS288.Fc (G4S)4-TGFβBRII to bind all tested ligands via ELISA and to trap and neutralize all tested ligands signaling activity via RGA. However, for αGDF8-hIgG1-(G4S)4-TGFβRII, because αGDF8 antibody only binds GDFs but not Activins, the binding and thus the neutralizing of this molecule to Activins is not expected. With the proposed finding, these two proteins can potentially be used as therapies for patients with DMD and increase their quality of life by improving muscle function.

The FS288.Fc (G4S)4-TGFβBRII and/or αGDF8-hIgG1-(G4S)4-TGFβRII molecules may also be used to treat a DMD animal model (e.g., the mdx mouse model) or human model.

### Example 6. Synthesis of Four-Armed and Three-Armed ActRIIB-TGFβRII Multimers

A “four-armed” homodimer comprising two ActRIIB-TGFBRII fusions proteins was generated. Each of the ActRIIB-TGFBRII fusions proteins (referred to as ActRIIB-Fc-(G4S)4-TGFBRII) in the homodimer included the binding domains of ActRIIB and TGFBRII and was designed in a single bifunctional construct. The design of ActRIIB-Fc-(G4S)4-TGFBRII included an ActRIIB polypeptide portion (SEQ ID NO: 51), followed by a GGG linker, followed by an Fc portion (SEQ ID NO: 163), followed by a linker (SEQ ID NO: 165), and then TGFβRII ECD (SEQ ID NO: 170) (FIG. 16A). The final ActRIIB-Fc-(G4S)4-TGFBRII construct comprised the amino acid sequence of SEQ ID NO: 183.

In addition, a “three-armed” heterodimer comprising: a) one fusion protein comprising a TGFBRII polypeptide portion and an ActRIIB polypeptide portion (referred to as ActRIIB-Fc-(G4S)4G-TGFBRII) and b) a fusion protein comprising a TGFBRII polypeptide portion but lacking an ActRIIB polypeptide portion (referred to as Fc1-(G4S)4G-TGFBRII) was generated. The ActRIIB-Fc-(G4S)4G-TGFBRII protein comprised an ActRIIB polypeptide portion (SEQ ID NO: 51), followed by a GGG linker, followed by an Fc portion (SEQ ID NO: 72, but lacking the C-terminal lysine), followed by a linker (SEQ ID NO: 165), followed by a TGFBRII polypeptide portion (SEQ ID NO: 170). The Fc1-(G4S)4G-TGFBRII protein comprised nine amino acids from CH1 (SNTKVDKRV-SEQ ID NO: 189), followed by a linker (TGGG), followed by an Fc portion (SEQ ID NO: 73), followed by a linker (SEQ ID NO: 165), followed by a TGFBRII polypeptide portion (SEQ ID NO: 170) (FIG. 16B). The final ActRIIB-Fc-(G4S)4G-TGFBRII protein comprised the amino acid sequence of SEQ ID NO: 184, and the final Fc1-(G4S)4G-TGFBRII protein comprised the amino acid sequence of SEQ ID NO: 185.

In addition, the following fusion proteins were also generated and characterized:
a soluble ActRIIB-Fc fusion protein (comprising the amino acid sequence of SEQ ID NO: 190) that forms a homodimer;
a soluble ActRIIB-Fc fusion protein (comprising the amino acid sequence of SEQ ID NO: 191) that heterodimerizes with an Fc protein (comprising the amino acid sequence of SEQ ID NO: 192) to form a “single-arm” ActRIIB heteromultimer;
a soluble Fc-TGFβRII fusion protein (comprising the amino acid sequence of SEQ ID NO: 193) to form a “two-arm” Fc-TGFβRII dimer; referred to herein as Fc1-(G4S)4G-TGFBRII, homodimer (FIG. 17A)
a soluble Fc-TGFβRII fusion protein (comprising the amino acid sequence of SEQ ID NO: 193) to form a “single-arm” Fc-TGFBRII monomer; referred to herein as Fc1hole-(G4S)4G-TGFBRII, monomer (FIG. 17B); and
a soluble TGFβRII-Fc fusion protein (comprising the amino acid sequence of SEQ ID NO: 193) to form a “two-arm” TGFβRII-Fc dimer.

(SEQ ID NO: 190)
GRGEAETRECIYYNANWELERTNQSGLERCEGEQDKRLHCYASWRNSSGTI
ELVKKGCWLDDFNCYDRQECVATEENPQVYFCCCEGNFCNERFTHLPEAGG
PEVTYEPPPTAPTGGGTHTCPPCPAPELLGGPSVFLFPPKPKDTLMISRTP
EVTCVVVDVSHEDPEVKFNWYVDGVEVHNAKTKPREEQYNSTYRVVSVLTV
LHQDWLNGKEYKCKVSNKALPVPIEKTISKAKGQPREPQVYTLPPSREEMT
KNQVSLTCLVKGFYPSDIAVEWESNGQPENNYKTTPPVLDSDGSFFLYSKL
TVDKSRWQQGNVFSCSVMHEALHNHYTQKSLSLSPGK
(SEQ ID NO: 191)

| | |
|---|---|
| 1 | GRGEAETREC IYYNANWELE RTNQSGLERC EGEQDKRLHC |
| | YASWRNSSGT |
| 51 | IELVKKGCWL DDFNCYDRQE CVATEENPQV YFCCCEGNFC |
| | NERFTHLPEA |
| 101 | GGPEVTYEPP PTAPTGGGTH TCPPCPAPEL LGGPSVFLFP |
| | PKPKDTLMIS |
| 151 | RTPEVTCVVV DVSHEDPEVK FNWYVDGVEV HNAKTKPREE |
| | QYNSTYRVVS |
| 201 | VLTVLHQDWL NGKEYKCKVS NKALPAPIEK TISKAKGQPR |
| | EPQVYTLPPS |
| 251 | RKEMTKNQVS LTCLVKGFYP SDIAVEWESN GQPENNYKTT |
| | PPVLKSDGSF |
| 301 | FLYSKLTVDK SRWQQGNVFS CSVMHEALHN HYTQKSLSLS |
| | PGK |

(SEQ ID NO: 192)

| | |
|---|---|
| 1 | SNTKVDKRVT GGGTHTCPPC PAPELLGGPS VFLFPPKPKD |
| | TLMISRTPEV |
| 51 | TCVVVDVSHE DPEVKFNWYV DGVEVHNAKT KPREEQYNST |
| | YRVVSVLTVL |
| 101 | HQDWLNGKEY KCKVSNKALP APIEKTISKA KGQPREPQVY |
| | TLPPSREEMT |
| 151 | KNQVSLTCLV KGFYPSDIAV EWESNGQPEN NYDTTPPVLD |
| | SDGSFFLYSD |
| 201 | LTVDKSRWQQ GNVFSCSVMH EALHNHYTQK SLSLSPGK |

(SEQ ID NO: 193)

| | |
|---|---|
| 1 | MDAMKRGLCC VLLLCGAVFV SPGASNTKVD KRVTGGGTHT |
| | CPPCPAPELL |
| 51 | GGPSVFLFPP KPKDTLMISR TPEVTCVVVD VSHEDPEVKF |
| | NWYVDGVEVH |
| 101 | NAKTKPREEQ YNSTYRVVSV LTVLHQDWLN GKEYKCKVSN |
| | KALPAPIEKT |
| 151 | ISKAKGQPRE PQVCTLPPSR EEMTKNQVSL SCAVKGFYPS |
| | DIAVEWESNG |
| 201 | QPENNYKTTP PVLDSDGSFF LVSKLTVDKS RWQQGNVFSC |
| | SVMHEALHNH |
| 251 | YTQKSLSLSP GAGGGGSGGG GSGGGGSGGG GSGTIPPHVQ |
| | KSDVEMEAQK |
| 301 | DEIICPSCNR TAHPLRHINN DMIVTDNNGA VKFPQLCKFC |
| | DVRFSTCDNQ |
| 351 | KSCMSNCSIT SICEKPQEVC VAVWRKNDEN ITLETVCHDP |
| | KLPYHDFILE |
| 401 | DAASPKCIMK EKKKPGETFF MCSCSSDECN DNIIFSEEYN |
| | TSNPD |

A CAGA12 reporter gene assay in A549 cells similar to that described in Example 4 was used to determine the ability of several of the above constructs to inhibit activity of TGFβ1 and TGFβ3. Specifically, the constructs tested in this assay were:
ActRIIB-Fc-(G4S)4G-TGFBRII homodimer (4-arm);
ActRIIB-Fc-(G4S)4G-TGFBRII+Fc1-(G4S)4G-TGFBRII (3-arm)
Fc1-(G4S)4G-TGFBRII, homodimer (2-arm)
Fc1-(G4S)4G-TGFBRII, monomer (1 arm)
TGFBRII-(G4S)4-hFc homodimer
   The IC₅₀ data from these experiments is indicated in FIG. 18. Surprisingly, the Fc1-(G4S)4G-TGFBRII homodimer (in which the TGFBRII portion is C-terminal to the Fc portion) inhibited TGFβ1 and TGFβ3 approximately 7-fold greater than the TGFBRII-(G4S)4-hFc homodimer (in which the TGFBRII portion is N-terminal to the Fc portion).

In a separate series of experiments, a CAGA12 reporter gene assay in A204 cells similar to that described in Example 4 was used to determine the ability of several of the above constructs to inhibit GDF11 and activin A. Specifically, the constructs tested in this assay were:
ActRIIB-Fc-(G4S)4G-TGFBRII homodimer (4-arm);
ActRIIB-Fc-(G4S)4G-TGFBRII+Fc1-(G4S)4G-TGFBRII (3-arm)
ActRIIB-Fc homodimer
ActRIIB-Fc Single Arm
   The IC₅₀ data from these experiments is indicated in FIG. 18.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

1-197. (canceled)

198. An isolated polynucleotide comprising:
(a) a nucleotide sequence encoding an ActRIIB-Fc polypeptide, wherein the nucleotide sequence is at least 95% identical to SEQ ID NO: 83, and
(b) a nucleotide sequence encoding a TβRII-Fc polypeptide, wherein the nucleotide sequence is at least 95% identical to SEQ ID NO: 86.

199. A recombinant polynucleotide comprising:
(a) a promoter sequence operably linked to the nucleic acid sequence encoding the ActRIIB-Fc polypeptide of claim 198,
(b) a promoter sequence operably linked to the nucleic acid sequence encoding the TβRII-Fc polypeptide of claim 198, or
(c) a promoter sequence operably linked to the nucleic acid sequence encoding the ActRIIB-Fc polypeptide and the nucleic acid sequence encoding the TβRII-Fc polypeptide of claim 198.

200. A vector comprising the recombinant polynucleotide of claim 199.

201. A cell transformed with the vector of claim 200.

202. The cell of claim 201, wherein the cell is a CHO cell.

203. A method of making a heteromultimer comprising an ActRIIB-Fc polypeptide and a TβRII-Fc polypeptide, the method comprising
(a) culturing a cell under conditions suitable for expression of the ActRIIB-Fc polypeptide and the TβRII-Fc polypeptide, wherein the cell comprises the recombinant polynucleotide of claim 199(c), and
(b) recovering the heteromultimer so expressed.

204. A method of making a heteromultimer comprising an ActRIIB-Fc polypeptide and a TβRII-Fc polypeptide, the method comprising:
(a) culturing a first cell under conditions suitable for expression of the ActRIIB-Fc polypeptide, wherein the cell comprises the recombinant polynucleotide of claim 199(a),
(b) recovering the ActRIIB-Fc polypeptide so expressed,
(c) culturing a second cell under conditions suitable for expression of the TβRII-Fc polypeptide, wherein the cell comprises the recombinant polynucleotide of claim 199(b),
(d) recovering the TβRII-Fc polypeptide so expressed; and
(e) combining the recovered ActRIIB-Fc polypeptide and the recovered TβRII-Fc polypeptide under conditions suitable for ActRIIB-Fc:TβRII-Fc heteromultimer formation.

205. An isolated polynucleotide comprising:
(a) a nucleotide sequence encoding an ActRIIB-Fc polypeptide, wherein the nucleotide sequence is at least 95% identical to SEQ ID NO: 89, and
(b) a nucleotide sequence encoding an TβRII-Fc polypeptide, wherein the nucleotide sequence is at least 95% identical to SEQ ID NO: 92.

206. A recombinant polynucleotide comprising:
(a) a promoter sequence operably linked to the nucleic acid sequence encoding the ActRIIB-Fc polypeptide of claim 205,
(b) a promoter sequence operably linked to the nucleic acid sequence encoding the TβRII-Fc polypeptide of claim 205, or
(c) a promoter sequence operably linked to the nucleic acid sequence encoding the ActRIIB-Fc polypeptide and the nucleic acid sequence encoding the TβRII-Fc polypeptide of claim 205.

207. A vector comprising the recombinant polynucleotide of claim 206.

208. A cell transformed with the vector of claim 207.

209. The cell of claim 208, wherein the cell is a CHO cell.

210. A method of making a heteromultimer comprising an ActRIIB-Fc polypeptide and a TβRII-Fc polypeptide, the method comprising:
(a) culturing a cell under conditions suitable for expression of the ActRIIB-Fc polypeptide and the TβRII-Fc polypeptide, wherein the cell comprises the recombinant polynucleotide of claim 206(c), and
(b) recovering the heteromultimer so expressed.

211. A method of making a heteromultimer comprising an ActRIIB-Fc polypeptide and a TβRII-Fc polypeptide, the method comprising:
(a) culturing a first cell under conditions suitable for expression of the ActRIIB-Fc polypeptide, wherein the cell comprises the recombinant polynucleotide of claim 206(a),
(b) recovering the ActRIIB-Fc polypeptide so expressed,
(c) culturing a second cell under conditions suitable for expression of the TβRII-Fc polypeptide, wherein the cell comprises the recombinant polynucleotide of claim 206(b),
(d) recovering the TβRII-Fc polypeptide so expressed, and
(e) combining the recovered ActRIIB-Fc polypeptide and the recovered ActRIIB-Fc polypeptide under conditions suitable for ActRIIB-Fc:TβRII-Fc heteromultimer formation.

212. An isolated polynucleotide comprising:
(a) a nucleotide sequence encoding an ActRIIB-Fc polypeptide, wherein the ActRIIB-Fc polypeptide has an amino acid sequence that is at least 95% identical to SEQ ID NO: 84, and
(b) a nucleotide sequence encoding an TβRII-Fc polypeptide, wherein the TβRII-Fc polypeptide has an amino acid sequence that is at least 95% identical to SEQ ID NO: 87; or
(c) a nucleotide sequence encoding an ActRIIB polypeptide, wherein the ActRIIB-Fc polypeptide has an amino acid sequence that is at least 95% identical to SEQ ID NO: 90, and
(d) a nucleotide sequence encoding an TβRII-Fc polypeptide, wherein the TβRII-Fc polypeptide has an amino acid sequence that is at least 95% identical to SEQ ID NO: 93.

213. The nucleotide sequence of claim 212, further comprising a nucleotide sequence encoding a leader peptide.

214. The nucleotide sequence of claim 213, wherein the leader peptide has the amino acid sequence of SEQ ID NO: 23.

215. A recombinant polynucleotide comprising:
(a) a promoter sequence operably linked to the nucleic acid sequence of claim 212(a), encoding an ActRIIB-Fc polypeptide,
(b) a promoter sequence operably linked to the nucleic acid sequence of claim 212(b), encoding a TβRII-Fc polypeptide,
(c) a promoter sequence operably linked to the nucleic acid sequence of claim 212(a), encoding an ActRIIB-Fc polypeptide, and the nucleic acid sequence of claim 212(b), encoding a TβRII-Fc polypeptide,
(d) a promoter sequence operably linked to the nucleic acid sequence of claim 212(c), encoding an ActRIIB-Fc polypeptide,
(e) a promoter sequence operably linked to the nucleic acid sequence of claim 212(d), encoding a TβRII-Fc polypeptide, or
(f) a promoter sequence operably linked to the nucleic acid sequence of claim 212(c), encoding an ActRIIB-Fc polypeptide, and the nucleic acid sequence of claim 212(d), encoding a TβRII-Fc polypeptide,
wherein in each of (a) to (f), the nucleotide sequence further comprises a nucleotide sequence encoding a leader peptide.

216. The recombinant polynucleotide of claim 215, wherein the leader peptide has the amino acid sequence of SEQ ID NO: 23.

217. A method of making a heteromultimer comprising an ActRIIB-Fc polypeptide and a TβRII-Fc polypeptide, the method comprising:
(a) culturing a cell under conditions suitable for expression of the ActRIIB-Fc polypeptide and the TβRII-Fc polypeptide, wherein the cell comprises the recombinant polynucleotide of claim 215(c) or 215(f), and
(b) recovering the heteromultimer so expressed.

218. A method of making a heteromultimer comprising an ActRIIB-Fc polypeptide and a TβRII-Fc polypeptide, the method comprising:
(a) culturing a first cell under conditions suitable for expression of the ActRIIB-Fc polypeptide, wherein the cell comprises the recombinant polynucleotide of claim 215(a),
(b) recovering the ActRIIB-Fc polypeptide so expressed,
(c) culturing a second cell under conditions suitable for expression of the TβRII-Fc polypeptide, wherein the cell comprises the recombinant polynucleotide of claim 215(b),
(d) recovering the TβRII-Fc polypeptide so expressed; and
(e) combining the recovered ActRIIB-Fc polypeptide and the recovered ActRIIB-Fc polypeptide under conditions suitable for ActRIIB-Fc:TβRII-Fc heteromultimer formation.

219. A method of making a heteromultimer comprising an ActRIIB-Fc polypeptide and a TβRII-Fc polypeptide, the method comprising:
(a) culturing a first cell under conditions suitable for expression of the ActRIIB-Fc polypeptide, wherein the cell comprises the recombinant polynucleotide of claim 215(d),
(b) recovering the ActRIIB-Fc polypeptide so expressed,
(c) culturing a second cell under conditions suitable for expression of the TβRII-Fc polypeptide, wherein the cell comprises the recombinant polynucleotide of claim 215(e),
(d) recovering the TβRII-Fc polypeptide so expressed; and
(e) combining the recovered ActRIIB-Fc polypeptide and the recovered ActRIIB-Fc polypeptide under conditions suitable for ActRIIB-Fc:TβRII-Fc heteromultimer formation.
